(19) United States
(12) Patent Application Publication
(10) Pub. No.: US 2005/0197375 A1
(43) Pub. Date: Sep. 8, 2005

(54) Use of benzimidazole analogs in the treatment of cell proliferation

(76) Inventors: Jagadish C Sircar, SanDiego, CA (US); Mark L. Richards, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/508,968

(22) PCT Filed: Mar. 6, 2003
(86) PCT No.: PCT/US03/06981

§ 371 Date: Sep. 24, 2004

Related U.S. Application Data
(60) Provisional application No. 60/367,686, filed on Mar. 25, 2002.

(51) Int. Cl. A61K 31/4439; A61K 31/433; A61K 31/4184

(52) U.S. Cl. 514/394; 514/341; 514/363

(57) ABSTRACT

The preferred embodiments are directed to small molecule inhibitors that are cellular proliferation inhibitors and thus are useful as anticancer agents. The small molecules have the general formulas that include a phenylbenzimidazole core ring.

### BACKGROUND OF THE INVENTION

1. Field of the Invention

This invention relates to phenylbenzimidazole analogs that inhibit proliferation of tumor cells in vitro and in vivo. This family of small molecules is useful in treating conditions associated with uncontrolled cell proliferation which characterizes many forms of cancer,

2. Description of the Related Art

Cellular proliferation is a normal process that is vital to the normal functioning of most biological processes. Cellular proliferation occurs in all living organisms and involves two main processes: nuclear division (mitosis), and cytoplasmic division (cytokinesis). Because organisms are continually growing and replacing cells, cellular proliferation is essential to the vitality of the healthy cell. However, disruption of normal cellular proliferation can result in a variety of disorders. For example, hyperproliferation of cells may cause psoriasis, thrombosis, atherosclerosis, coronary heart disease, myocardial infarction, stroke, smooth muscle neoplasms, uterine fibroid or fibroma, and obliterative diseases of vascular grafts and transplanted organs. Abnormal cell proliferation is most commonly associated with tumor formation and cancer.

Cancer is a major disease and is one of the leading causes of mortality world-wide. Indeed, cancer is the second leading cause of death in the United States. According to the National Institute of Health, the overall annual cost for cancer is approximately $107 billion, which includes $37 billion for direct medical costs, $11 billion for indirect costs of lost productivity due to illness and $59 billion for indirect costs of lost productivity due to premature death. Not surprisingly considerable efforts are underway to develop new treatments and preventative measures to comb this devastating illness.

Currently, cancer is primarily treated using a combination of surgery, radiation and chemotherapy. Chemotherapy involves the use of chemical agents to disrupt the replication and metabolism of cancerous cells. Chemotherapeutic agents which are currently being used to treat cancer can be classified into the following main groups: alkylating drugs, antimetabolites [symbol] antibiotics, plant alkaloids, and steroid hormones.

One embodiment relates to a family of phenylbenzimidazole derivatives the inhibit cell proliferation. These phenylbenzimidazole derivatives were first described in U.S. Pat. Nos. 6,271,390; 6,303,645; and 6,369,091 and co-pending U.S. application Ser. Nos. 09/983,054; and 10/103,258. They have been shown to down-regulate IgE levels.

Other classes of phenylbenzimidazole analogs have also been described in European Patent No. 719,765 and U.S. Pat. No. 5,821,258. These other classes of compounds are structurally different from the phenylbenzimidazole derivatives of the preferred embodiments, and are reported to exert their biological effects by inducing DNA alkylation. There is no suggestion in the references that these other phenylbenzimidazole analogs inhibit cell proliferation. Instead, the compounds disclosed in European Patent No. 719,765 and U.S. Pat. No. 5,821,258 are described as having anticancer, antiviral, or antimicrobial activities.

### SUMMARY OF THE INVENTION

The preferred embodiments are related to the use of families of related compounds for the treatment of cancer. The phenylbenzimidazole inhibitors of tumor growth in accordance with the preferred embodiments are represented by Genuses A-F, as shown below.

One family of small molecule inhibitors, designated Genus A, in accordance with preferred embodiments includes compounds defined by Formula IX:

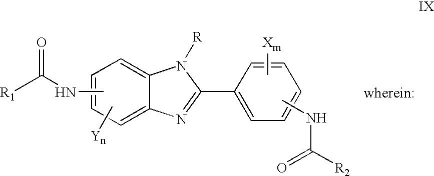

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to four;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ and R₂ are independently selected from the group consisting of H, alkyl, substituted allyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom.

Another family of small molecule inhibitors, designated Genus B, in accordance with preferred embodiments includes compounds defined by Formula IX:

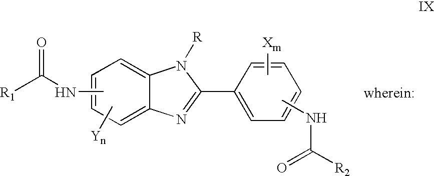

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to four,

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ and R₂ are independently selected from the group consisting of H, allyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, heterocyclic ring, substituted heterocyclic ring, heteroatom, substituted heteroatom, aryl, and substituted aryl, wherein at least one of R₁ and R₂ is selected from aryl or substituted aryl.

Another family of small molecule inhibitors, designated Genus C, in accordance with preferred embodiments includes compounds defined by Formula X:

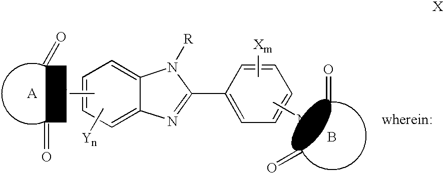

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to four;

m is an integer from one to four,

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

A and B rings independently comprise unsubstituted or substituted carbon atoms ranging from four carbon atoms to ten carbon atoms.

One family of small molecule inhibitors, designated Genus D, in accordance with preferred embodiments includes compounds defined by Formula XI:

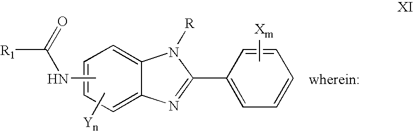

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to five;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ is selected from the group consisting of H, allyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, aryl, substituted aryl, heterocyclic ring, substituted heterocyclic ring, heteroatom, and substituted heteroatom.

One family of small molecule inhibitors, designated Genus E, in accordance with preferred embodiments includes compounds defined by Formula XII:

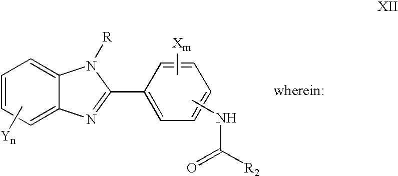

X and Y may be different or the same and are independently selected from the group consisting of H, halogen, alkyl, alkoxy, aryl, substituted aryl, benzo, hydroxy, amino, alkylamino, cycloalkyl, morpholine, thiomorpholine, nitro, cyano, CF₃, OCF₃, COR₂, COOR₂, CONH₂, CONHR₂, and NHCOR₂;

n is an integer from one to four;

m is an integer from one to four;

R is selected from the group consisting of H, CH₃, C₂H₅, C₃H₇, C₄H₉, CH₂Ph, CH₂C₆H₄—F(P-), COCH₃, COCH₂CH₃, CH₂CH₂N(CH₃)₂, and CH₂CH₂CH₂N(CH₃)₂; and

R₂ is selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, aryl, substituted aryl, heterocyclic ring, substituted heterocyclic ring, heteroatom, and substituted heteroatom.

One family of small molecule inhibitors, designated Genus F, in accordance with preferred embodiments includes compounds defined by Genuses A, B, and C collectively.

A method for treating a disease condition associated with abnormal cell proliferation in a mammal is disclosed. In one aspect, the method comprises the step of administering to a mammal an effective amount of a pharmaceutical formulation for treating a disease condition associated with abnormal cell proliferation comprising at least one benzimidazole compound from the above-disclosed small molecule families of Genuses A-F.

In accordance with a variation of the method of treatment, the small molecule anti-cell proliferation compound may be administered in conjunction with at least one additional agent, which is active in reducing a symptom associated with cell proliferation. In one embodiment, the small molecule inhibitor may be mixed with at least one additional active ingredient to form a pharmaceutical composition. Alternatively, the small molecule inhibitor may be co-administered at the same time or according to different treatment regimens with the at least one additional active agent.

In another embodiment, the benzimidazole compound may be administered in conjunction with at least one additional active agent. These active agents include antifungals, antivirals, antibiotics, anti-inflammatories, and anticancer agents. Anticancer agents include, but are not limited to, alkylating agents (lomustine, carmustine, streptozocin, mechlorethamine, melphalan, uracil nitrogen mustard, chlorambucil cyclophosphamide, iphosphamide, cisplatin, carboplatin mitomycin thiotepa dacarbazine procarbazine, hexamethyl melamine, triethylene melamine, busulfan, pipobroman, and mitotane); antimetabolites (methotrexate, trimetrexate pentostatin, cytarabine, ara-CMP,. fludarabine phosphate, hydroxyurea, fluorouracil, floxuridine, chlorodeoxyadenosine, gemcitabine, thioguanine, and 6-mercaptopurine); DNA cutters (bleomycin); topoisomerase I poisons (topotecan, irinotecan, and camptothecin); topoisomerase II poisons (daunorubicin, doxorbicin, idarubicin, mitoxantrone, teniposide, and etoposide); DNA binders (dactinomycin and mithramycin); and spindle poisons (vinblastine, vincristine, navelbine, paclitaxel and docetaxel).

In another embodiment, the benzimidazole compounds of the preferred embodiments are administered in conjunction with one or more other therapies. These therapies include, but are not limited to radiation, immnunotherapy, gene therapy and surgery. These combination therapies may be administered simultaneously or sequentially. For example, radiation may be administered along with the administration of benzimidazole compounds, or may be administered at any time before or after administration of benzimidazole compounds.

A dose of about 0.01 mg to about 100 mg per kg body weight per day of the small molecule anti-cell proliferation compound is preferably administered in divided doses daily.

The methods provided herein for treating diseases and processes mediated by undesired, uncontrolled or abnormal cell proliferation, such as cancer, involve administering to a mammal a composition of the benzimidazole compounds disclosed herein to inhibit cell proliferation. The method is particularly useful for preventing or treating tumor formation and progression. In one embodiment of the invention, the compounds and methods disclosed are especially useful in treating estrogen receptor positive and estrogen receptor negative type breast cancers.

Other variations within the scope of the preferred embodiments may be more fully understood with reference to the following detailed description.

### BRIEF DESCRIPTION OF THE DRAWINGS

FIG. 1 shows a graph of the suppression of spleen cell proliferation responses by AVP XXX. Spleen cell cultures were established from naive BALB/c mice and incubated for about 4 days in the presence of stimulus and active compound. Cultures were pulsed for about 4 hours with ³H-thymidine and harvested.

FIG. 2 shows a graph of the suppression of spleen cell proliferation responses by AVP YYY. Spleen cell cultures were established from naive BALB/c mice and incubated for about 4 days in the presence of stimulus and active compound. Cultures were pulsed for about 4 hours with ³H-thymidine and harvested.

FIG. 3 shows a graph of effect of AVP YYY on the proliferation of M12.4.1 cells in vitro. M12.4.1 cells were cultured at about 3000,000 per ml in the presence and absence of active compound and stimulus for about 2 days. ³H-thymidine was added to the cultures for about the final 6 hours before harvesting.

FIG. 4 shows a graph of effect of AVP XXX and AVP YYY on the proliferation of M12.4.1 cells in the presence of IL4/anti-CD40 antibody. ³H-thymidine was added to the cultures for about the final 6 hours before harvesting.

FIG. 5 shows a table of the cell lines used in the cell proliferation experiments.

FIG. 6 shows a graph of the cell line proliferation to AVP XXX and AVP YYY in vitro. Cells were cultured overnight in the presence of active compound and pulsed with ³H-thymidine for about 4 to about 12 hours before harvesting. IC₅₀ of 800 nM denote greater than or equal to 800 nM.

FIG. 7 shows a graph of the proliferation response of human breast cancer cell lines to AVP XXX and AVP YYY. Cells were cultured overnight in the presence of active compound and pulsed with ³H-thymidine for about 4 to about 12 hours before harvesting. IC₅₀S of 800 nM denote greater than or equal to 800 nM.

### DETAILED DESCRIPTION OF THE PREFERRED EMBODIMENTS

The preferred embodiments are directed to small molecules which are useful in the treatment of diseases associated with abnormal cellular proliferation, including, but not limited to, tumorigenesis and other proliferative diseases such as, but not limited to, cancers, inflammatory disorders, and circulatory diseases. For example, hyperproliferation of cells can cause psoriasis, thrombosis, atherosclerosis, coronary heart disease, myocardial infarction, stroke, smooth muscle neoplasms, uterine fibroid or fibroma, and obliterative diseases of vascular grafts and transplanted organs. Abnormal cell proliferation is most commonly associated with tumor formation and cancer. The particular compounds disclosed herein were identified by their ability to suppress abnormal cellular proliferation.

Cell Proliferation Studies of the Preferred Embodiments

Materials and Methods

A variety of experiments were performed in an effort to determine the effect of the phenylbenzimidazole compounds of the preferred embodiments on cellular proliferation. These procedures were performed either in vitro or ex vivo; the latter involving administration of the drug in vivo and measuring the effect on the cells in vitro.

In Vitro Experiments

These experiments ultimately measured ³H-thymidine incorporation into proliferating cell DNA. The specific procedure varied with the cells and the stimuli. Cells derived from mouse spleen were cultured at 3 million per ml; M12.4.5 cells (mouse B cell lymphoma) at 1 million per ml; Vero cells (monkey kidney-derived cell line) at 100,000 per ml. Splenic B cells were isolated by T cell depletion and stimulated with LPS (5 or 50 μg/ml) or anti-CD40 Ab (100 ng/ml). T cells were depleted prior to culture by incubating spleen cells first with a cocktail of anti-Thy1 ascites (10%), anti-CD4 Ab (0.5 μg/ml) and anti-CD8 Ab (0.5 μg/ml), followed by guinea pig complement (adsorbed). M12.4.5 cells and Vero cells were unstimulated. All cells were cultured for 2 days and pulsed with ³H-thymidine during the final 4 to 6 hrs of culture.

Ex Vivo Experiments

Two types of experiments were performed. The mixed lymphocyte reaction (MLR) involved administration of 2 mg/kg/day or 5 mg/kg/day of AVP XXX (a representative compound of Genus B) or AVP YYY (a representative compound of Genus A), or vehicle daily for 4 days to BALB/c mice and removing their spleens 24 hr after the last dose. Spleen cells from C57BL/6 mice were prepared for use, as stimulator cells following removal of red blood cells by ACK treatment and irradiating for 2.6 min (250 rads). Stimulator cells (C57BL/6) were cultured at 5×10⁵ cells/mi and responder cells (BALB/c) at 2×10⁵ cells/ml. Cells were cultured for 4 days then pulsed overnight with ³H-thymidine.

The second ex vivo experiment involves sensitizing of BALB/c mice with DNP-KLH and followed two weeks later with a 5 day course of AVP XXX or AVP YYY i.p. for 5 days. DNP-KLH was re-administered on day 3 of the drug injections. Four weeks after the second antigen dose, the mice were sacrificed, the spleens removed and spleen cell cultures initiated. T cell proliferation was stimulated by co-culturing spleen cells for 4 days with KLH. B cells were stimulated with LPS for 2 days. Cells were harvested after a 6 hr pulse with ³H-thymidine.

Spleen Cells

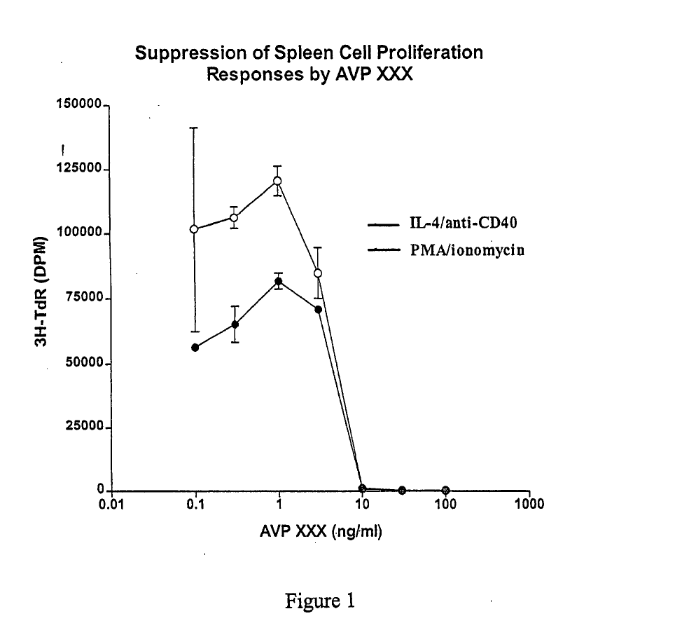

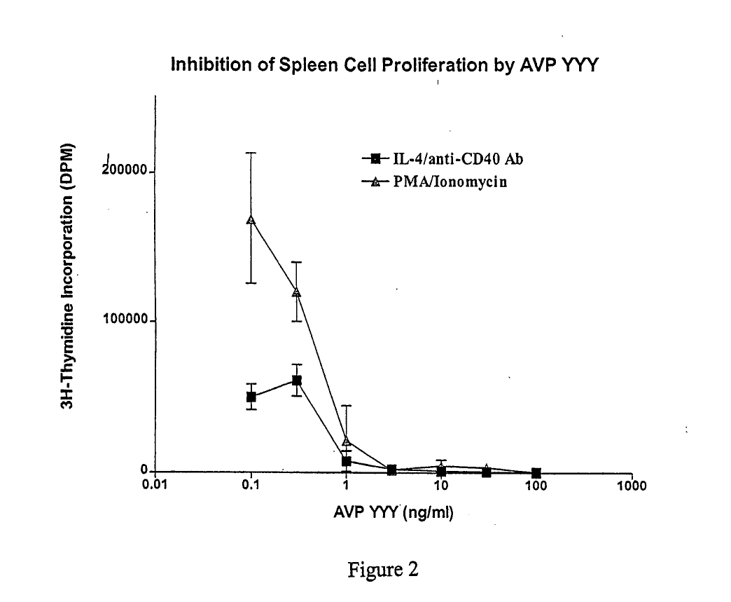

Certain compounds of the preferred embodiments suppressed B cell proliferation responses to PMA/ionomycin and IL-4/anti-CD40 Ab (FIGS. 1 and 2) with approximately the same potencies as they suppress in vitro responses to IL-4/anti-CD40 Ab (not shown). Similar inhibition potencies were obtained for AVP XXX in ConA-stimulated T cell proliferation and LPS-stimulated B cell proliferation, suggesting a lack of specificity in the action of these drugs. On the other hand, a battery of immunological tests performed with AVP XXX demonstrated little other effects other than inhibition of ConA-stimulated cytokine release.

Tumor Cells

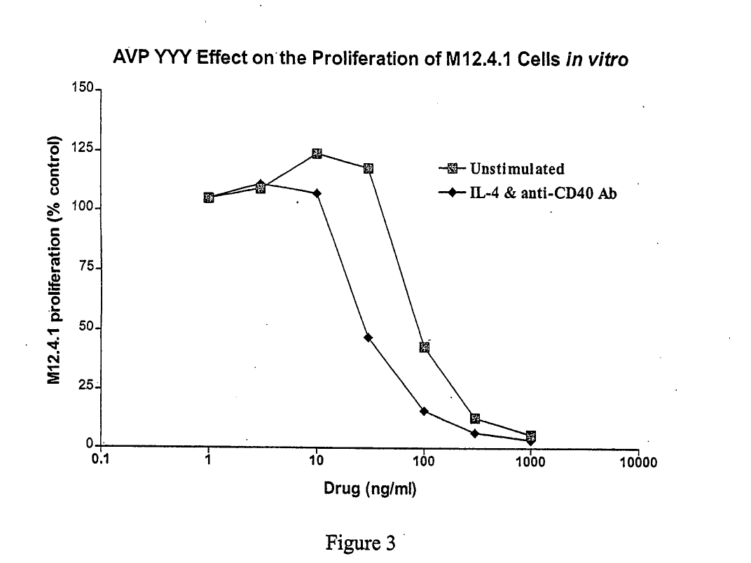

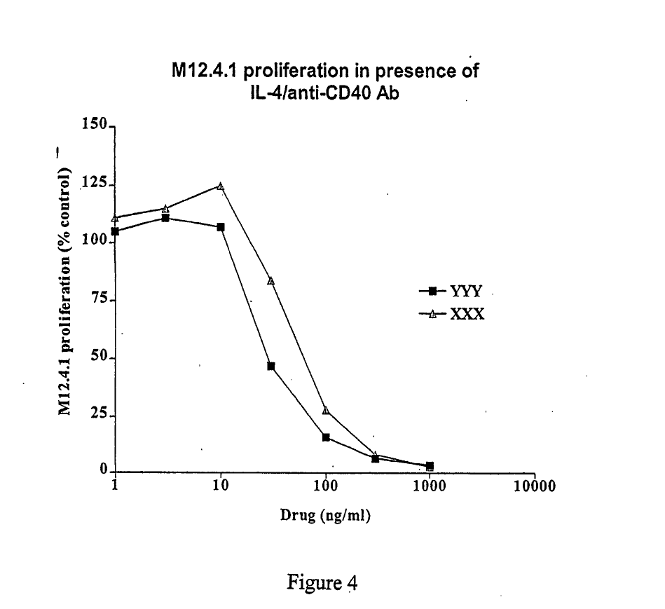

The results with splenic lymphocytes led to a further analysis of cellular proliferation by measuring the growth of tumor cells in the presence of these drugs. The initial analysis was performed with murine M12.4.1 lymphoma cells, either unstimulated or stimulated with IL-4/anti-CD40 Ab. As shown in FIGS. 3 and 4, both AVP XXX and AVP YYY suppressed the proliferation of M12.4.1 cells but with lower potency that observed in stimulated spleen cells. However, the potency of both compounds increased when the cells were cultured with IL-4/anti-CD40 Ab. This stimulation is known to induce the activity of NF-κB in M12.4.1 cells.

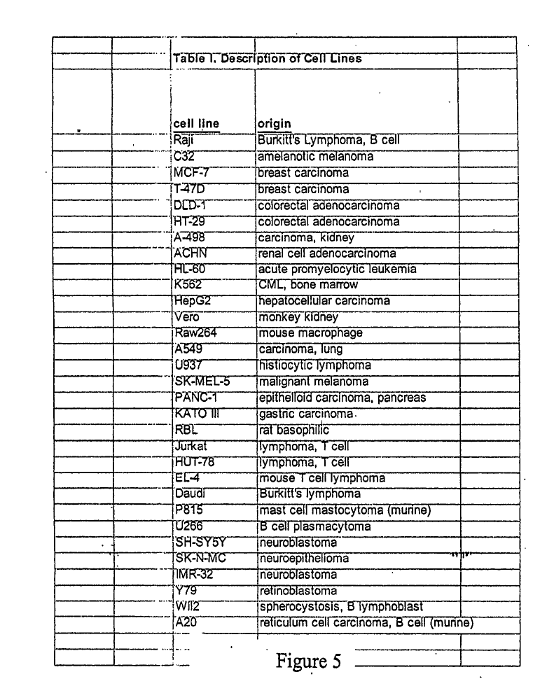

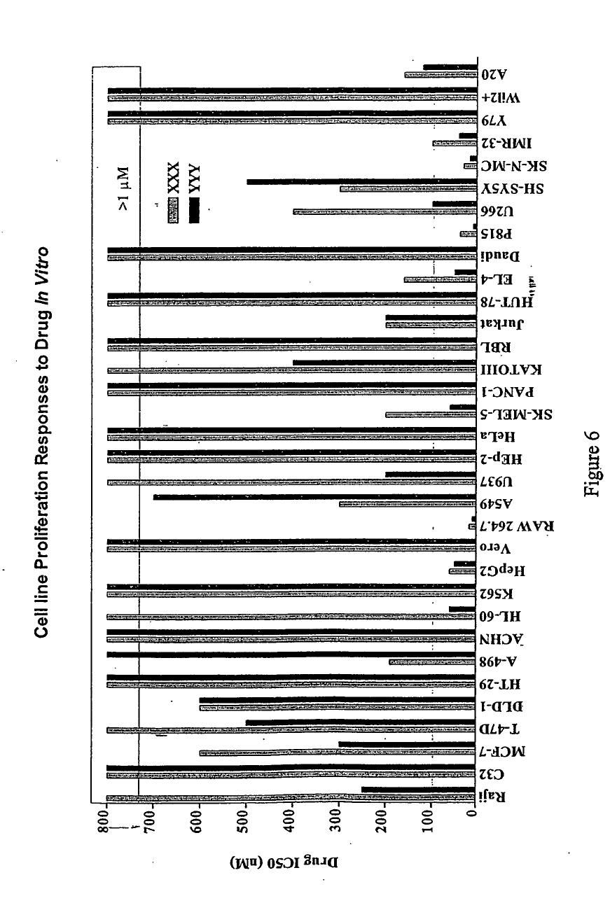

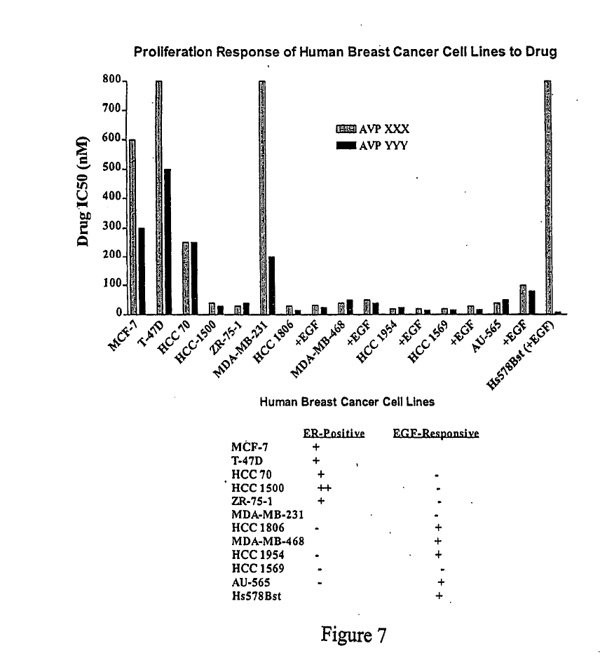

A similar approach was used to establish selectivity of the anti-proliferative activity by testing a battery of tumor lines derived from a variety of tissues, mostly human in origin. An attempt was made to generate proliferation data from at least 2 cell lines from each tissue selected (FIG. 5). As noted in FIG. 6, only a handful of cell lines were inhibited by 100 nM or less of each compound while most the balance of the cells required much higher concentrations. Because of the known character of some of the tested cell lines and previous Western blot results with the compounds, there is evidence to suggest a link between NF-κB inhibition and the action of the drugs. Breast cancer cells offer a good model for testing this phenomenon because they are predominantly of 2 types; estrogen receptor (ER) -positive and ER-negative. The latter cells tend to be less differentiated, have a higher density of EGF receptor expression, and are more resilient to treatment. Proliferation of ER-negative/EGFR-positive cells also tends to be driven by NF-κB and thus a selection of these cells were tested for proliferation responses to drug in vitro. As noted in FIG. 7, proliferation of all of the EGF-responsive cell lines was potently inhibited by AVP XXX and AVP YYY in vitro. Conversely, only two of the five ER-positive cell lines were potently inhibited by drug.

AVP XXX and AVP YYY exert an anti-proliferative activity to T and B lymphocytes exposed to a variety of immunogenic stimuli in vitro. These actions are highly potent and parallel their IgE-suppression activity. Although the mechanism of this action is unresolved, much is known about the mechanism of IL-4/anti-CD40 Ab-induced IgE production. A major factor in this response is the transcription activator, NF-κB. This factor has been implicated in the proliferation of a number of tumor cells and thus these drugs were tested for activity on the proliferation of various tumor cell lines in vitro. The results show that a number of tumor cell lines are sensitive to the effects of AVP XXX and AVP YYY, and that proliferation of many of the sensitive lines may be driven by NF-κB factors. However, other cell lines known to be driven by Factors other than NF-κB (e.g., the ER-positive HCC 1500 and ZR-75-1). Thus although AVP XXX and AVP YYY appear to selectively act on certain tumor cells, as yet there is no accurate way to predict which cells will be affected.

Compounds Involved with Inhibition of Cellular Proliferation

The term “alkyl” used herein refers to a monovalent straight or branched chain radical of from one to ten carbon atoms, including, but not limited to, methyl, ethyl, n-propyl, isopropyl, n-butyl, isobutyl, tert-butyl, n-hexyl, and the like.

The term “alkoxy” used herein refers to straight or branched chain alkyl group covalently bonded to the parent molecule through an ——O—— linkage. Examples of alkoxy radicals include, but are limited to, methoxy, ethoxy, propoxy, isopropoxy, butoxy, n-butoxy, sec-butoxy, t-butoxy and the like.

The term “alkenyl” used herein refers to a monovalent straight or branched chain radical of from two to six carbon atoms containing a carbon double bond including, but not limited to, 1-propenyl, 2-propenyl, 2-methyl-i-propenyl, 1-butenyl, 2-butenyl, and the like.

The term “alkynyl” used herein refers to a monovalent straight or branched chain radical of from two to six carbon atoms containing a carbon triple bond including, but not limited to, 1-propynyl, 1-butynyl, 2-butynyl, and the like.

The term “aryl” used herein refers to homocyclic aromatic radical whether fused or not fused. Examples of aryl groups include, but are not limited to, phenyl, naphthyl, biphenyl, phenanthrenyl, naphthacenyl, and the like.

The term “cycloalkyl” used herein refers to saturated aliphatic ring system radical having 3 to 10 carbon atoms including, but not limited to, cyclopropyl, cyclopentyl, cyclohexyl and the like.

The term “cycloalkenyl” used herein refers to aliphatic ring system radical having 3 to 10 carbon atoms having at least one carbon-carbon double bond in the ring. Examples of cycloalkenyl groups include, but are not limited to, cyclopropenyl, cyclopentenyl, cyclohexenyl, and the like.

The term “polycycloalkyl” used herein refers to saturated aliphatic ring system radical having at least two rings that are fused with or without bridgehead carbons. Examples of polycycloalkyl groups include, but are not limited to, bicyclo[4.4.0]decanyl, bicyclo[2.2.1]heptanyl, adamantanyl, norbornyl, and the like.

The term “polycycloalkenyl” used herein refers to aliphatic ring system radical having at least two rings that are fused with or without bridghead carbons in which at least one of the rings has a carbon-carbon double bond. Examples of polycycloalkenyl groups include, but are not limited to, norbornylenyl, 1,1′-bicyclopentenyl, and the like.

The term “heterocyclic” used herein refers to cyclic ring system radical having at least one ring system in which one or more ring atoms are not carbon, namely heteroatom Heterocycles can be nonaromatic or aromatic. Examples of heterocyclic groups include, but are not limited to, morpholinyl, oxazolyl, pyranyl, pyridyl, pyrimidinyl, pyrrolyl, and the like.

The term “heteroaryl” used herein refers to heterocyclic radical formally derived from an arene by replacement of one or more methine and/or vinylene groups by trivalent or divalent heteroatoms, respectively, in such a way as to maintain the aromatic system. Examples of heteroaryl groups include, but are not limited to, pyridyl, pyrrolyl, oxazolyl, indolyl, and the like.

The term “arylalkyl” used herein refers to one or more aryl groups appended to an alkyl radical. Examples of arylalkyl groups include, but are not limited to, benzyl, phenethyl, phenpropyl, phenbutyl, and the like.

The term “heteroarylalkyl” used herein refers to one or more heteroaryl groups appended to an alkyl radical.

The term “arylcycloalkyl” used herein refers to one or more aryl groups appended to a cycloalkyl radical.

The term “heteroarylcycloalkyl” used herein refers to one or more heteroaryl groups appended to a cycloalkyl radical.

The following series of compounds, identified under subheadings Genuses A-F were found to be potent inhibitors of cellular proliferation. These compounds also exhibit anti-proliferative effects, and, as such, can be used as agents to treat hyper proliferative disorders, including, but not limited to, cancer.

Genus A

One family of small molecule inhibitors, designated Genus A, in accordance with preferred embodiments includes compounds defined by Formula IX:

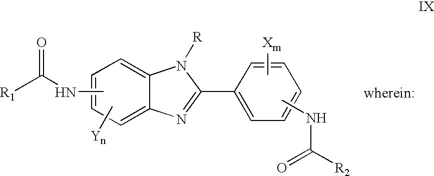

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to four;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ and R₂ are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom.

Genus B

Another family of small molecule inhibitors, designated Genus B, in accordance with preferred embodiments includes compounds defined by Formula IX:

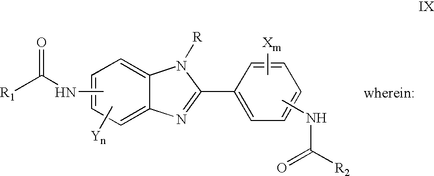

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to four;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ and R₂ are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, heterocyclic ring, substituted heterocyclic ring, heteroatom, substituted heteroatom, aryl, and substituted aryl, wherein at least one of R₁ and R₂ is selected from aryl or substituted aryl.

Genus C

Another family of small molecule inhibitors, designated Genus C, in accordance with preferred embodiments includes compounds defined by Formula X:

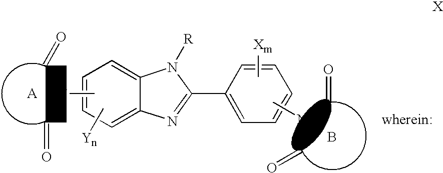

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to four;

m is an integer from one to four;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

A and B rings independently comprise unsubstituted or substituted carbon atoms ranging from four carbon atoms to ten carbon atoms.

Genus D

One family of small molecule inhibitors, designated Genus D, in accordance with preferred embodiments includes compounds defined by Formula XI:

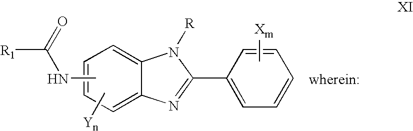

X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;

n is an integer from one to three;

m is an integer from one to five;

R₁ is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₁ is selected from the group consisting of H, allyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, aryl, substituted aryl, heterocyclic ring, substituted heterocyclic ring, heteroatom, and substituted heteroatom.

Genus E

One family of small molecule inhibitors, designated Genus E, in accordance with preferred embodiments includes compounds defined by Formula XII:

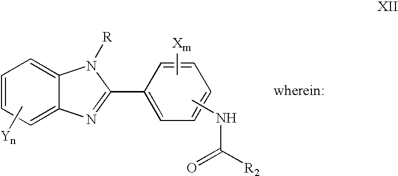

X and Y may be different or the same and are independently selected from the group consisting of H, halogen, alkyl, alkoxy, aryl, substituted aryl, benzo, hydroxy, amino, alkylamino, cycloalkyl, morpholine, thiomorpholine, nitro, cyano, CF₃, OCF₃, COR₂, COOR₂, CONH₂, CONHR₂, and NHCOR₂;

n is an integer from one to four;

m is an integer from one to four;

R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and

R₂ is selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, aryl, substituted aryl, heterocyclic ring, substituted heterocyclic ring, heteroatom, and substituted heteroatom.

Genus F

One family of small molecule inhibitors, designated Genus F, in accordance with preferred embodiments includes compounds defined by Genuses A, B, and C collectively.

The substituents on the preceding groups listed in Genuses A-F can be selected from alkyl, alkenyl, alkynyl, aryl, heterocyclic ring, trihalomethyl, carboxy, oxo, alkoxycarbonyl, alkoxylate, formyl, amido, halo, hydroxy, alkoxy, amino, alkylamino, cyano, nitro, imino, azido, thio, thioalkyl, sulfoxide, sulfone, or sulfate.

Specific compounds of the preferred embodiments of Genus A which are preferred are represented by the following structural formulae or a pharmaceutically acceptable salt or solvate thereof.

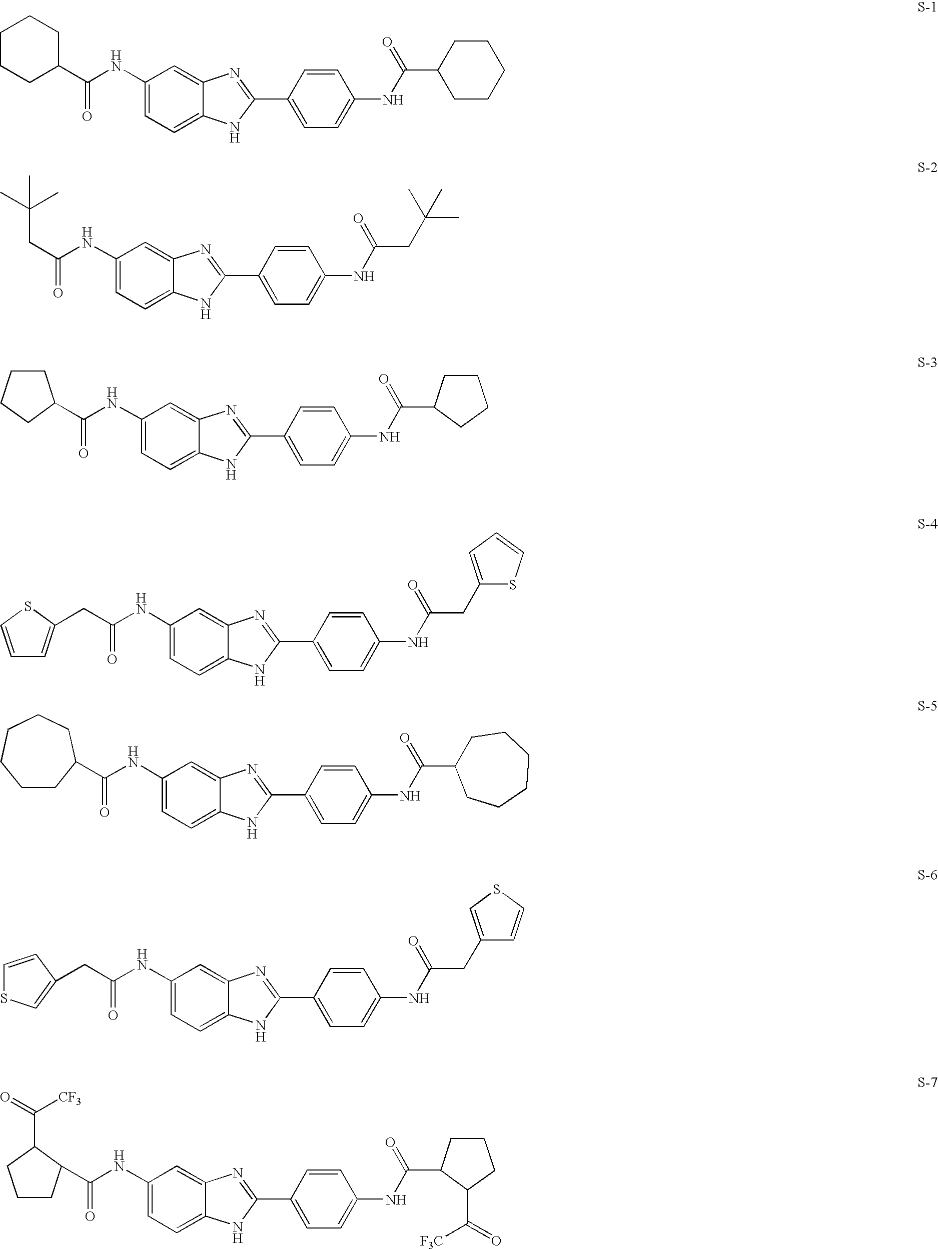

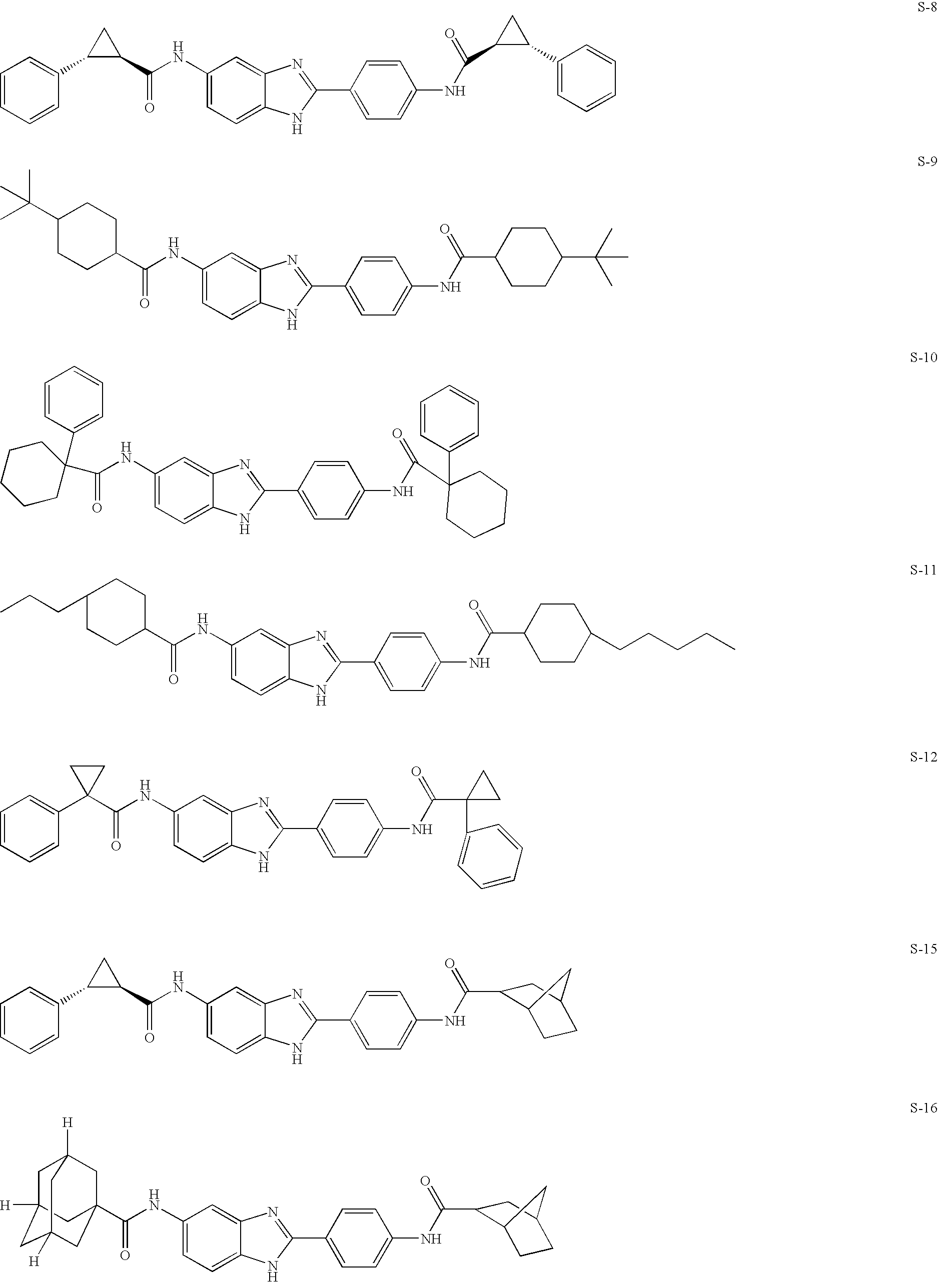

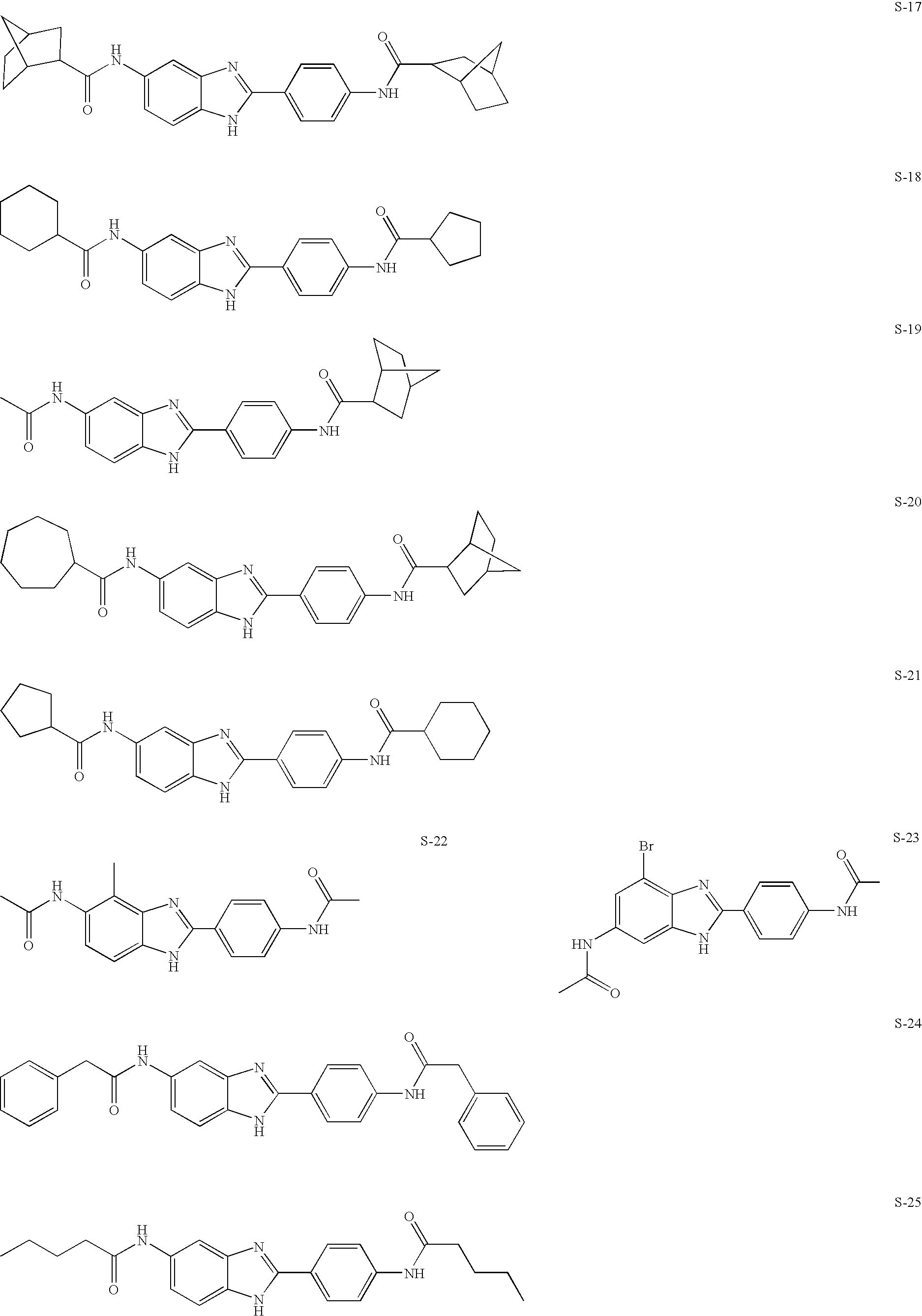

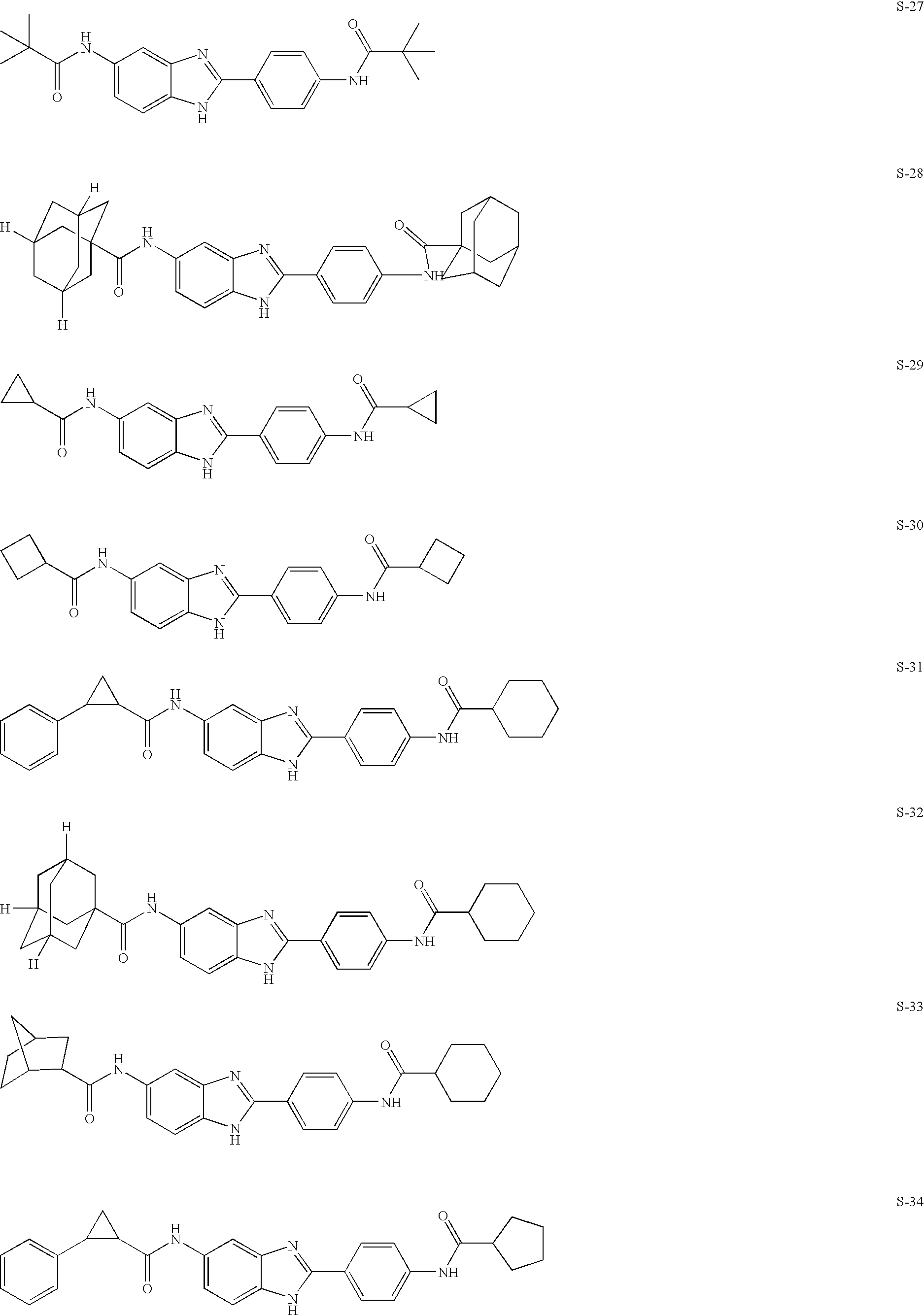

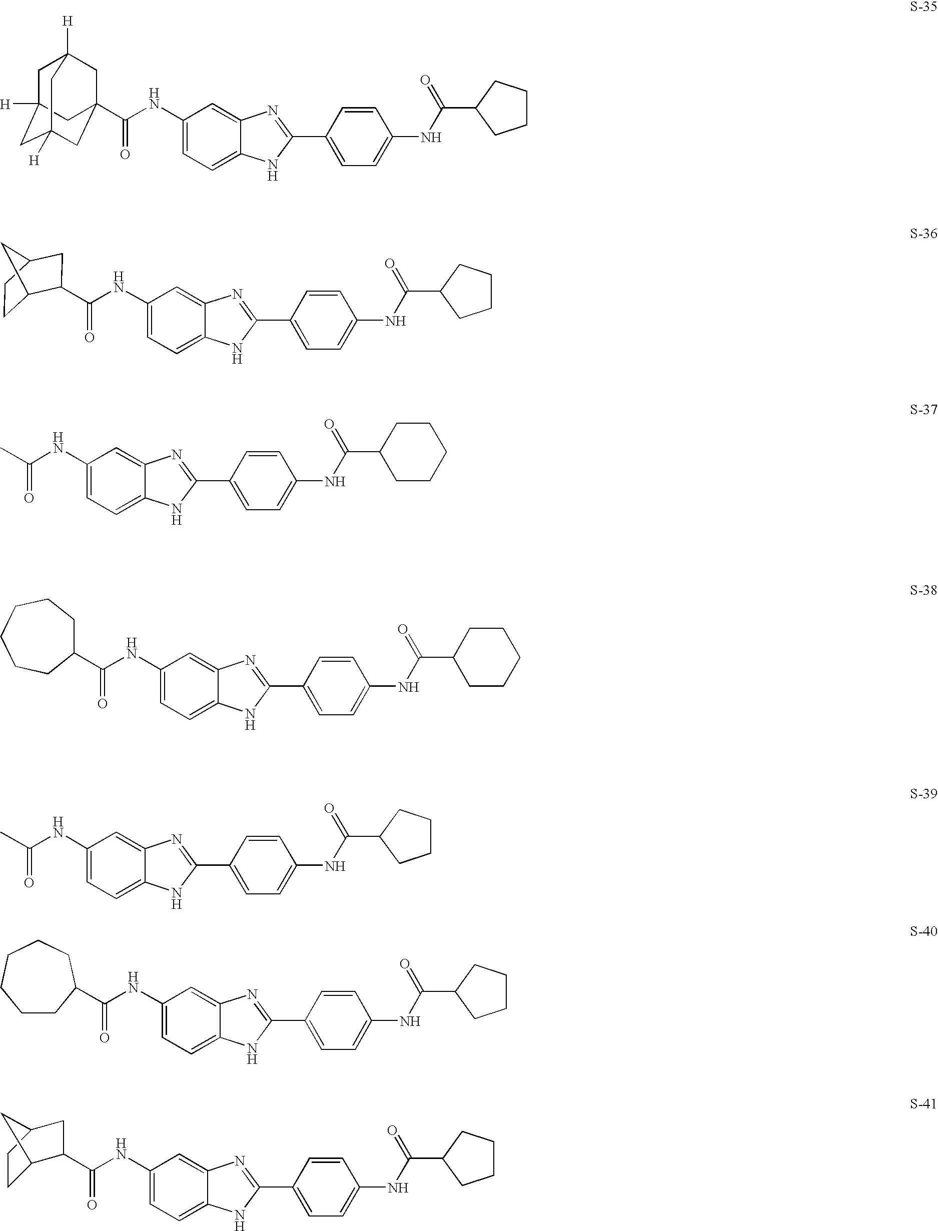

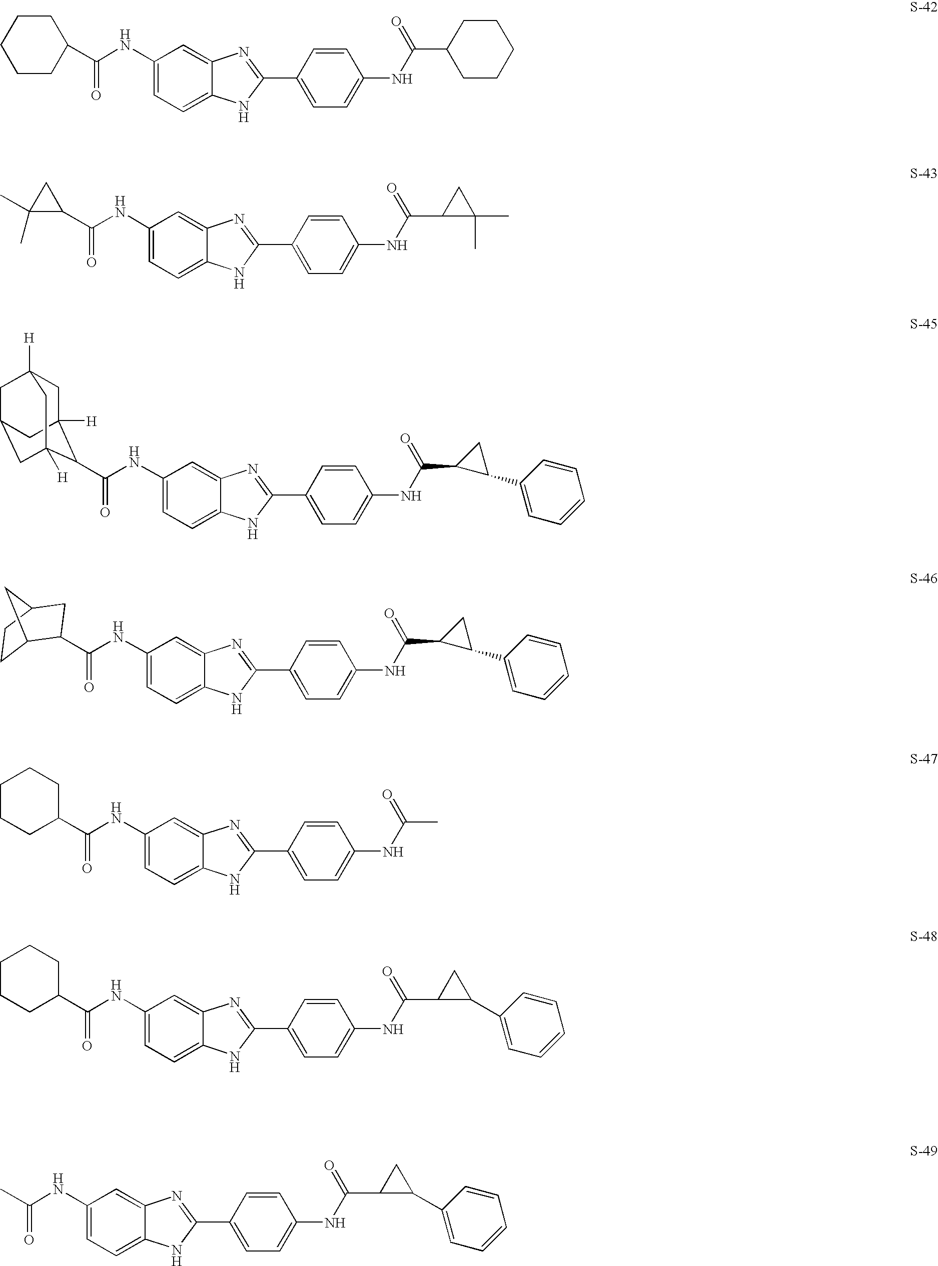

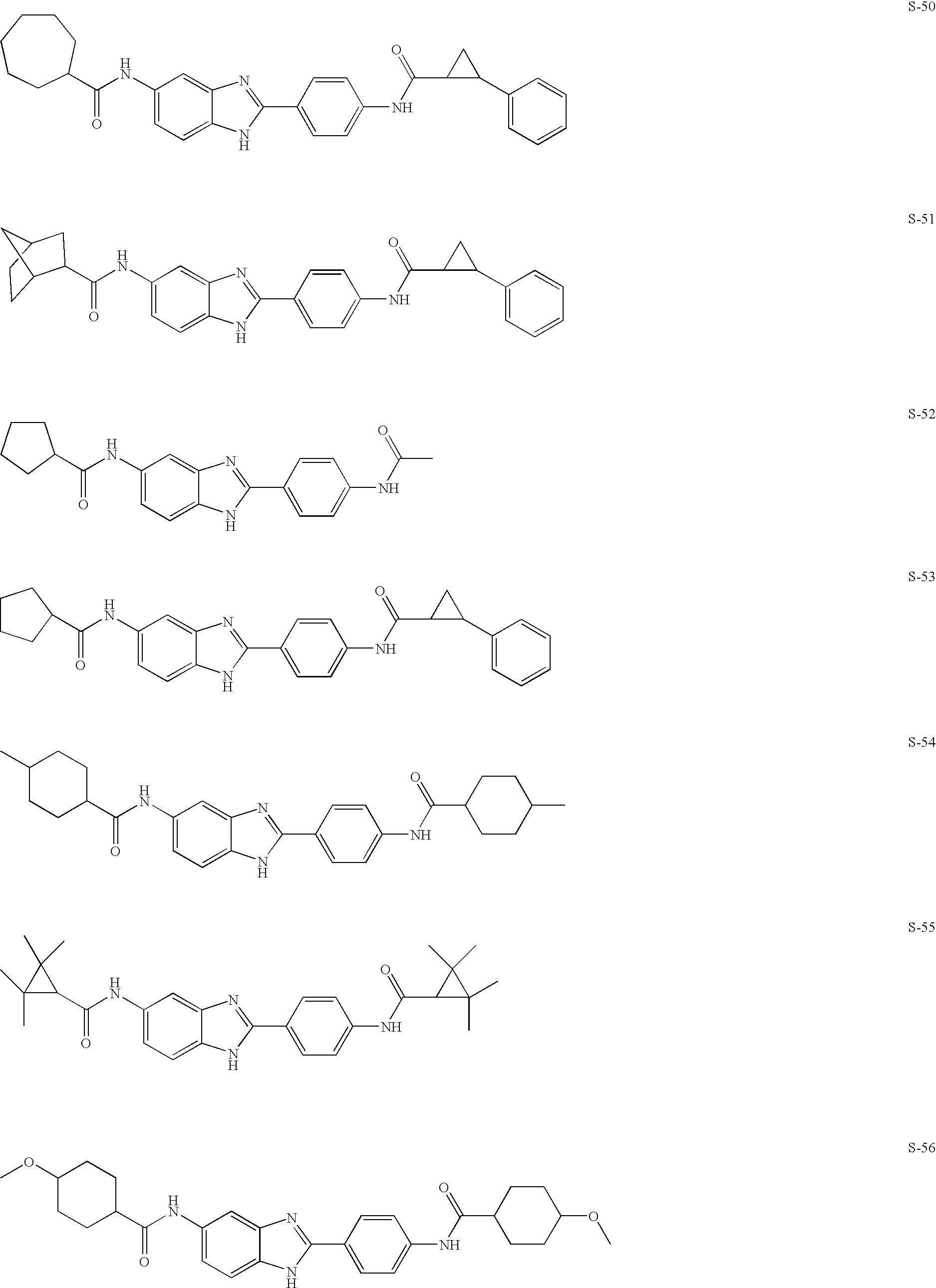

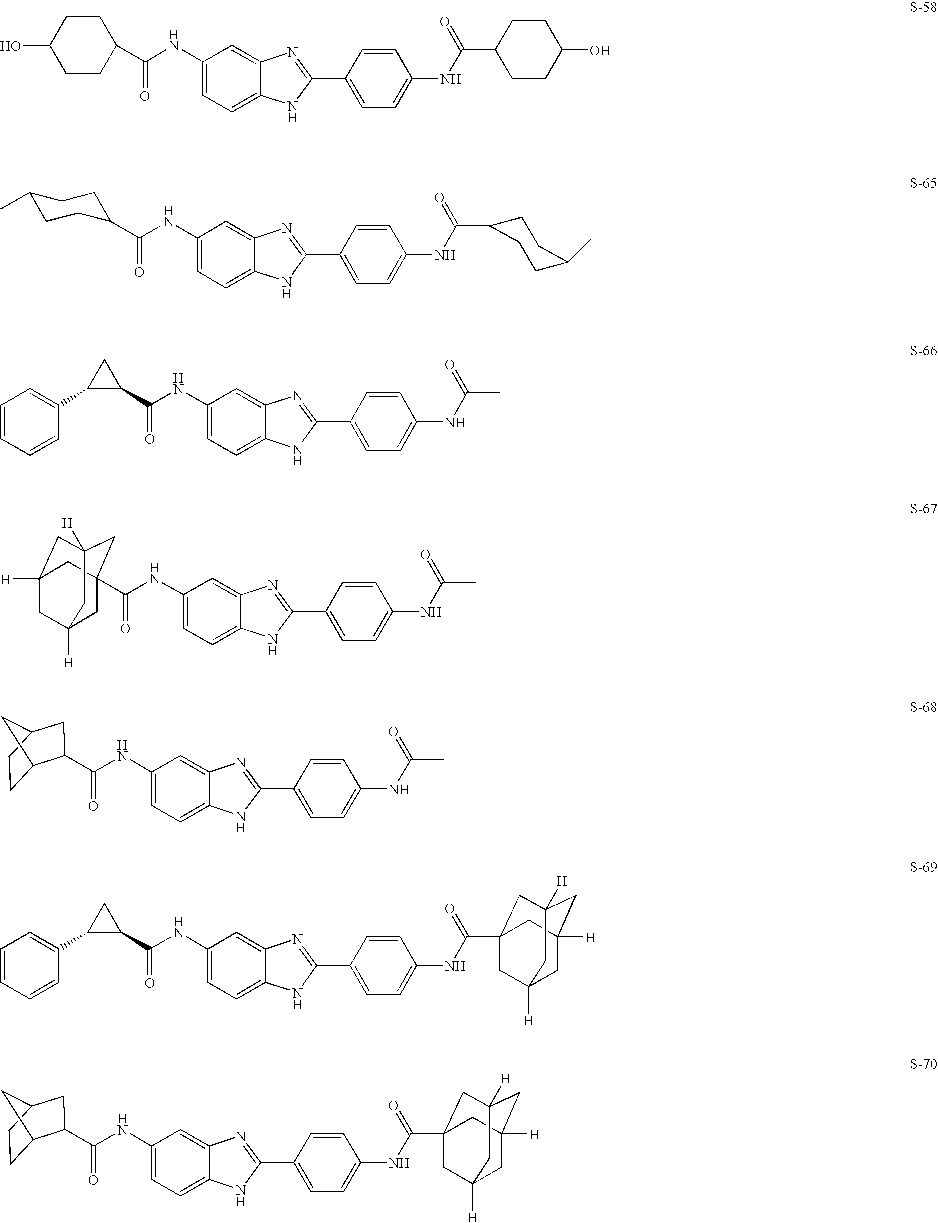

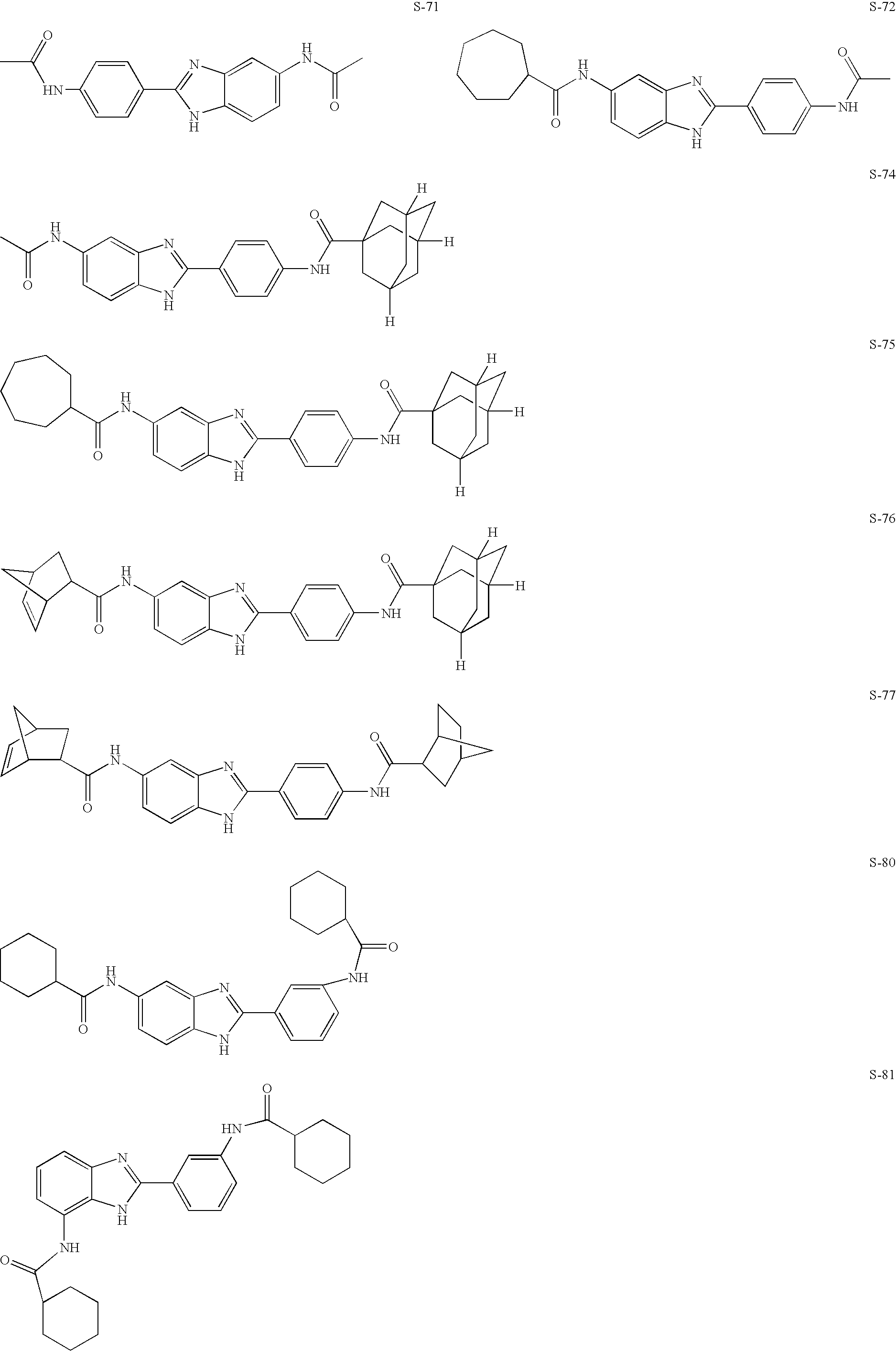

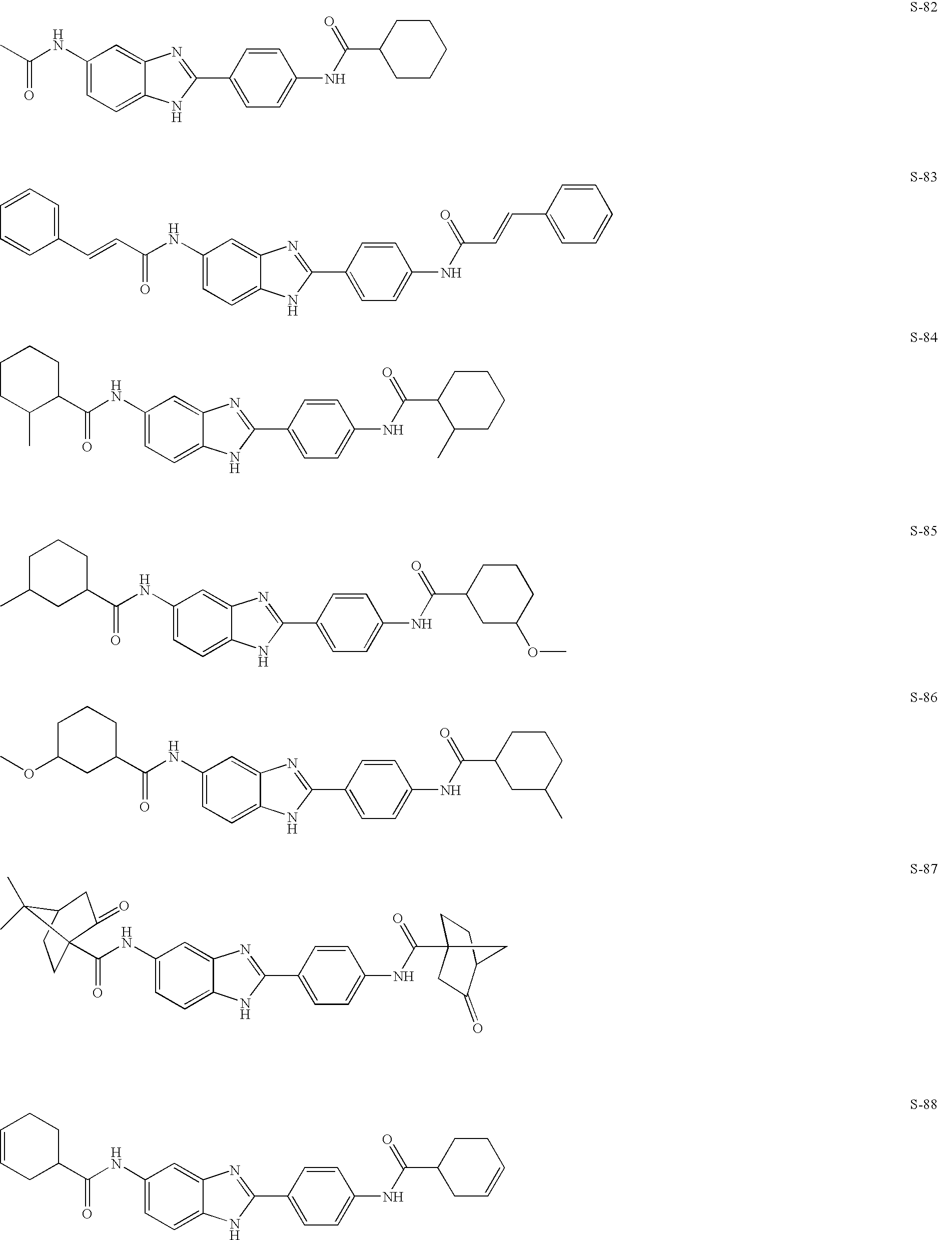

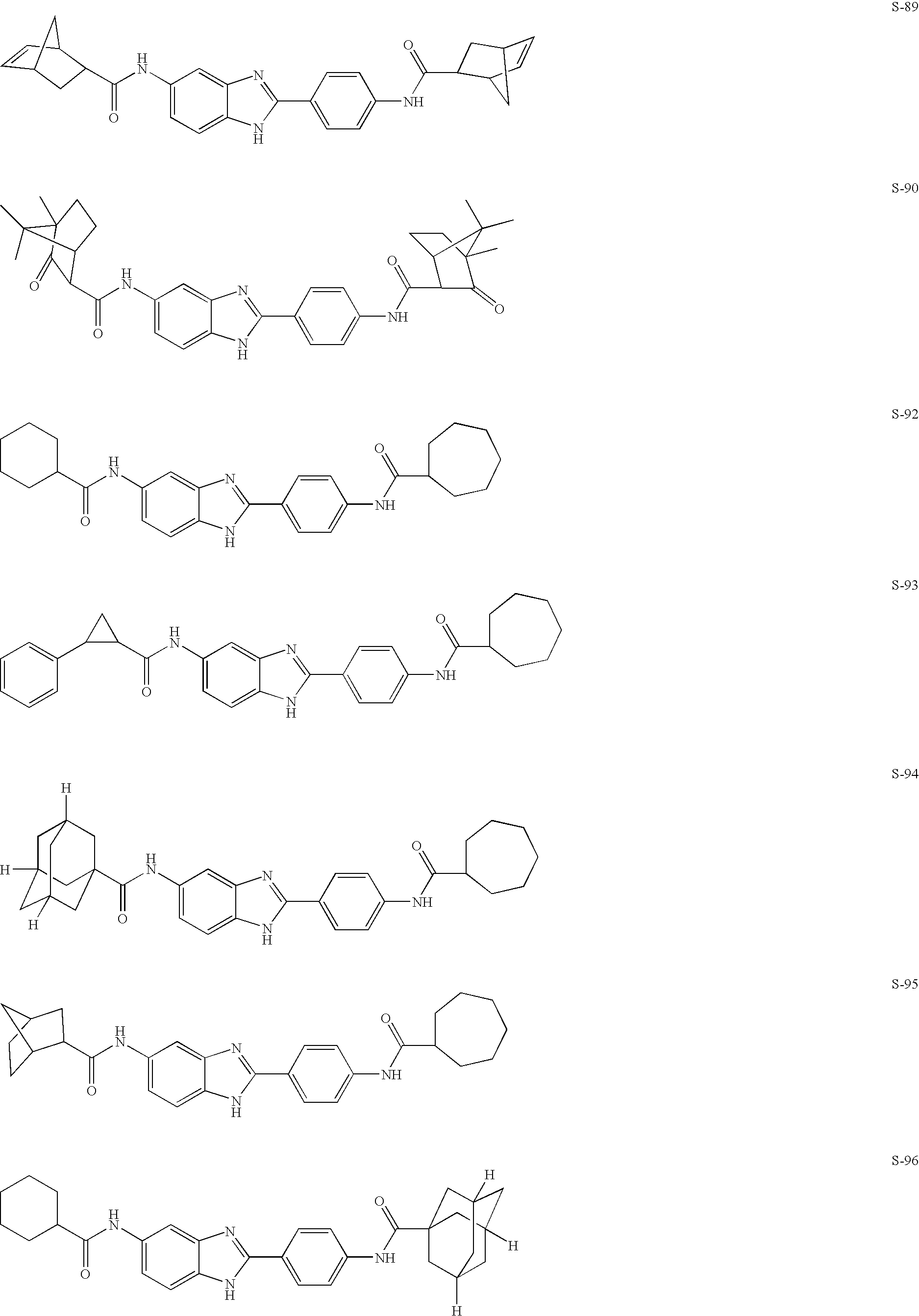

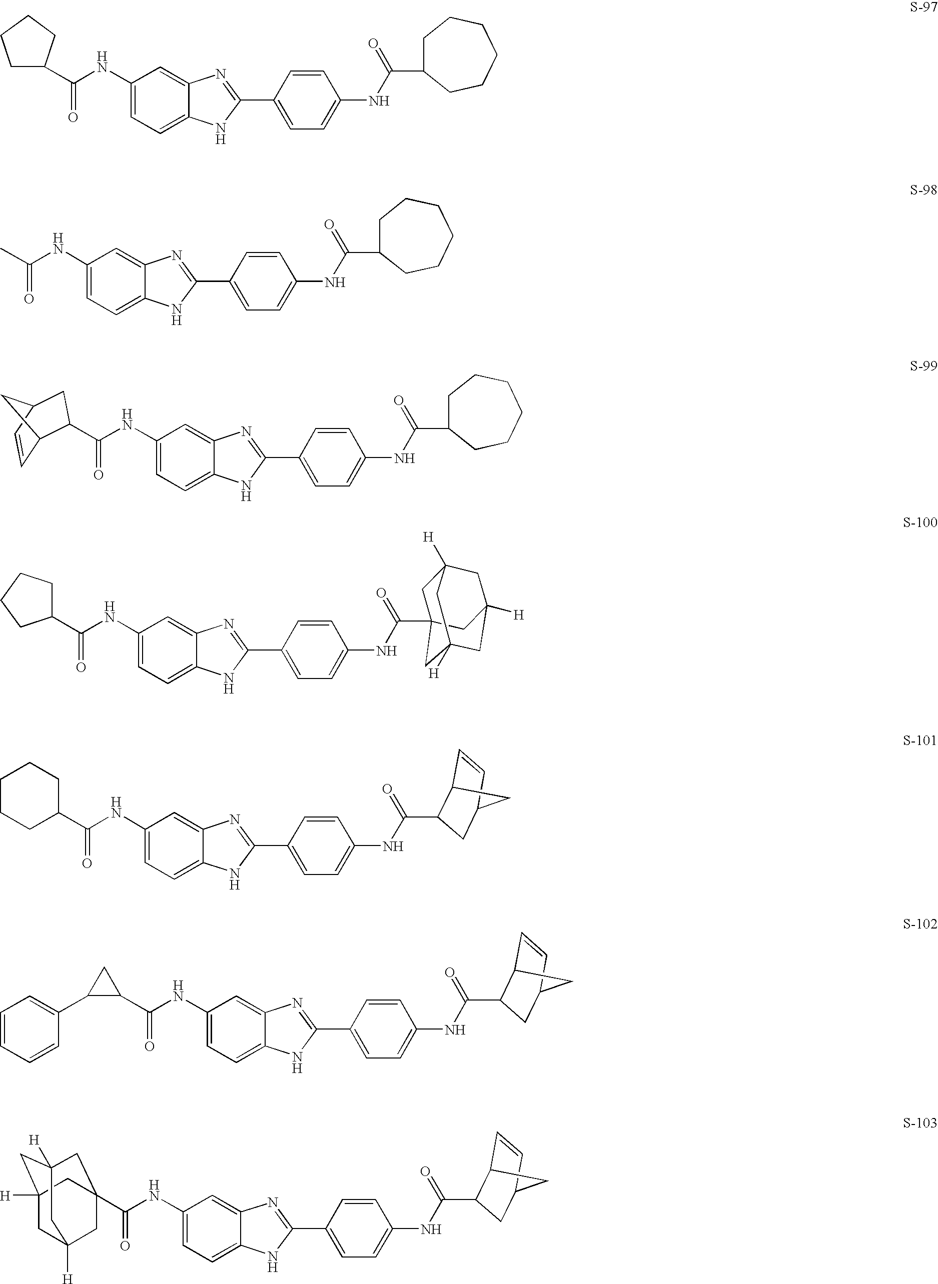

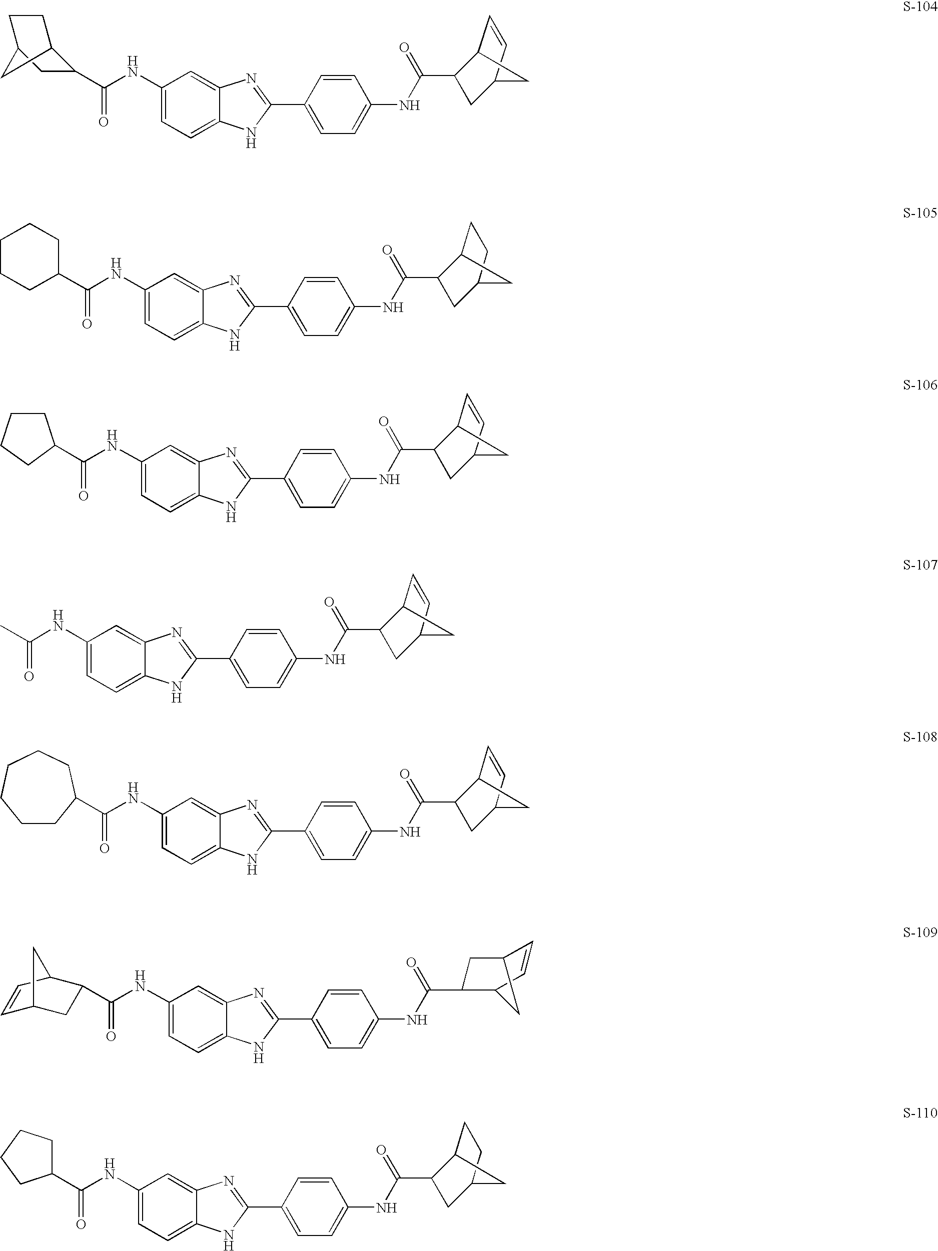

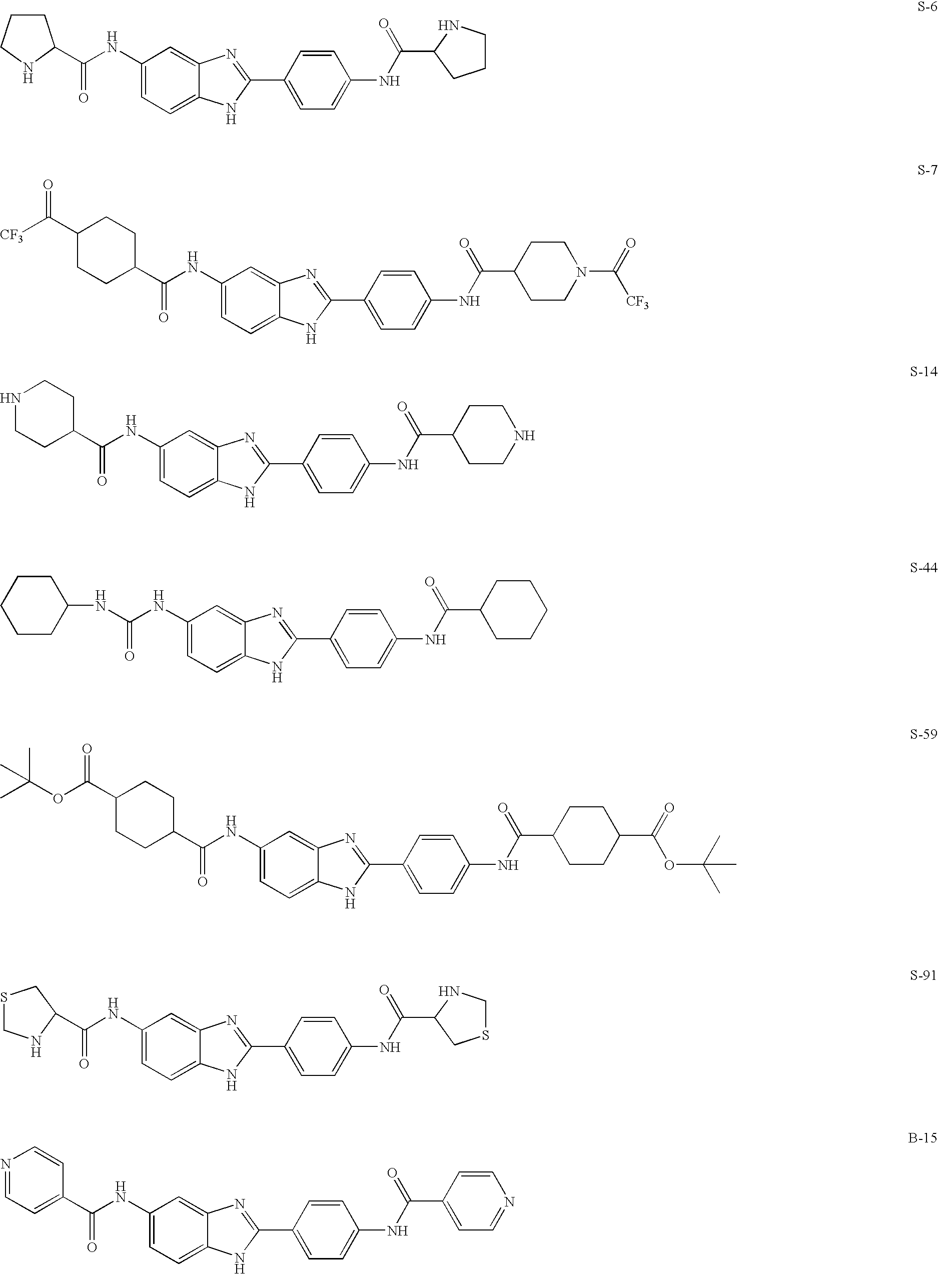

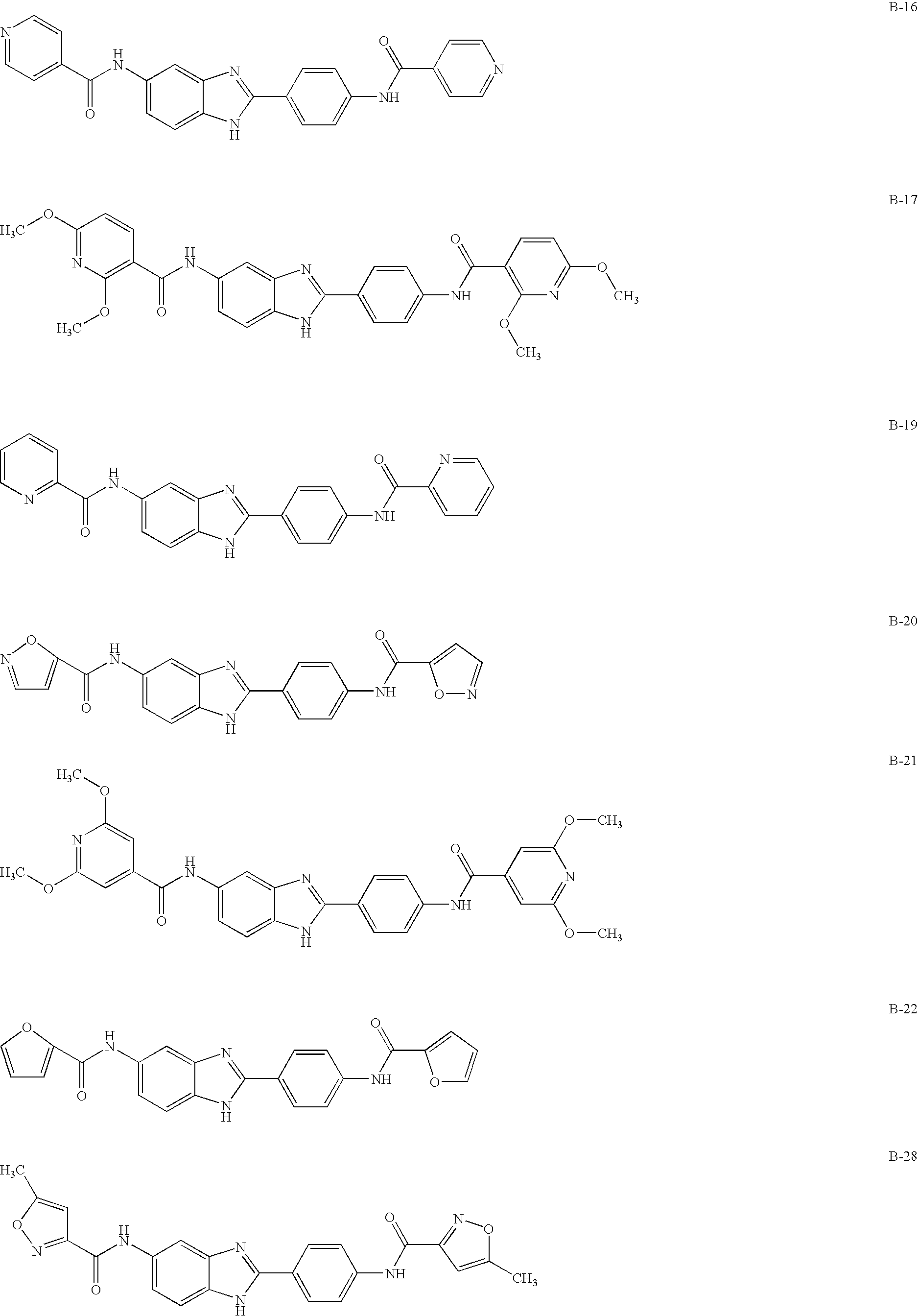

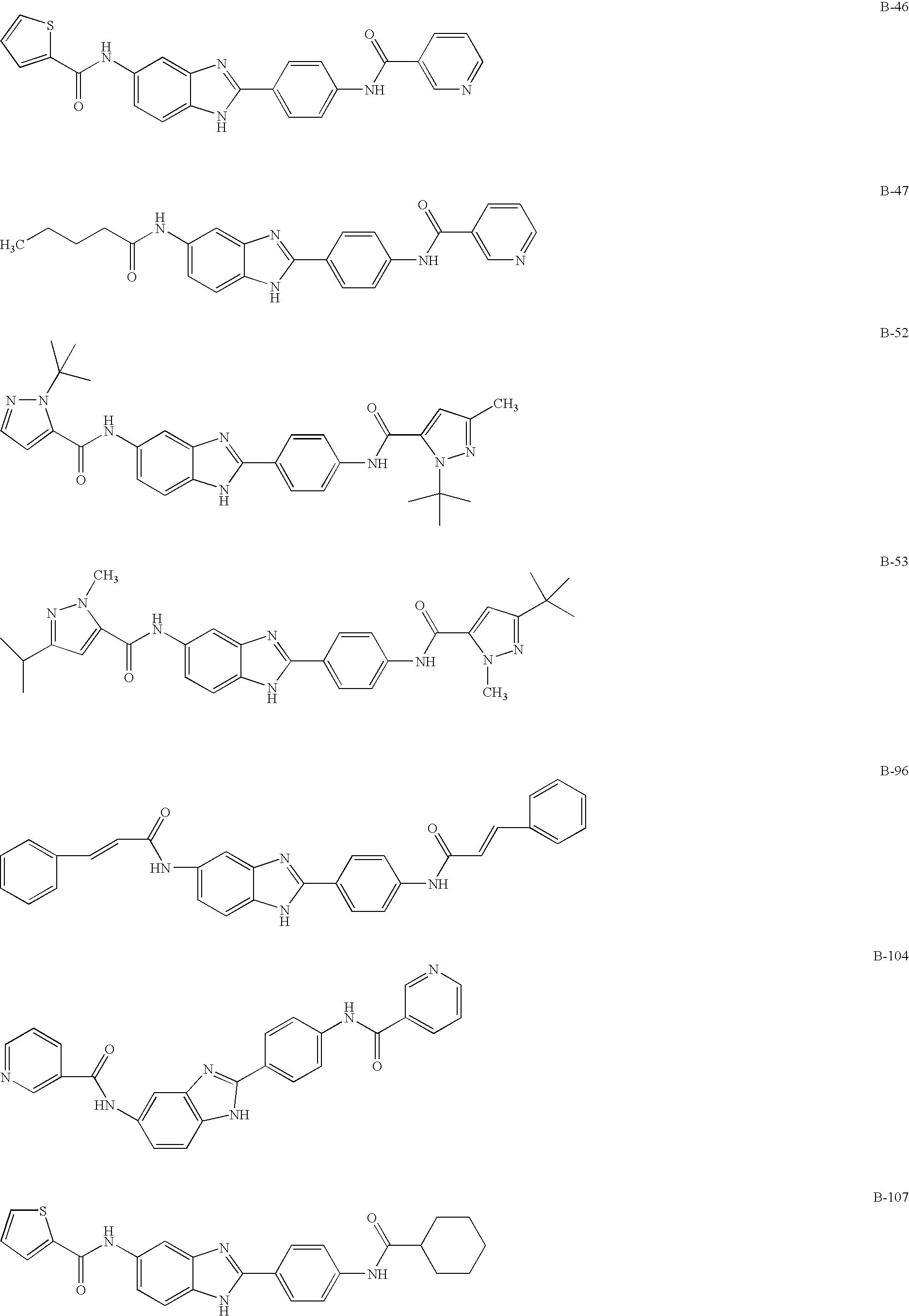

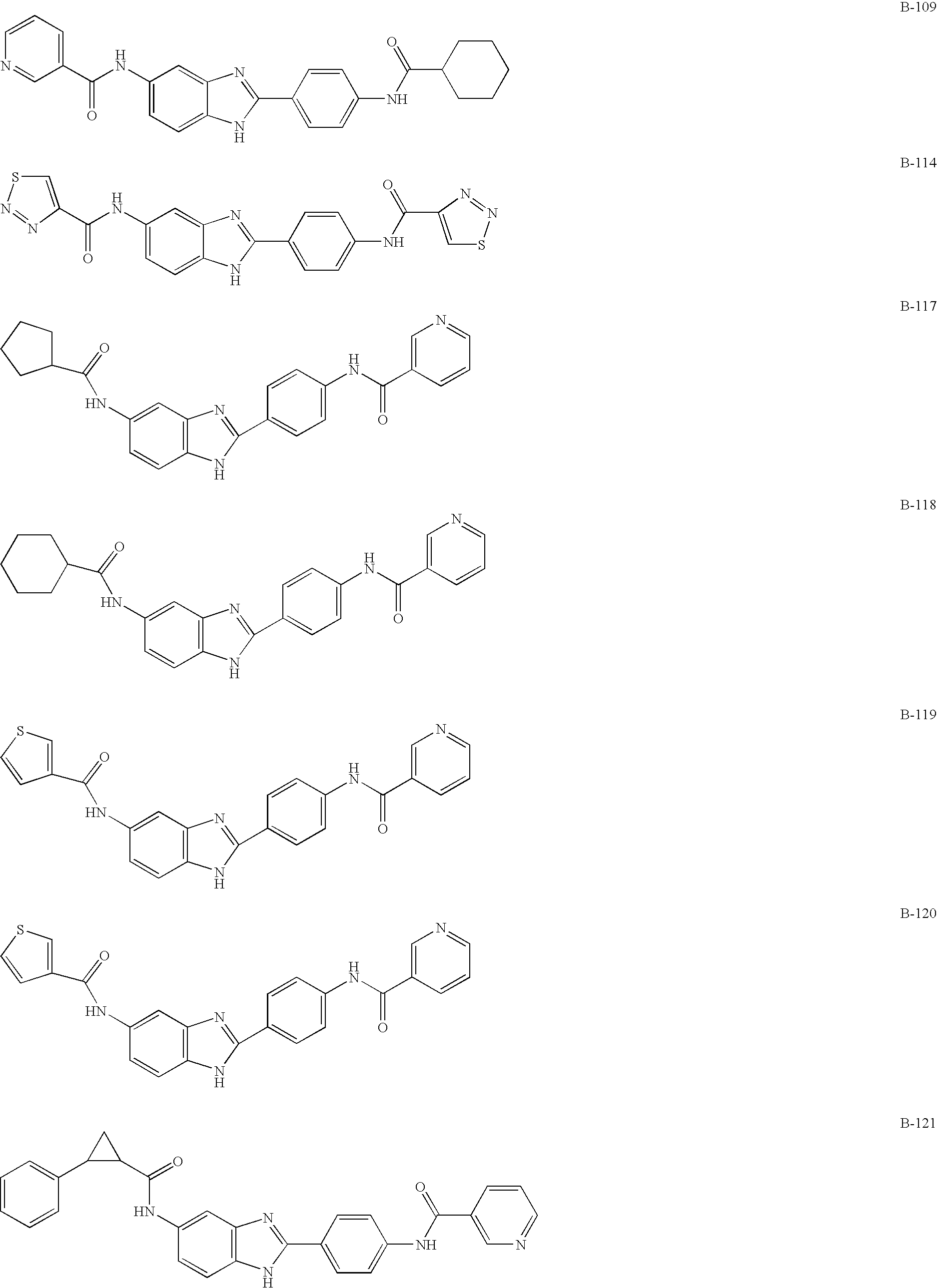

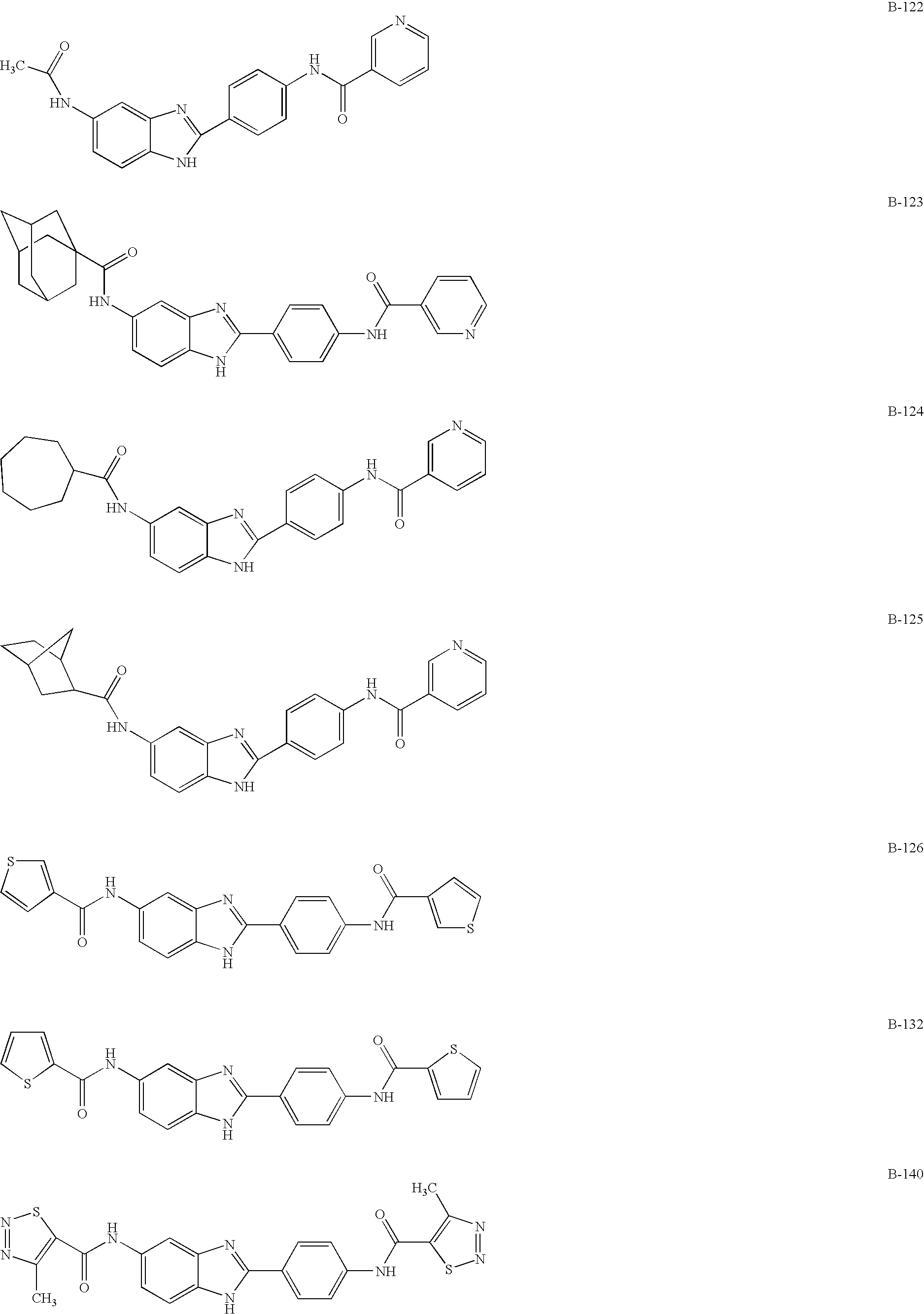

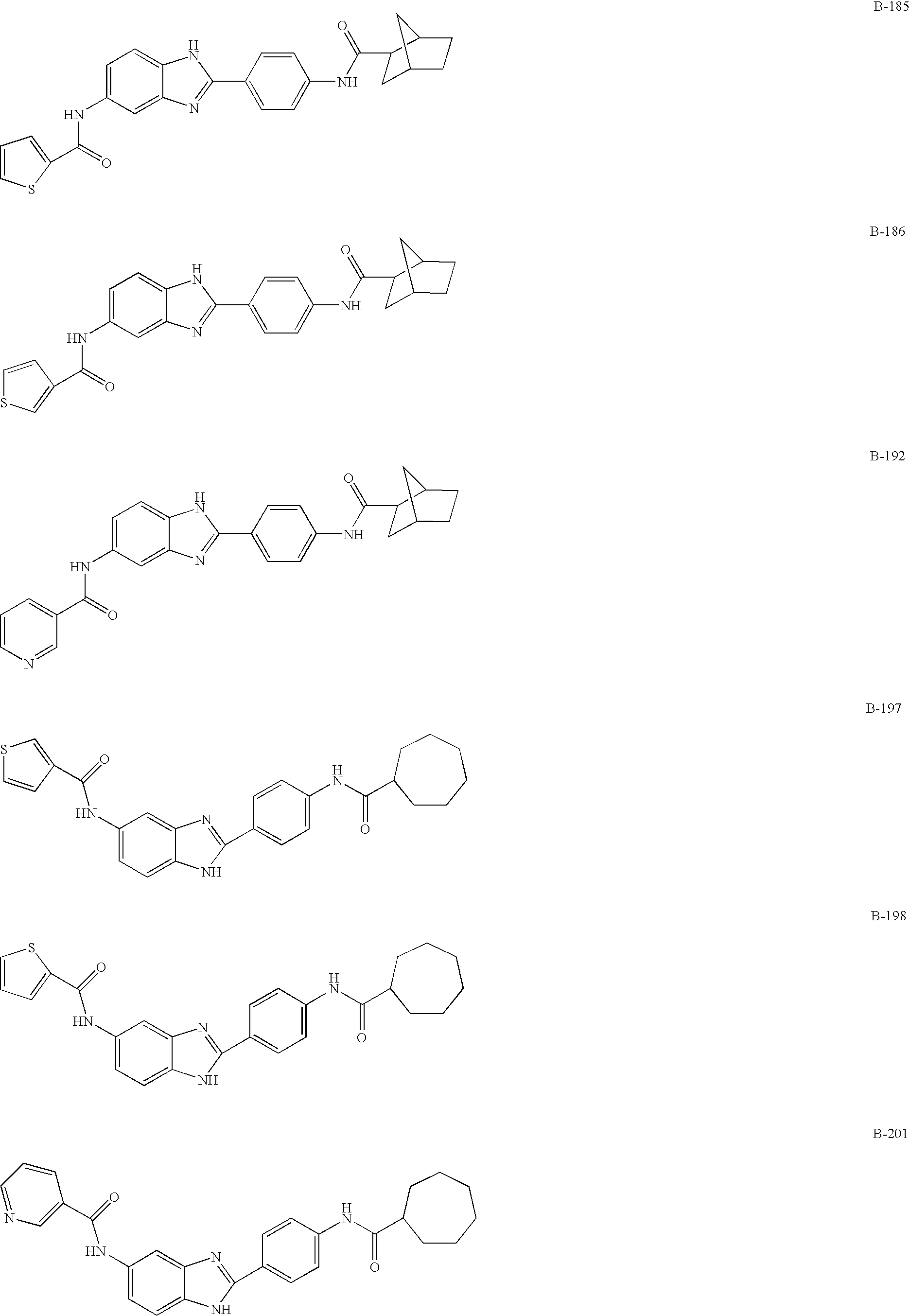

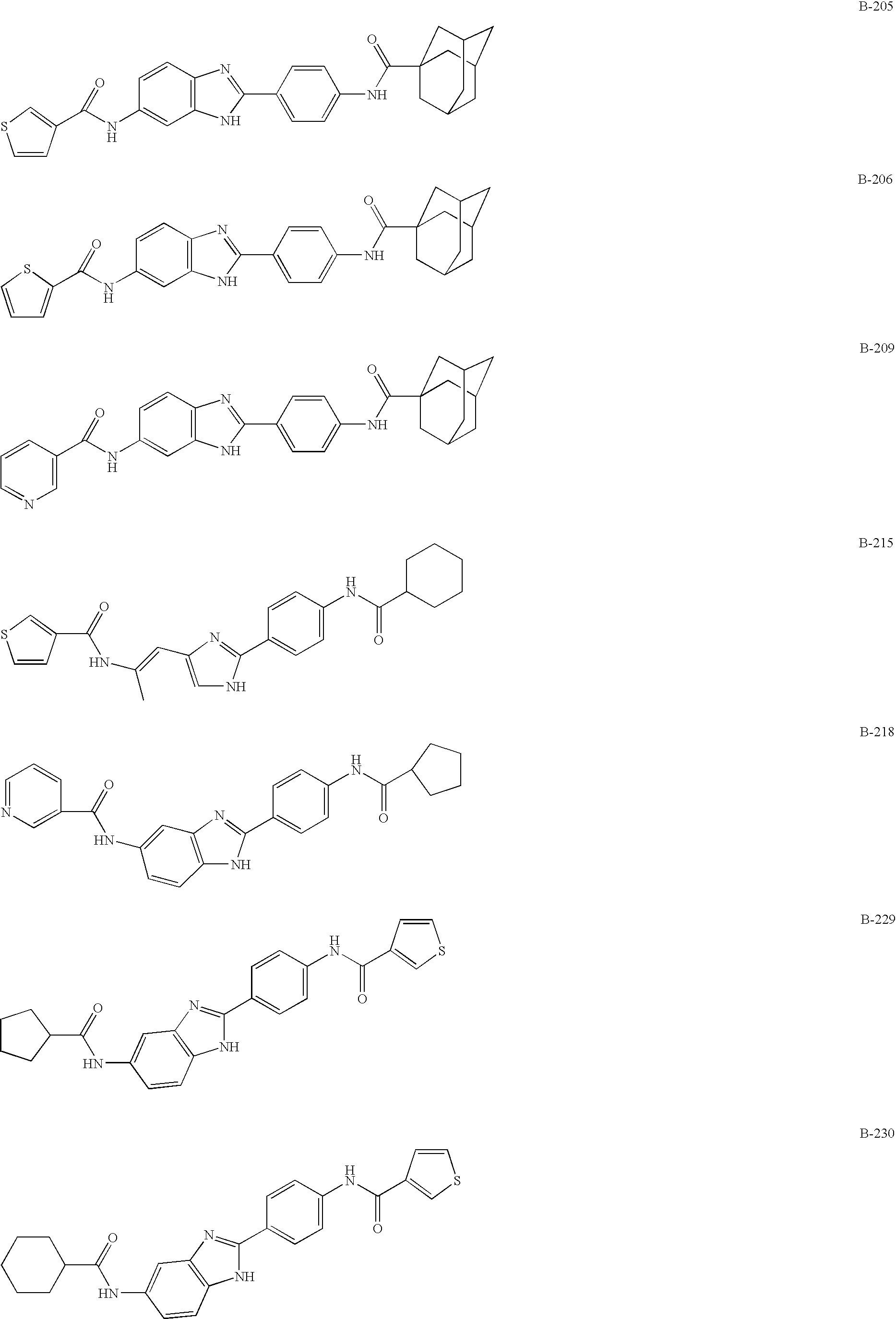

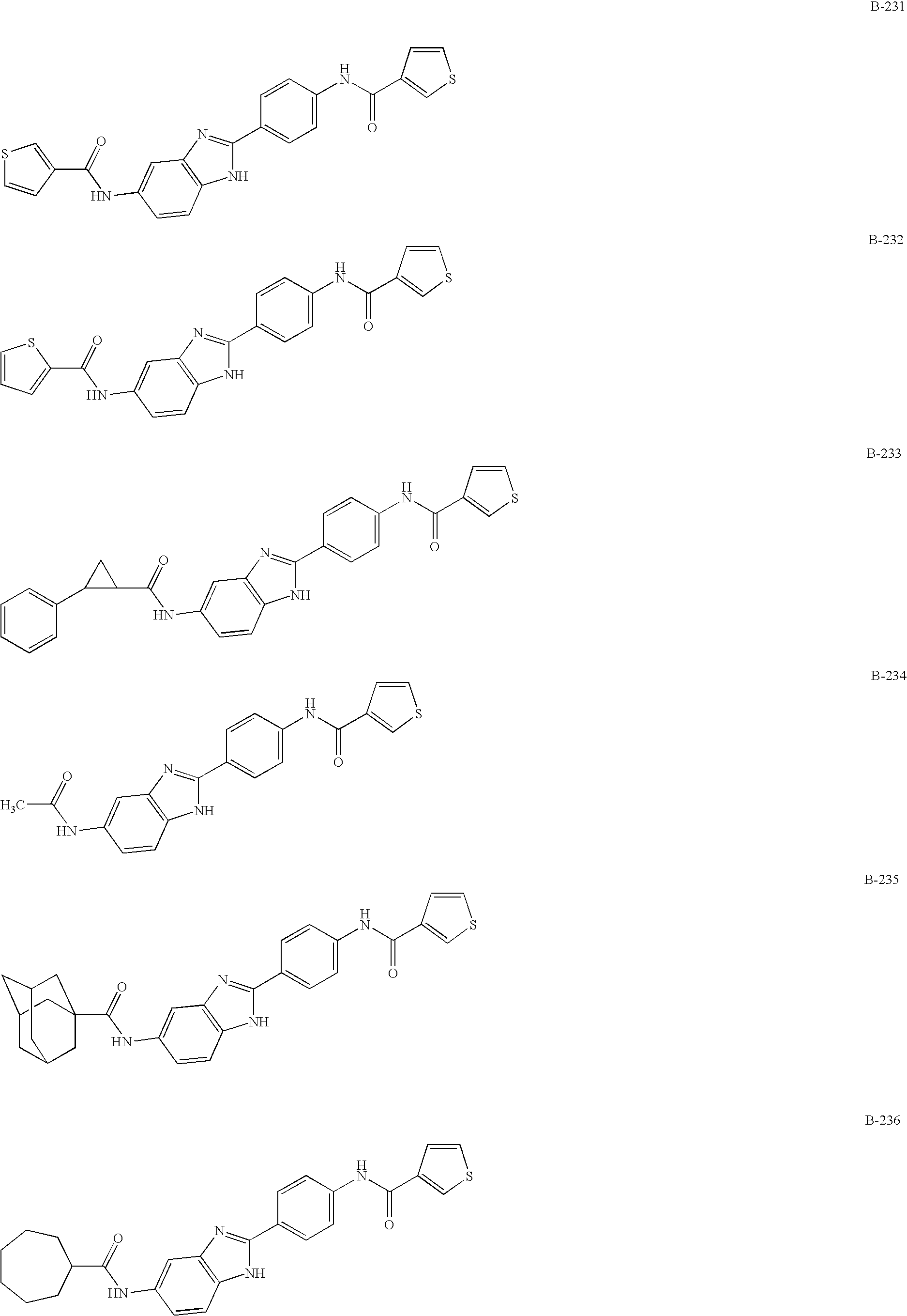

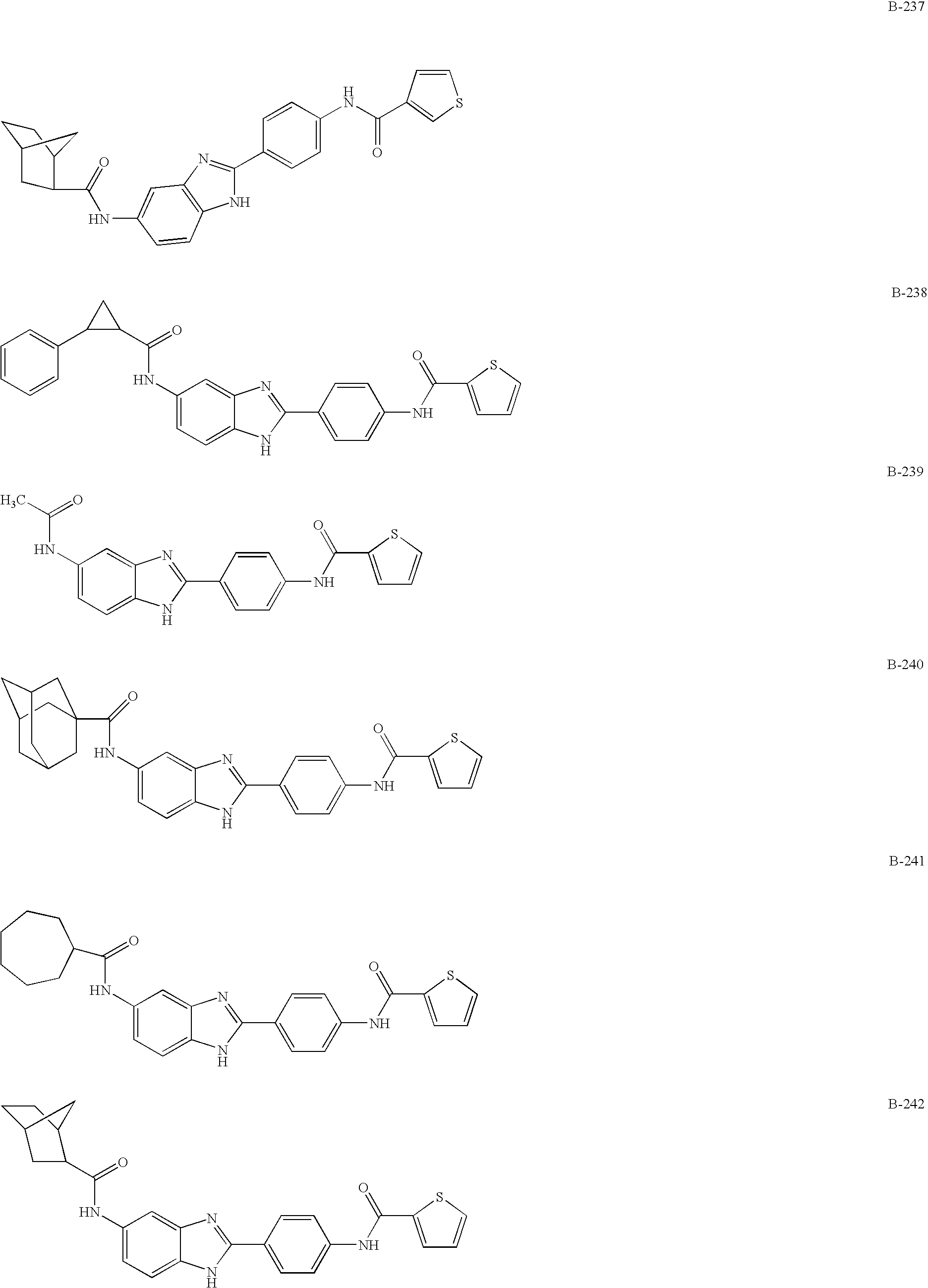

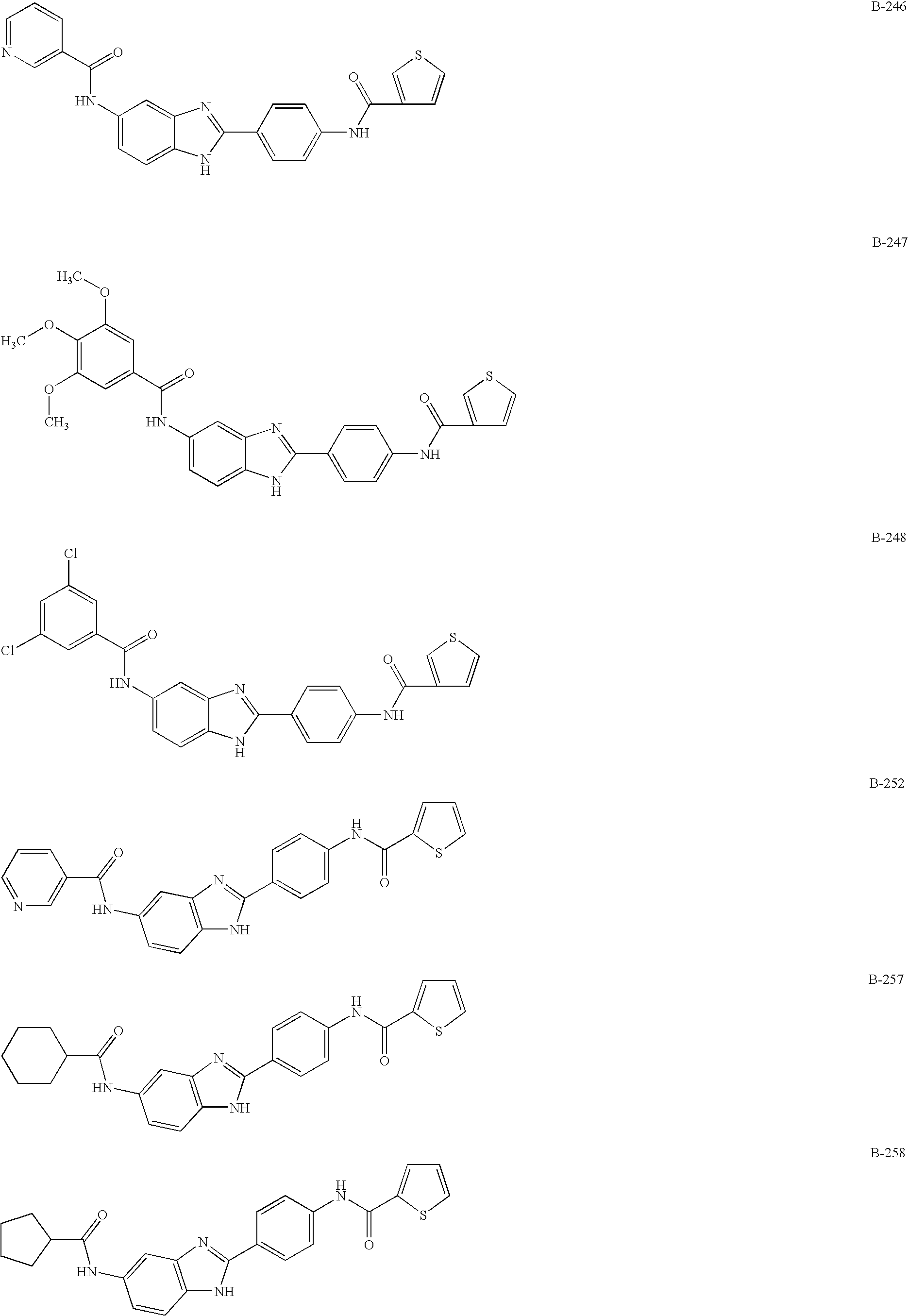

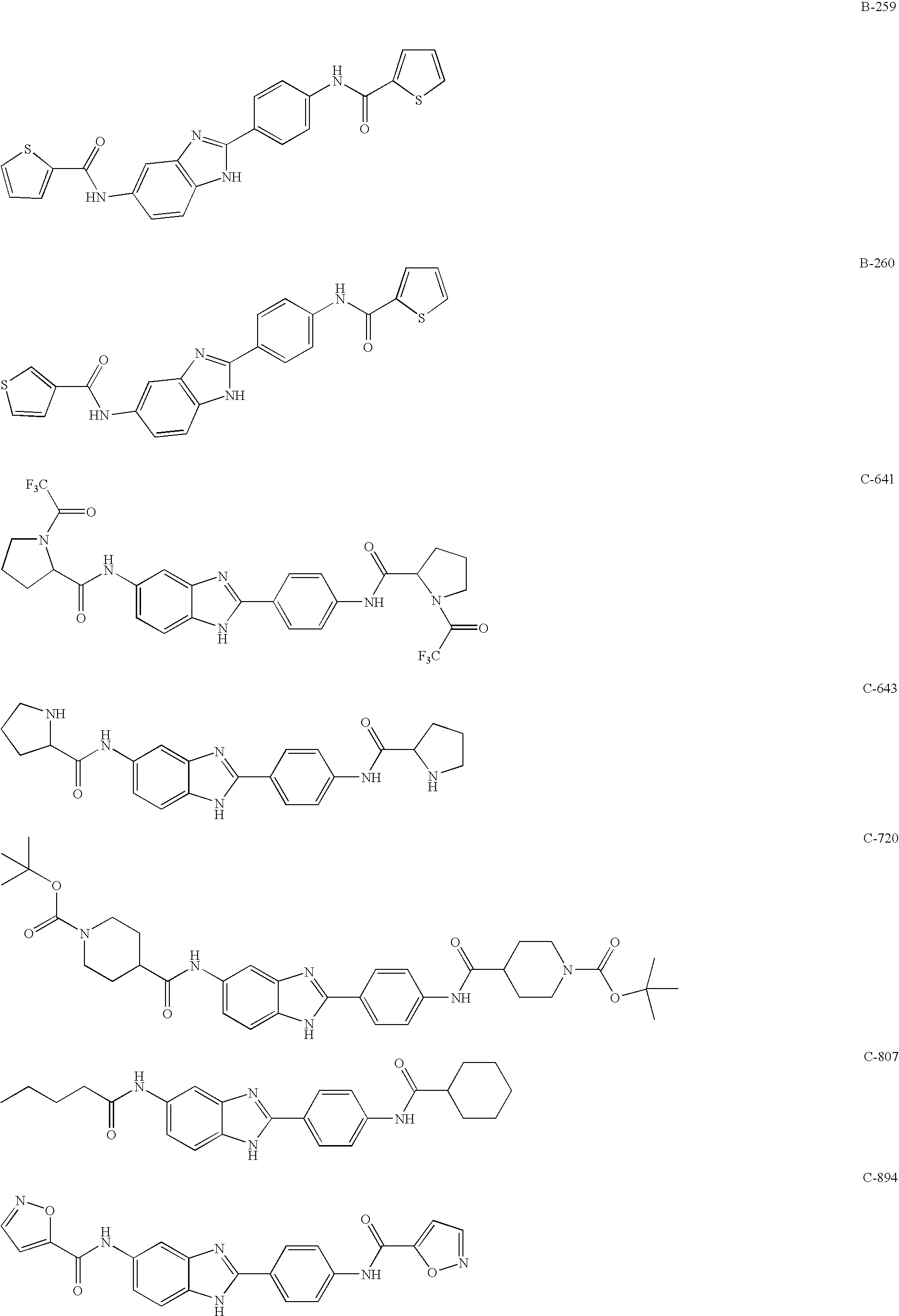

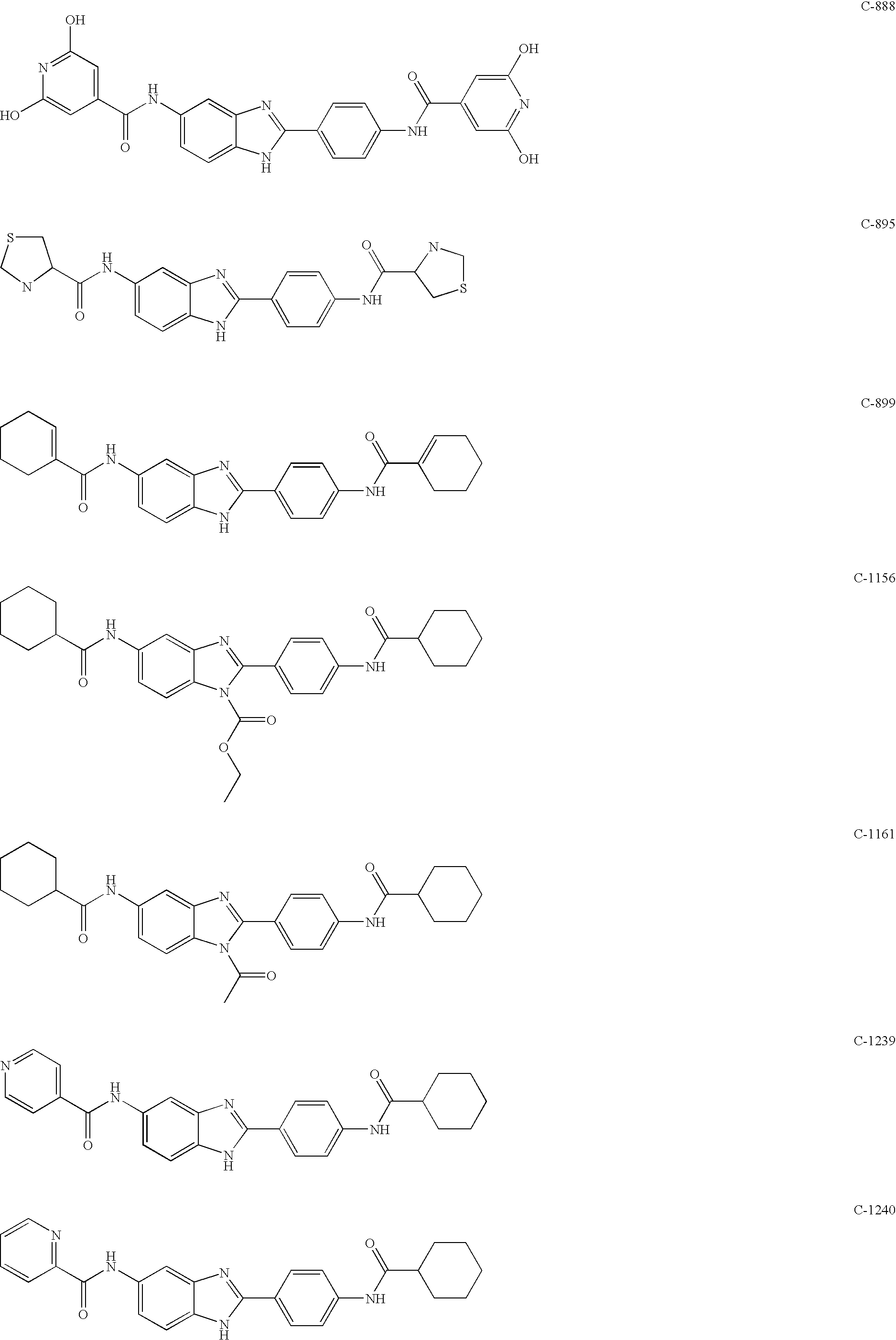

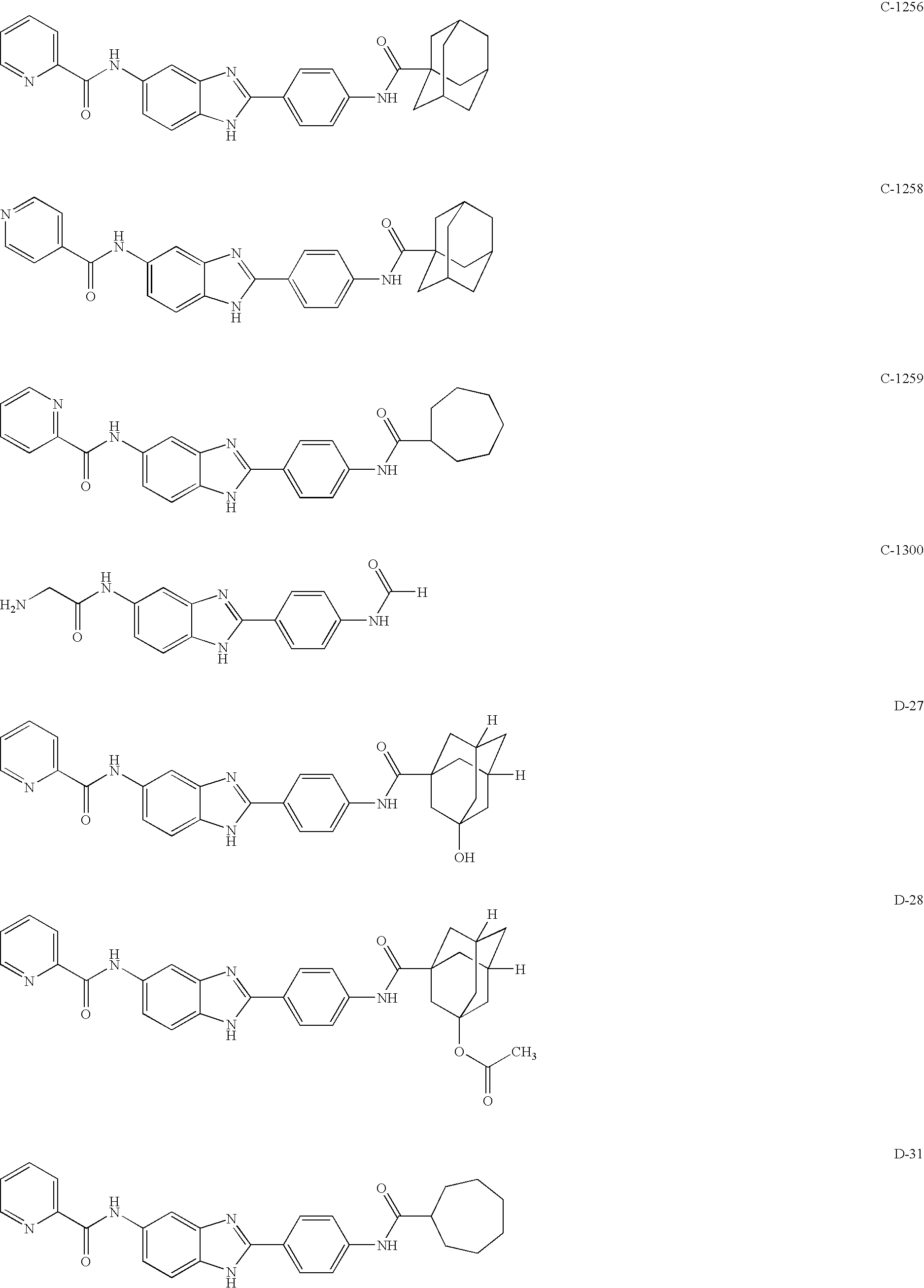

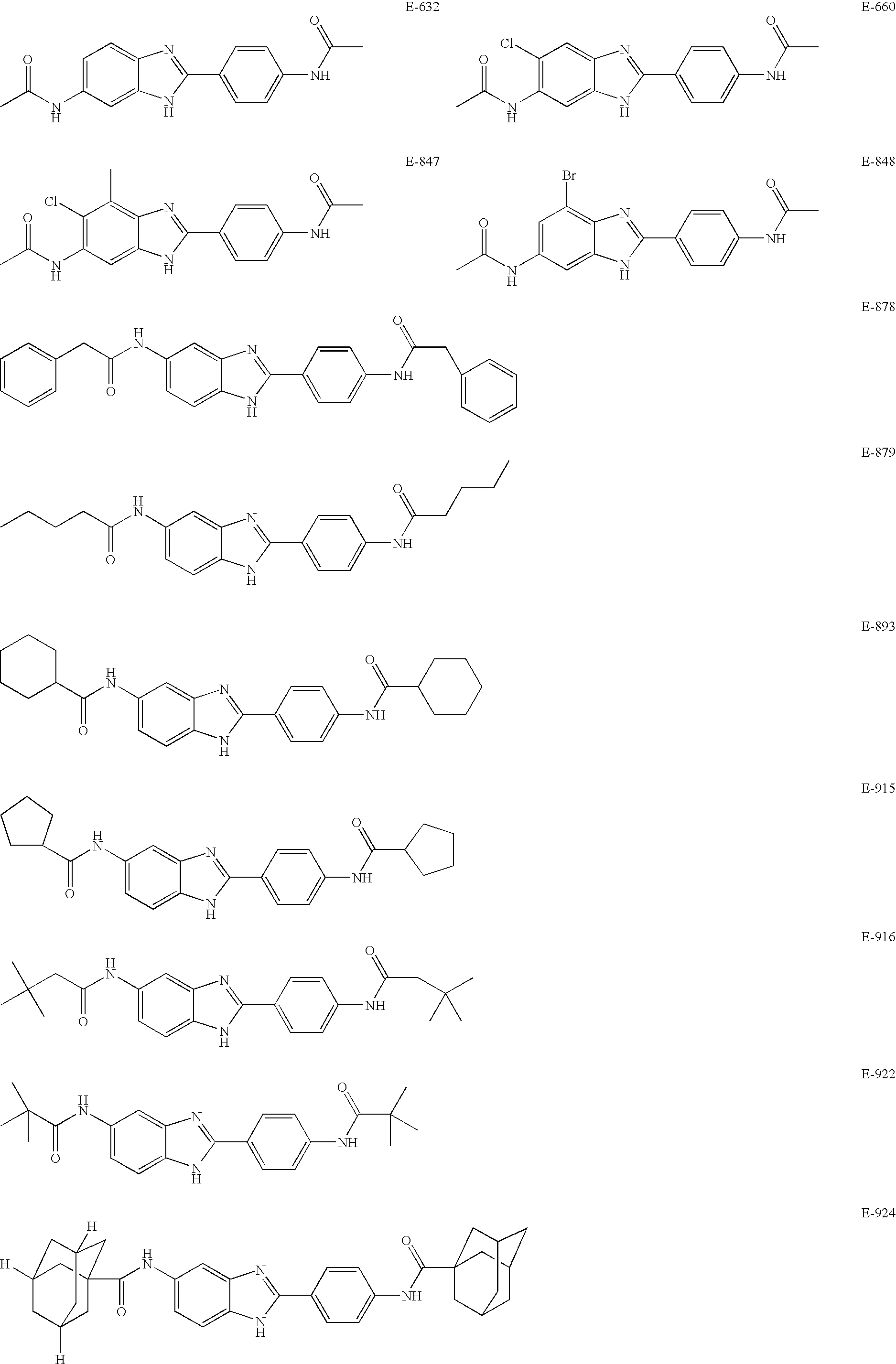

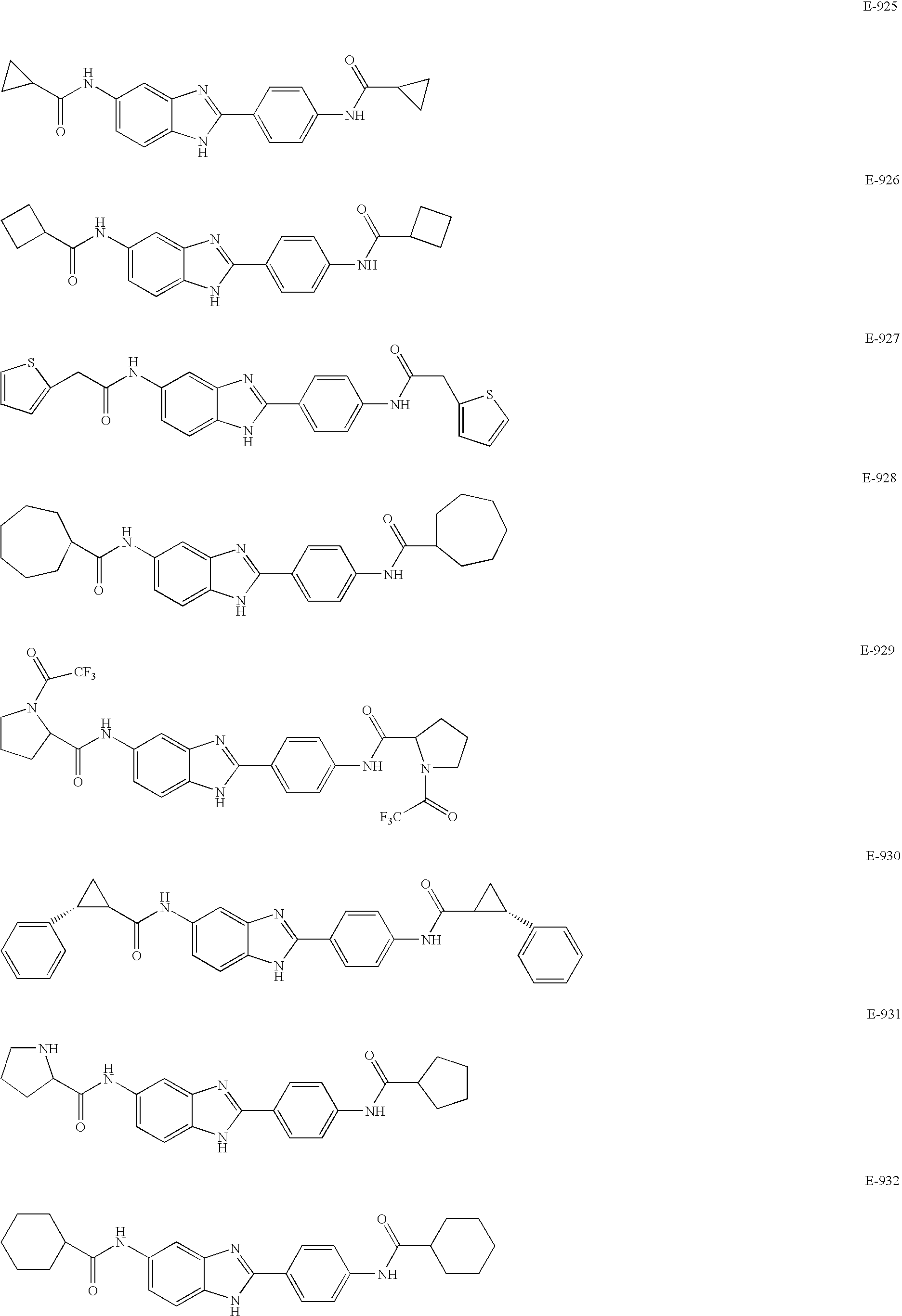

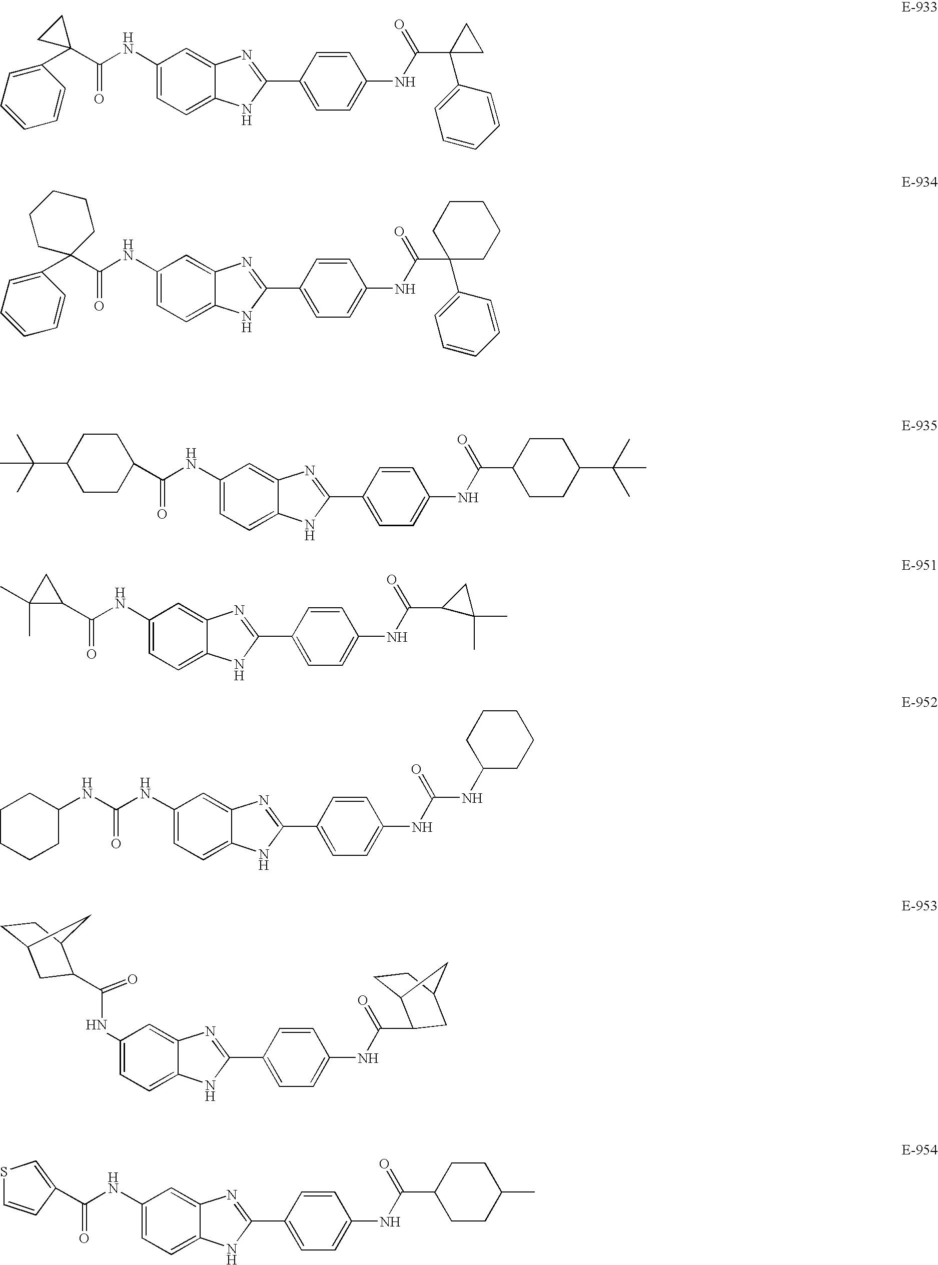

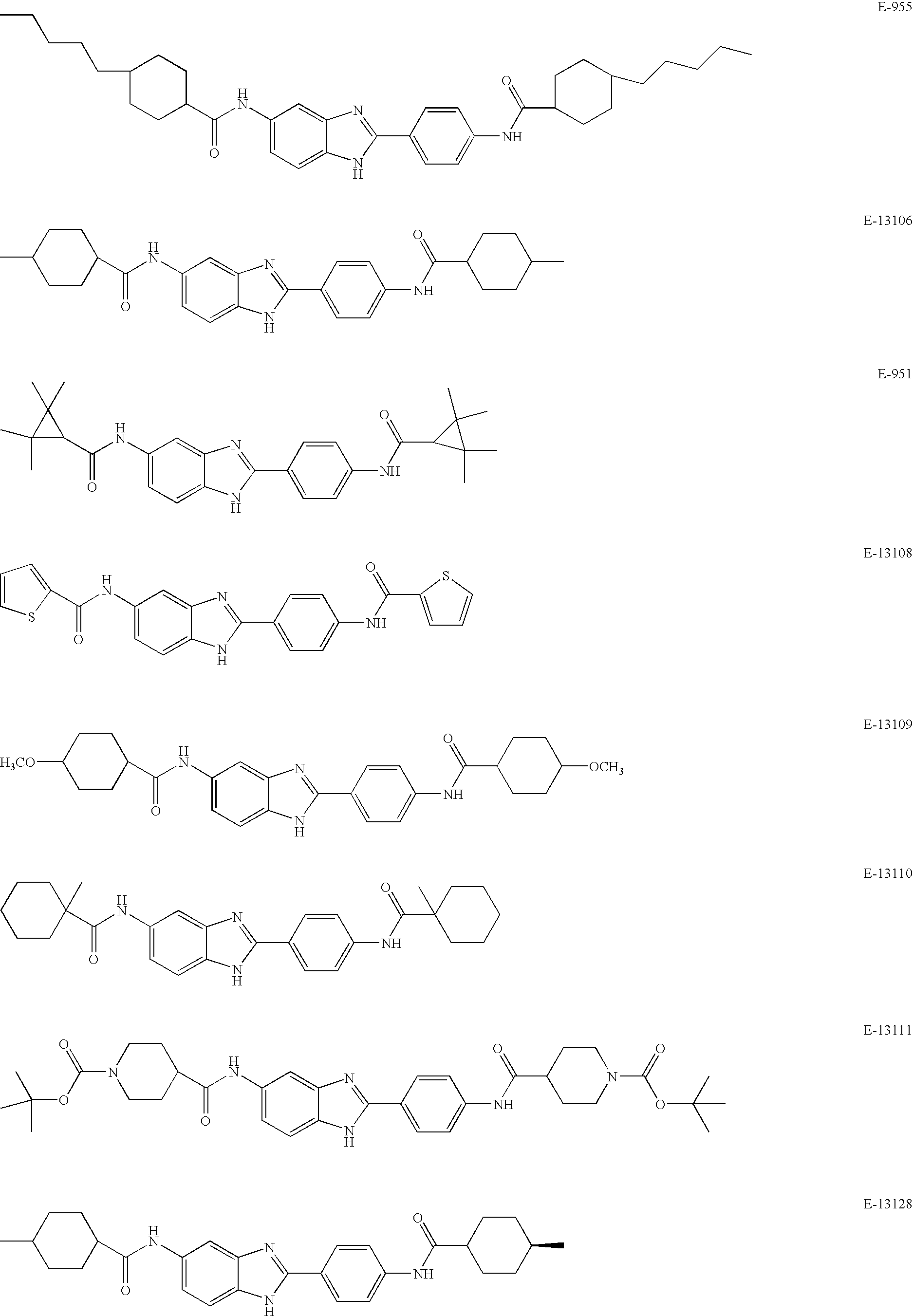

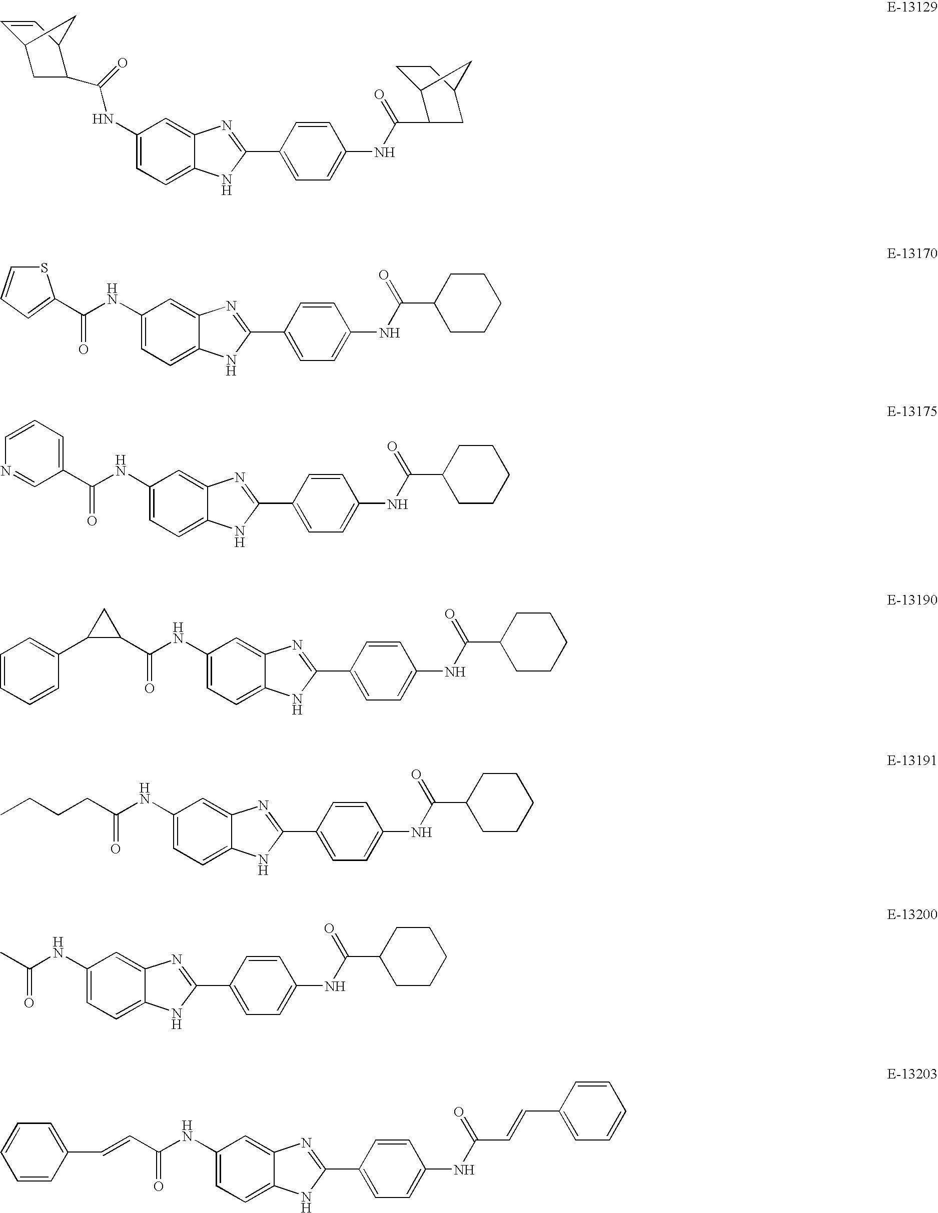

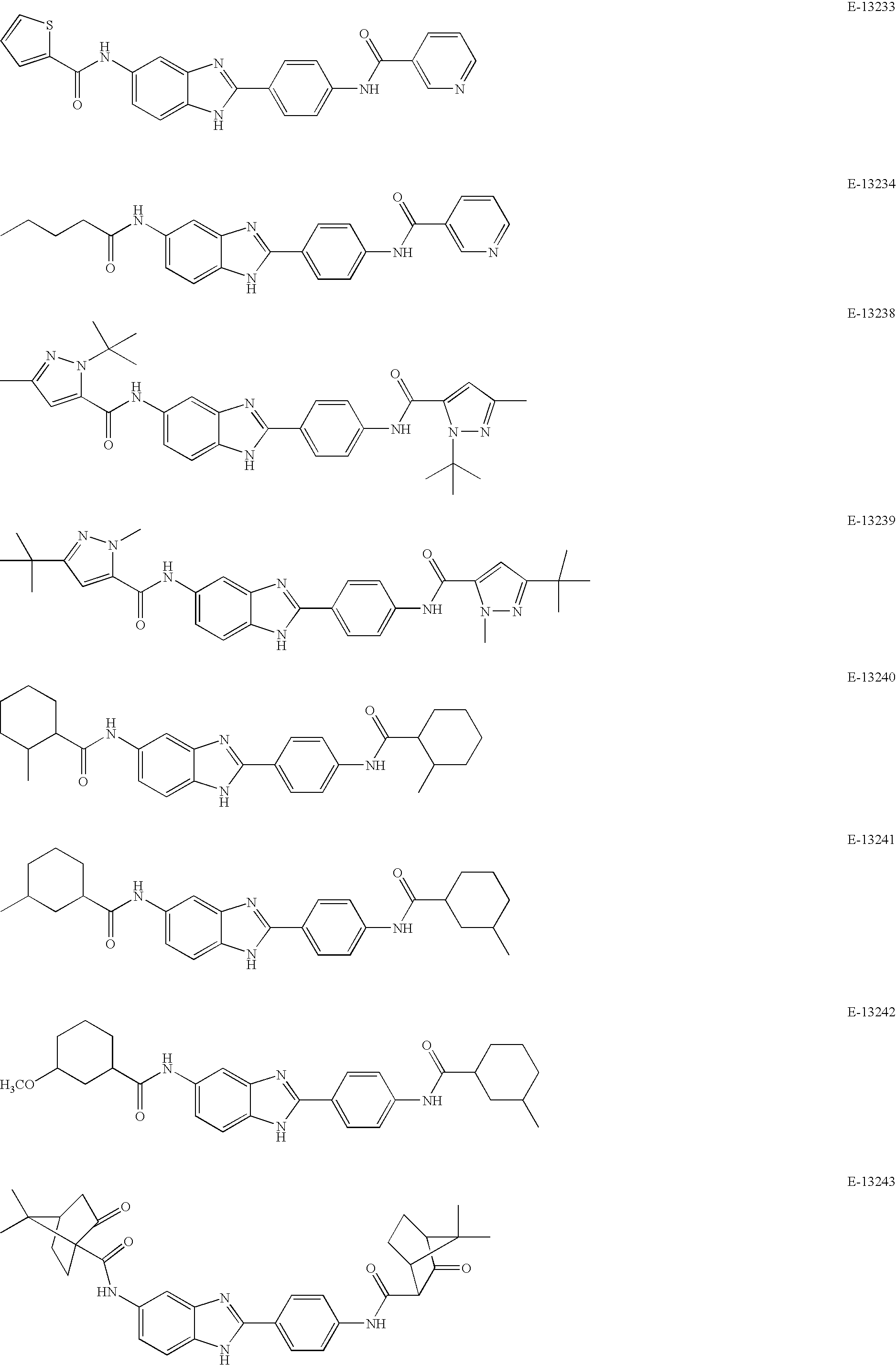

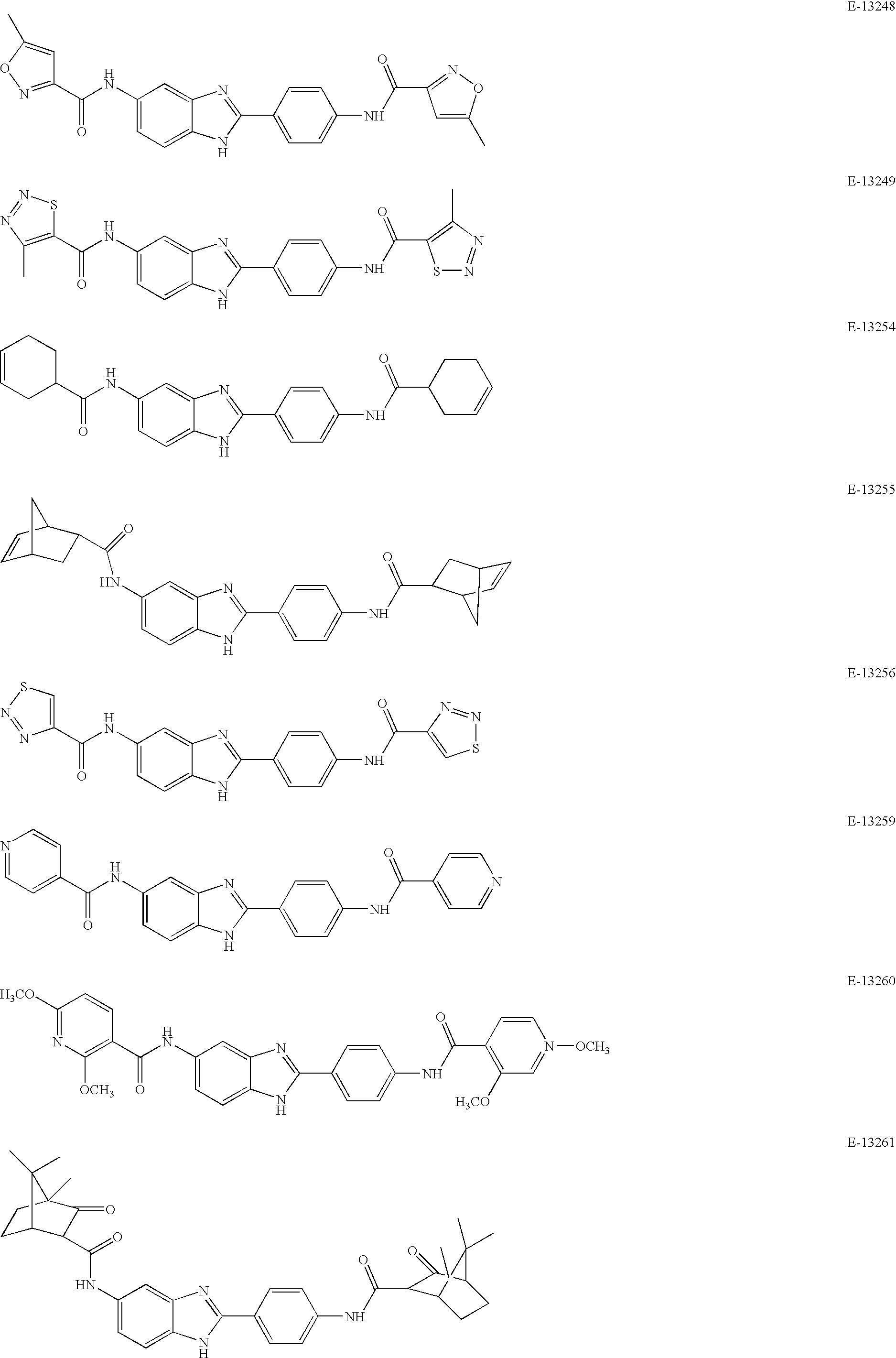

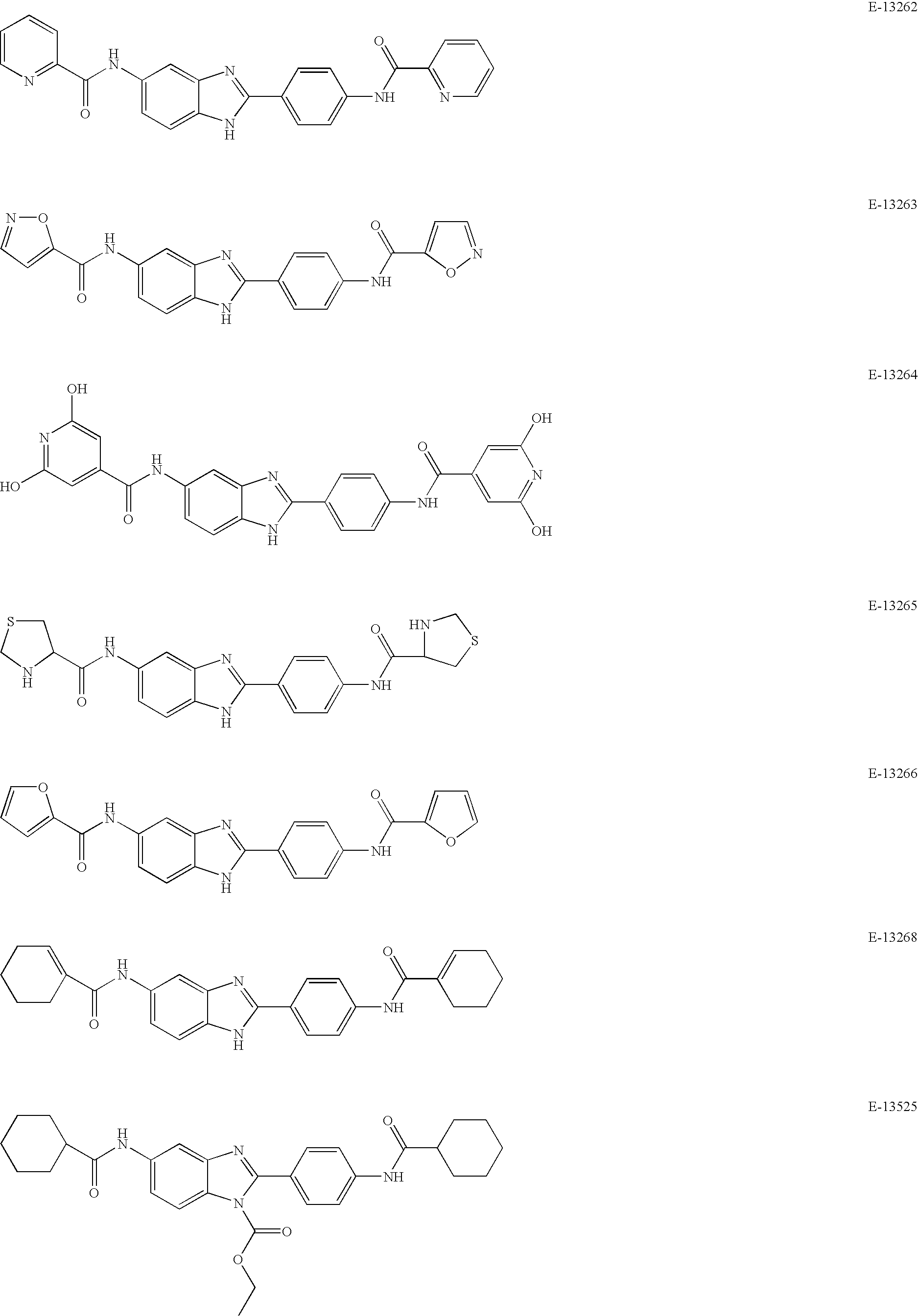

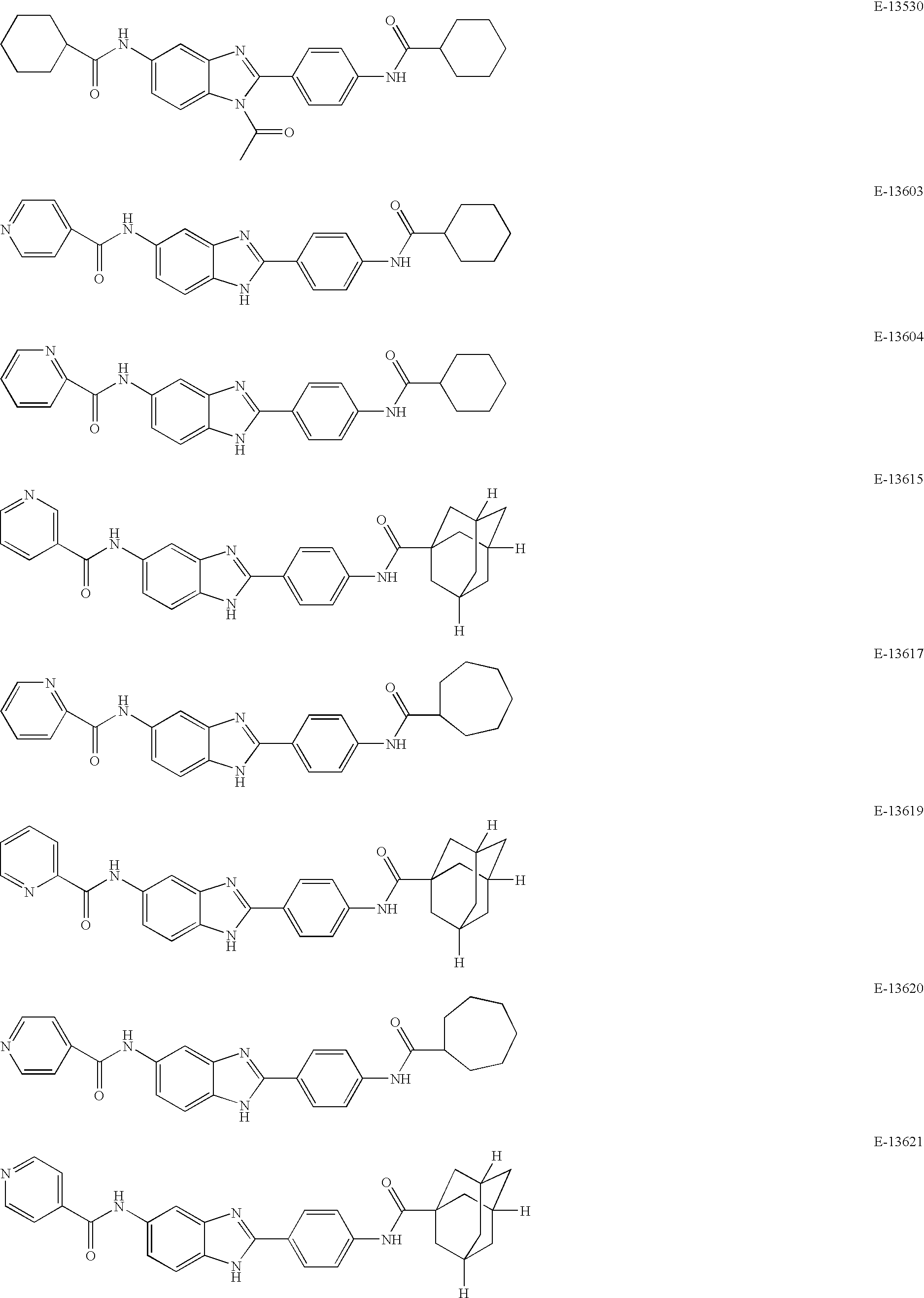

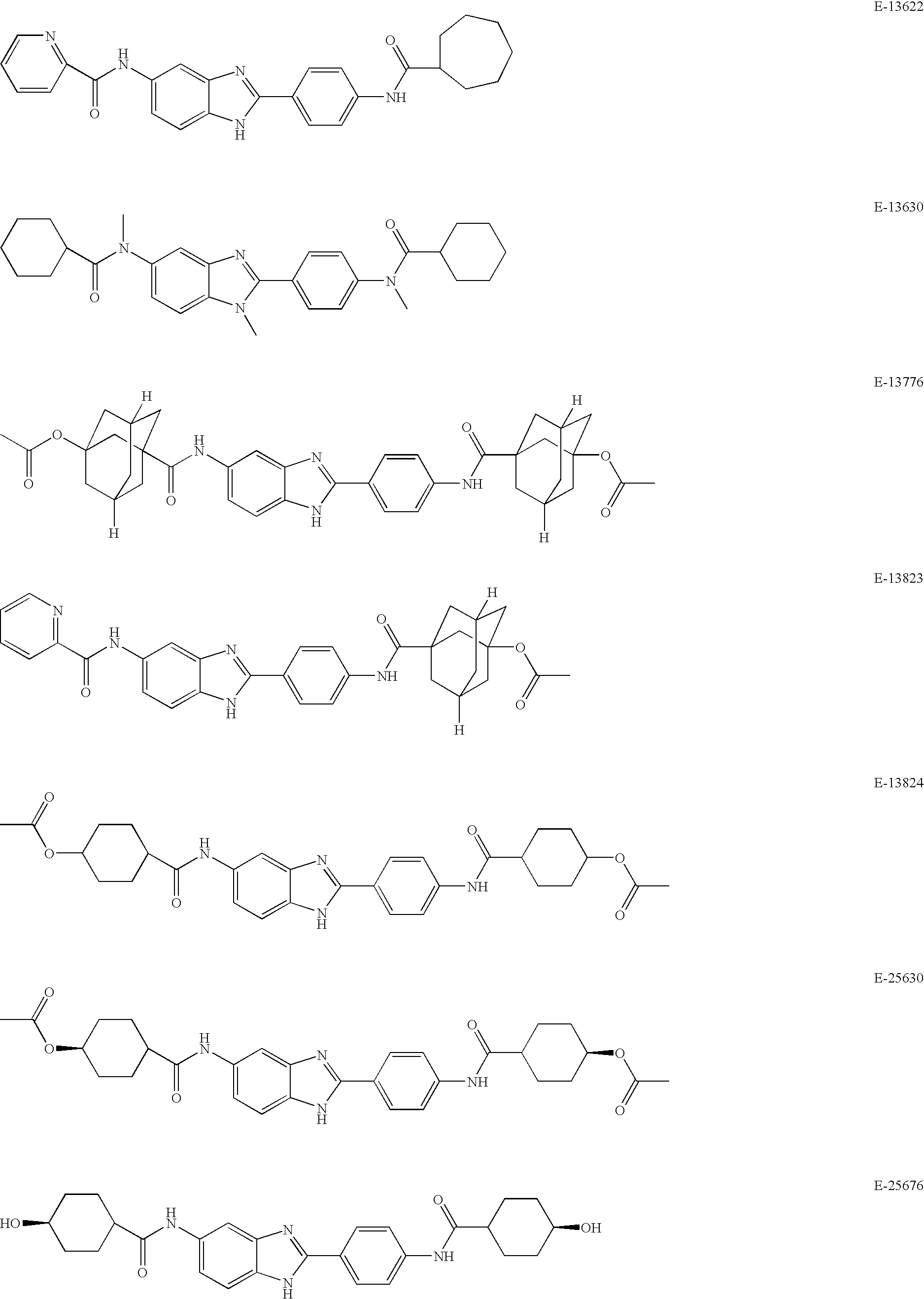

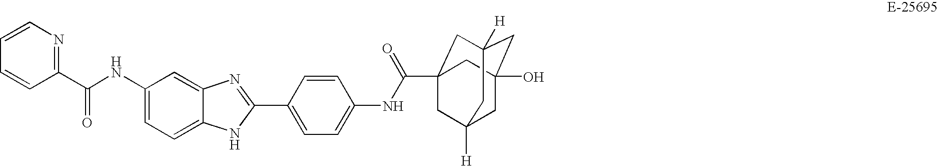

Specific compounds of the preferred embodiments of Genus B which are preferred are represented by the following structural formulae or a pharmaceutically acceptable salt or solvate thereof.

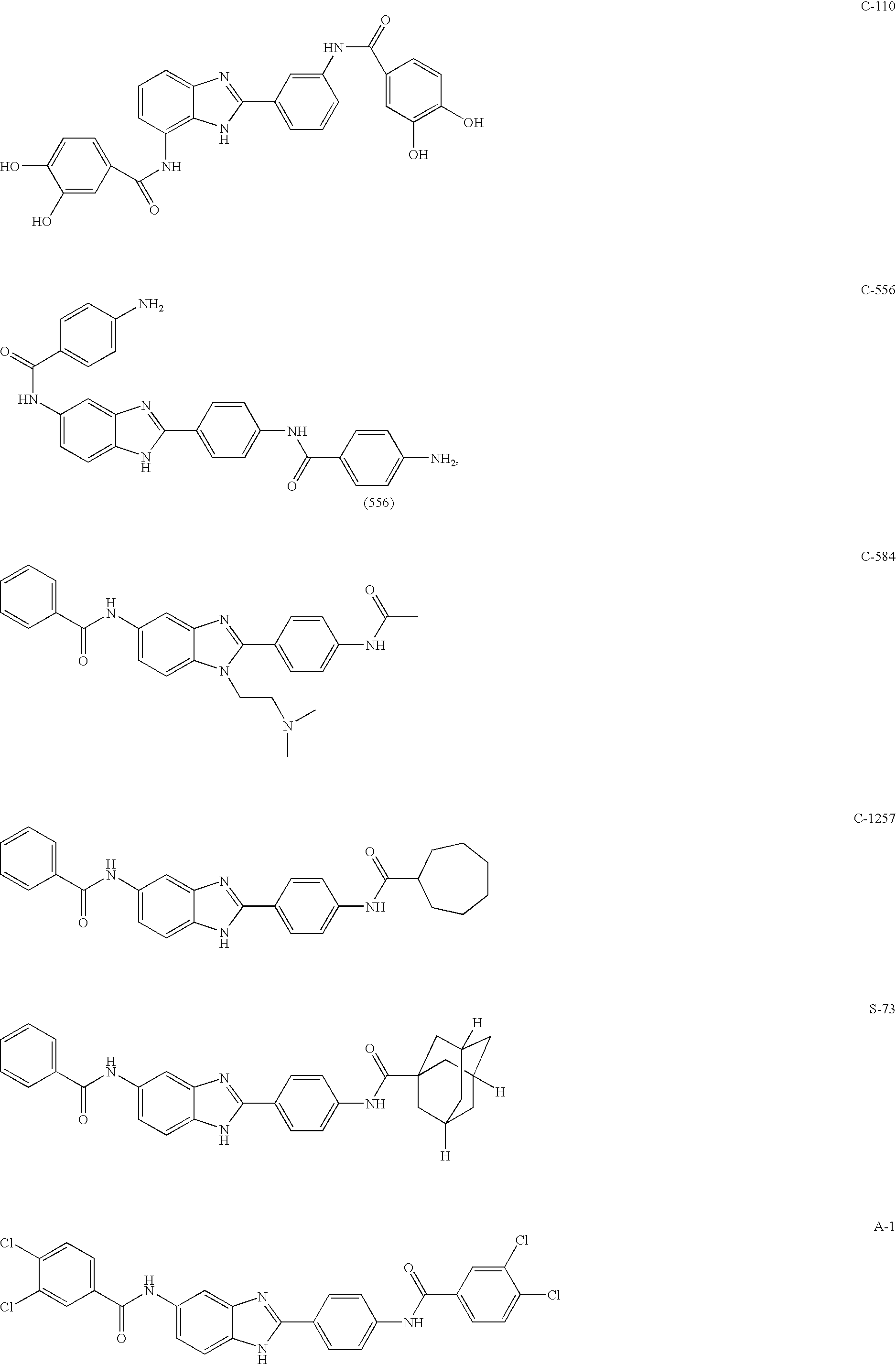

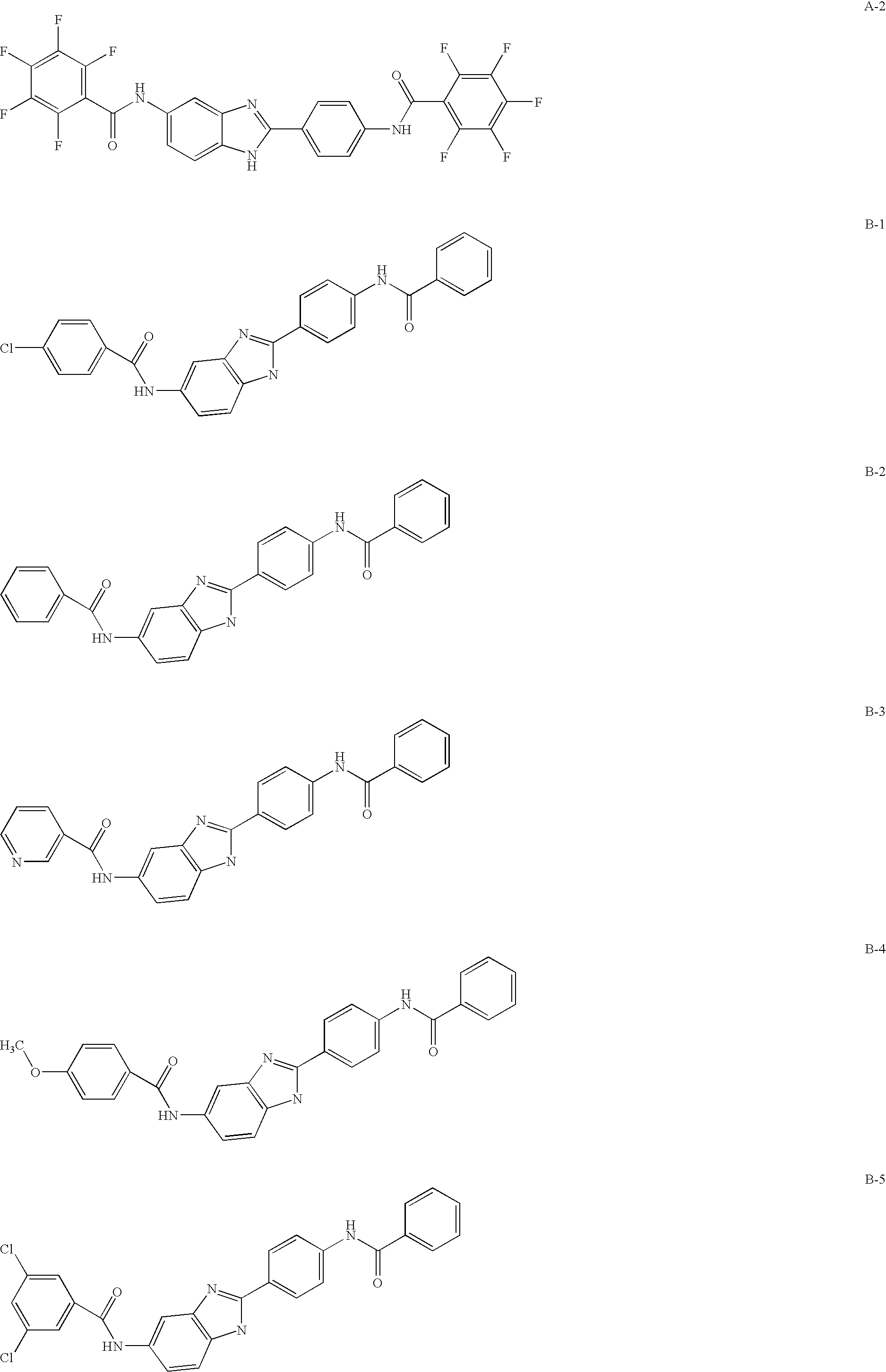

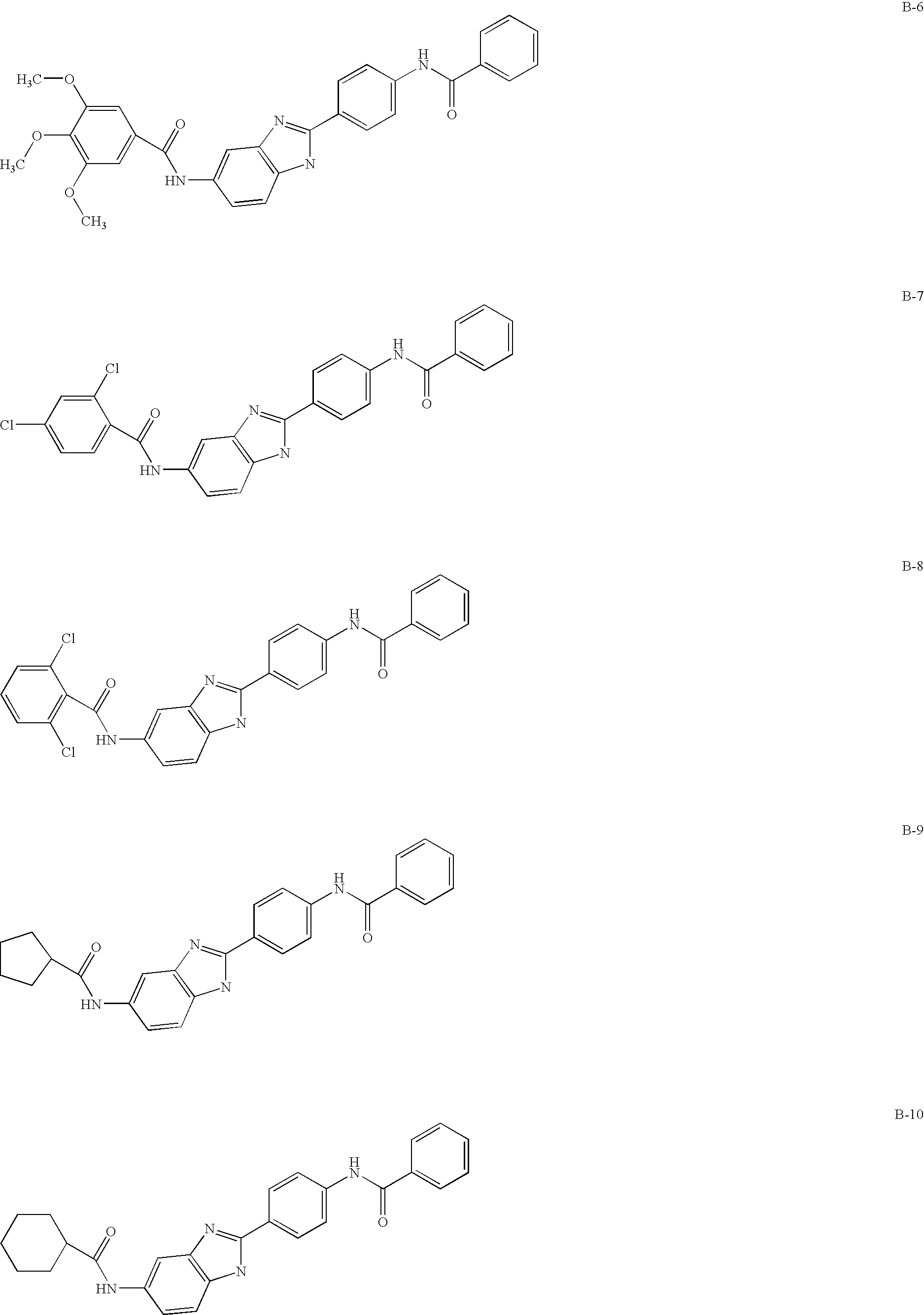

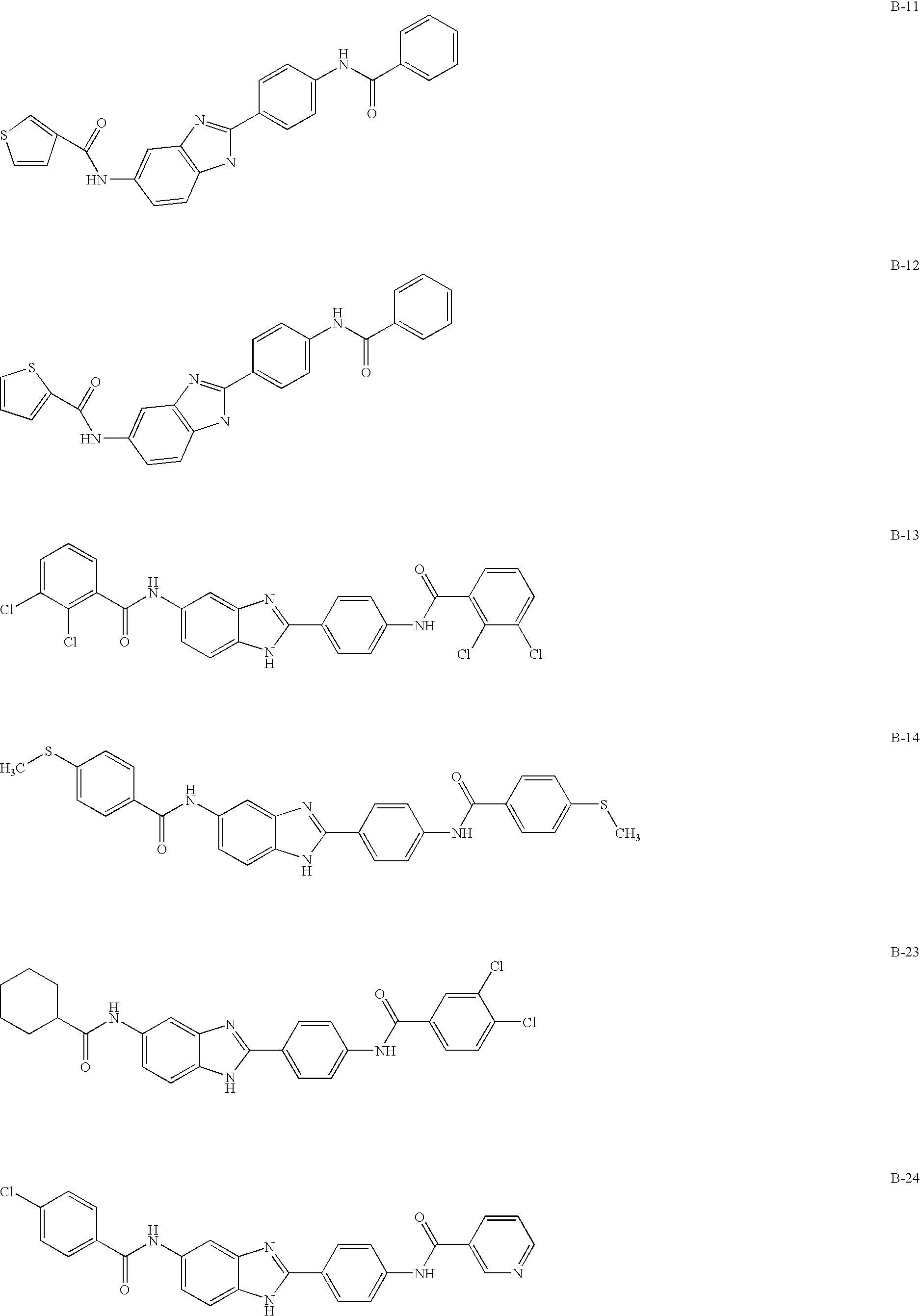

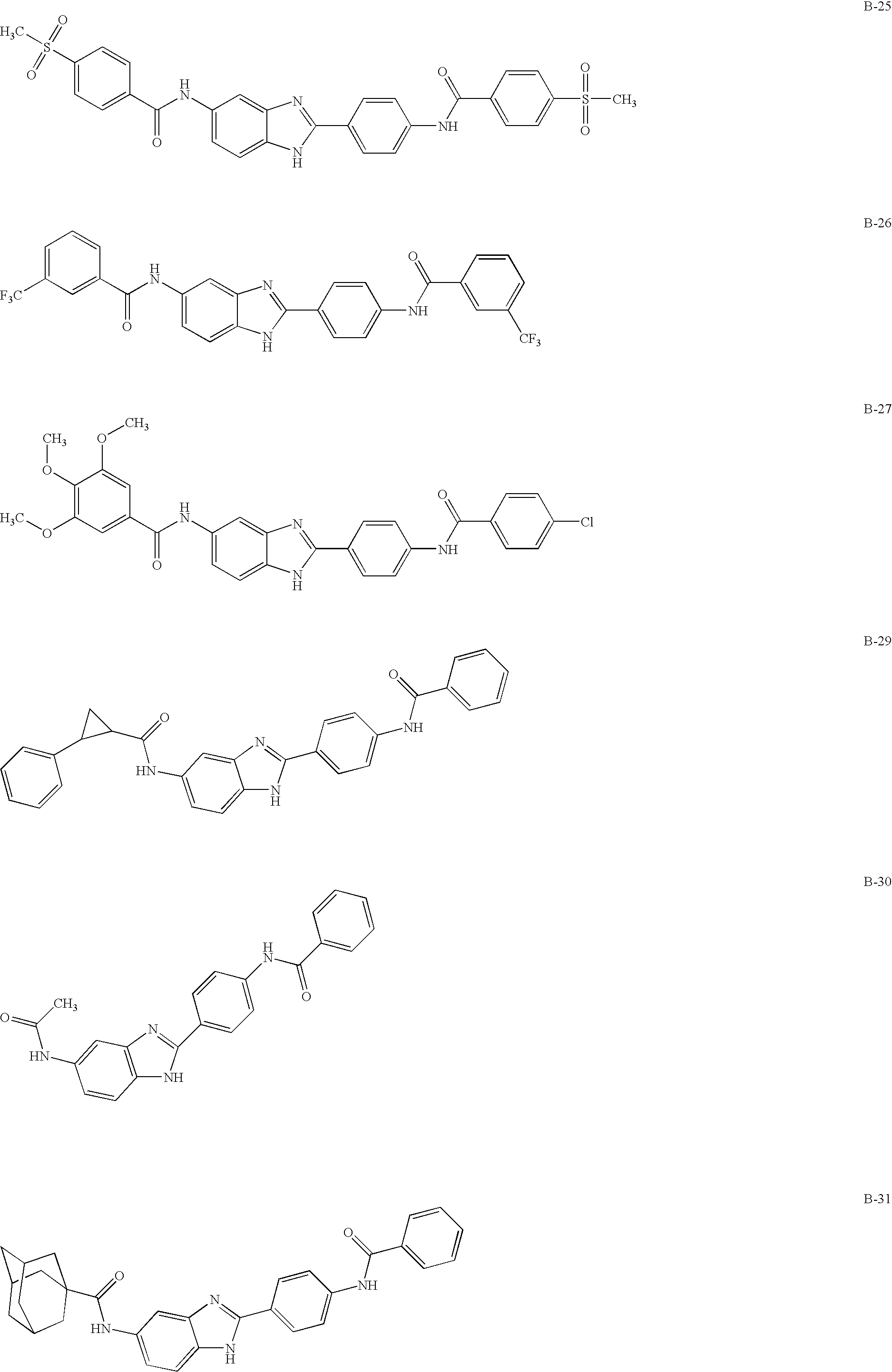

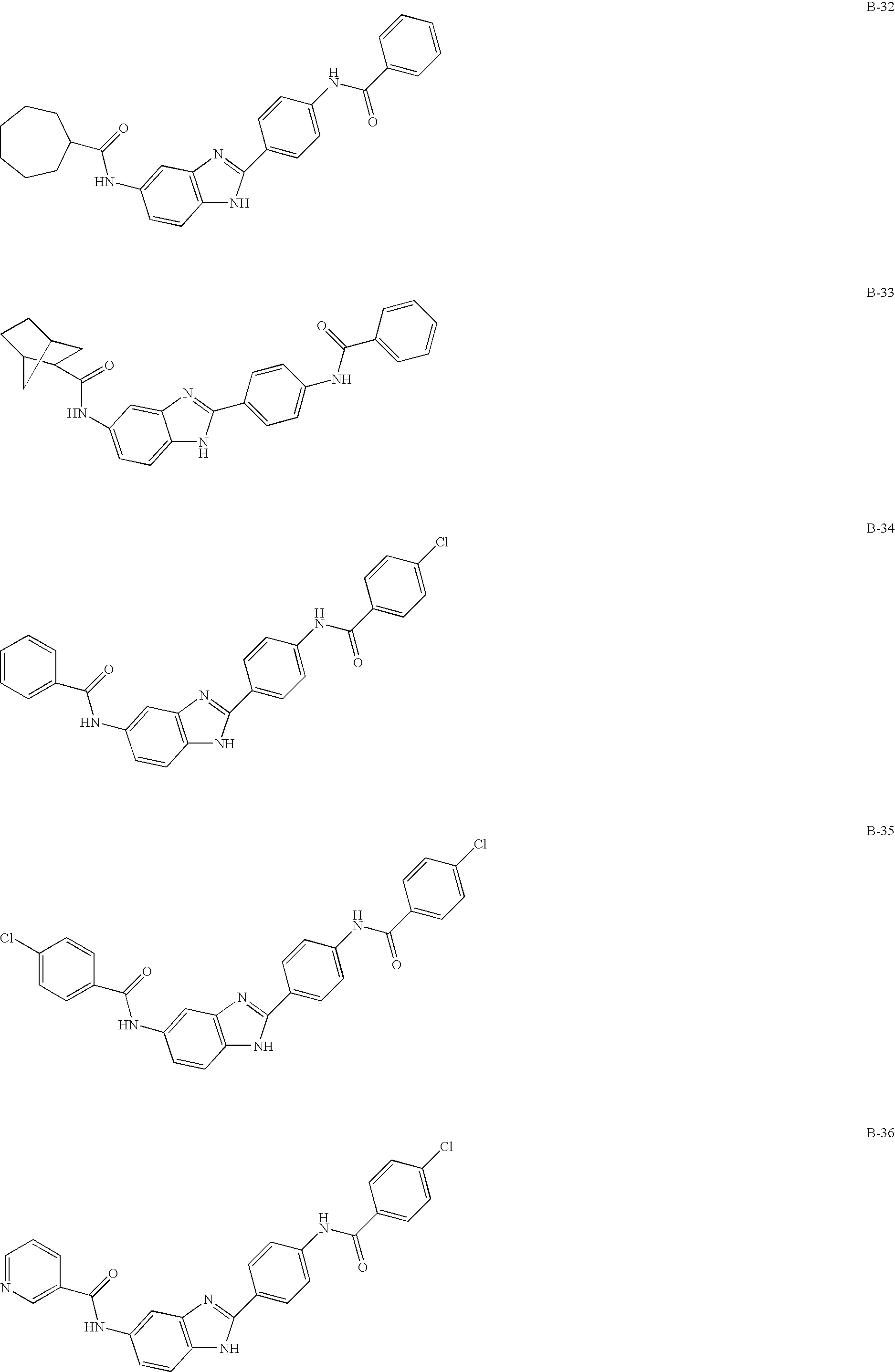

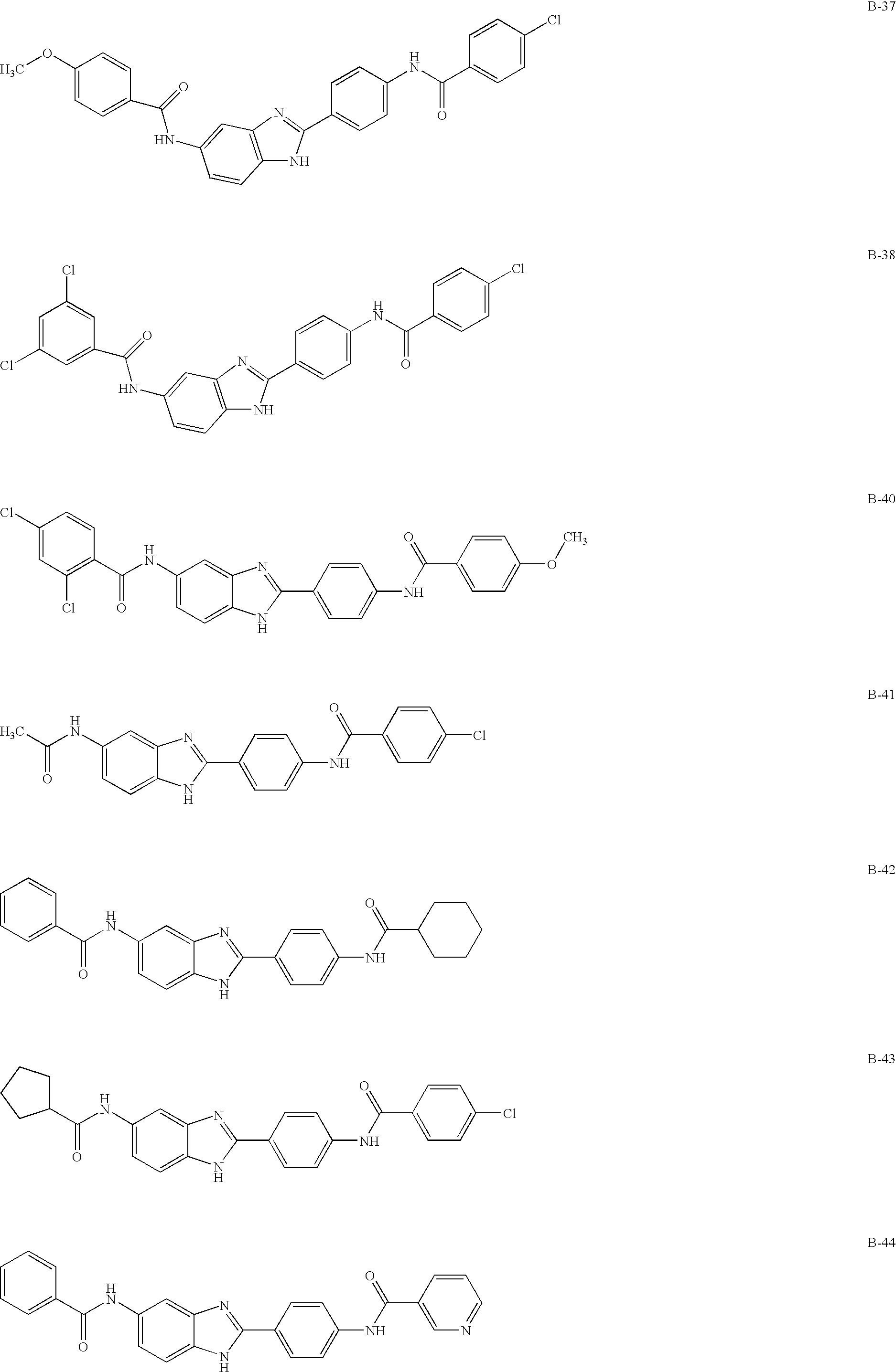

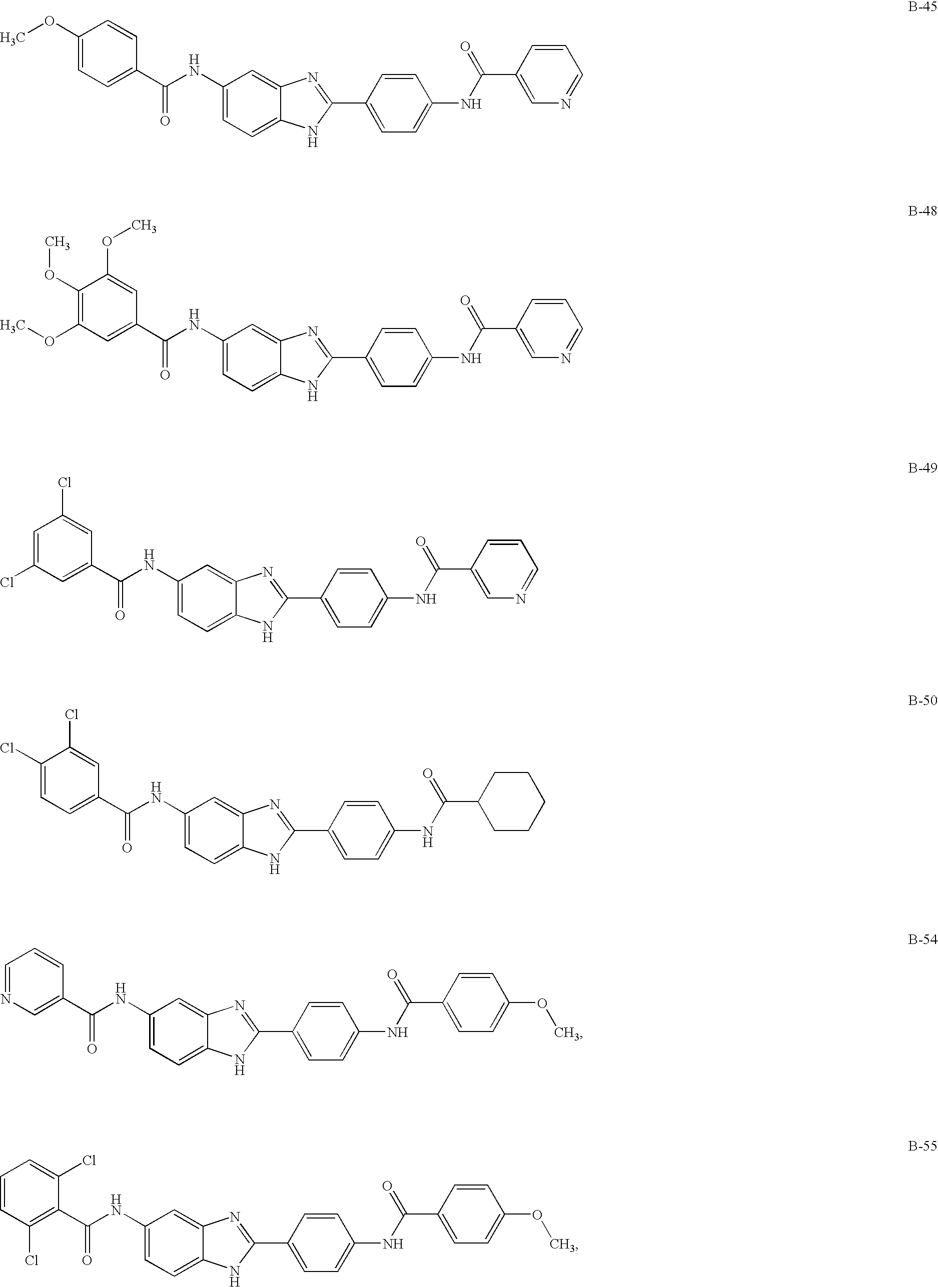

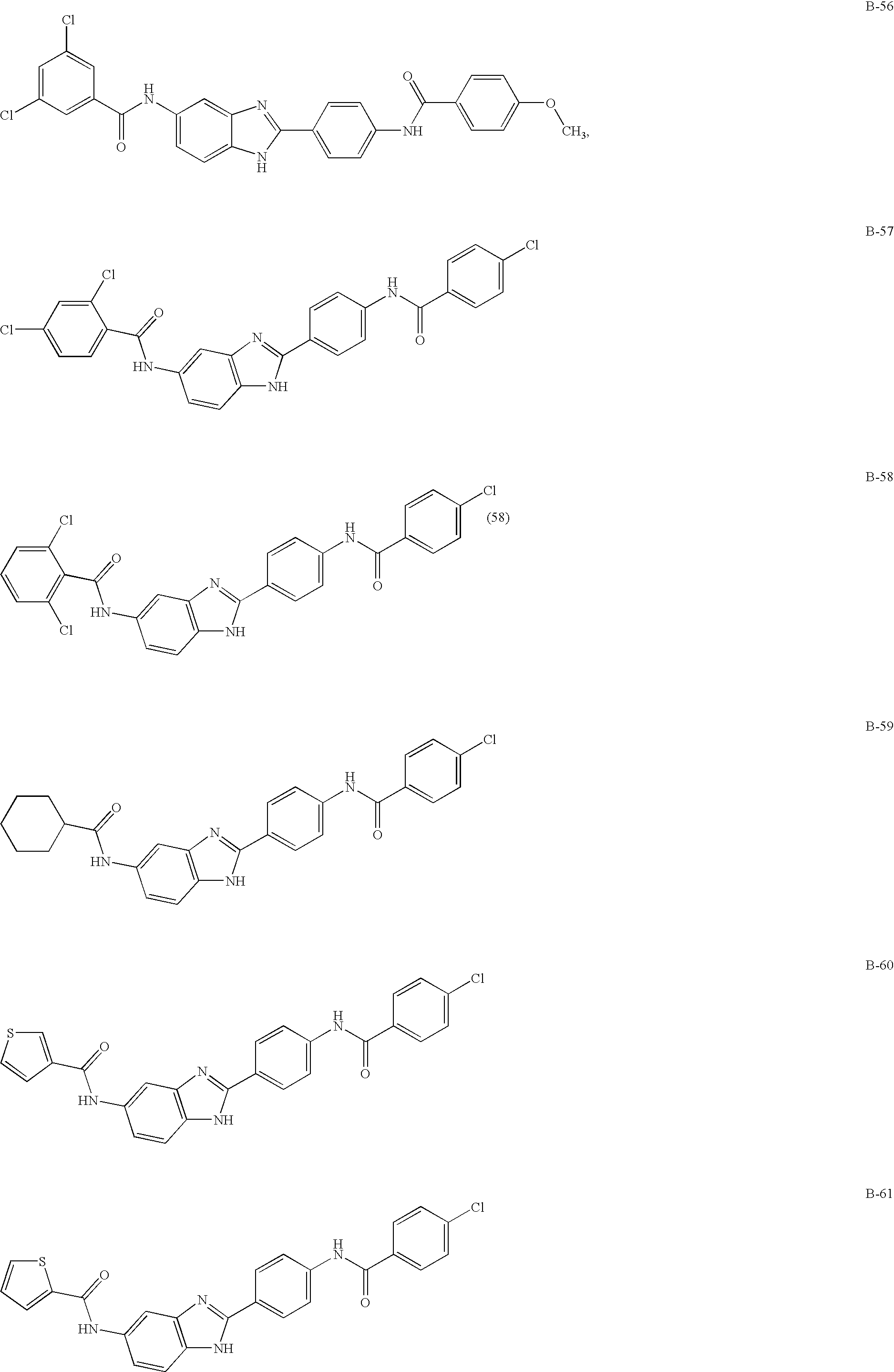

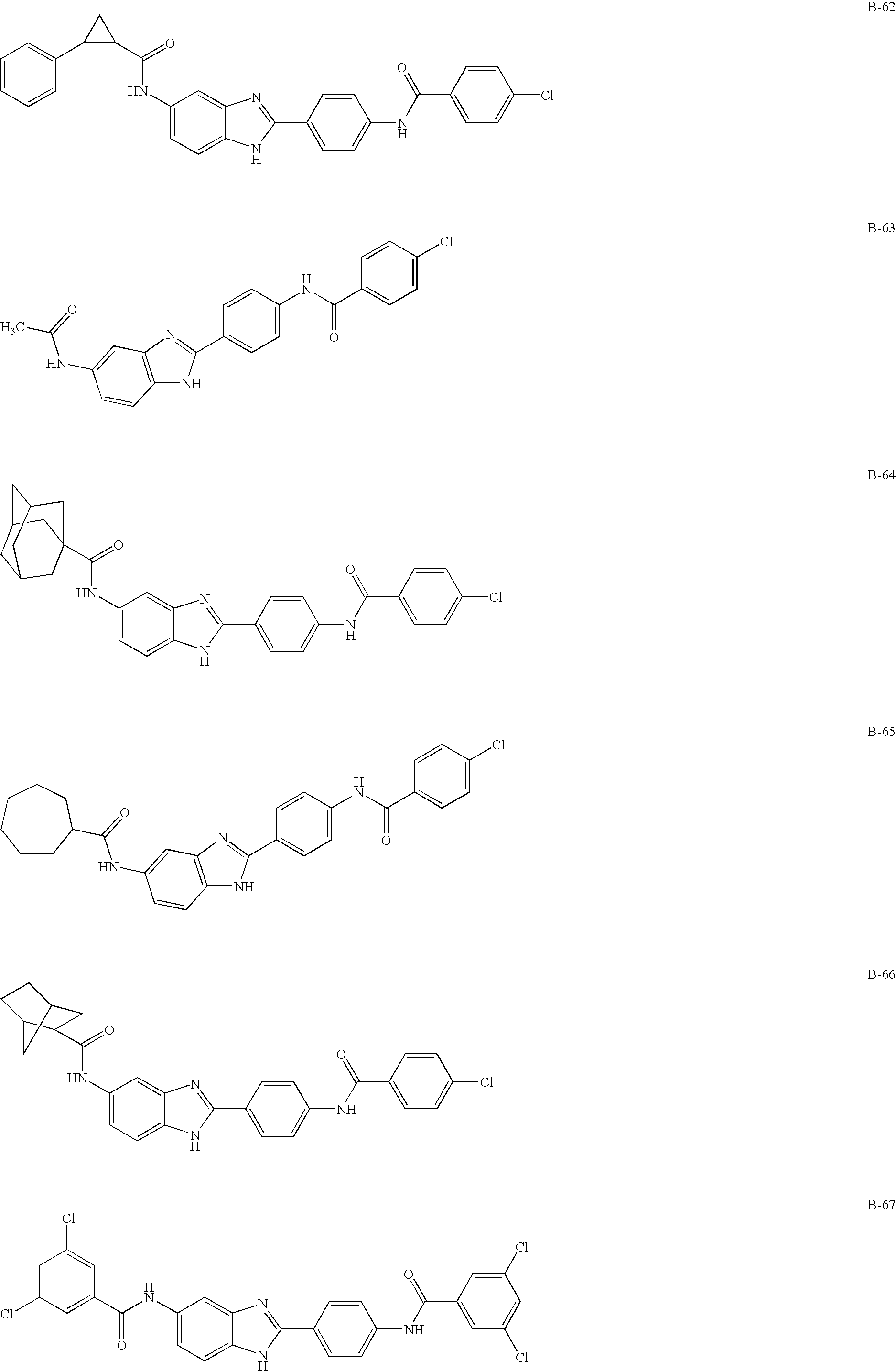

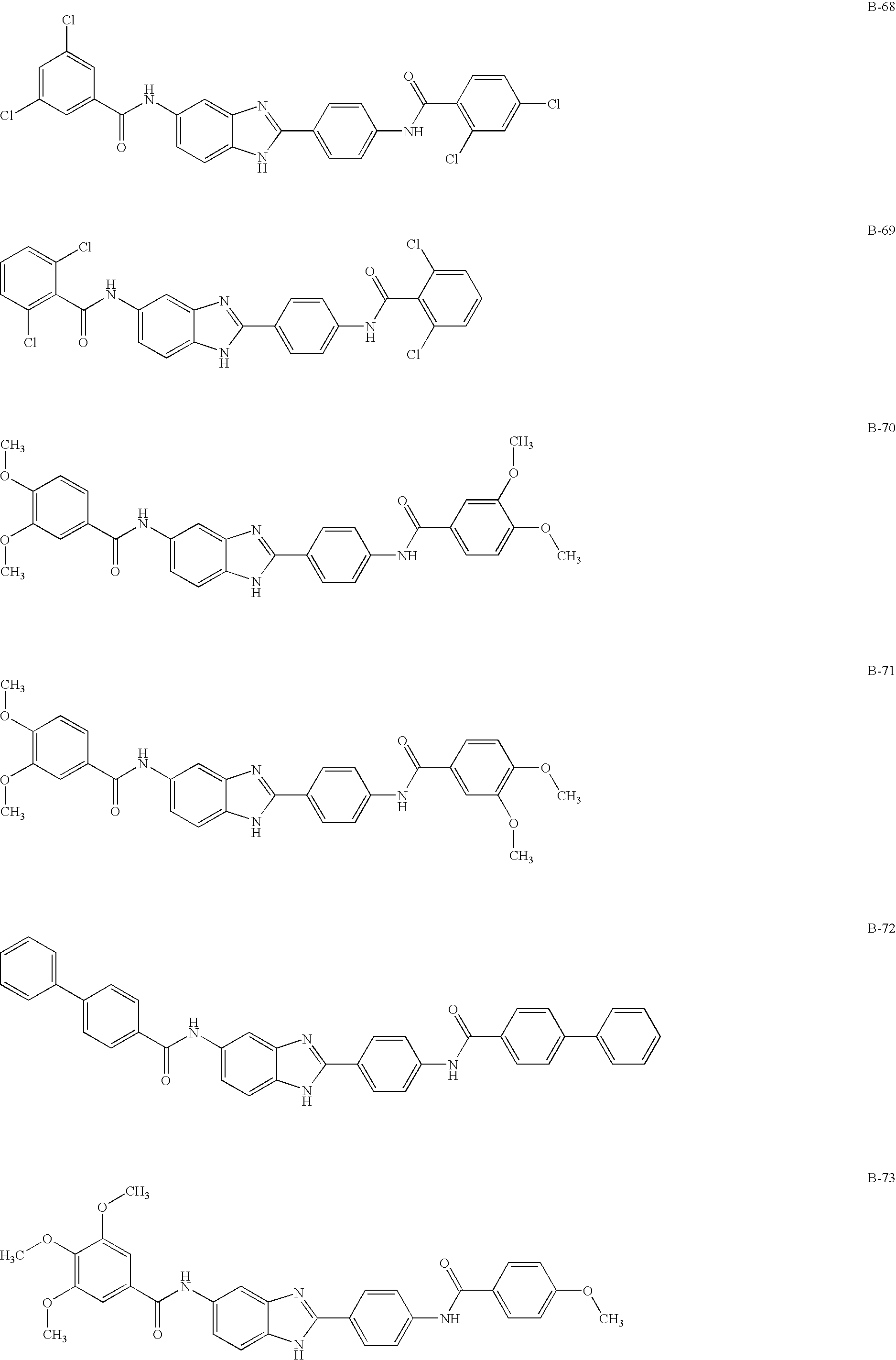

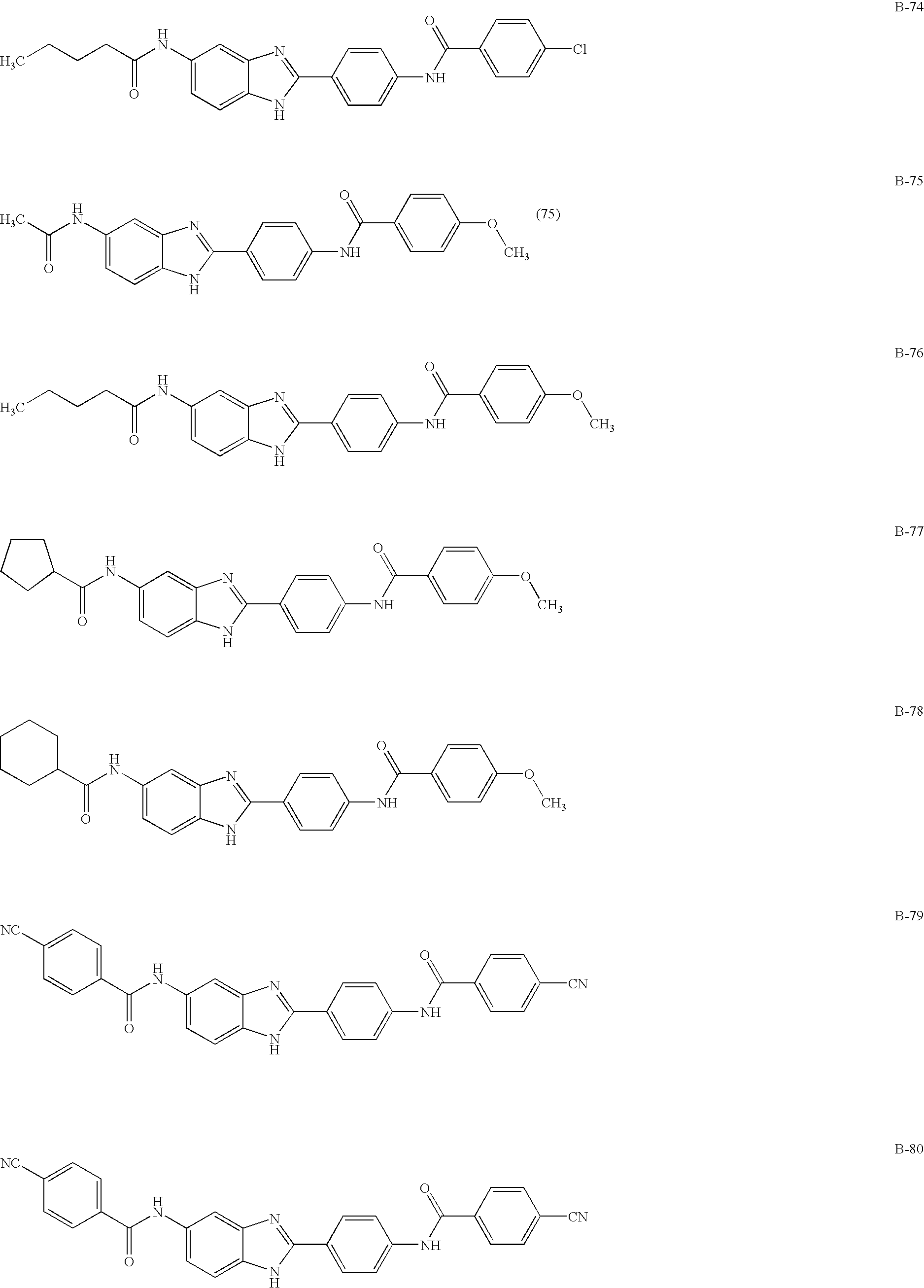

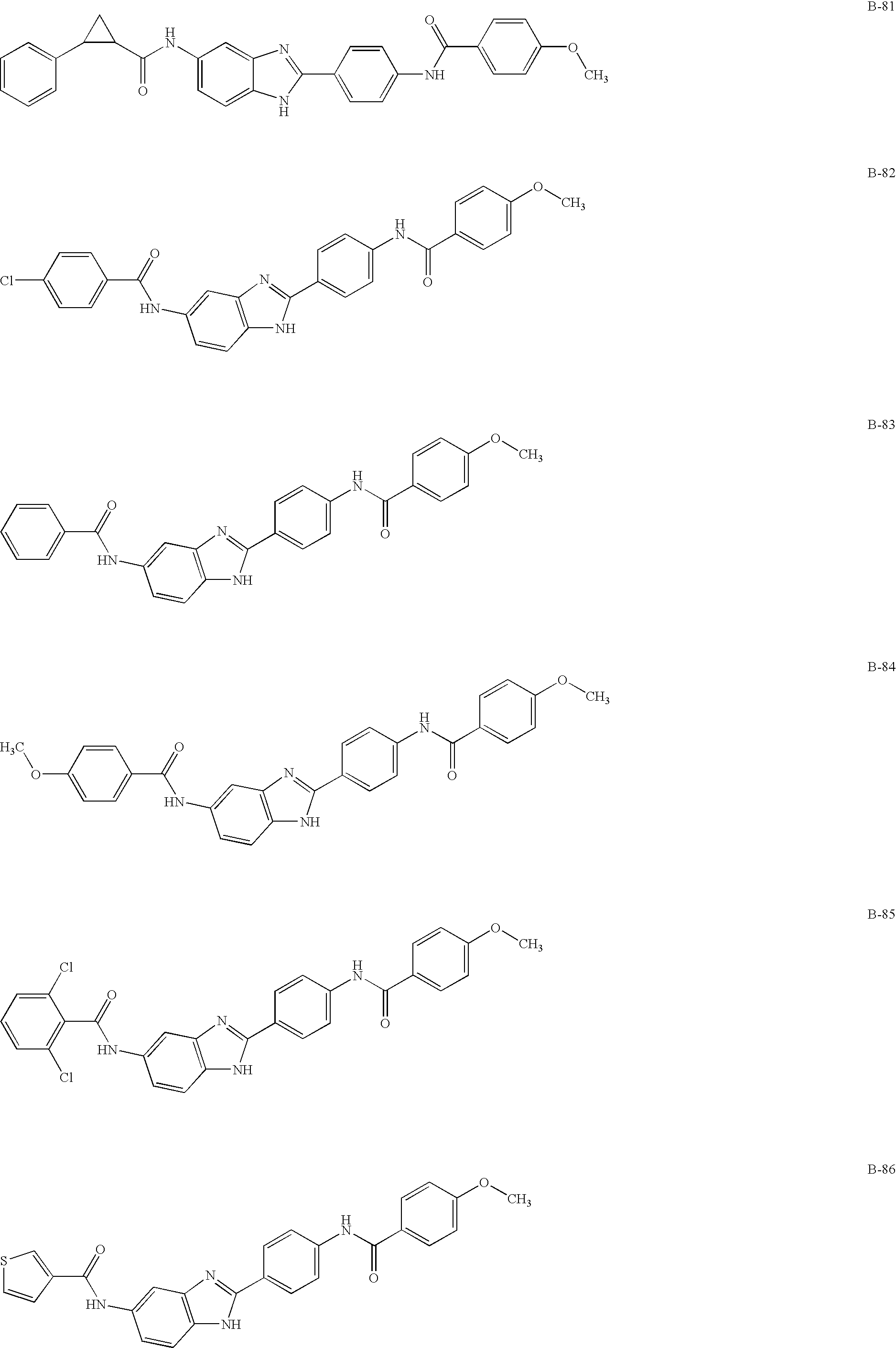

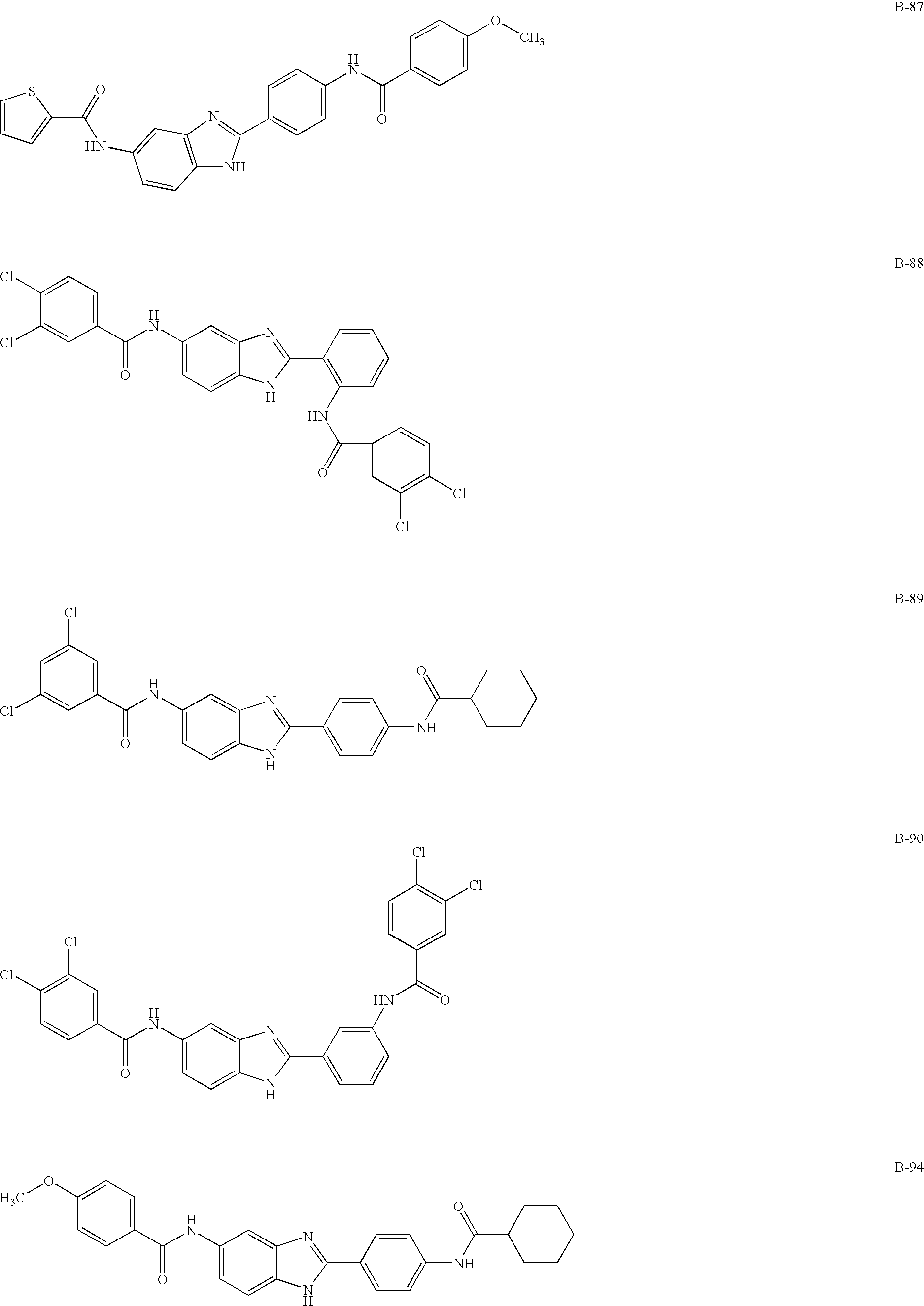

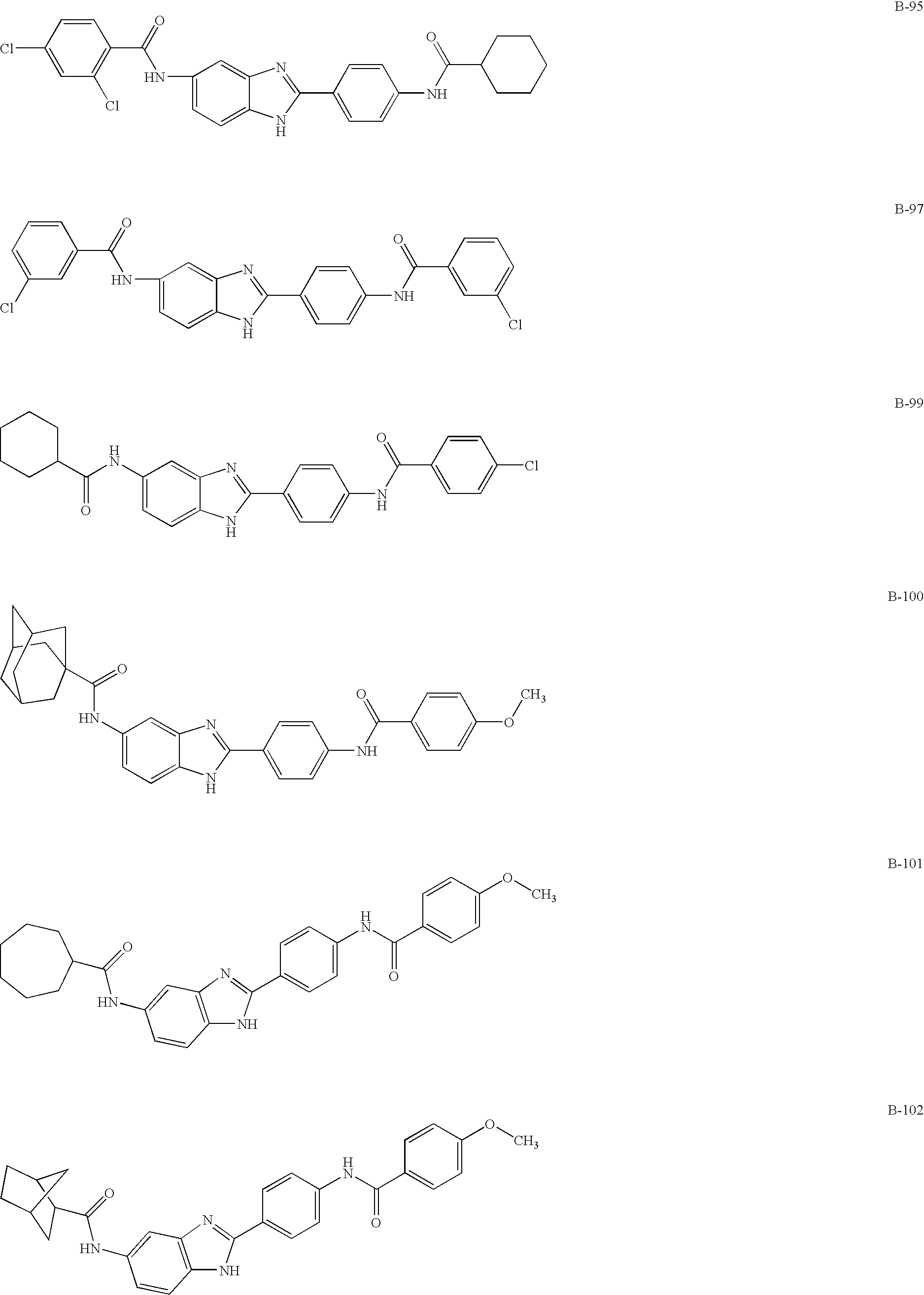

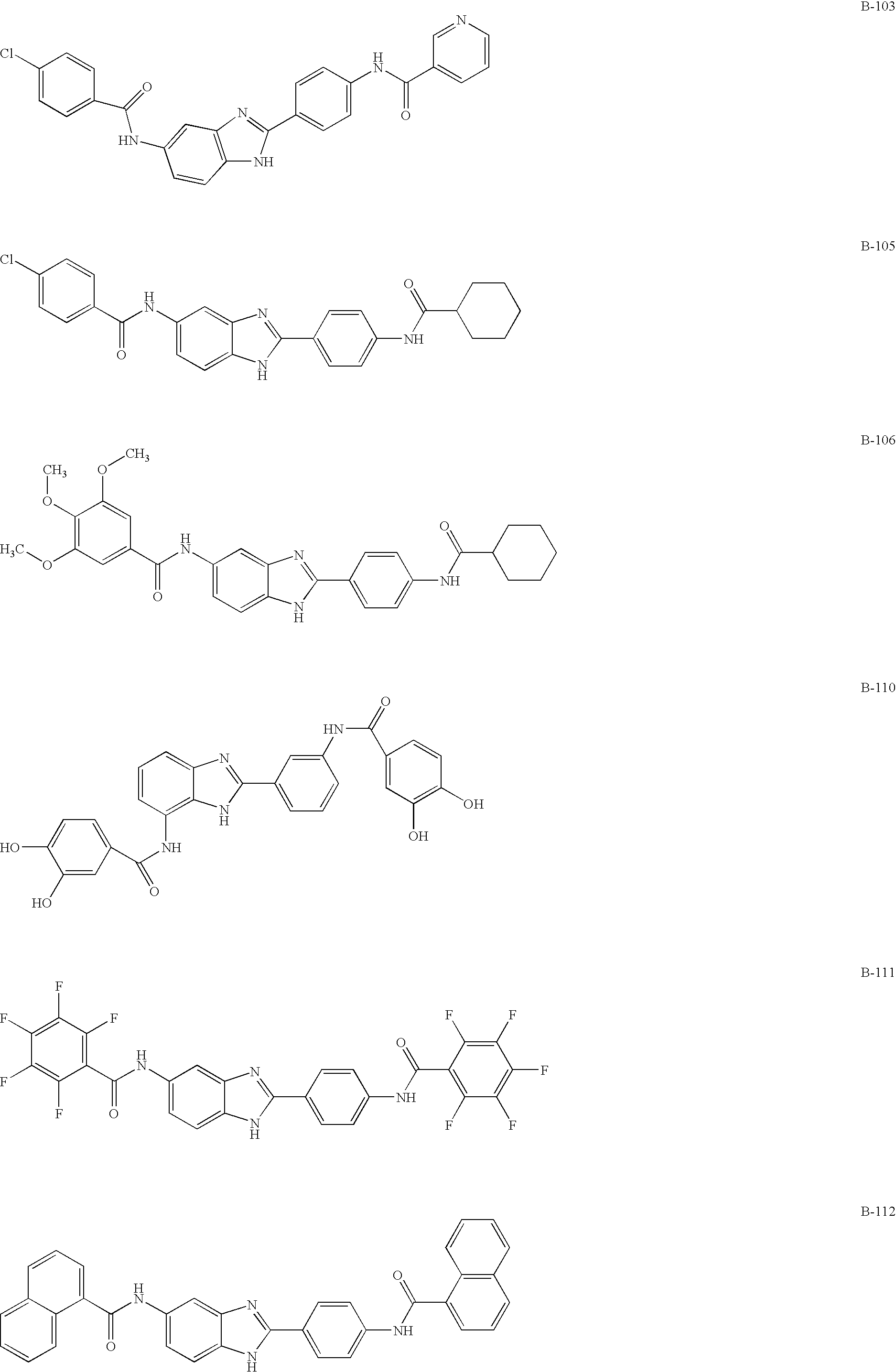

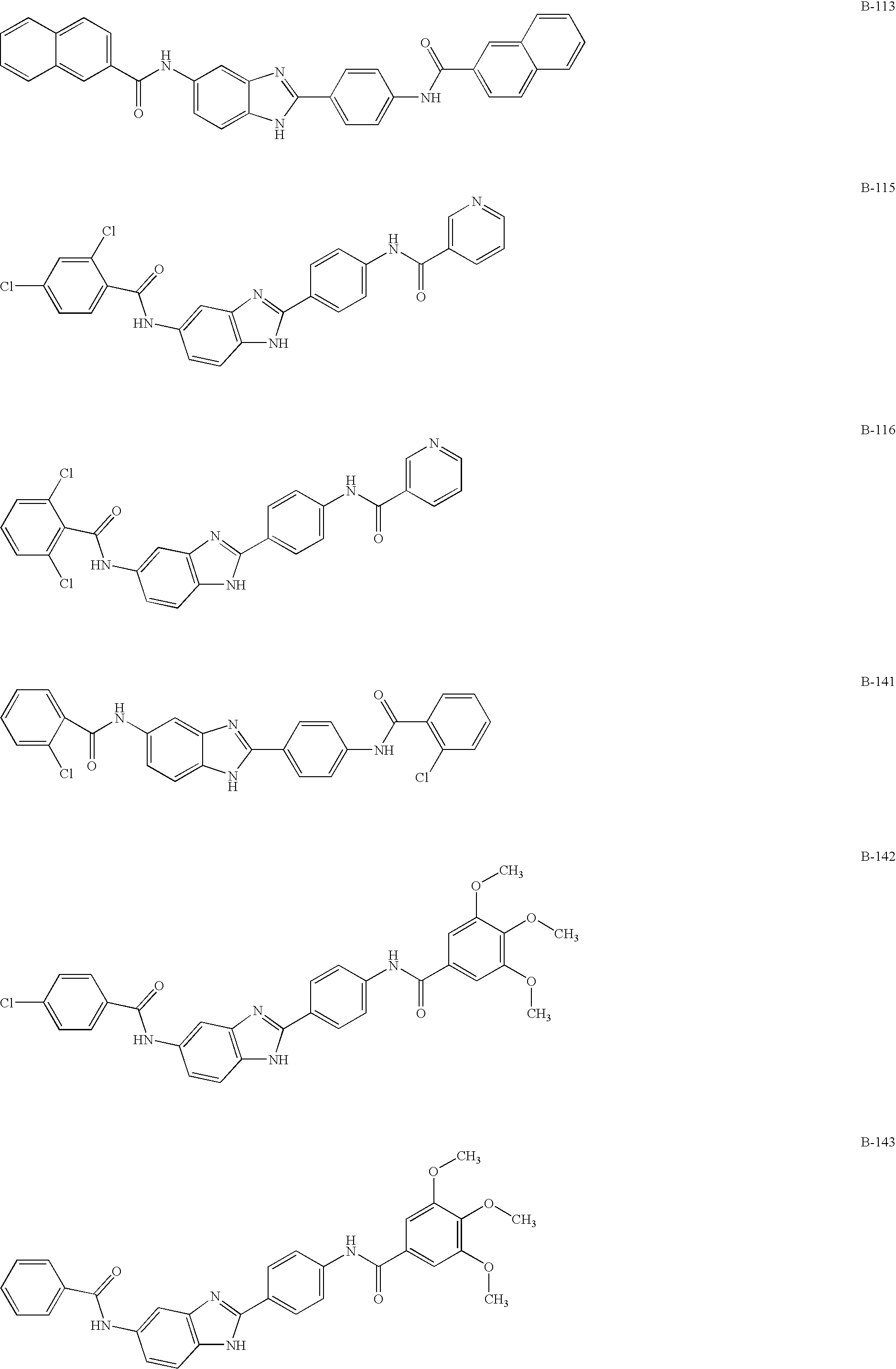

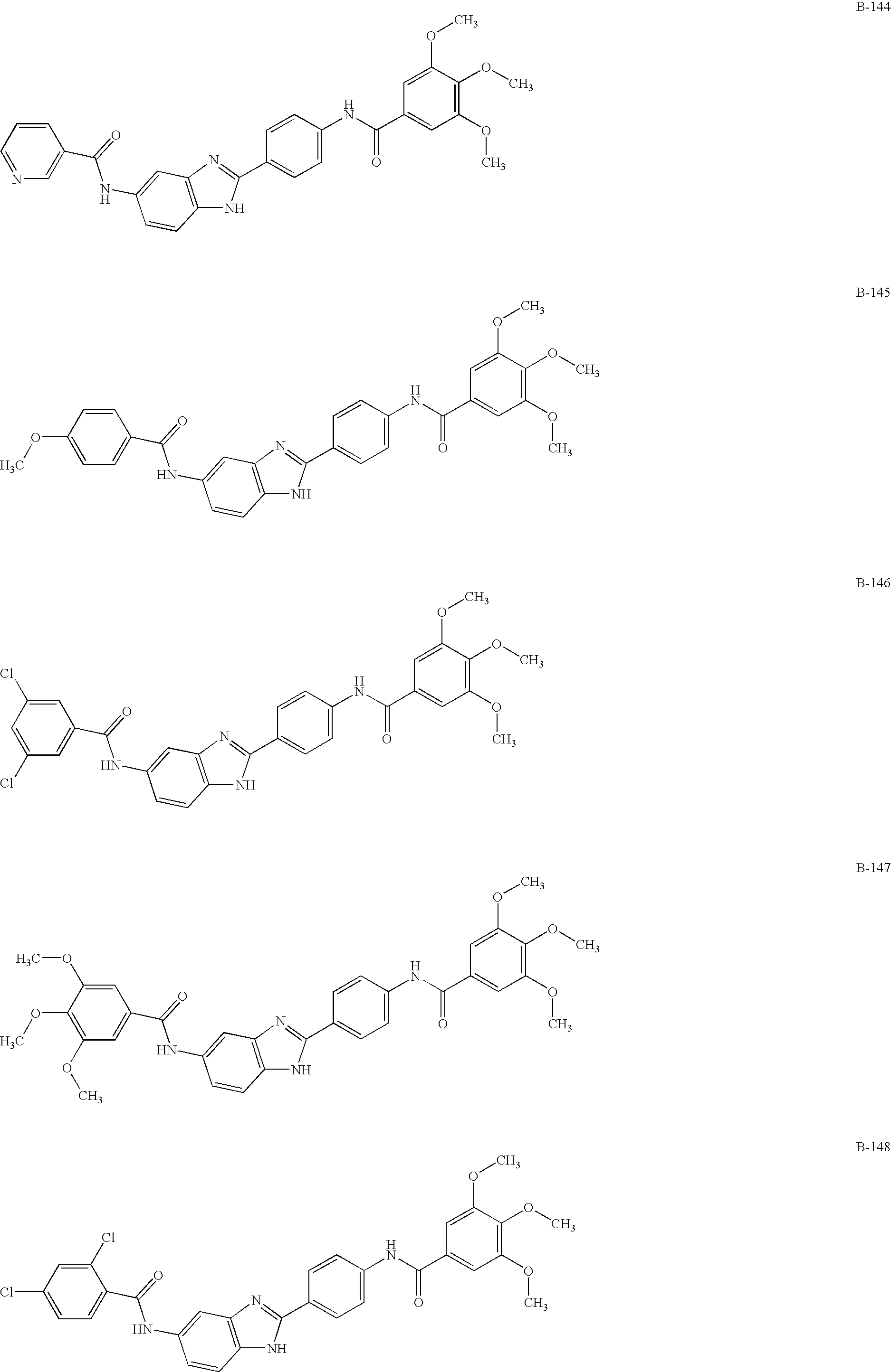

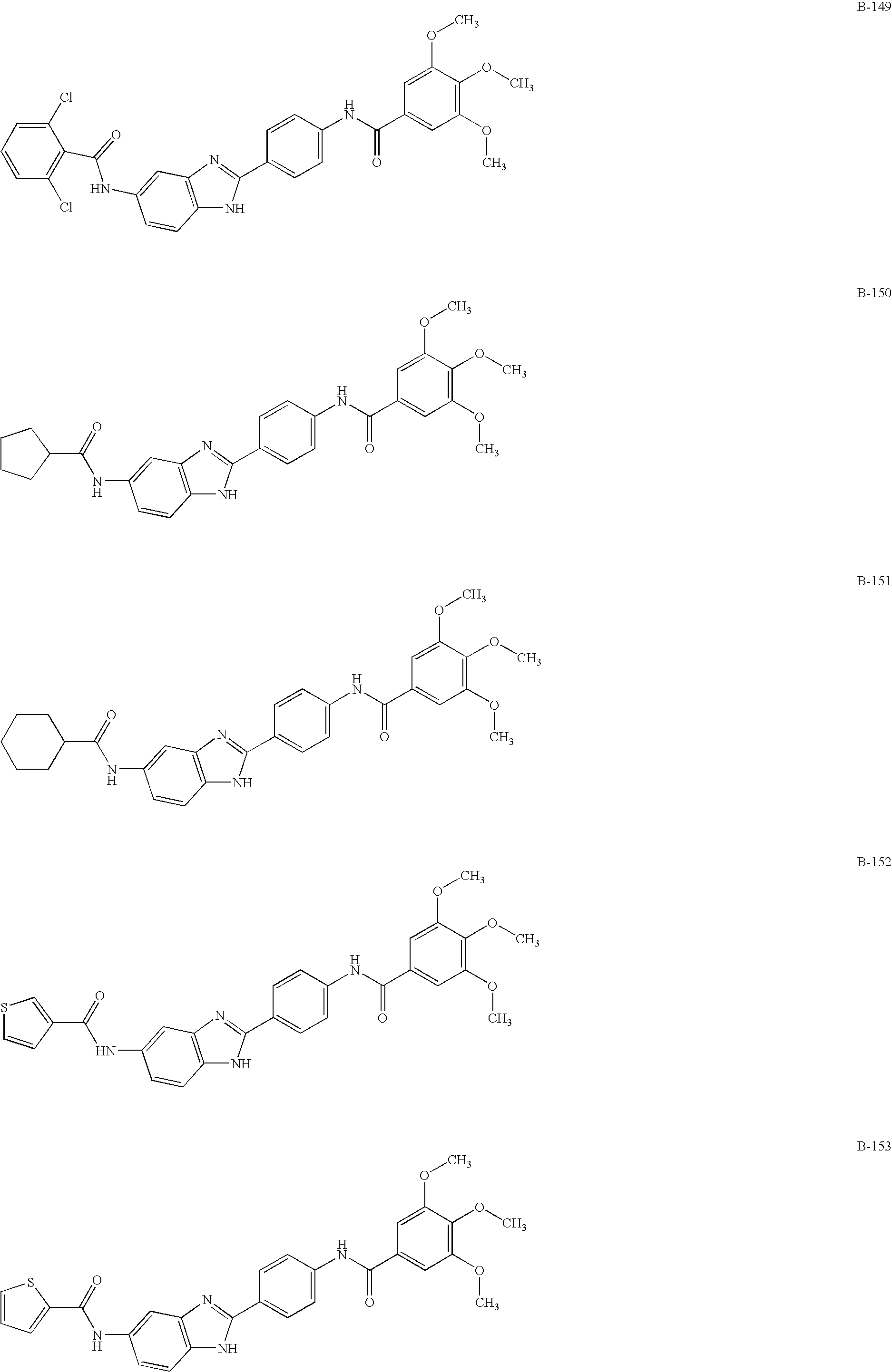

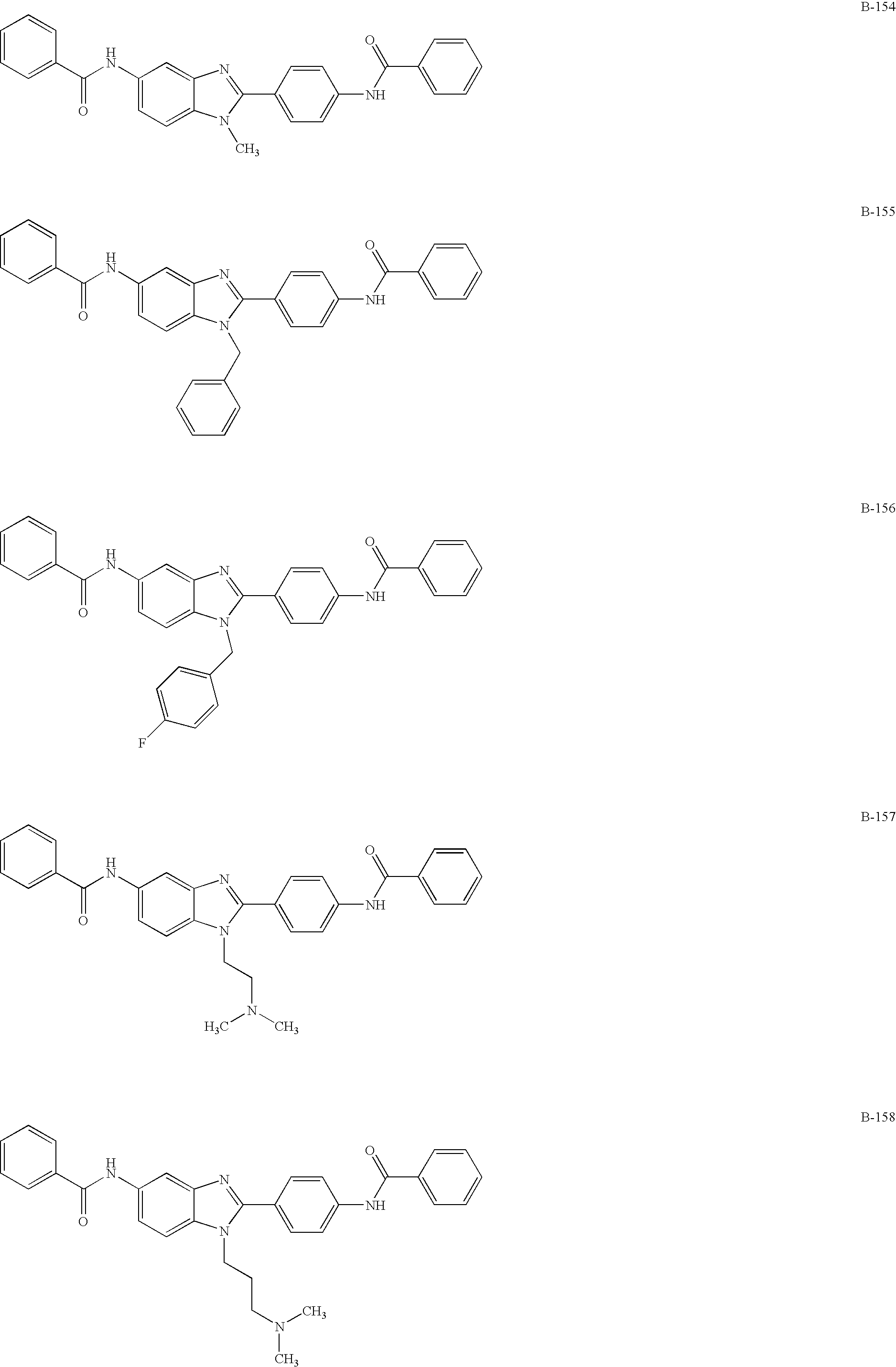

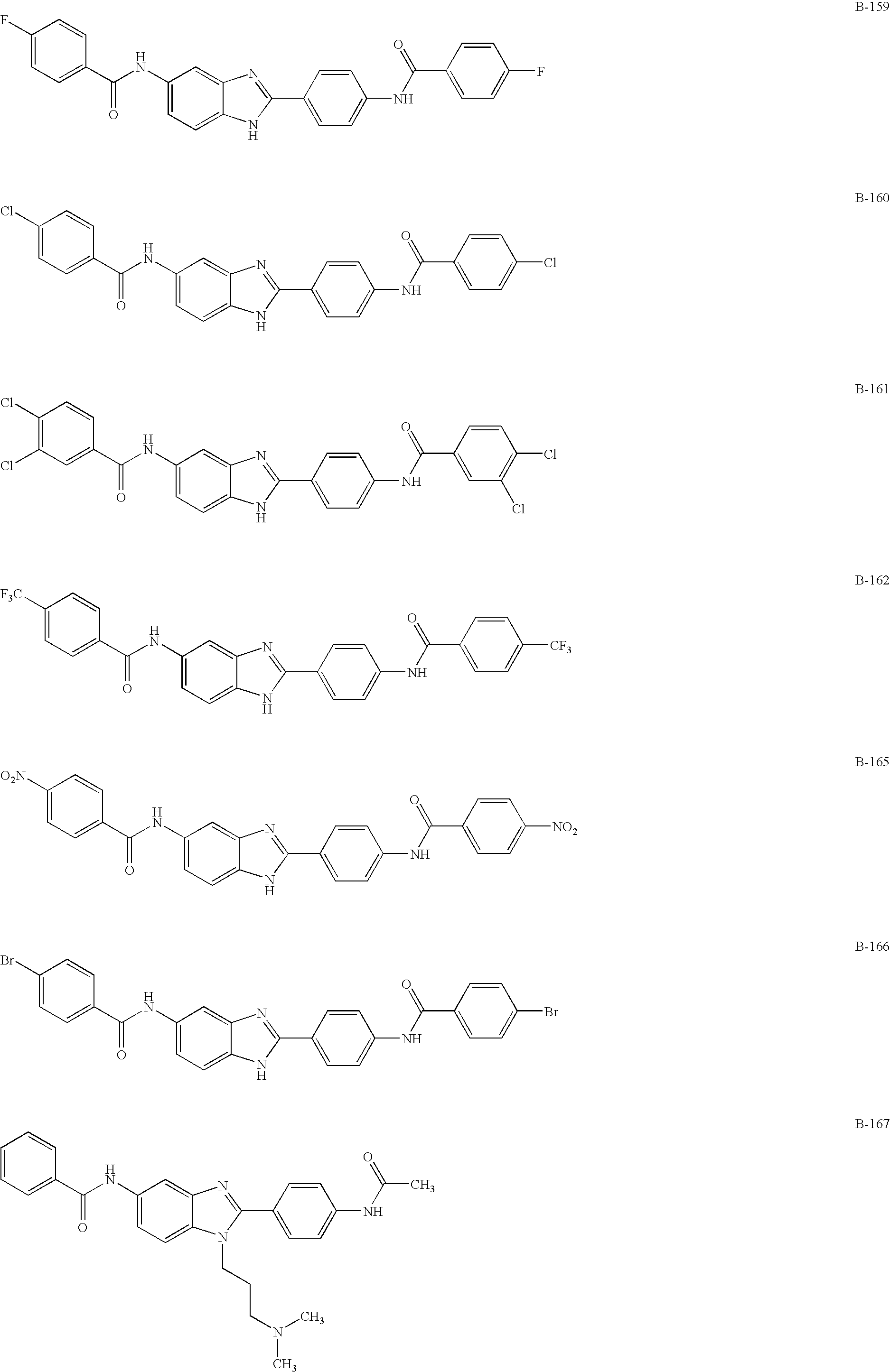

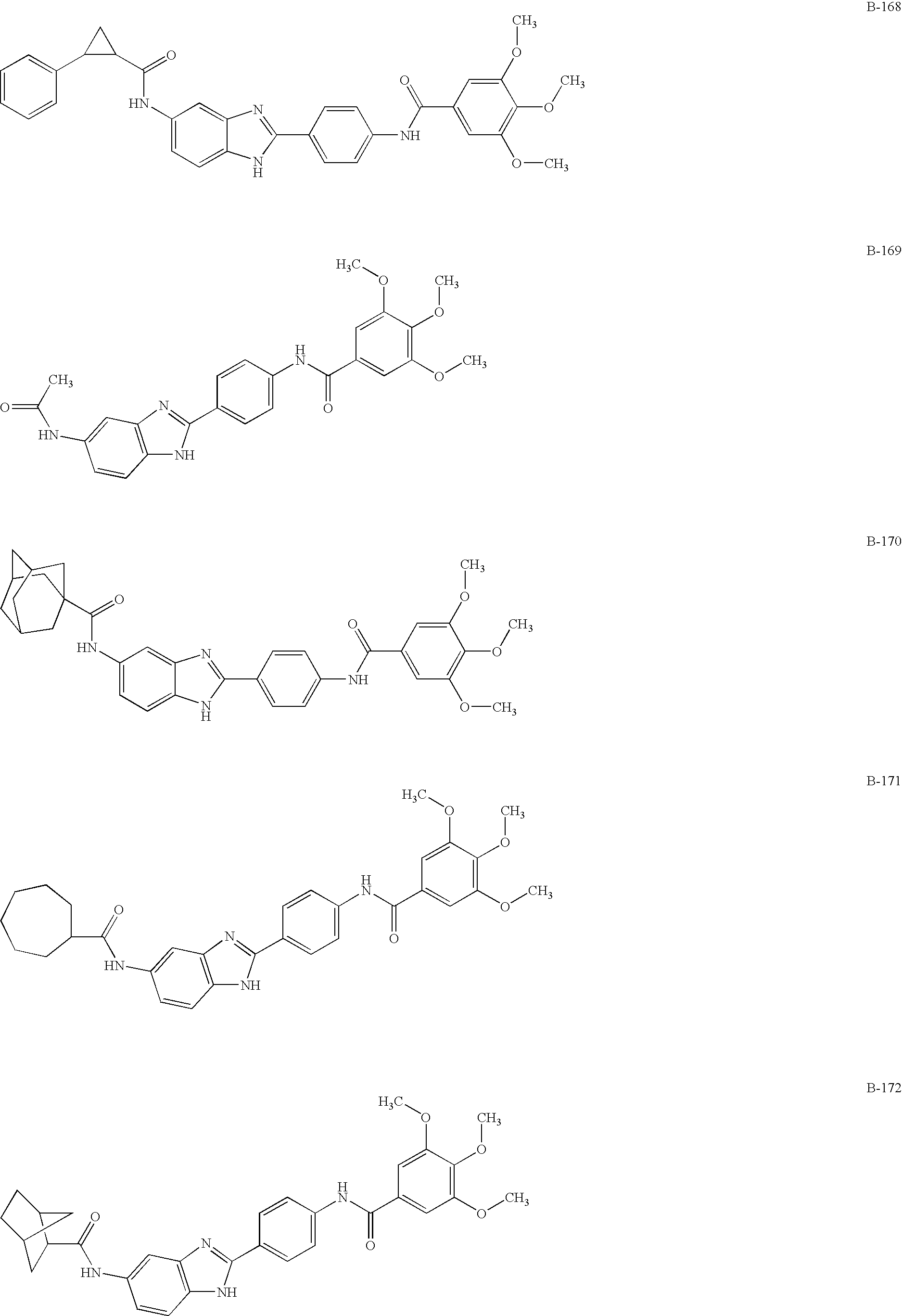

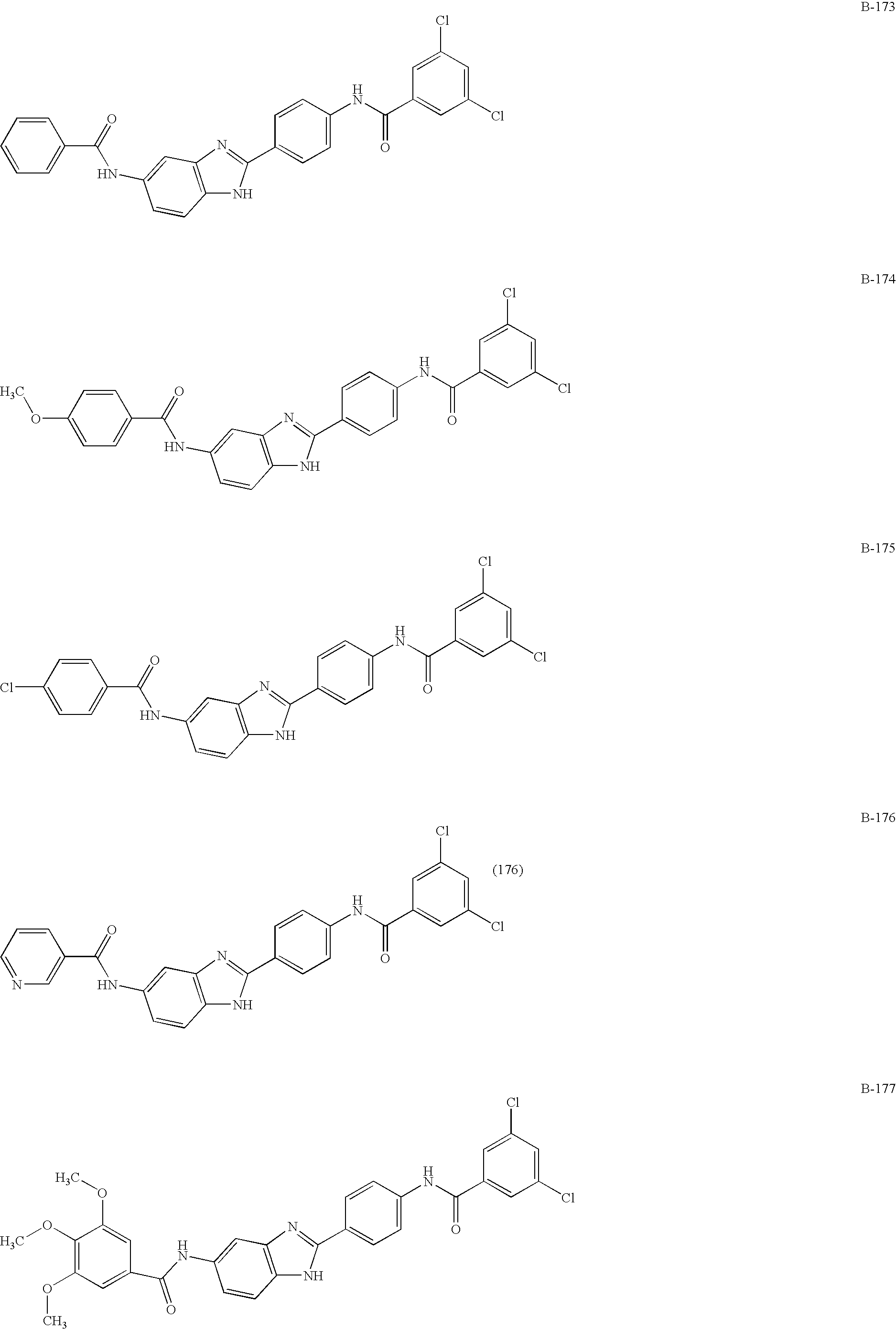

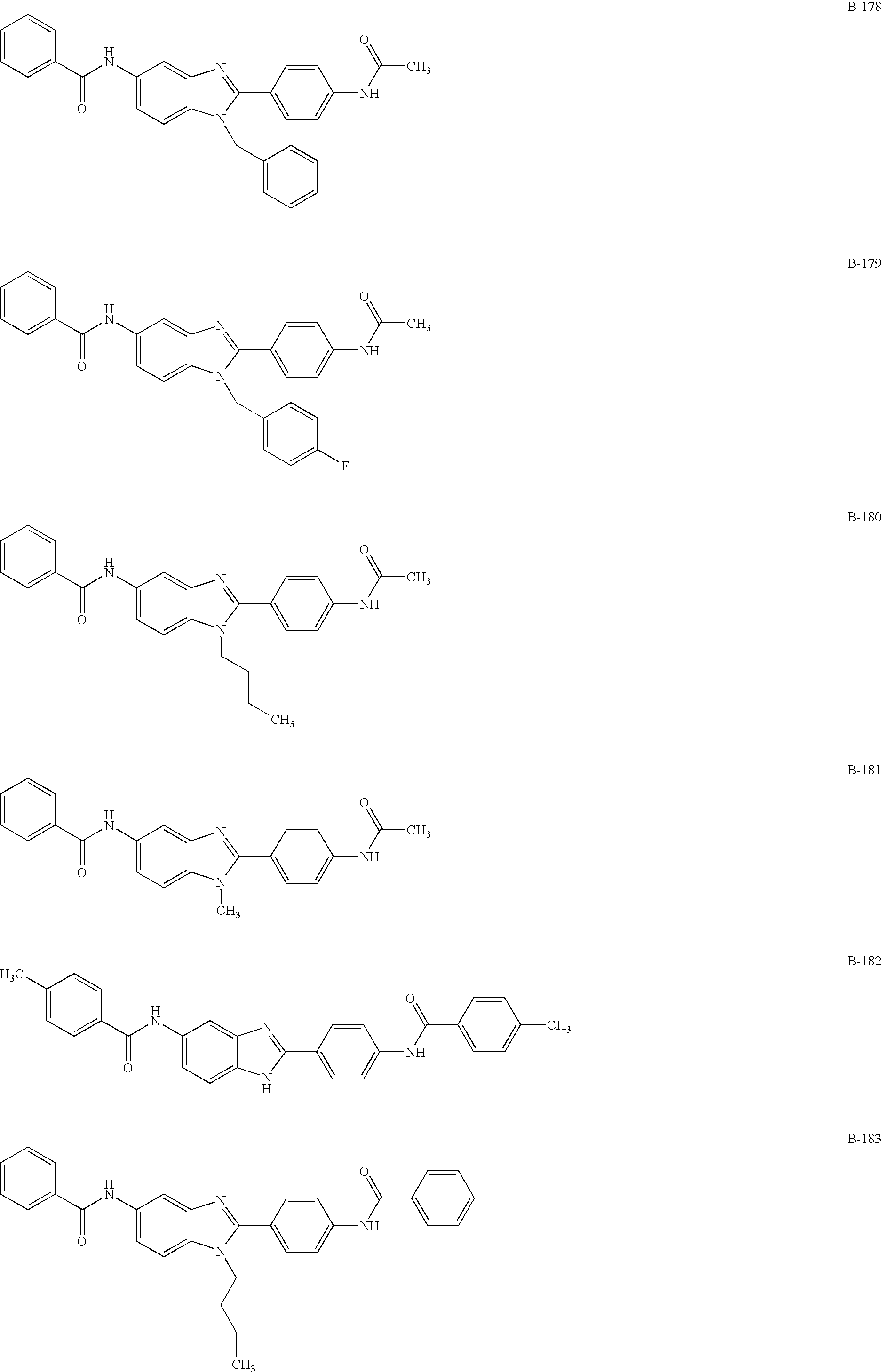

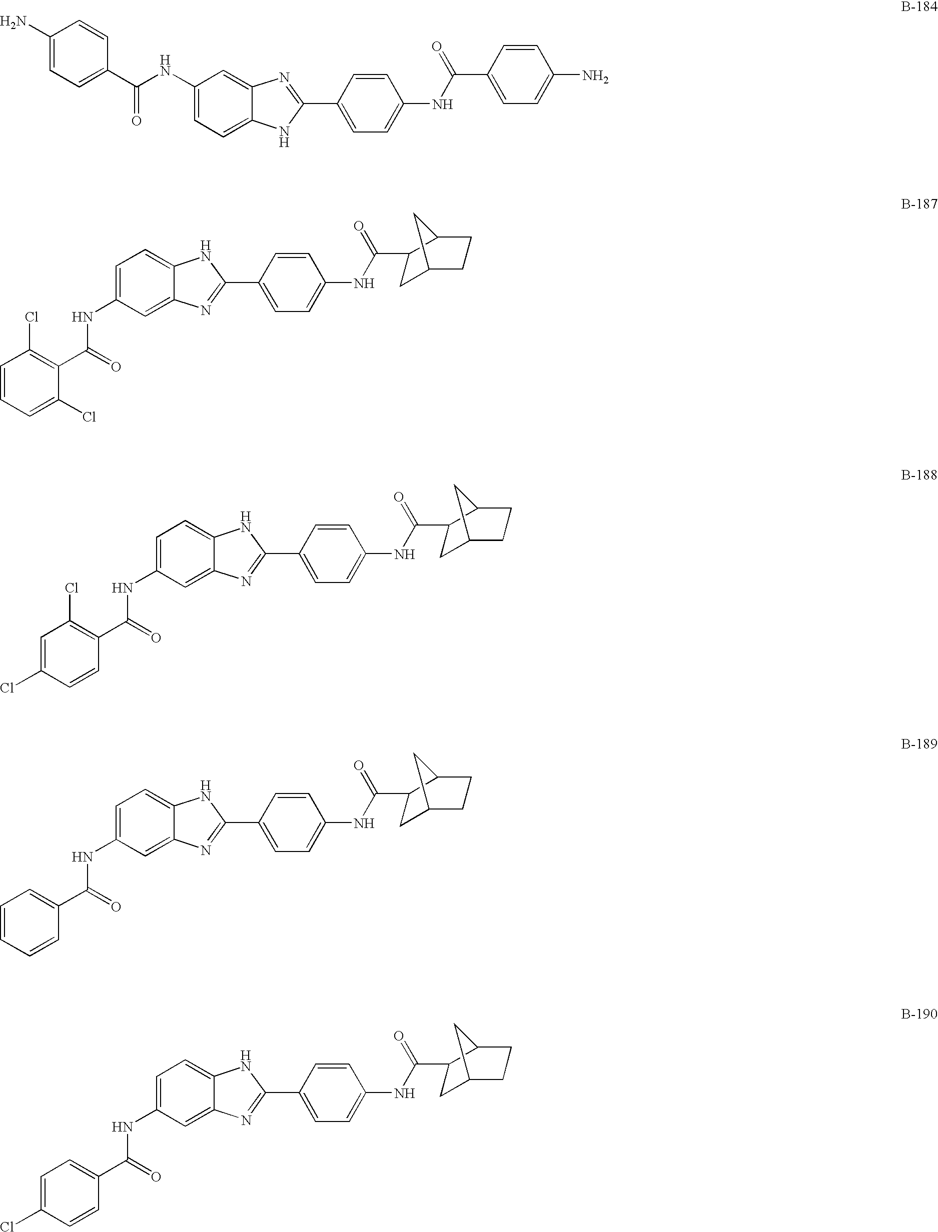

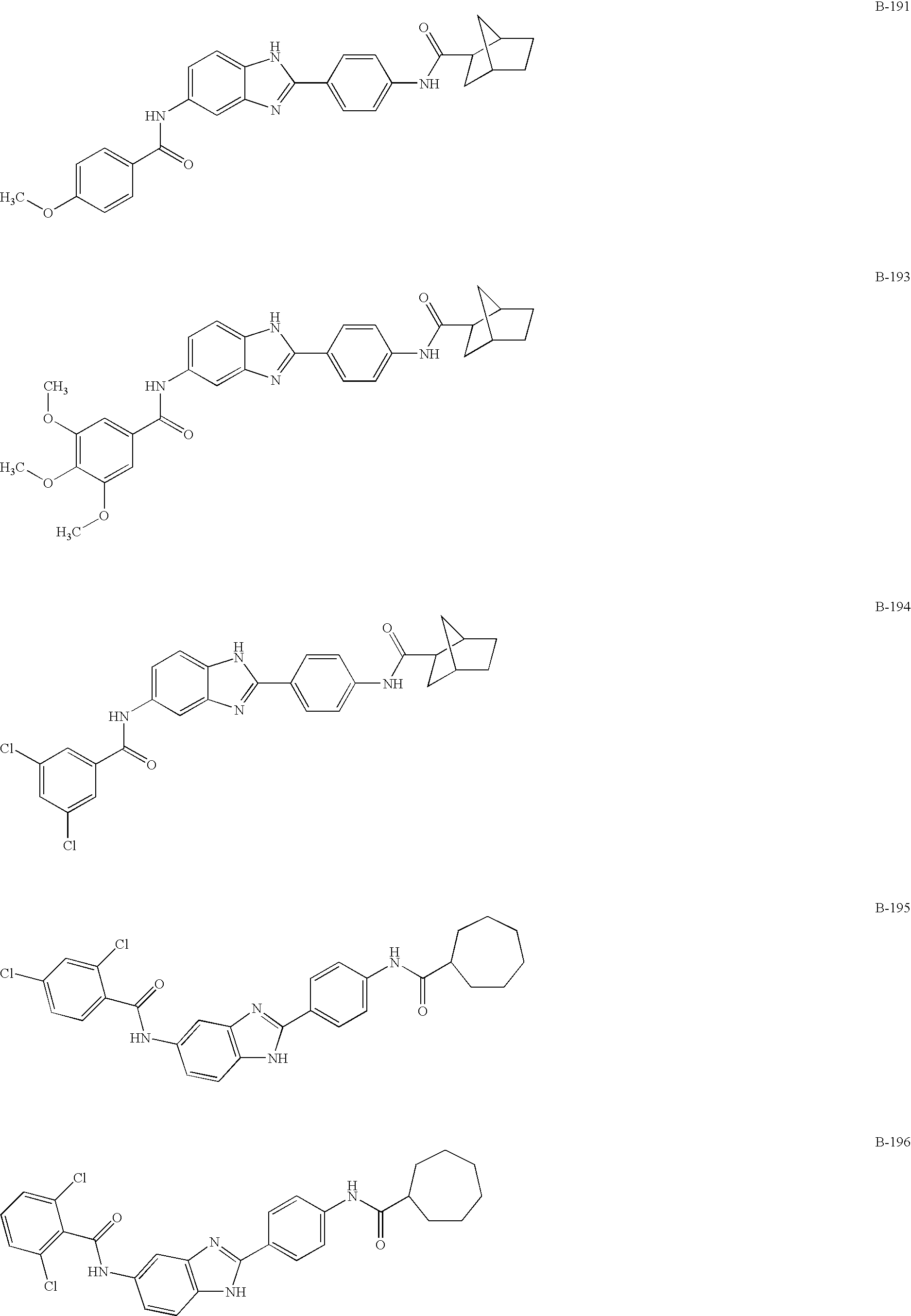

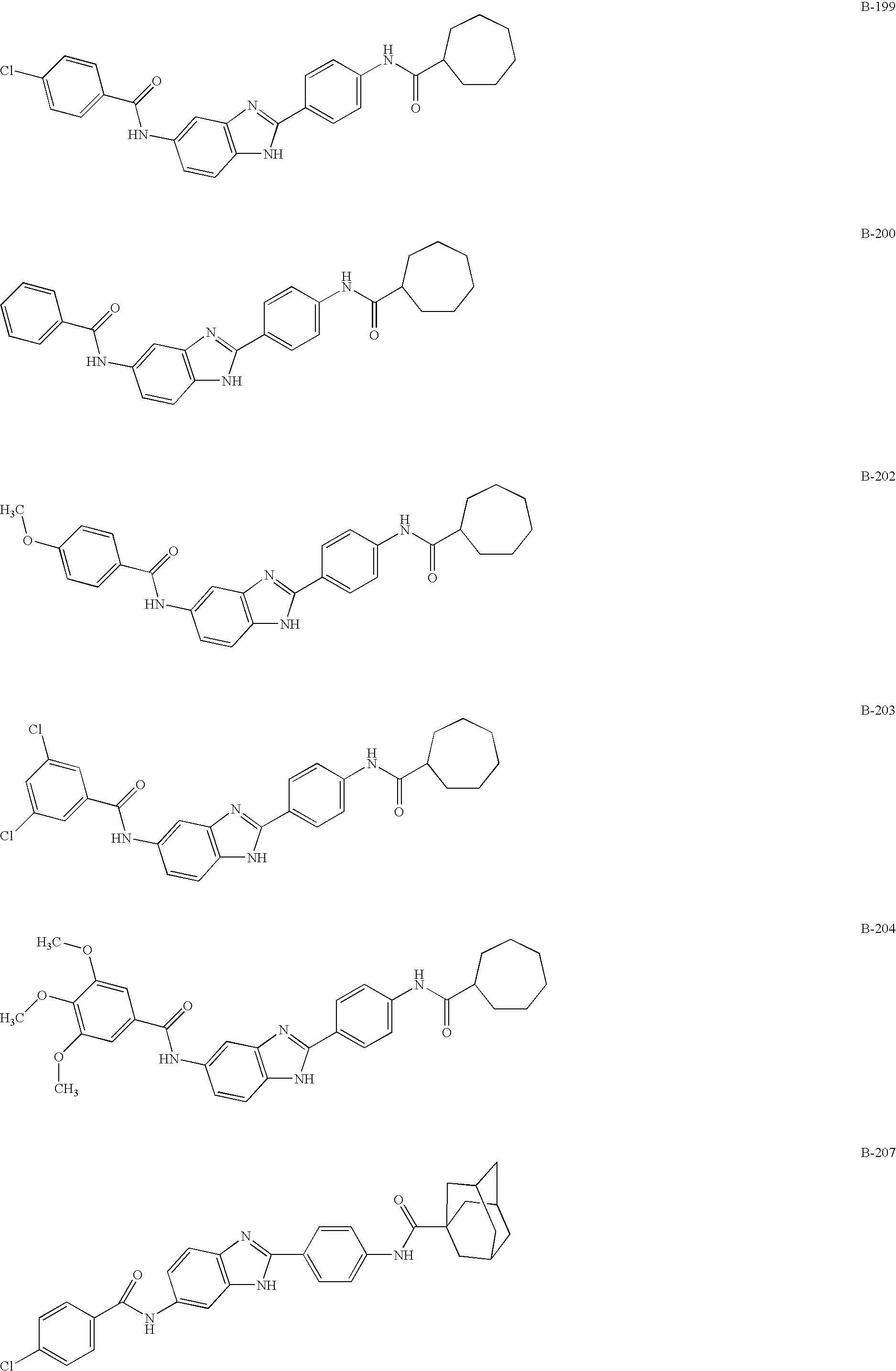

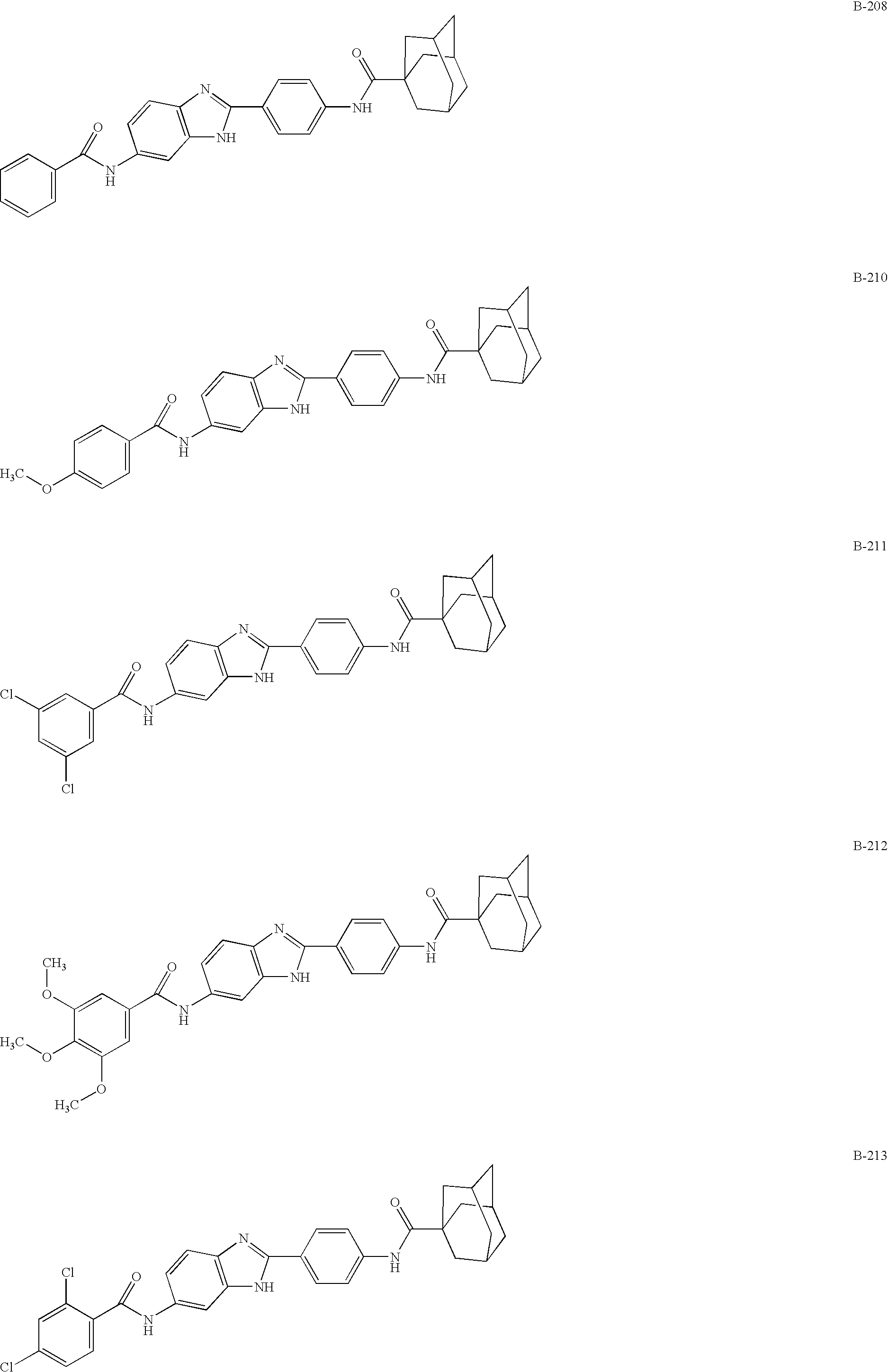

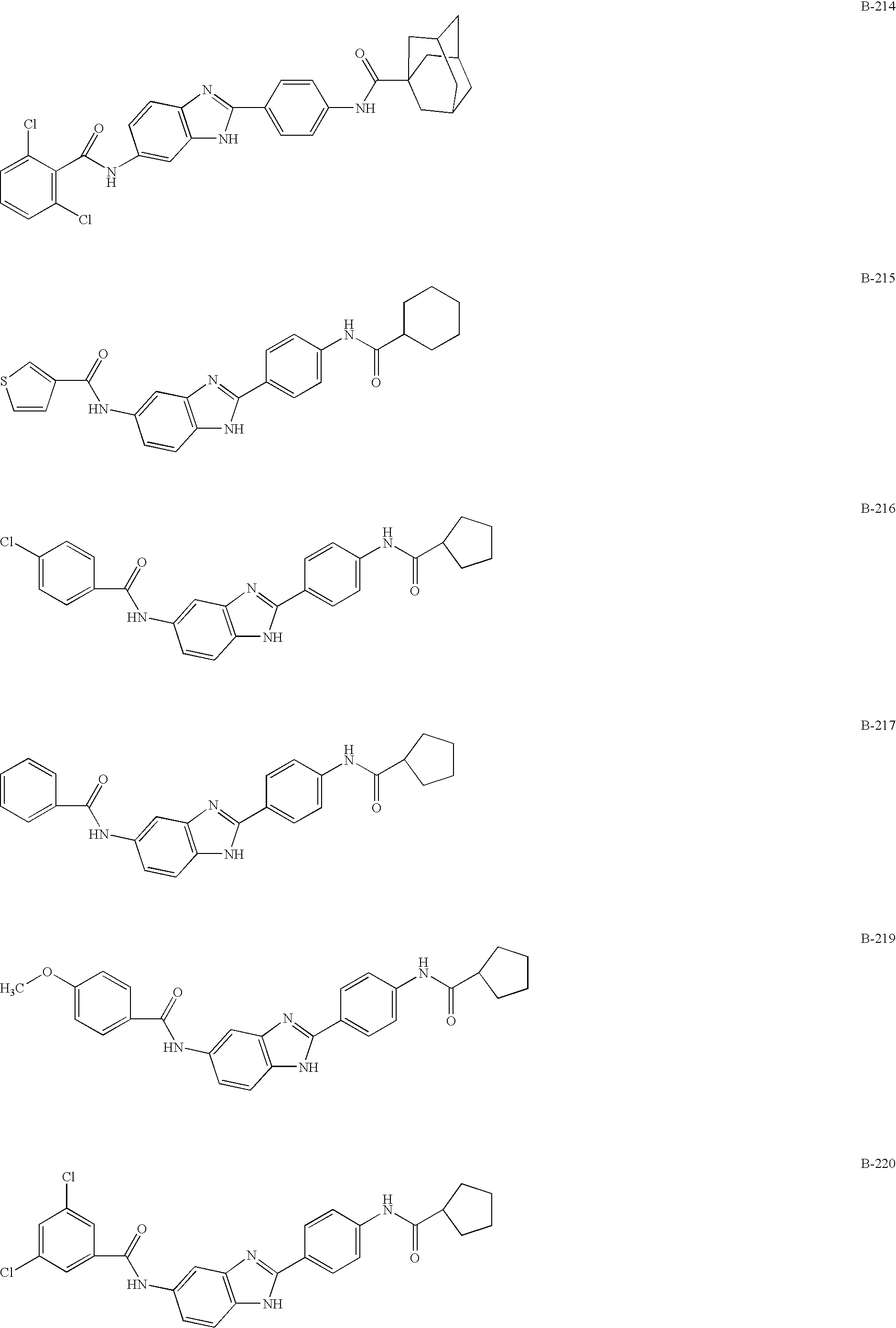

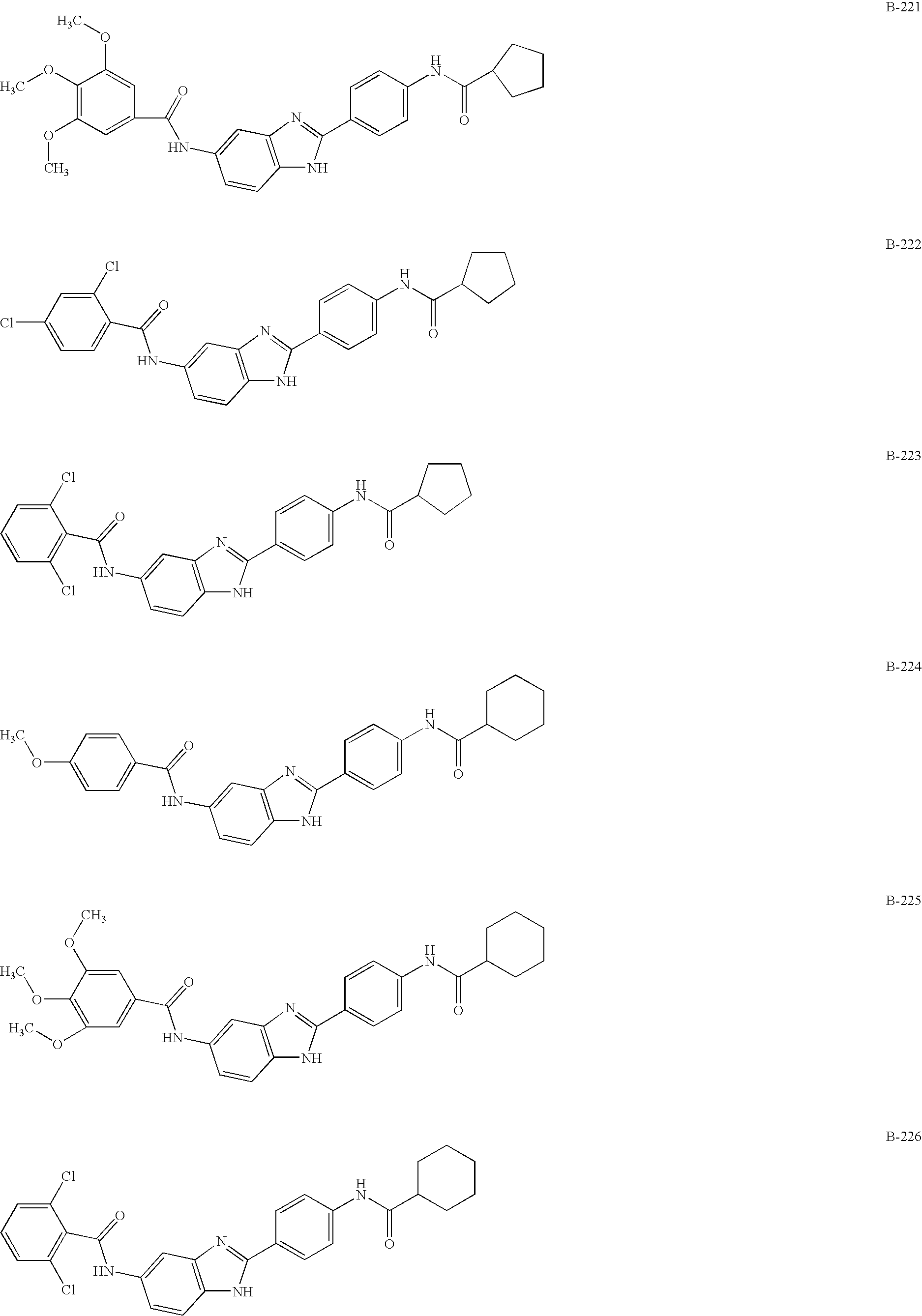

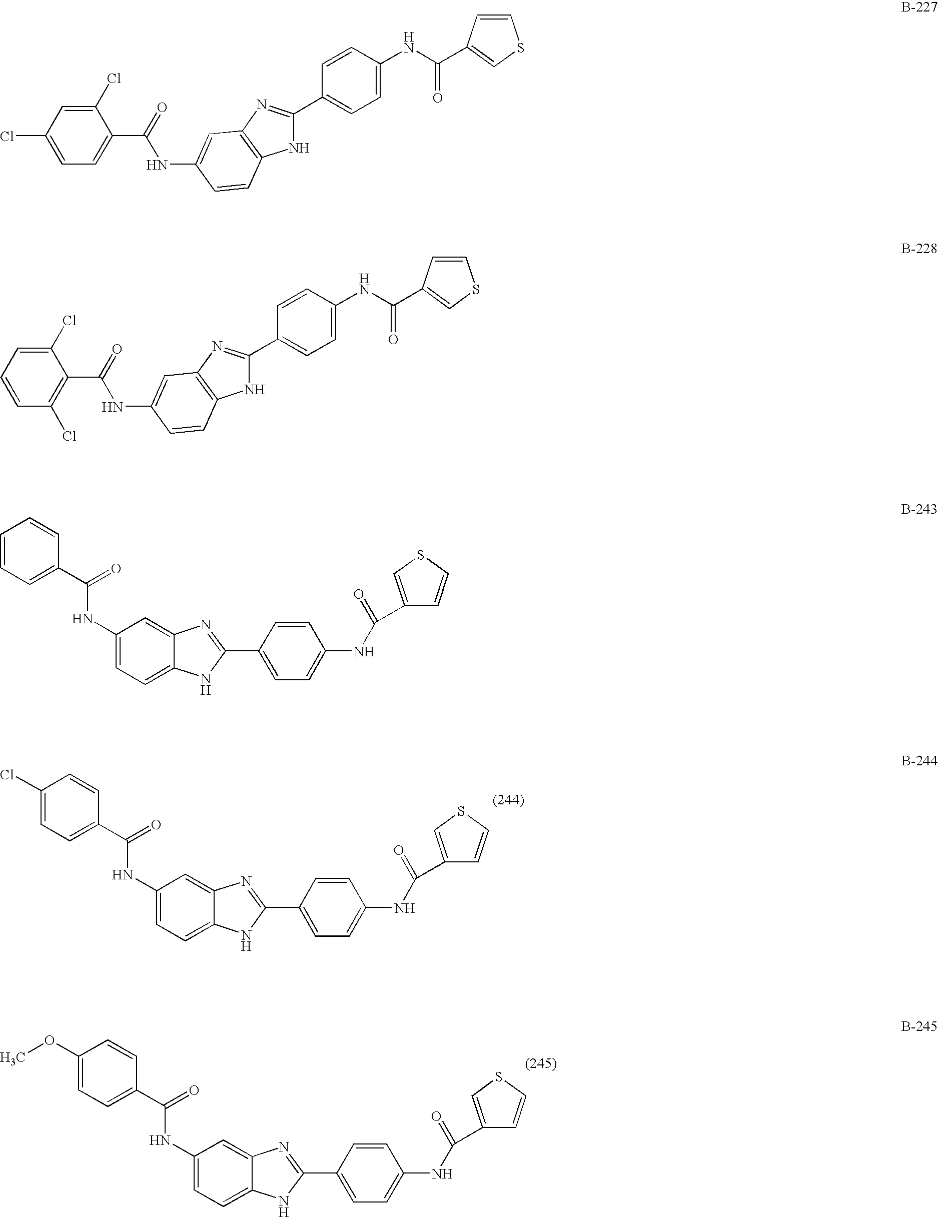

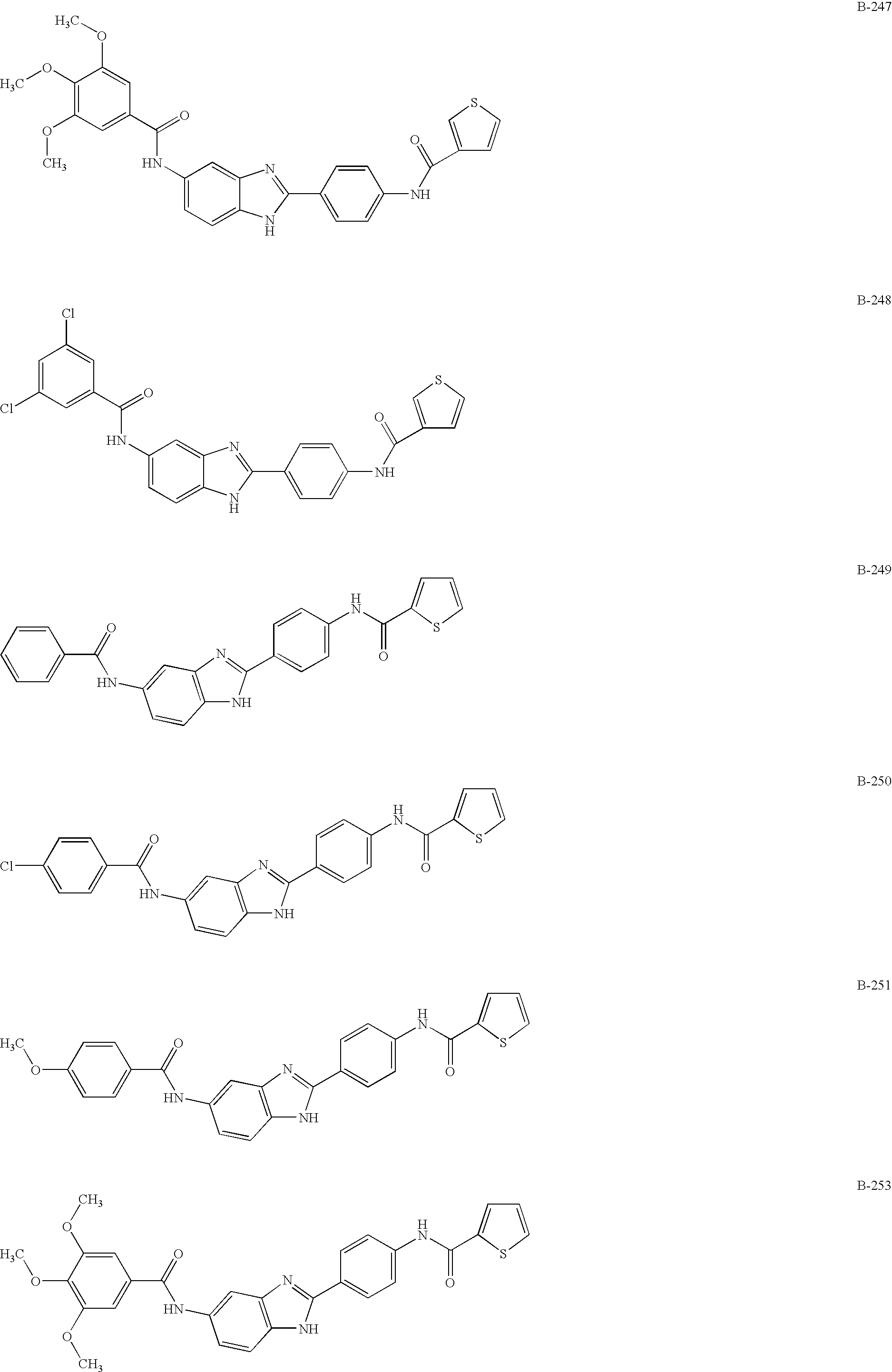

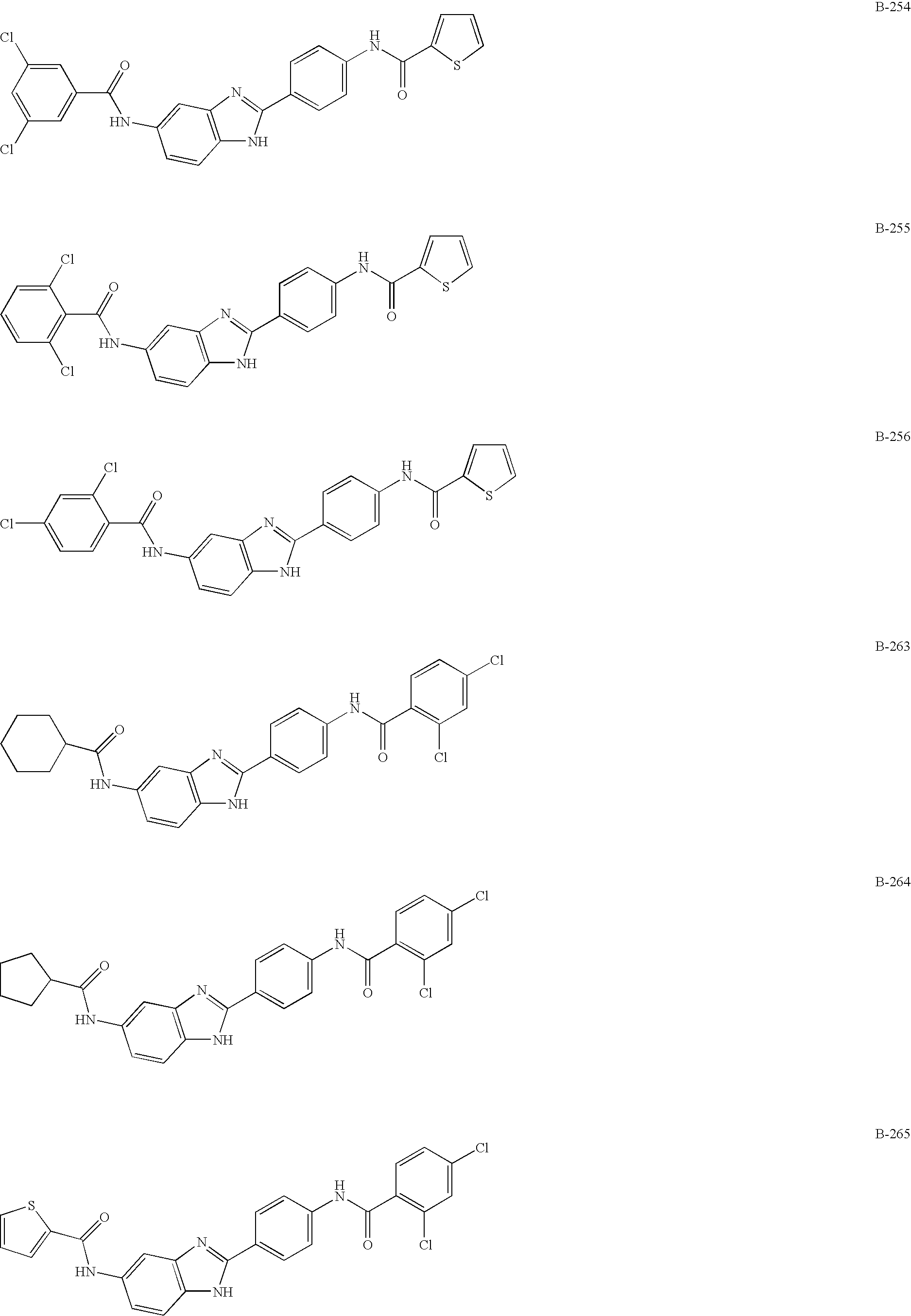

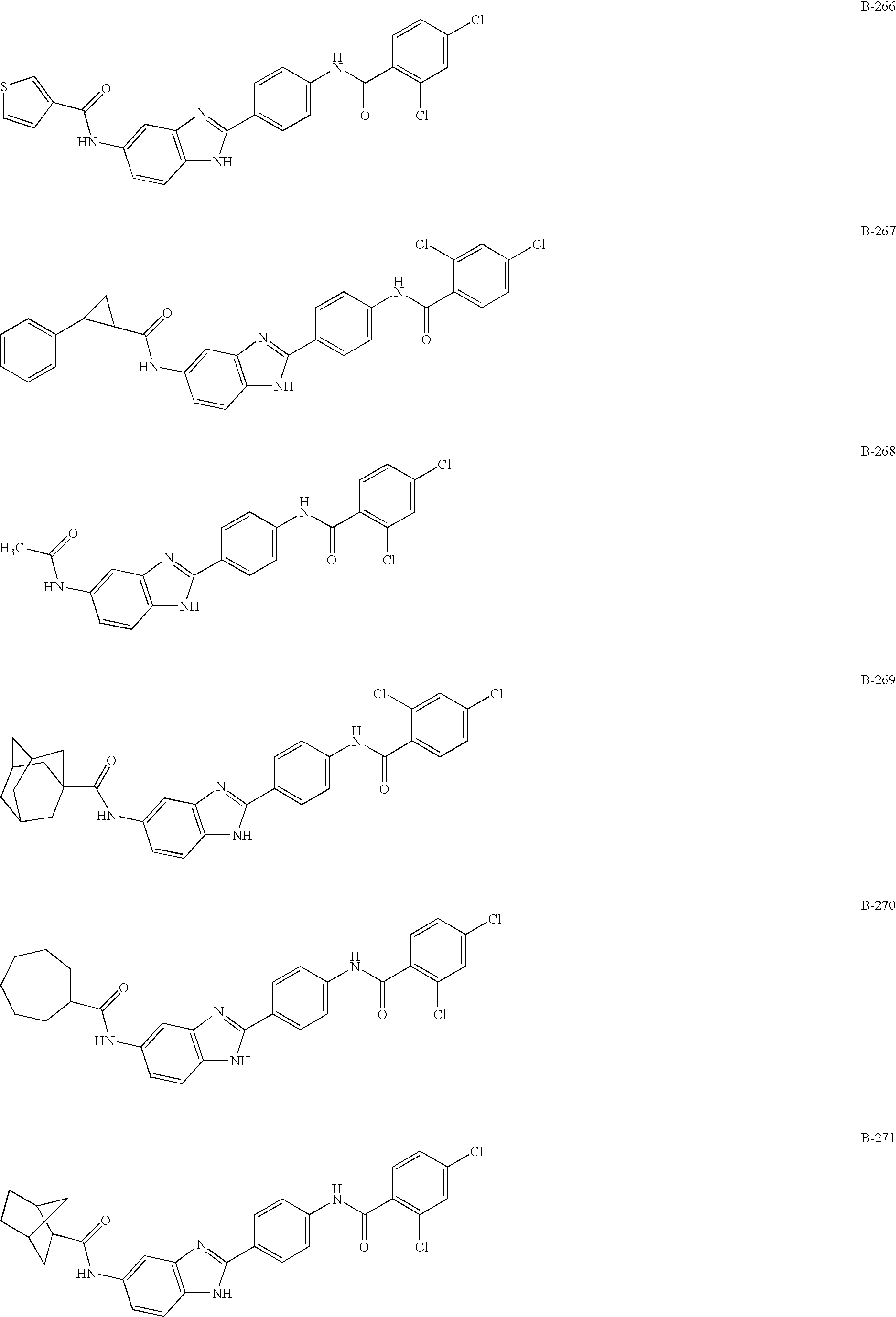

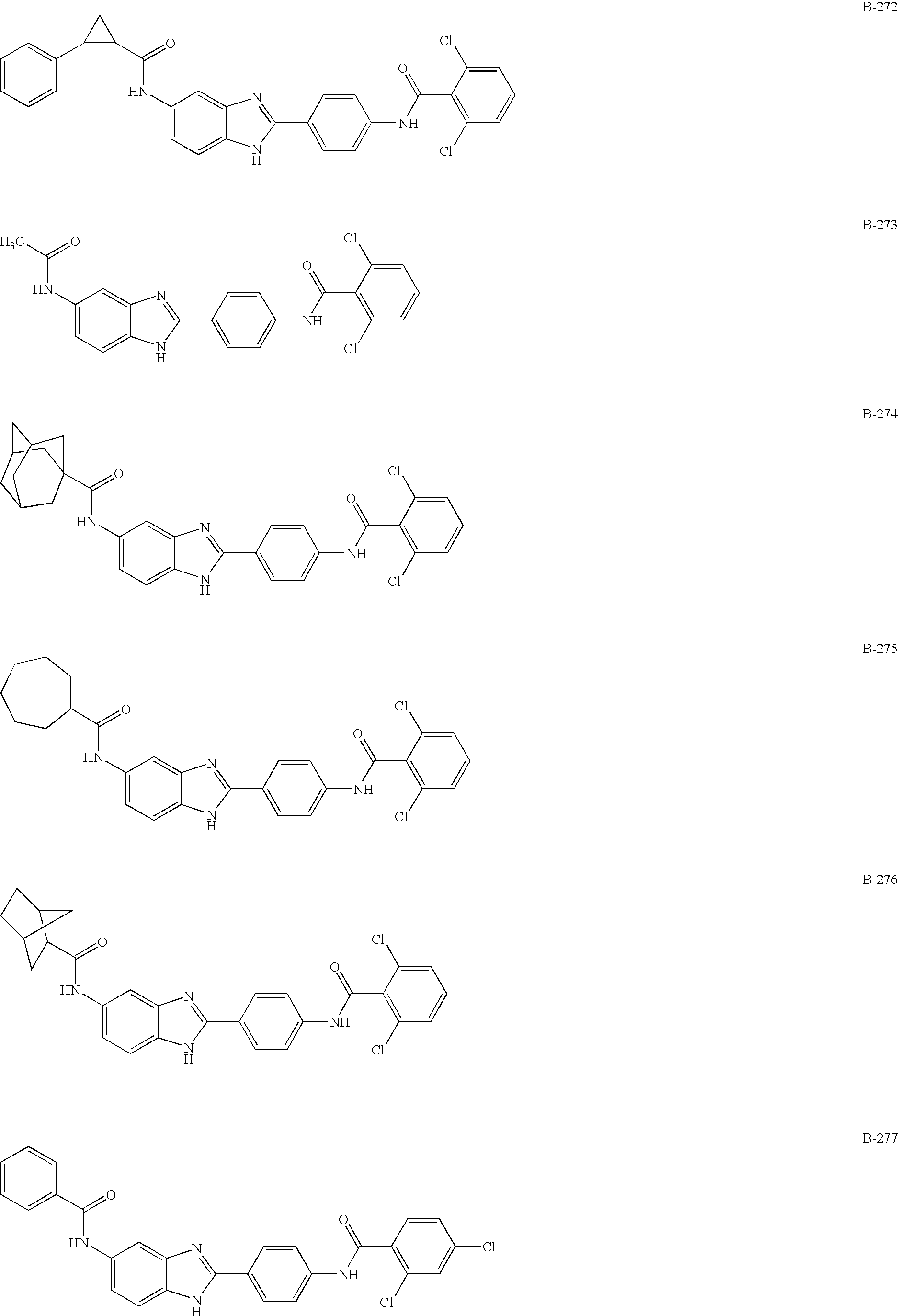

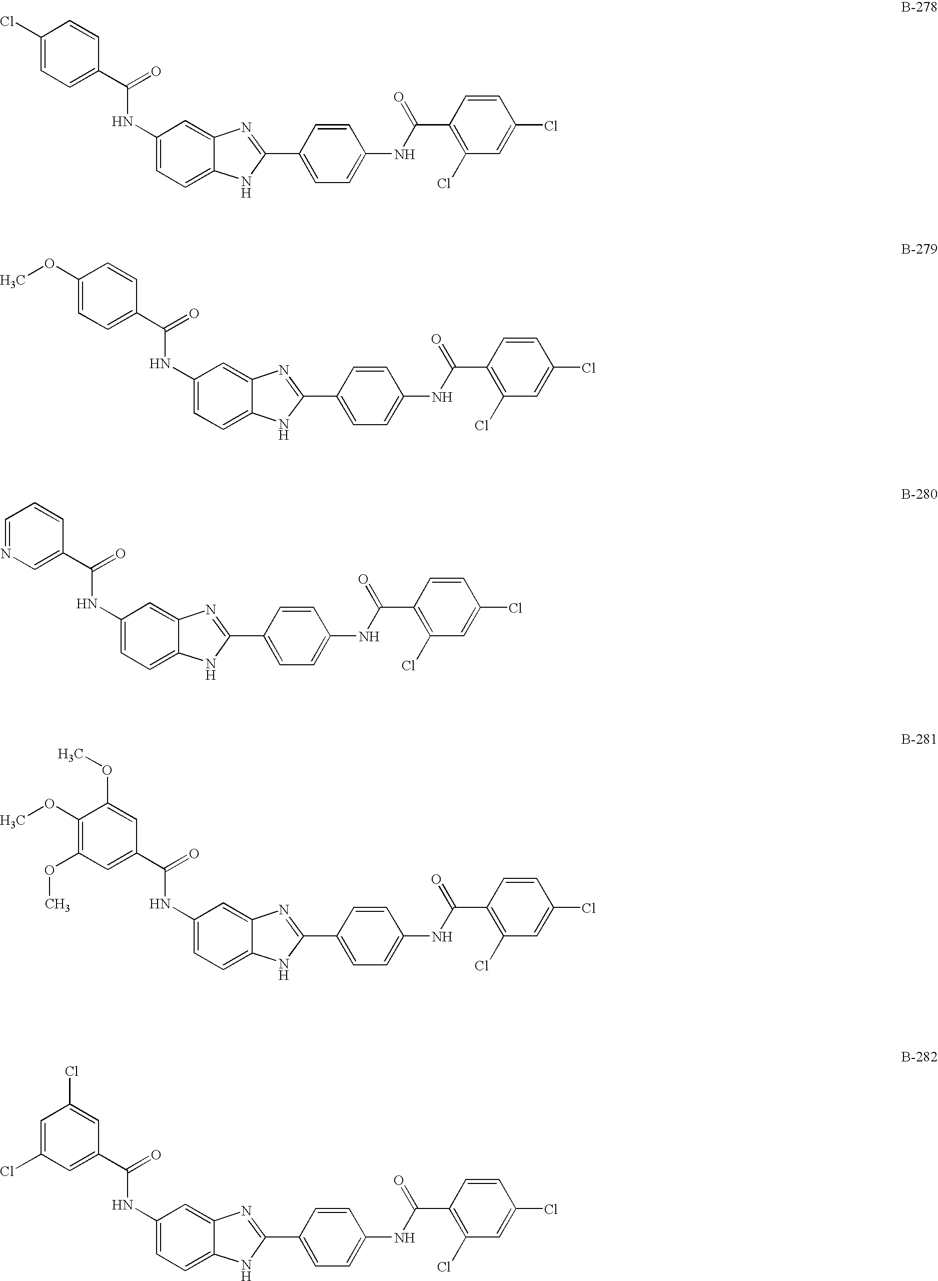

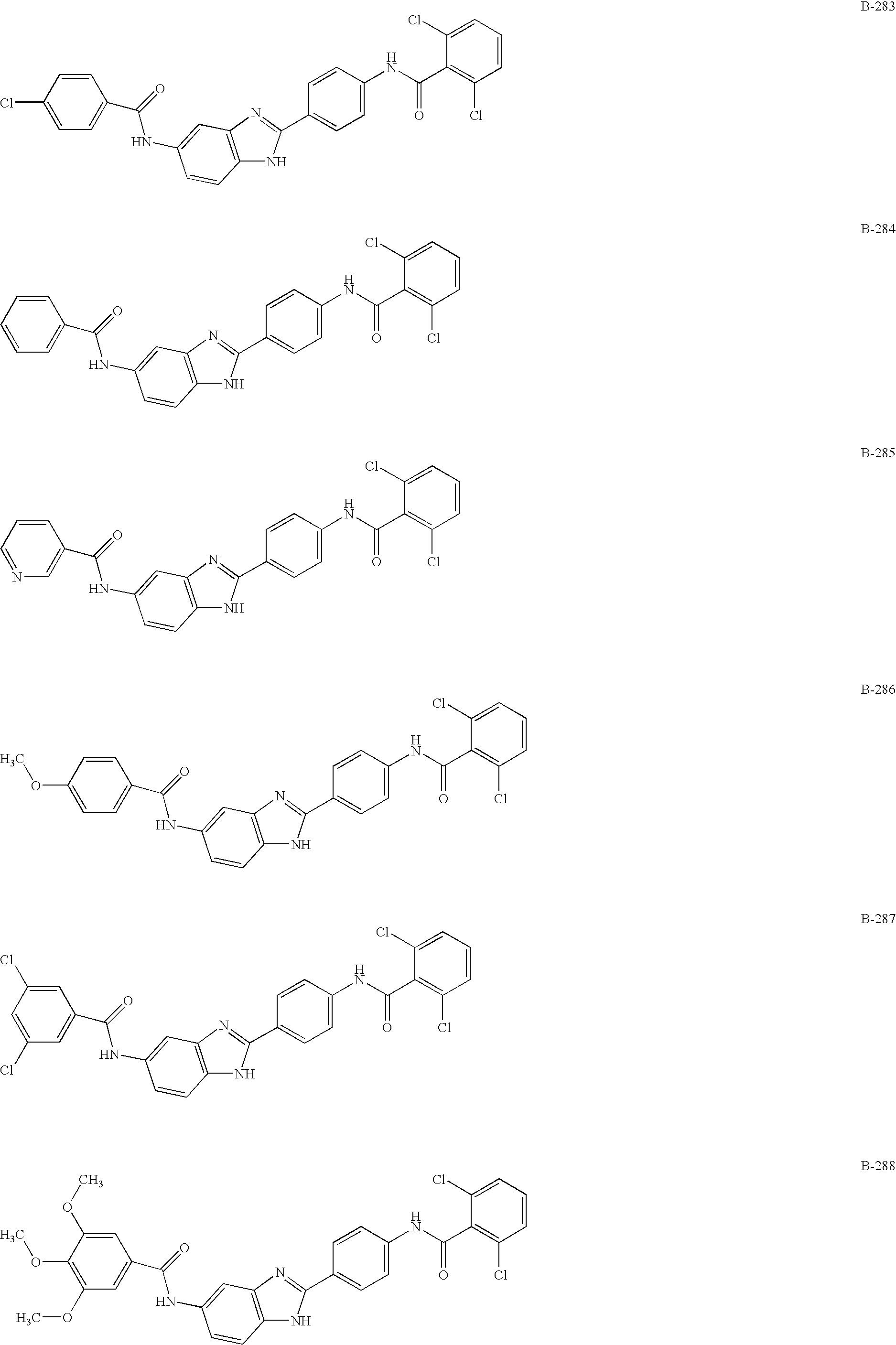

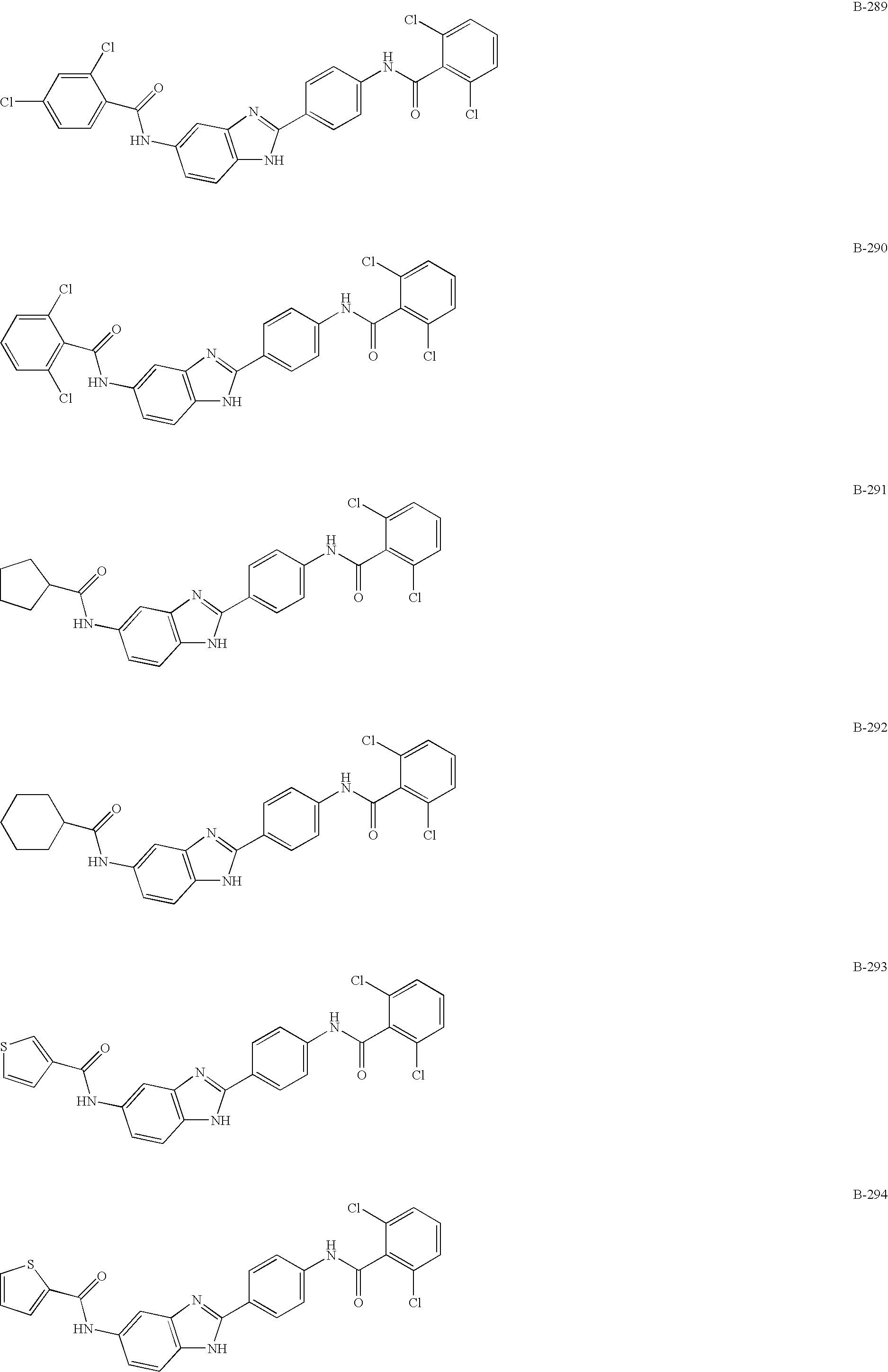

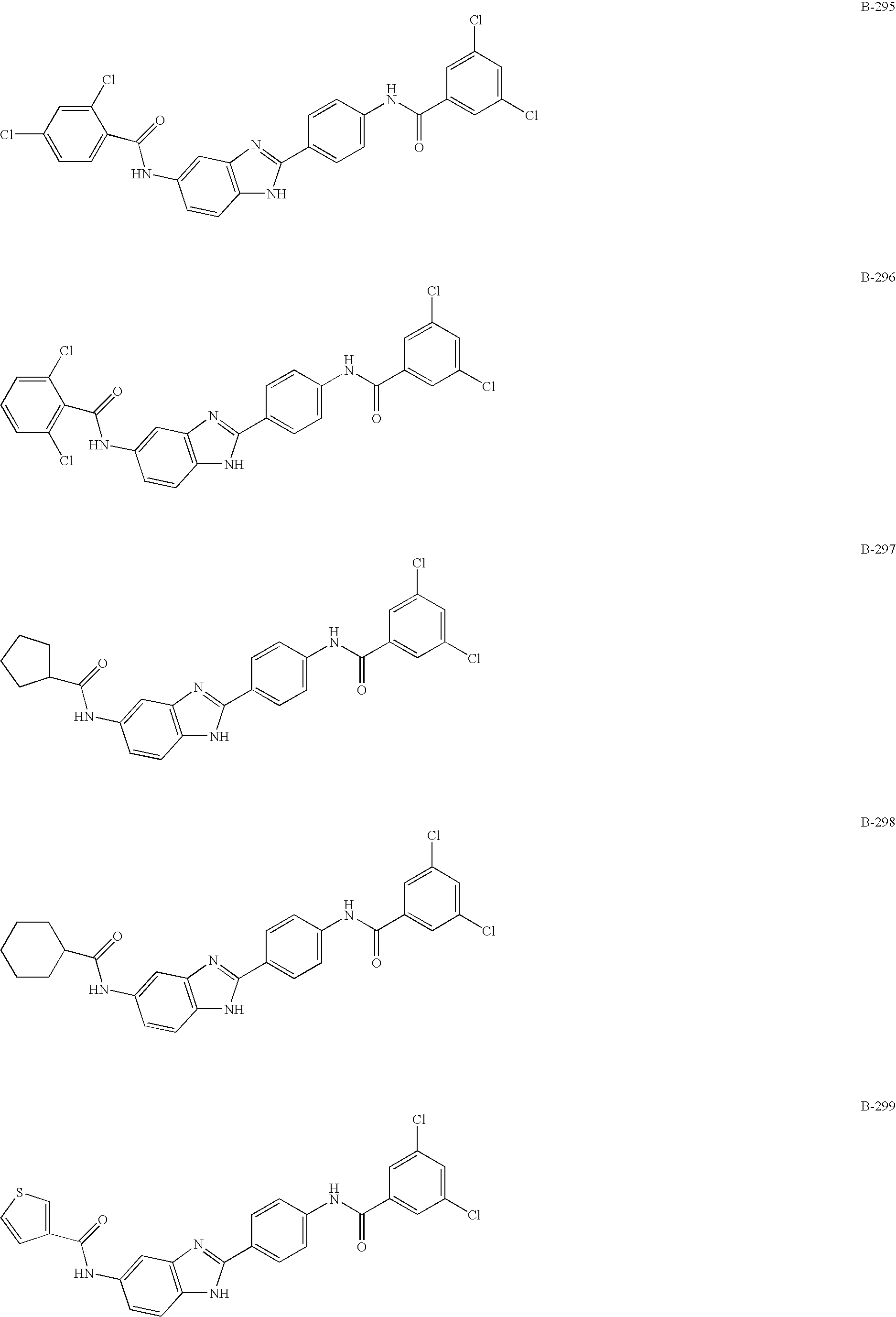

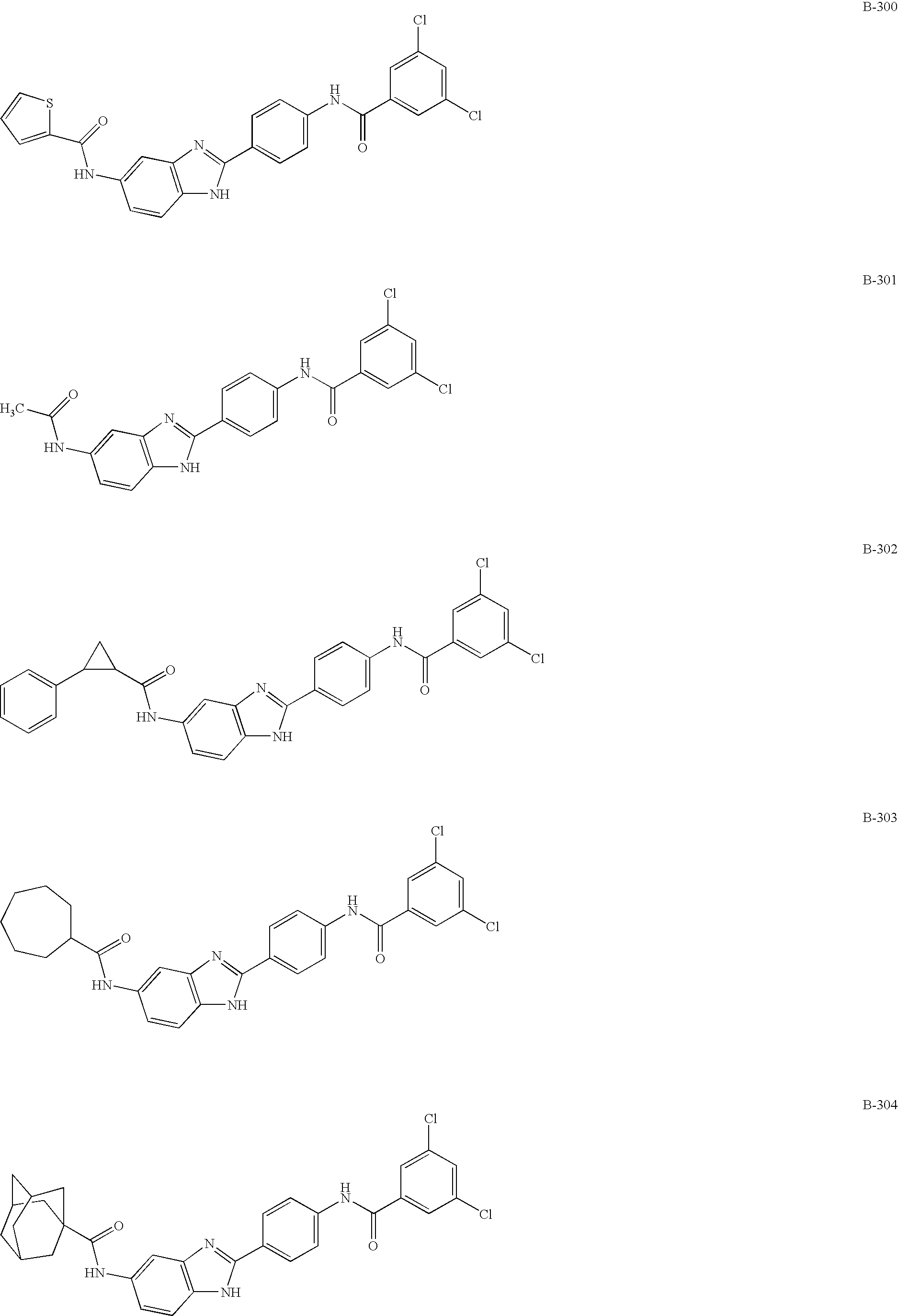

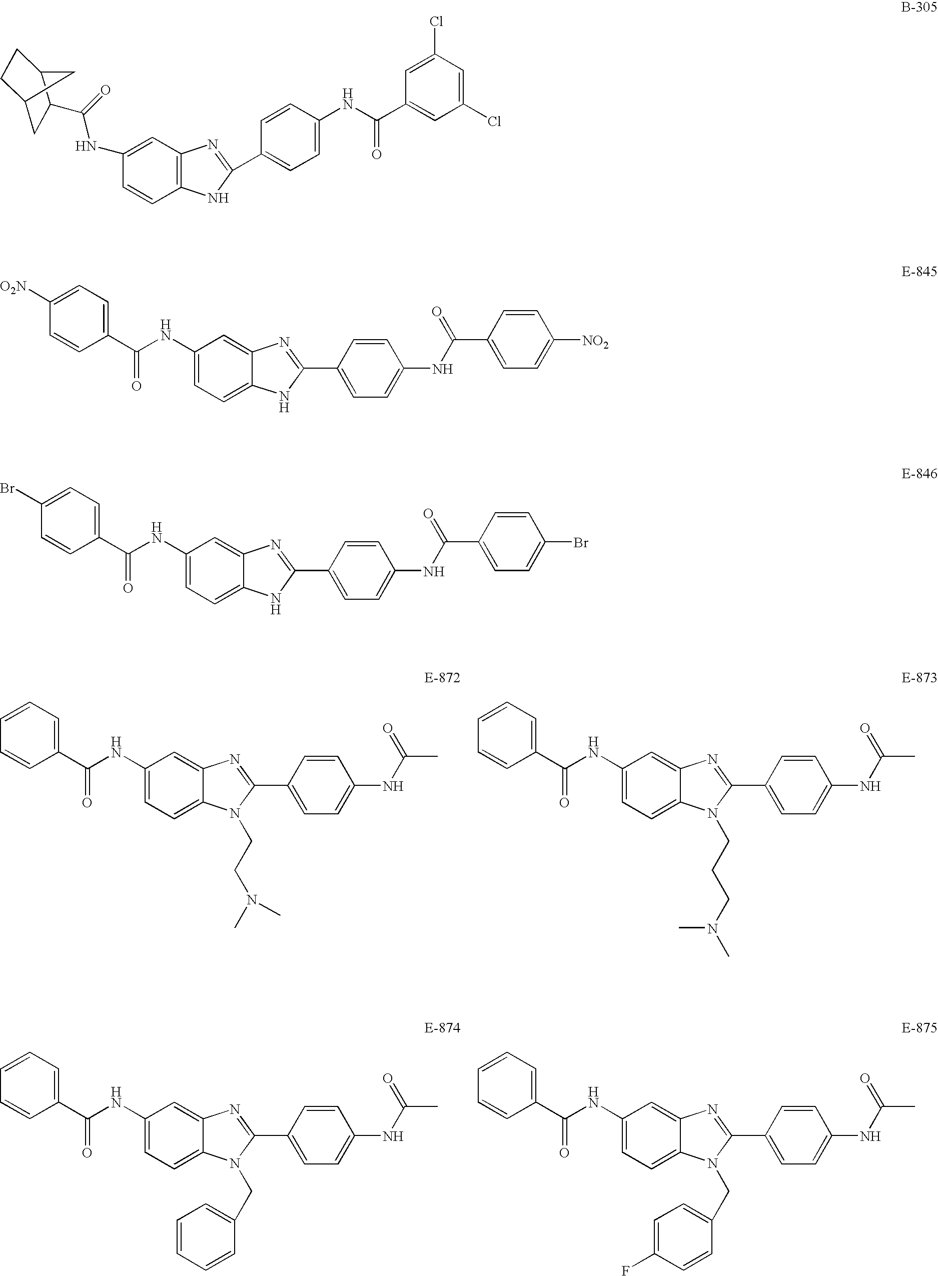

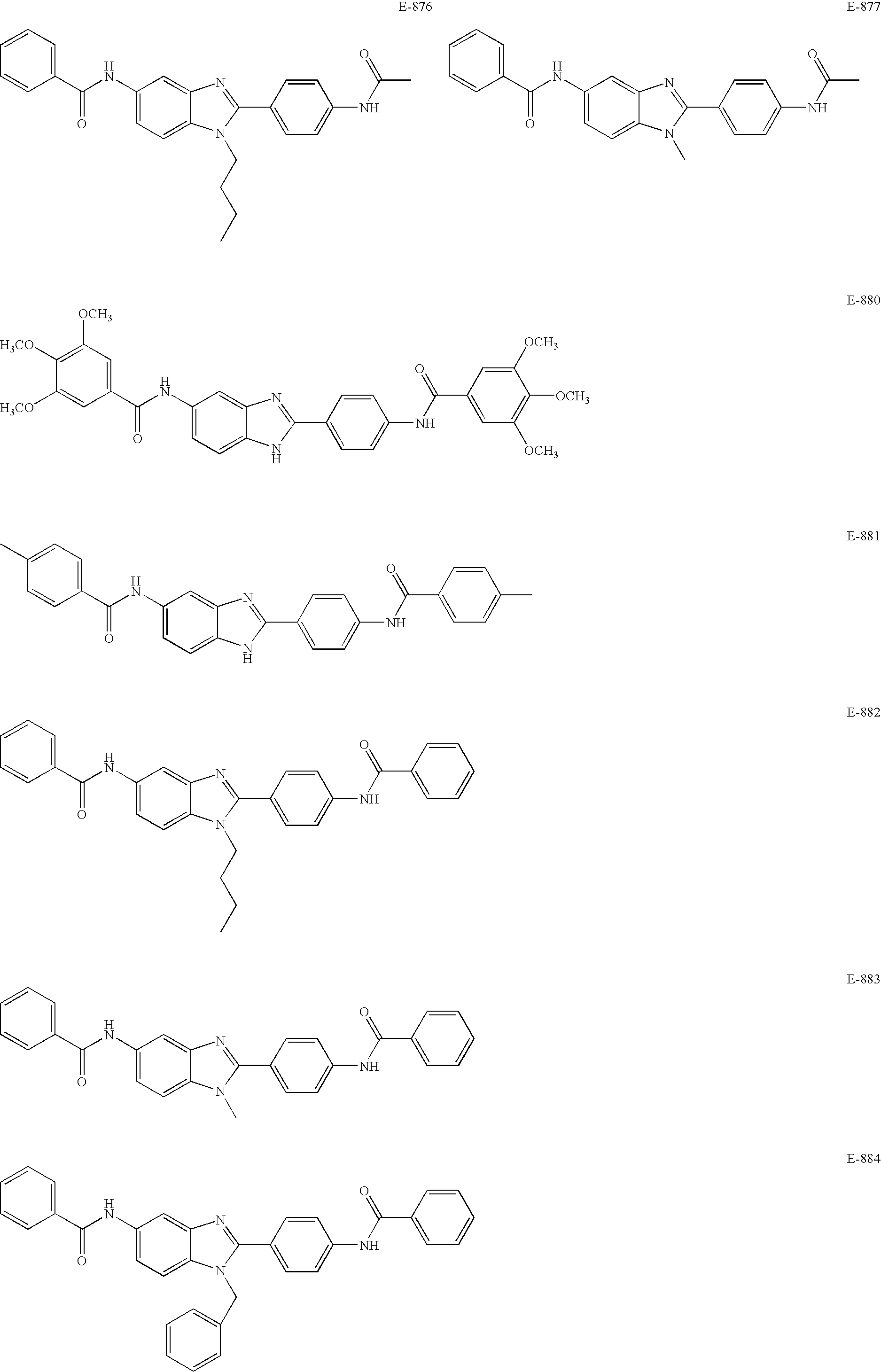

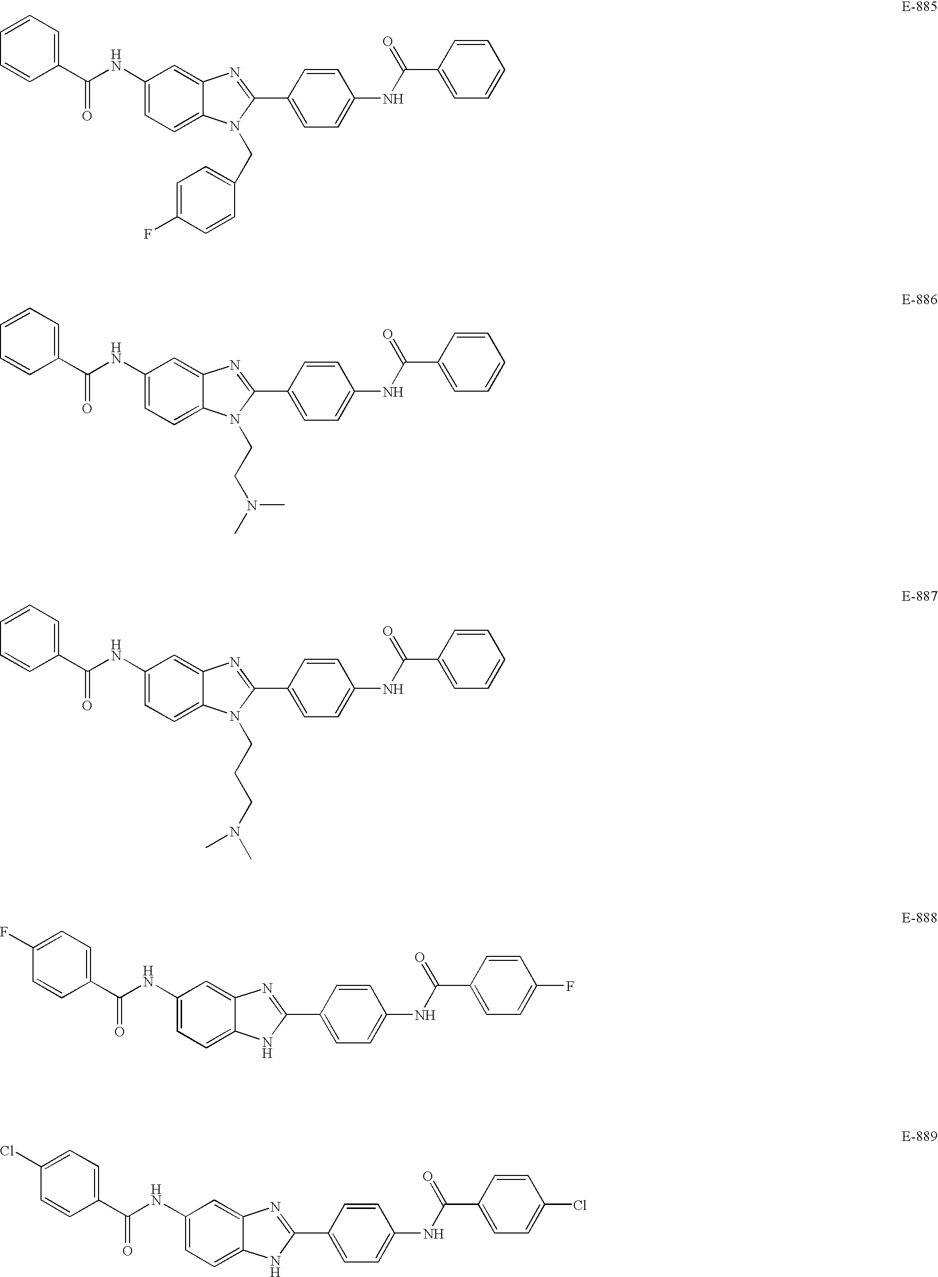

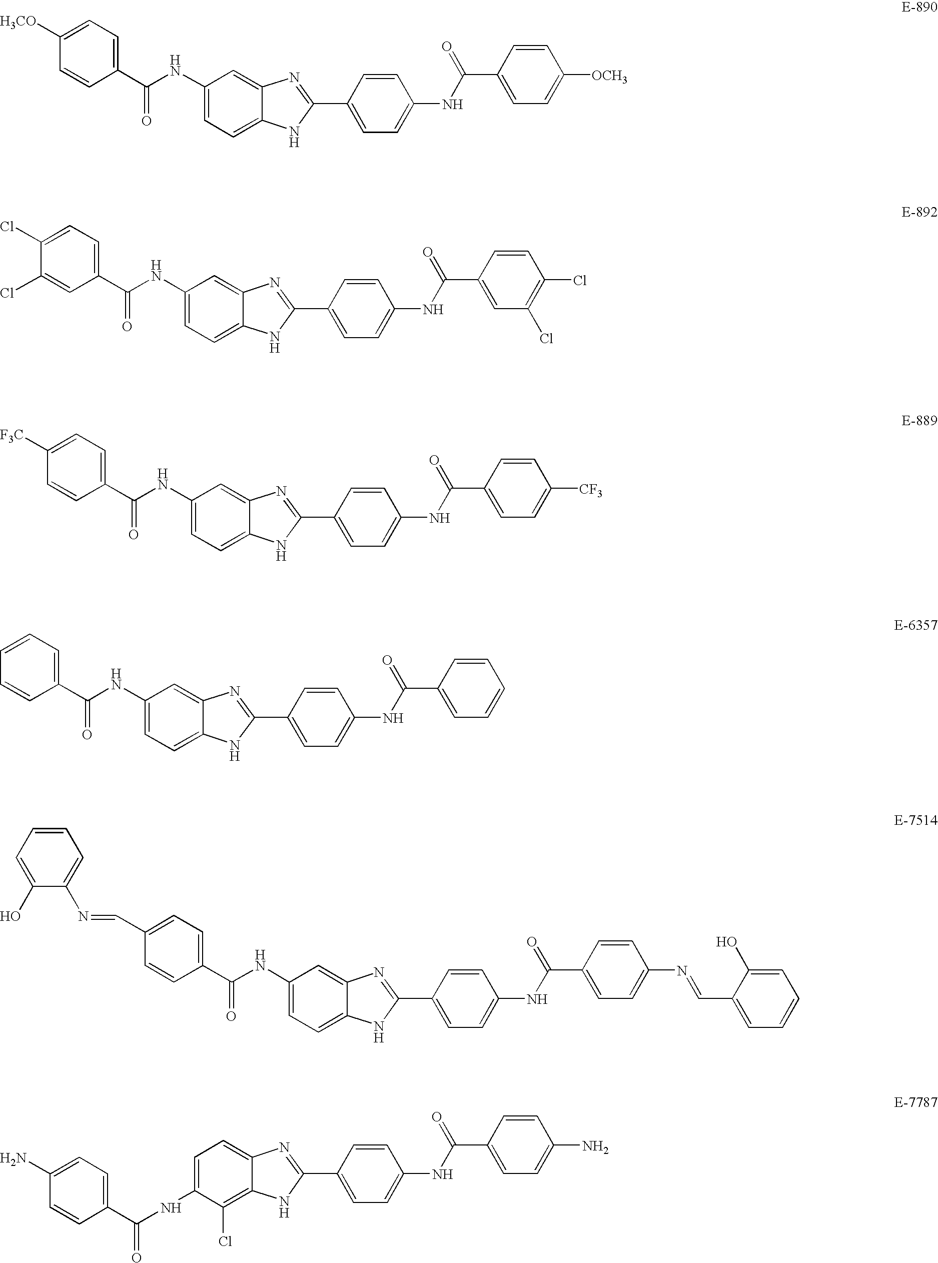

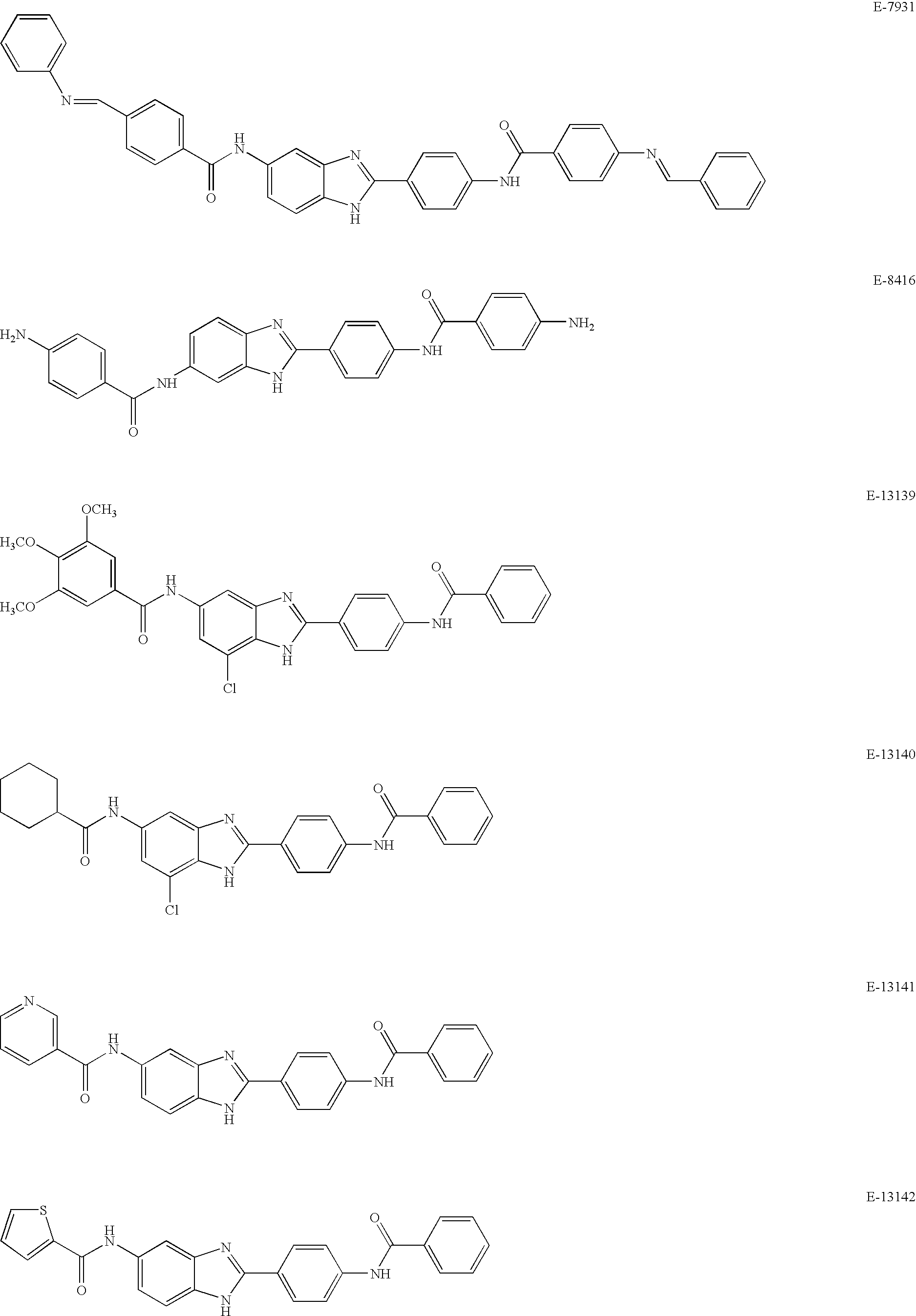

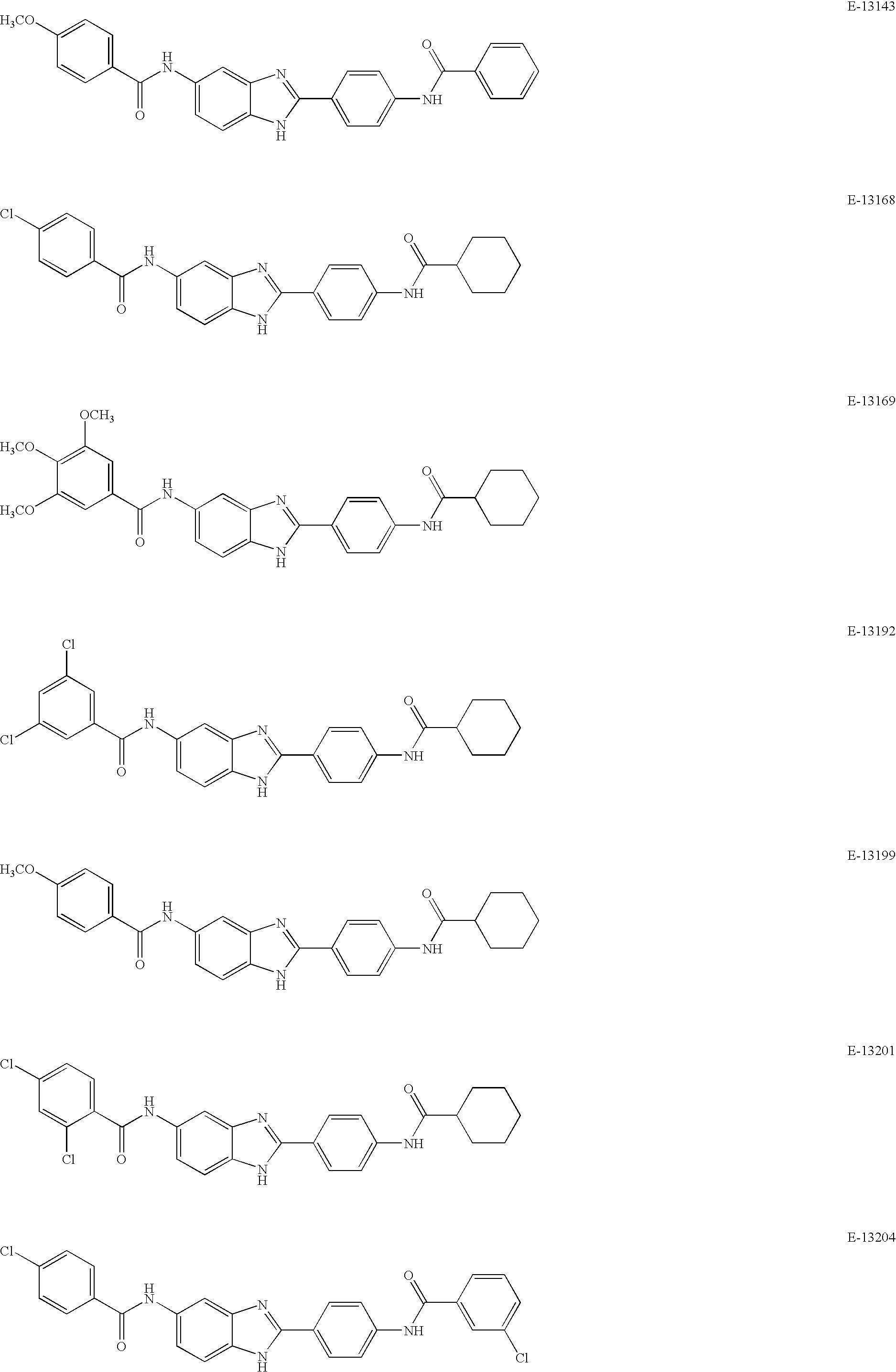

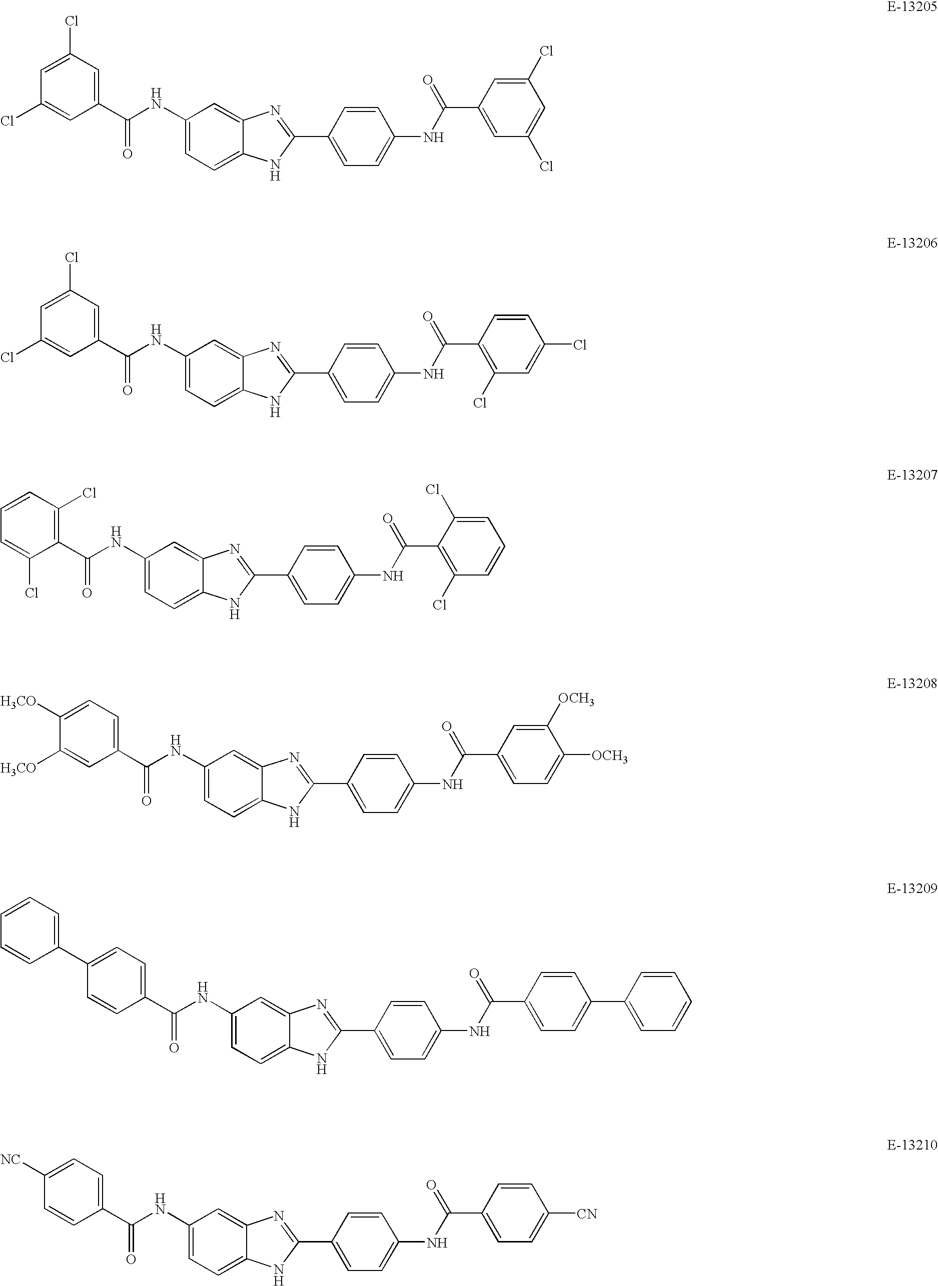

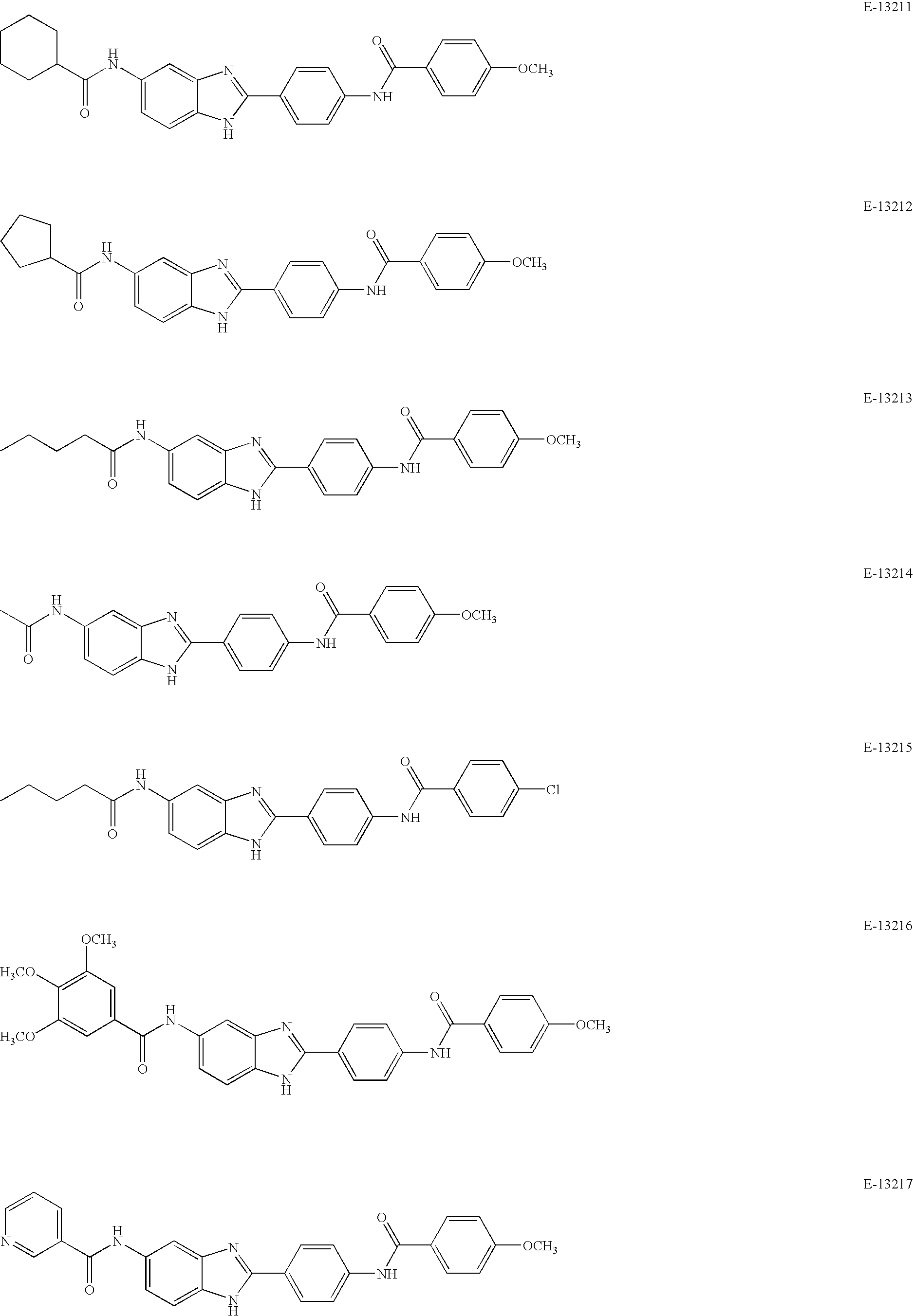

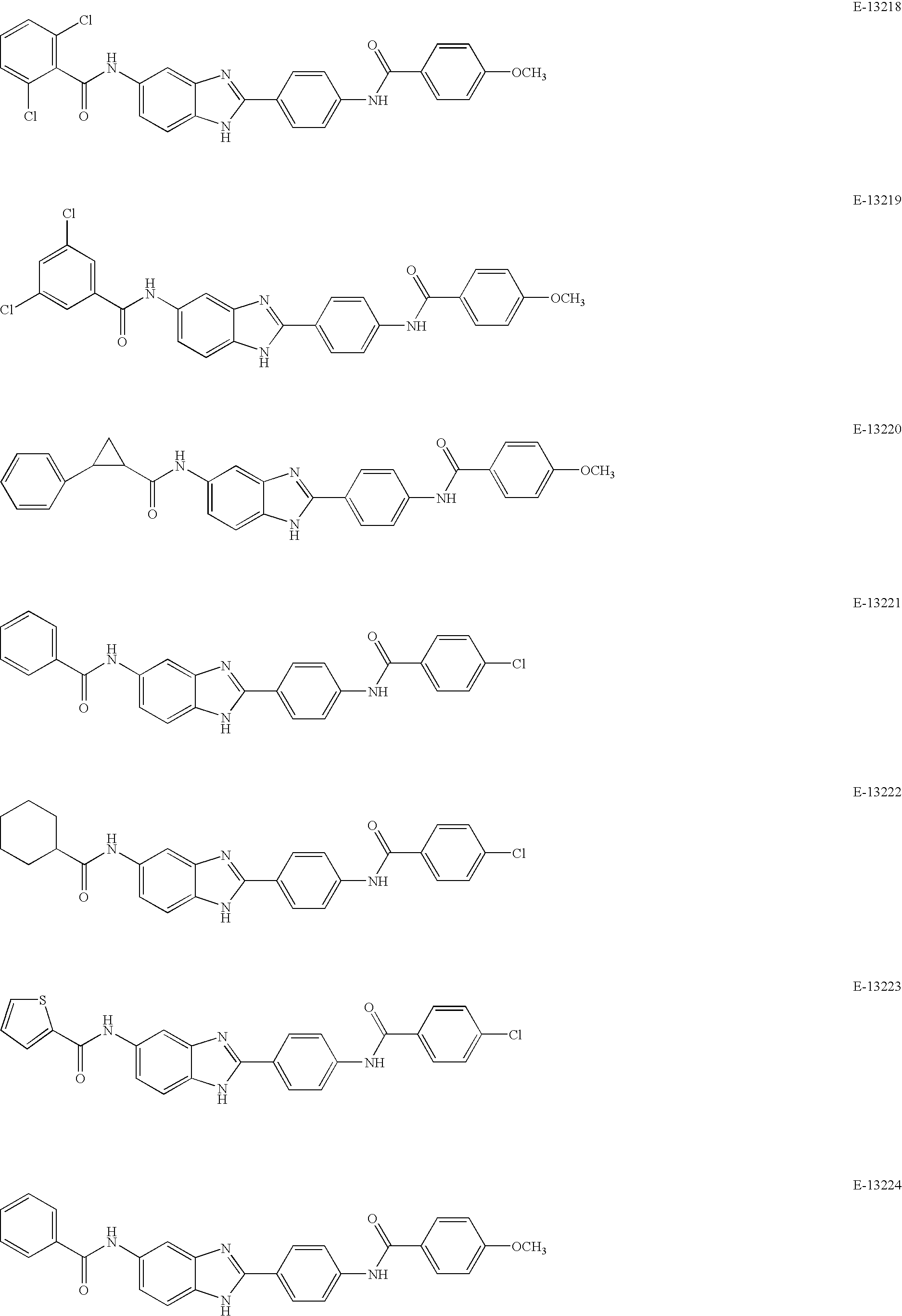

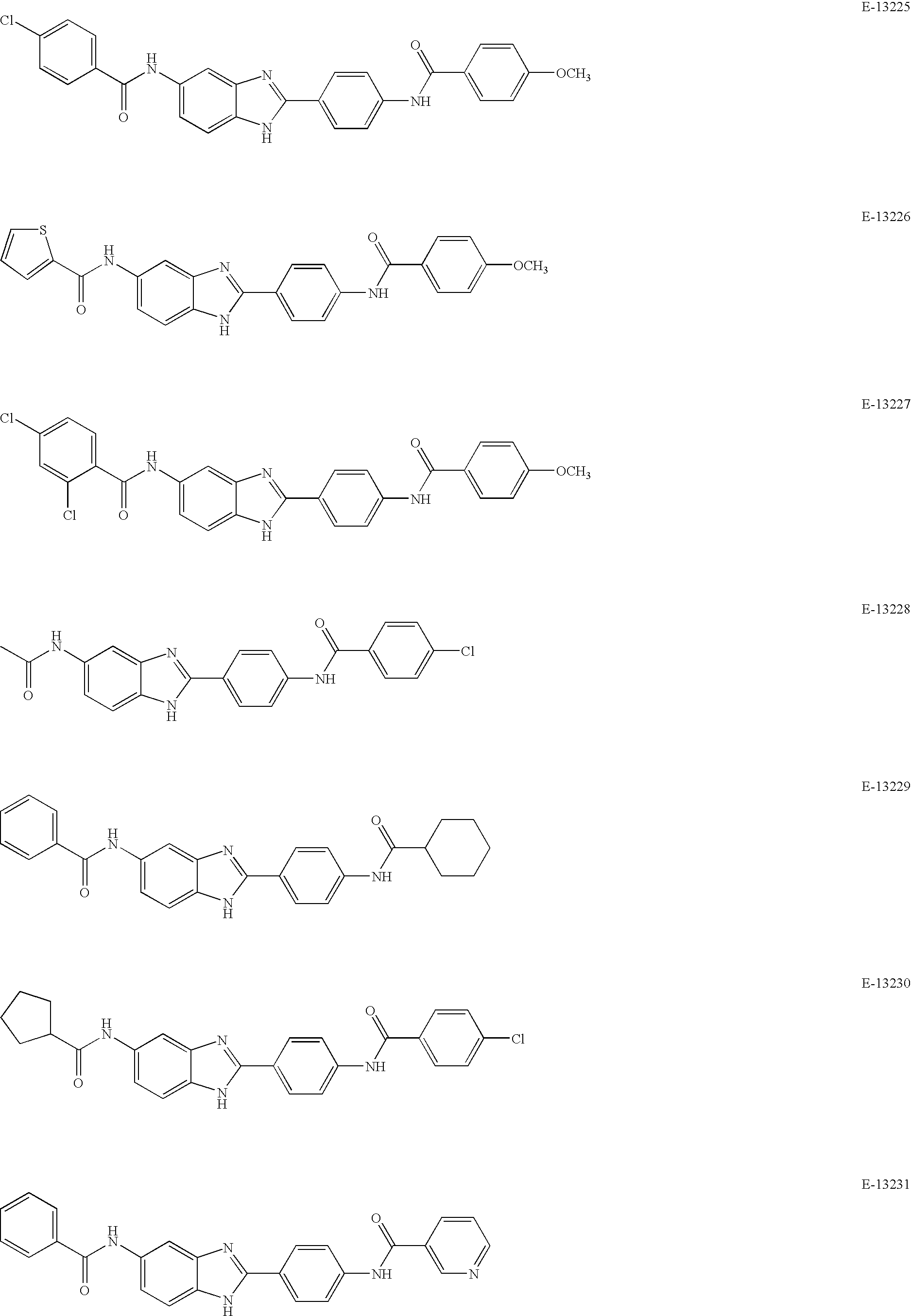

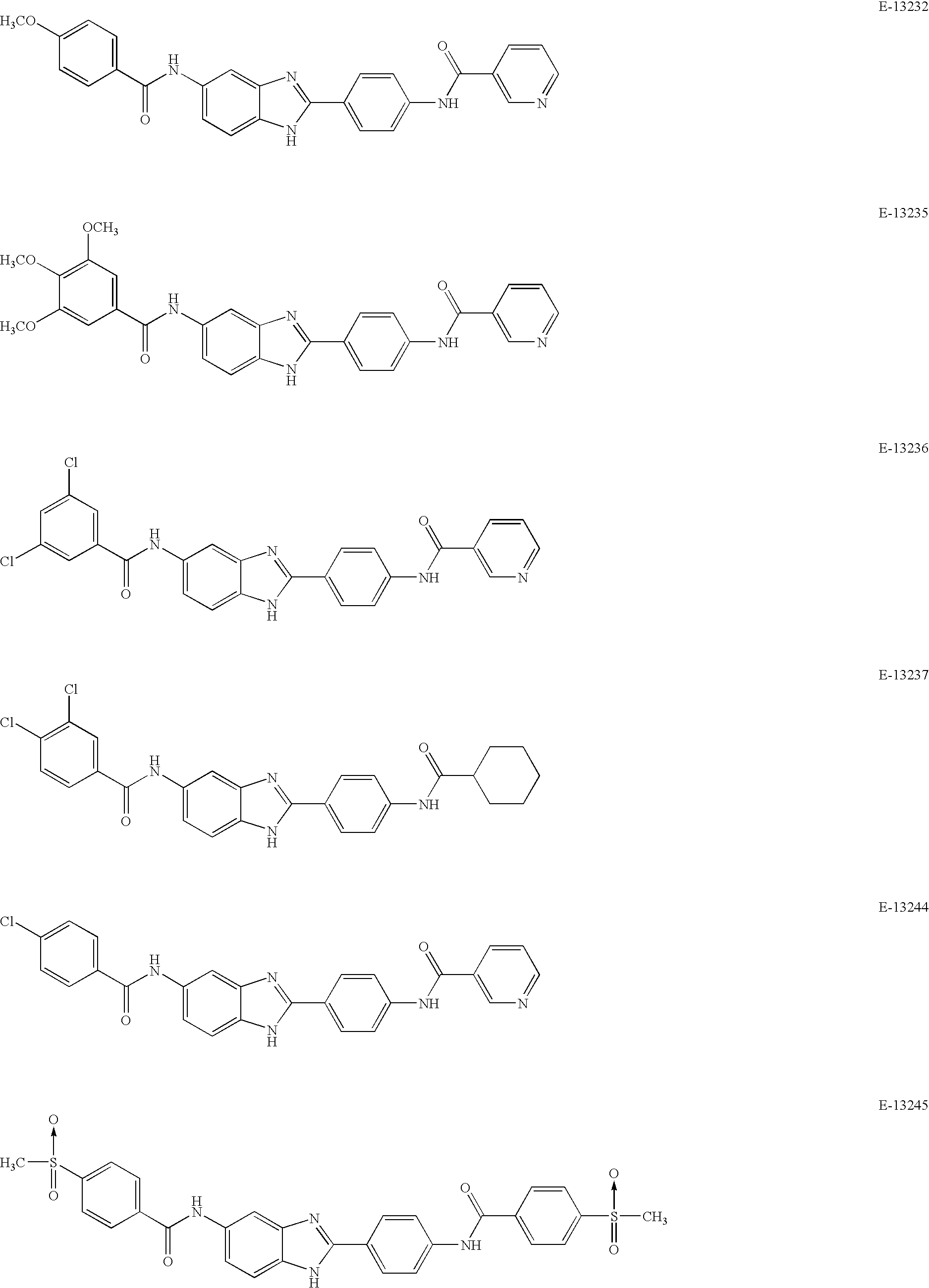

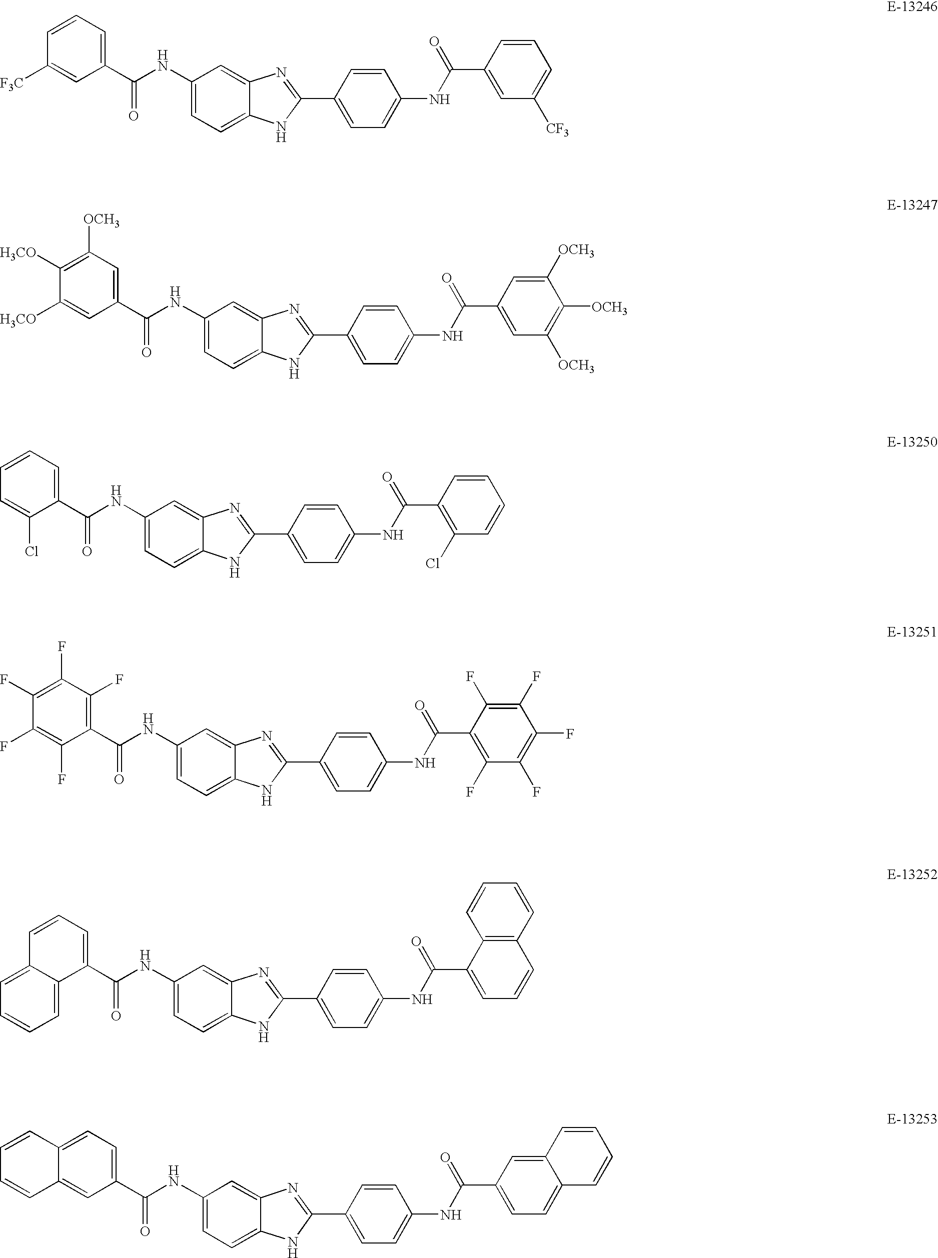

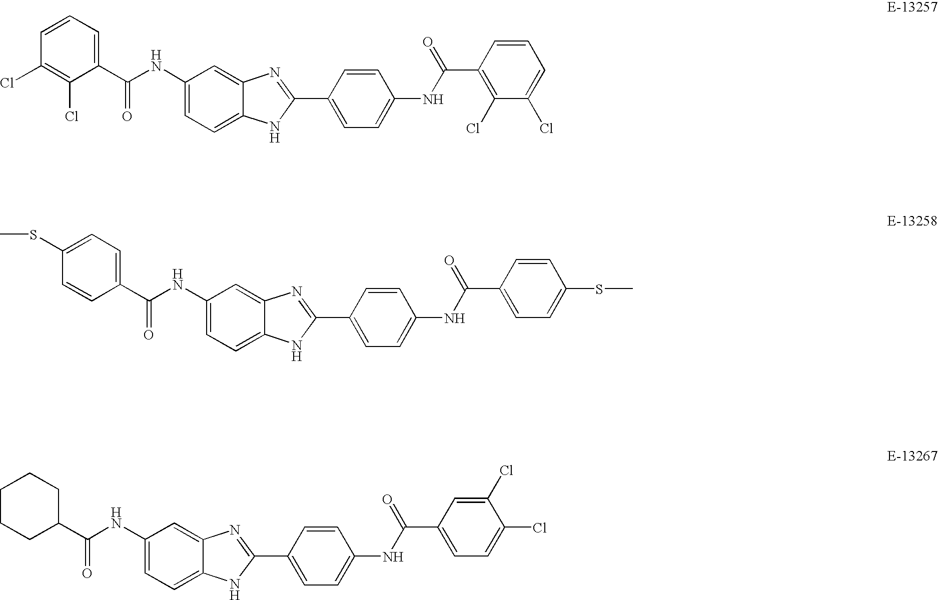

Specific compounds of the preferred embodiments of Genus C which are preferred are represented by the following structural formulae or a pharmaceutically acceptable salt or solvate thereof.

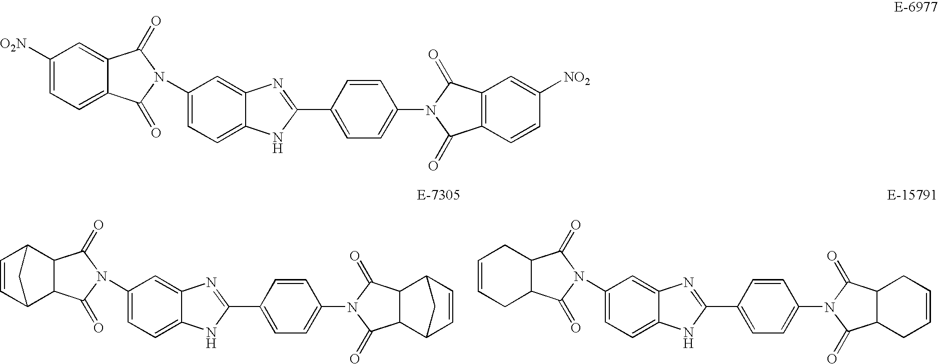

Specific compounds of the preferred embodiments of Genus D which are preferred are represented by the following structural formulae or a pharmaceutically acceptable salt or solvate thereof.

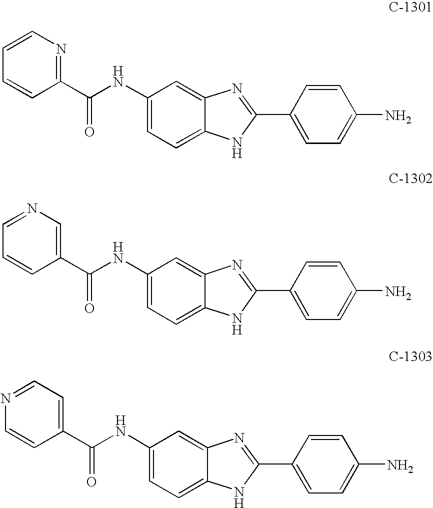

Specific compounds of the preferred embodiments of Genus E which are preferred are represented by the following structural formulae or a pharmaceutically acceptable salt or solvate thereof.

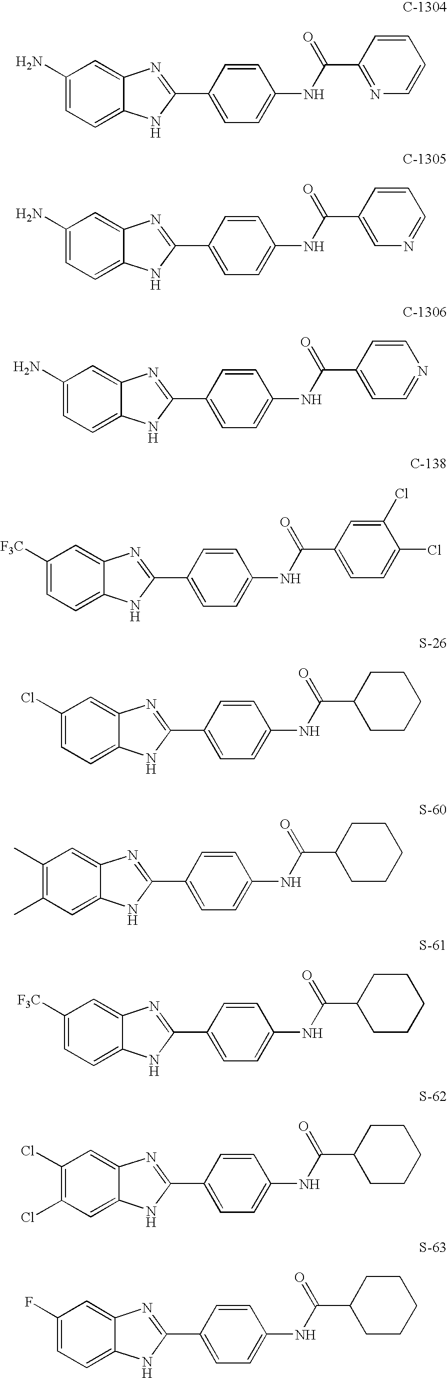

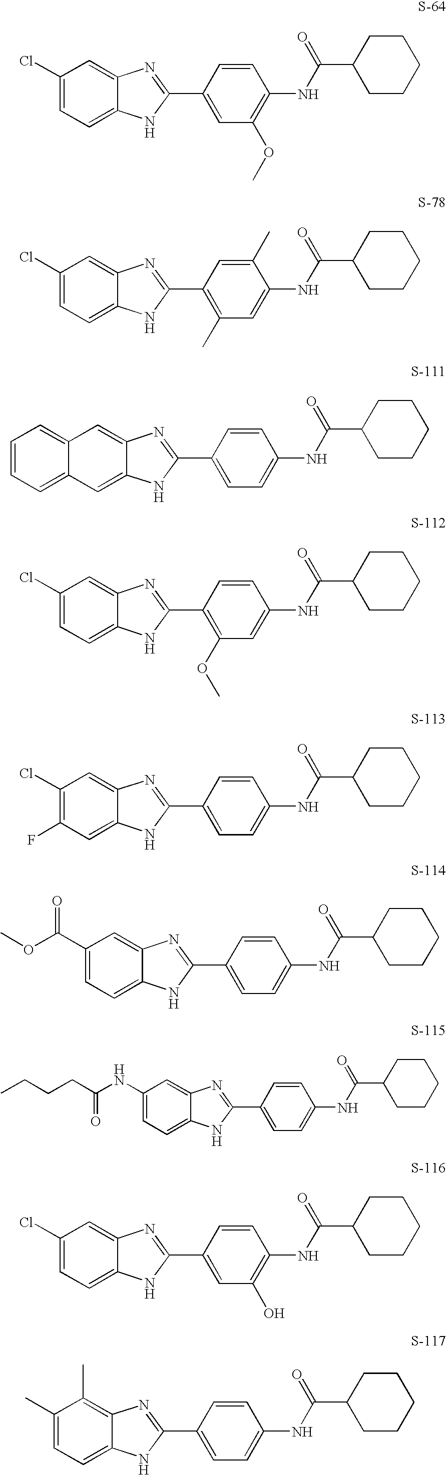

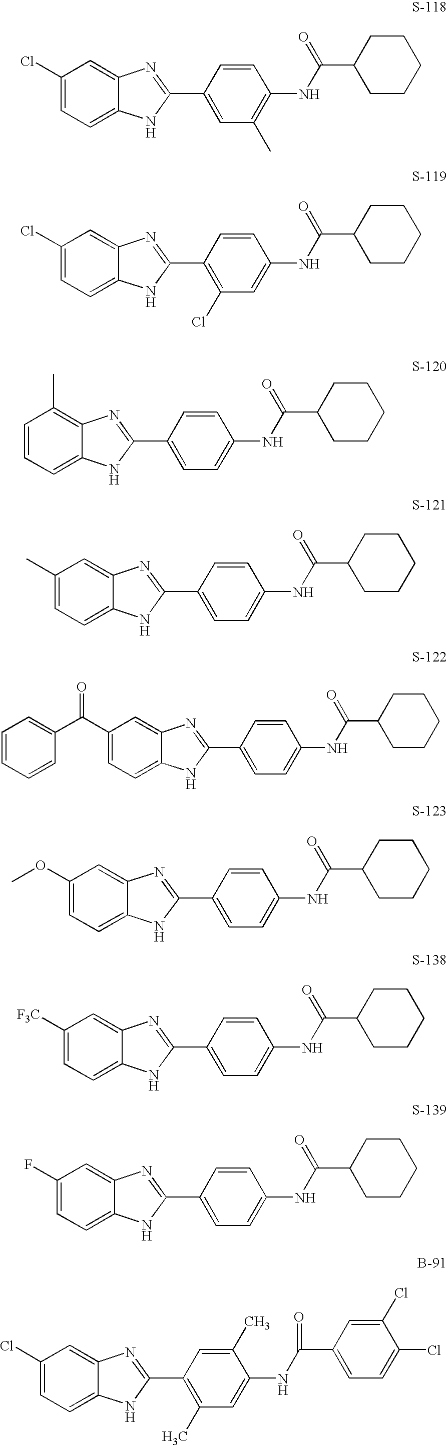

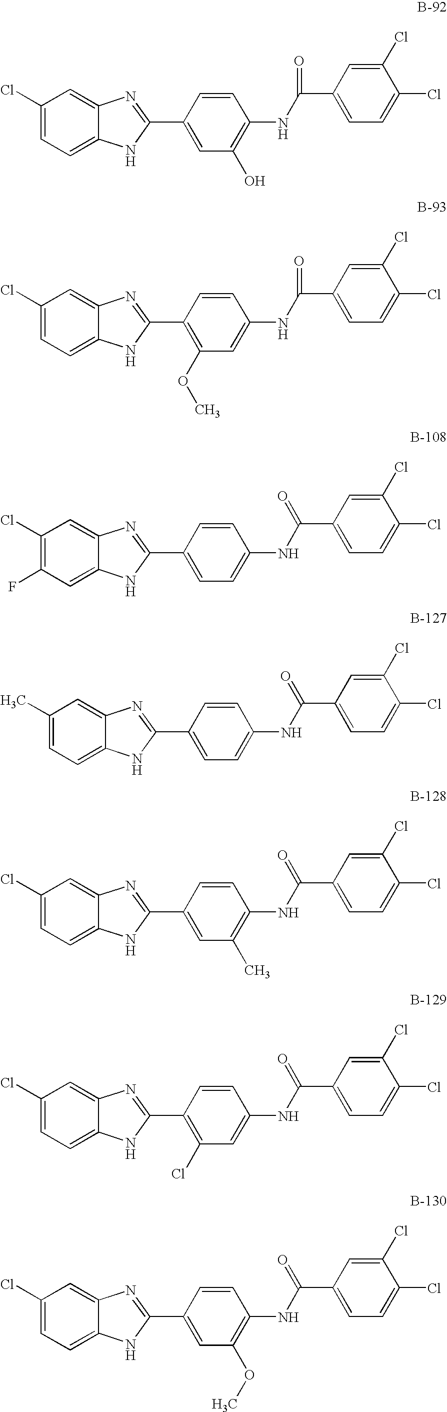

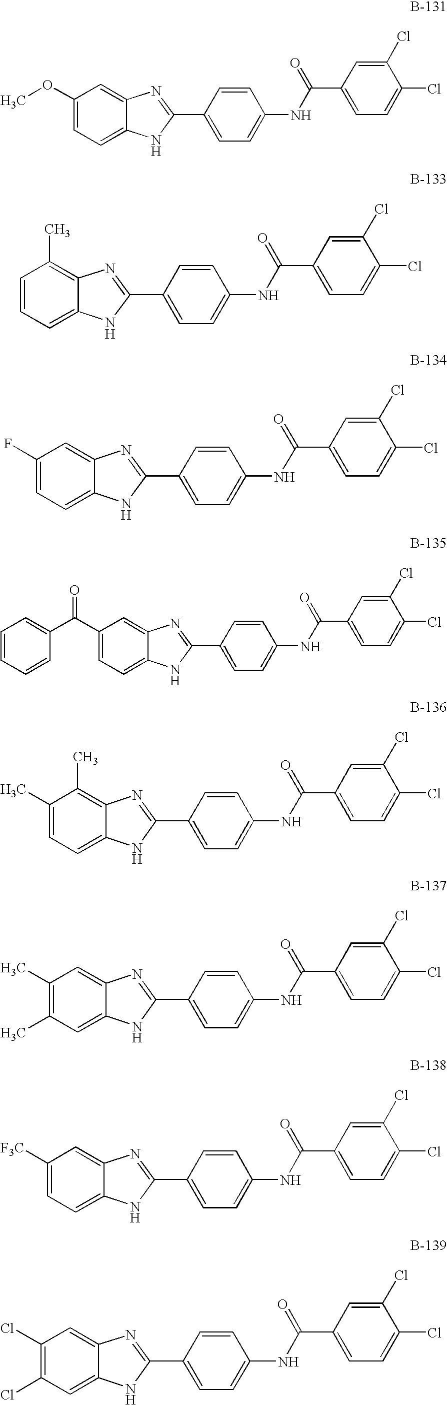

The compounds of the preferred embodiments can possess at least one basic functional substituent and, as such, are capable of forming salts. Included in the definition of pharmaceutically acceptable salts are the relatively non-toxic, inorganic, and organic base or acid addition salts of the compounds of the preferred embodiments. Representative salts include those selected from the group comprising; acetate, benzenesulfonate, benzoate, bicarbonate, bisulfate, bitartrate, borate, camsylate, carbonate, chloride, clavulanate, dihydrochloride, edetate, edisylate, estolate, esylate, fumarate, gluceptate, gluconate, glutamate, glycoilylarsanllate, hexylresorcinate, hydrabamine, hydrobromide, hydrochloride, hydroxynaphthoate, iodide, isothionate, lactate, lactobionate, laurate, malate, malseate, mandelate, mesylate, methylbromide, methylnitrate, methylsulfate, mucate, napsylate, nitrate, oleate, oxalate, palmirate, pantothenate, phosphate, polygalacturonate, salicylate, stearate, subacetate, succinate, tannate, tartrate, tosylate, trifluoroacetate, trifluoromethane sulfonate, and valerate.

Certain compounds of the invention possess one or more chiral centers and may thus exist in optically active forms. Likewise, when the compounds contain an alkenyl or alkenylene group there exists the possibility of cis- and trans- isomeric forms of the compounds. The R- and S-isomers and mixtures thereof, including racemic mixtures as well as mixtures of cis- and transisomers, are contemplated. Additional asymmetric carbon atoms can be present in a substituent group, such as an alkyl group. All such isomers as well as the mixtures thereof are intended to be included in the preferred embodiments. If a particular stereoisomer is desired, it can be prepared by methods well known in the art by using stereospecific reactions with starting materials which contain the asymmetric centers and are already resolved or, alternatively by methods which lead to mixtures of the stereoisomers and subsequent resolution by known methods.

Method of Making Compounds of Preferred Embodiments

General Organic Methods HPLC/MS data was obtained using a Gilson semi-prep HPLC with a Gilson 170 Diode Array UV detector and PE Sciex API 100LC MS based detector. A Waters 600E with a Waters 490E UV detector was also used for recording HPLC data. The compounds were eluted with a gradient of CH₃CN (with 0.0035% TFA) and H20 (with 0.01% TFA). Both HPLC instruments used Advantage C18 60A 5μ 50 mm×4.6 mm columns from Thomson Instrument Company. Mass spectra were obtained by direct injection and electrospray ionization on a PE Sciex API 100LC MS based detector. Thin layer chromatography was performed using Merck 60F-254 aluminum backed pre-coated plates. Flash chromatography was carried out on Merck silica gel 60 (230-400 mesh) purchased from EM Scientific.

Synthesis of the Combinatorial Library

The compounds of the preferred embodiments were prepared using the following synthetic reactions shown in Synthetic Scheme 1, wherein the desired acid chlorides are selected from the R₁ and R₂ groups provided in Table 1. The numbers that refer to the compounds in the text below correspond to those in the diagram. Compounds 1 and 2 can have the appropriate substituents to ultimately give a desired product 6 with the corresponding substituents. Likewise, the positions of the amides on the phenylbenzimidazole ring in a desired product 6 can be varied according the position of the nitrogen on the rings in the starting materials. Table 1 discloses representative acid chlorides and does not represent all the possible acid chlorides that can be used.

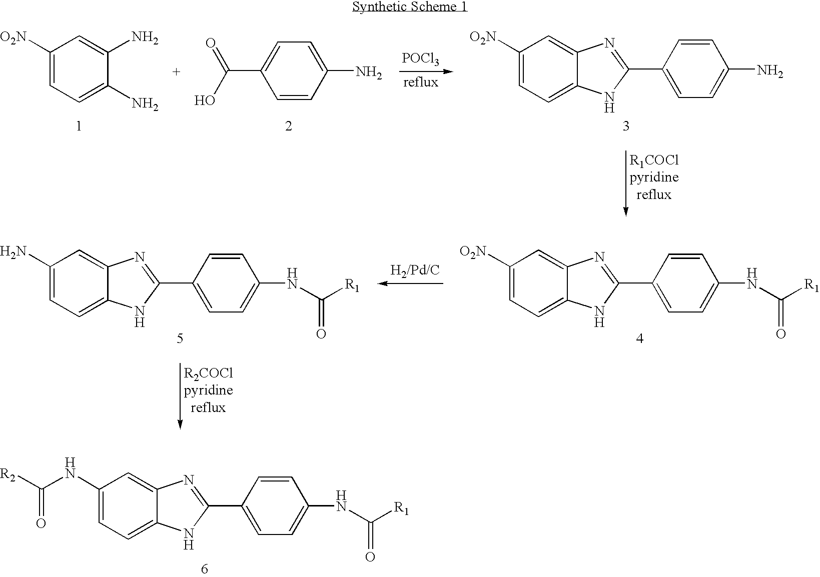

TABLE 1

| | I. R1 | | II. R2 |
|---|---|---|---|
| A | 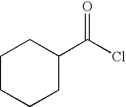 | A | 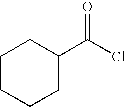 |
| B | 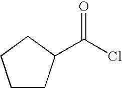 | B | 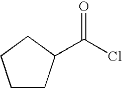 |
| C | 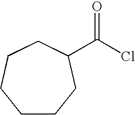 | C | 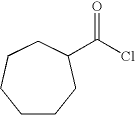 |
| D | 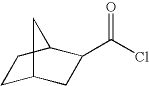 | D | 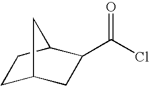 |
| E | 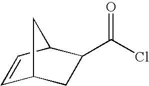 | E | 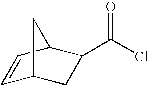 |
| F | 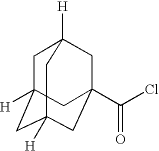 | F | 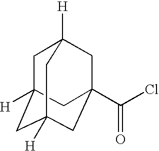 |
| H | 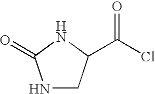 | H | 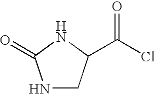 |
| I | 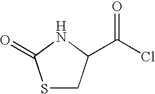 | I | 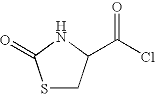 |
| J | 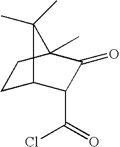 | J | 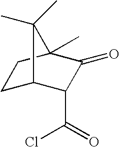 |
| K | 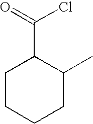 | K | 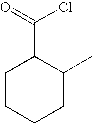 |
| L | 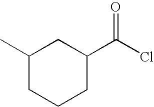 | L | 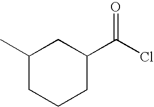 |
| M | 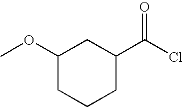 | M | 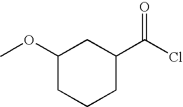 |
| N | 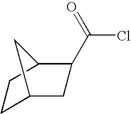 | N | 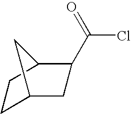 |
| O | 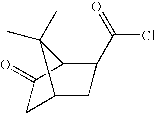 | O | 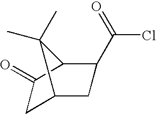 |
| P | 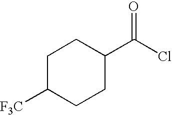 | P | 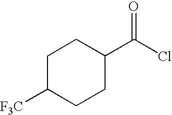 |
| Q | 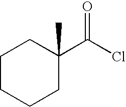 | Q | 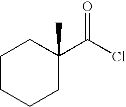 |
| R | 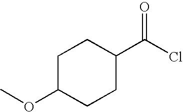 | R | 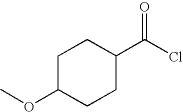 |
| S | 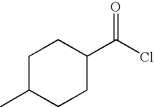 | S | 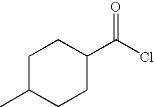 |
| T | 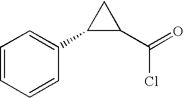 | T | 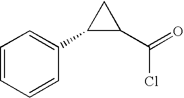 |
| U | 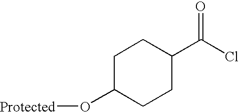 | U | 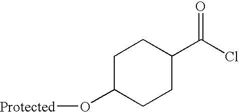 |

Synthesis of 3 4-Nitro-1,2-phenylenediamine (10 g, 65.3 mmol) and 4-aminobenzoic acid (8.95 g, 65.3 mmol) were taken in a round bottomed flask and phosphorus oxychloride (95 ml) was added slowly. The reaction mixture was allowed to stir under reflux conditions. After 18 h, the reaction was allowed to cool and then poured slowly into an ice water mixture in an Erlenmeyer flask with vigorous stirring. Greenish yellow precipitate fell out which was then filtered and washed with copious amounts of water. The residue was then dried to obtain 16.9 g of crude desired product. Mass spectrum analysis (positive ion) indicated presence of phenylbenzimidazole 3.

Synthesis of 4 Phenylbenzimidazole 3 (800 mg, 3.14 mmol) was dissolved in dry pyridine (5 ml) in a scintillation vial and a desired acid chloride (1.1 eq) was added slowly. The reactions were carried out in an oven at 60° C. After 16 h, the reaction was cooled to RT and DI water was added. Precipitation took place, which was filtered off, washed with water and air-dried. The aqueous layer was extracted with EtOAc (6×50 ml), dried over anhydrous Na₂SO₄ and the solvent was removed in vacuo to result in a colored solid. By positive ion MS the desired monoamido product was found to be present in the initial precipitate as well as in the organic layer. Hence the solid residues obtained were combined and used as such for the reduction step.

Synthesis of 5 Crude monoamido-nitrobenzimidazole 4 (1.22 g, 3.40 mmol) was dissolved in MeOH (20 ml) and minimum amount of THF was added for complete dissolution to occur. Catalytic amount of 10% Pd on C was added and the solution was degassed and allowed to stir at 3.4 atm pressure under H₂ atmosphere for 4 h. Upon completion of reaction as observed via TLC, the reaction mixture was filtered through celite and the solvent was removed under reduced pressure to afford 979 mg of crude residue.

Synthesis of 6 Phenylbenzimidazole 5 was dissolved in dry pyridine in a scintillation vial and a desired acid chloride (1.1 eq) was added slowly. The reactions were carried out in an oven at 60° C. After 16h, the reaction was cooled to RT and DI water was added. Precipitation took place, which was filtered off, washed with water and air-dried. The aqueous layer was extracted with EtOAc, dried over anhydrous Na₂SO₄ and the solvent was removed in vacuo to result in diamido product 6.

Alternatively, the diamido-phenylbenzimidazole compounds of the preferred embodiments can also be prepared using the following synthetic reactions shown in Synthetic Scheme 2, wherein the desired acid chlorides are selected from the RI groups provided in Table 1. The numbers that refer to the compounds in the text below correspond to those in the diagram Compounds 11 and 12 can have the appropriate substituents to ultimately give a desired product 15 with the corresponding substituents. Likewise, the positions of the amides on the phenylbenzimidazole ring in the desired product 15 can be varied according tot he position of the nitrogen on the rings of the starting-materials. Table 1 discloses representative acid chlorides and does not represent all the possible acid chlorides that can be used. In the Synthetic Scheme 2, the one type of acid chloride is used to form the amides on both amines of 14.

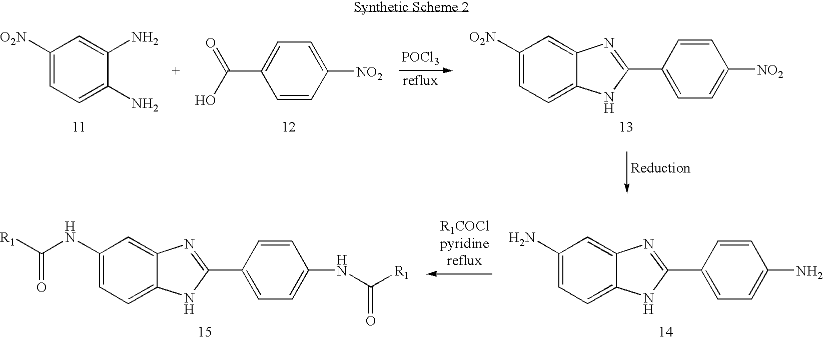

The compounds of the preferred embodiments were generally prepared from 2-(4-aminophenyl)-5-aminobenzimidazole, which was obtained by reduction of 2-(4-nitrophenyl)-5-nitrobenzimidazole.

The dinitro phenylbenzimidazole 13 was prepared as follows: a mixture of 4-nitrophenylenediamine (6.4 g, 41.83 mmol) and 4-nitrobenzoic acid (7.86 g, 47 mmol) was dissolved in POCl₃ (250 ml) and heated to reflux for 2 h. The reaction mixture was cooled, poured on to ice, and stirred for 30 min. The resulting solid was filtered and washed with methanol and sodium bicarbonate to remove unreacted acid and allowed to dry overnight to give the desired product as a brown solid (5.8 g). The product was characterized by electrospray mass spectroscopy (mp>300° C.).

2-(4-Aminophenyl)-5-aminobenzimidazole 14 was prepared by suspending the above solid (75 g) in THF (75 ml), to which was added Pd-C (10% Pd by weight). The flask was purged with hydrogen and stirred under a balloon of hydrogen overnight. TLC and MS showed starting material was still present so the reaction was allowed to continue over the weekend. TLC indicated complete reaction, the reaction was filtered through celite and washed with methanol. The solvent was removed under reduced pressure to give a dark brown solid (0.37 g) that was used without Ether purification.

Alternatively, the 2-(4-aminophenyl)-5-aminobenzimidazole 14 was prepared by the following reduction: 2-(4-nitrophenyl)-6-nitrobenzimidazole (8.9 g, 31 mmole) was suspended in concentrated HCl (100 ml) to which was added stannous chloride (42.3 g 180 mmole). The reaction mixture was heated to reflux for 5 hrs. The mixture was cooled to RT and the HCl salt of the desired product was precipitated by the addition of ethanol. The resulting solid was filtered, redissolved in water and the solution made basic by the addition of concentrated ammonium hydroxide. The resulting precipitate was filtered and dried overnight under vacuum to yield the desired product as a gray solid (6.023 g, 26.9 mmole, 87%). The product was characterized by electrospray mass spectroscopy and HPLC (mp. 222-227° C.).

To obtain the product 15, the intermediate 14 is diacylated to form the diamidophenylbenzimidazole by the above procedures according to Synthetic Scheme 1.

The monoamido-phenylbenzimidazole compounds of the preferred embodiments can be prepared using the following synthetic reactions shown in Synthetic Scheme 3, wherein the desired acid chlorides are selected from the R₁ groups provided in Table 1. The numbers that refer to the compounds in the text below correspond to those in the diagram. Compounds 21 and 22 can have the appropriate substituents to ultimately give a desired product 25 with the corresponding substituents. Likewise, the position of the amide on the phenylbenzimidazole ring in the desired product 25 can be varied according to the position of the nitrogen on She ring in the starting materials. Table 1 discloses representative acid chlorides and does not represent all the possible acid chlorides that can be used. Alternatively, the intermediate 24 can be formed from the condensation of phenylenediamine and 4-aminobenzoic acid

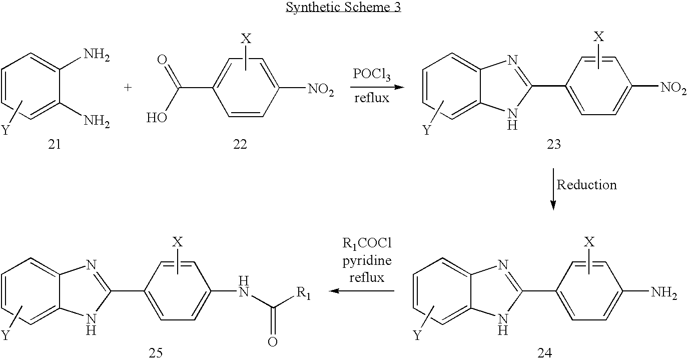

The monoamido-phenylbenzimidazole compounds of the preferred embodiments can also be prepared using the following synthetic reactions shown in Synthetic Scheme 4, wherein the desired acid chlorides are selected from the R₁ groups provided in Table 1. The numbers that refer to the compounds in the text below correspond to those in the diagram. Compounds 31 and 32 can have the appropriate substituents to ultimately give a desired product 35 with the corresponding substituents. Table 1 discloses representative acid chlorides and does not represent all the possible acid chlorides that can be used. Alternatively, the intermediate 34 can be formed from the condensation of nitro-phenylenediamine and benzoic acid

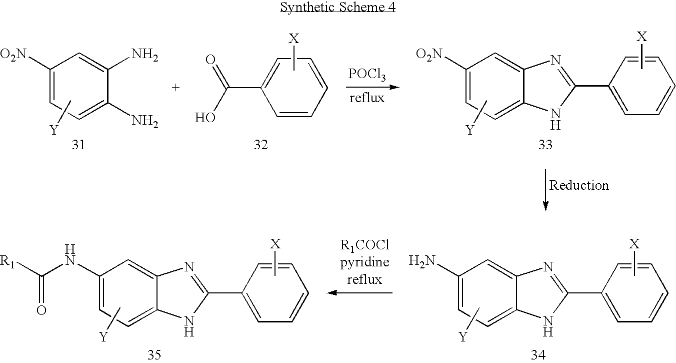

The compounds of the Genus C of preferred embodiments can be prepared using the following synthetic reactions shown in Synthetic Scheme 5. In the synthetic scheme, an amino substituent of compound 3 or 42 is reacted with an acyl chloride with a latent carboxylic acid at the other end. The carboxylic acid is revealed and coupled with the amide in the presence of 2-dimethylaminoisopropyl chloride hydrochloride (DIC), 1-hydroxybenzotriazole hydrate (HOBt), triethylamine and methylene chloride. In the scheme, n and m are integers representing the number of unsubstituted or substituted methylene groups. 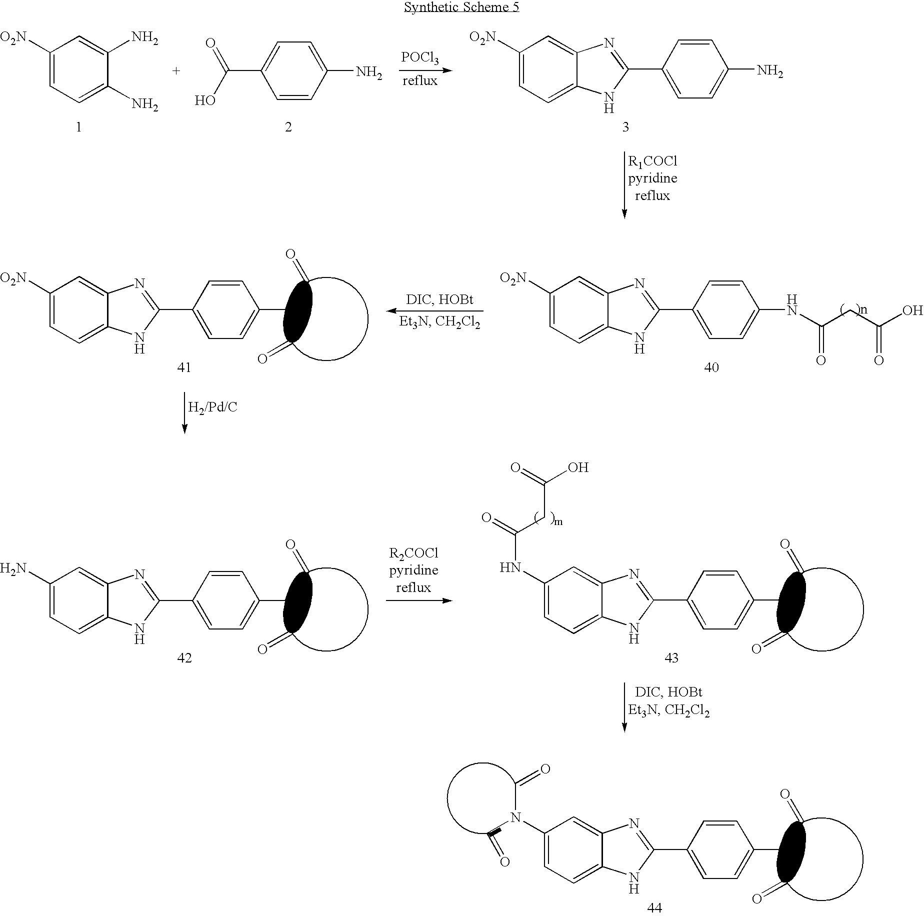
Pharmaceutical Compositions

The compounds of the preferred embodiments can be administered to a patient either alone or a part of a pharmaceutical composition. The compositions can be administered to patients either orally, rectally, parenterally (intravenously, intramuscularly, or subcutaneously), intracistemally, intravaginally, intraperitoneally, intravesically, locally (powders, ointments, or drops), or as a buccal or nasal spray.

Compositions suitable for parenteral injection can comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include, but are not limited to, water, ethanol, polyols (propyleneglycol, polyethyleneglycol, glycerol, and the like), suitable mixtures thereof, vegetable oils (such as olive oil) and injectable organic esters, such as ethyl oleate. Proper fluidity can be maintained, for example, by the use of a coating, such as lecithin, by the maintenance of the required particle size in the case of dispersions and by the use of surfactants.

These compositions can also contain adjuvants, such as preserving, wetting, emulsifying, and dispensing agents. Prevention of the action of microorganisms can be ensured by various antibacterial and antifungal agents, for example, parabens, chlorobutanol, phenol, sorbic acid, and the like. It can also be desirable to include isotonic agents, for example, sugars, sodium chloride, and the like. Prolonged absorption of the injectable pharmaceutical form can be brought about by the use of agents delaying absorption, for example, aluminum monostearate and gelatin.

Solid dosage forms for oral administration include capsules, tablets, pills, powders, and granules. In such solid dosage forms, the active compound is admixed with at least one inert customary excipient (or carrier), such as sodium citrate or dicalcium phosphate or (a) fillers or extenders, as for example, starches, lactose, sucrose, glucose, mannitol, and silicic acid, (b) binders, as for example, carboxymethylcellulose, alignates, gelatin, polyvinylpyrrolidone, sucrose, and acacia, (c) humectants, as for example, glycerol, (d) disintegrating agents, as for example, agar-agar, calcium carbonate, potato or tapioca starch, alginic acid, certain complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, (h) adsorbents, as for example, kaolin and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms can also comprise buffering agents.

Solid compositions of a similar type can also be employed as fillers in soft and hard-filled gelatin capsules using such excipients as lactose or milk sugar as well as high molecular weight polyethyleneglycols, and the like.

Solid dosage forms such as tablets, dragees, capsules, pills, and granules can be prepared with coatings and shells, such as enteric coatings and others well known in the art. They can contain opacifying agents, and can also be of such composition that they release the active compound or compounds in a certain part of the intestinal tract in a delayed manner. Examples of embedding compositions which can be used are polymeric substances and waxes. The active compounds can also be in micro-encapsulated form, if appropriate, with one or more of the above-mentioned excipients.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan or mixtures of these substances, and the like.

Besides such inert diluents, the composition can also include adjuvants, such as wetting agents, emulsifying and suspending agents, sweetening, flavoring, and perfuming agents;

Suspensions, in addition to the active compounds, can contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are preferably suppositories which can be prepared by mixing the compounds of the present invention with suitable non-irritating excipients or carriers, such as cocoa butter, polyethyleneglycol or a suppository wax, which are solid at ordinary temperatures but liquid at body temperature and therefore, melt in the rectum or vaginal cavity and release the active component.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope.

In addition, the compounds of the preferred embodiments can exist in unsolvated as well as solvated forms with pharmaceutically acceptable solvents such as water, ethanol, and the like.

The compounds of the preferred embodiments can exist in different stereoisomeric forms by virtue of the presence of asymmetric centers in the compounds. It is contemplated that all stereoisomeric forms of the compounds, as well as mixtures thereof including racemic mixtures, form part of the preferred embodiments.

In addition, it is intended that the preferred embodiments cover compounds made either using standard organic synthetic techniques, including combinatorial chemistry or by biological methods, such as through metabolism.

In accordance with a variation of the method of treatment, the small molecule anti-cell proliferation compound may be administered in conjunction with at least one additional agent, which is active in reducing a symptom associated with cell proliferation. In one embodiment, the small molecule inhibitor may be mixed with at least one additional active ingredient to form a pharmaceutical composition. Alternatively, the small molecule inhibitor may be co-administered at the same time or according to different treatment regimens with the at least one additional active agent.

In another embodiment, the benzimidazole compound may be administered in conjunction with at least one additional active agent. These active agents include antifungals, antivirals, antibiotics, anti-inflammatories, and anticancer agents. Anticancer agents include, but are not limited to, alkylating agents (lomustine, carmustine, streptozocin, mechlorethamine, melphalan, uracil nitrogen mustard, chlorambucil cyclophosphamide, iphosphamide, cisplatin, carboplatin mitomycin thiotepa dacarbazine procarbazine, hexamethyl melamine, triethylene melamine, busulfan, pipobroman, and mitotane); antimetabolites (methotrexate, trimetrexate pentostatin, cytarabine, ara-CMP, fludarabine phosphate, hydroxyurea, fluorouracil, floxuridine, chlorodeoxyadenosine, gemcitabine, thioguanine, and 6-mercaptopurine); DNA cutters (bleomycin); topoisomerase I poisons (topotecan, irinotecan, and camptothecin); topoisomerase II poisons (daunorubicin, doxorubicin, idarubicin, mitoxantrone, teniposide, and etoposide); DNA binders (dactinomycin and mithramycin); and spindle poisons (vinblastine, vincristine, navelbine, paclitaxel, and docetaxel).

In another embodiment, the benzimidazole compounds of the preferred embodiments are administered in conjunction with one or more other therapies. These therapies include, but are not limited to radiation, immunotherapy, gene therapy and surgery. These combination therapies may be administered simultaneously or sequentially. For example, radiation may be administered along with the administration of benzimidazole compounds, or may be administered at any time before or after administration of benzimidazole compounds.

Method of Treatment

In accordance with preferred embodiments, the compounds and pharmaceutical compositions can be used in the treatment of hyperproliferative disorders in mammals, including humans. Such disorders include, but are not limited to, tumorigenesis and other proliferative diseases such as, but not limited to, cancers, inflammatory disorders, and circulatory diseases. For example, hyperproliferation of cells can cause psoriasis, thrombosis, atherosclerosis, coronary heart disease, myocardial infarction, stroke, smooth muscle neoplasms, uterine fibroid or fibroma, and obliterative diseases of vascular grafts and transplanted organs. Abnormal cell proliferation is most commonly associated with tumor formation and cancer. The particular compounds disclosed herein were identified by their ability to suppress abnormal cellular proliferation. Methods of use include a step of administering a therapeutically effective amount of an active ingredient to a mammal in need thereof.

Preferably, the compounds of the preferred embodiments are administered in the form of a pharmaceutical formulation. Thus, the compounds can be administered orally, parenterally, topically, rectally, etc., in appropriate dosage units, as desired.

Actual dosage levels of active ingredients in the pharmaceutical compositions can be S varied so as to administer an amount of the active compound(s) that is effective to achieve the desired therapeutic response for a particular patient.

The compounds of the preferred embodiments can be administered to a patient at dosage levels in the range of about 0.1 to about 1000 mg per day. For a normal human adult having a body weight of about 70 kilograms, a dosage in the range of about 0.01 to about 100 mg per kilogram of body weight per day is preferable. The selected dosage level will depend upon the activity of the particular compound, the route of administration, the severity of the condition being treated, and the condition and prior medical history of the patient being treated. If desired, the effective daily dose can be divided into multiple doses for purposes of administration, e.g., two to four separate doses per day. It will be understood, however, that the specific dose level for any particular patient will depend upon a variety of factors, including, body weight, general health, diet, time and route of administration, combination with other drugs, and severity of the particular disease being treated. The determination of optimum dosages for a particular patient is well known to those skilled in the art.

Many modifications and variations of the embodiments described herein can be made without departing from the scope, as is apparent to those skilled in the art. The specific embodiments described herein are offered by way of example only.

1. A method for the treatment of cancer comprising administration of a formulation comprising at least one compound of a pharmaceutical composition of the formulae: 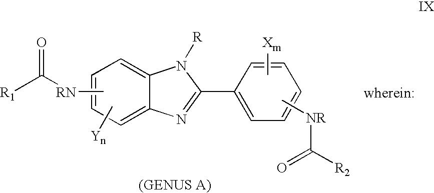
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;
n is an integer from one to three;
m is an integer from one to four;
R is selected from the group consisting of H, CH₃, C₂H₅, C₃H₇, C₄H₉, CH₂Ph, CH₂C₆H₄—F(p-), COCH₃, COCH₃, COOCH₂CH₃, CH₂CH₂N(CH₃)₂, and CH₂CH₂CH₂N(CH₃)₂; and
R₁ and R₂ are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom.

2. The method of claim 1, wherein method for the treatment of cancer comprises administering a formulation comprising at least one compound selected from the group consisting of

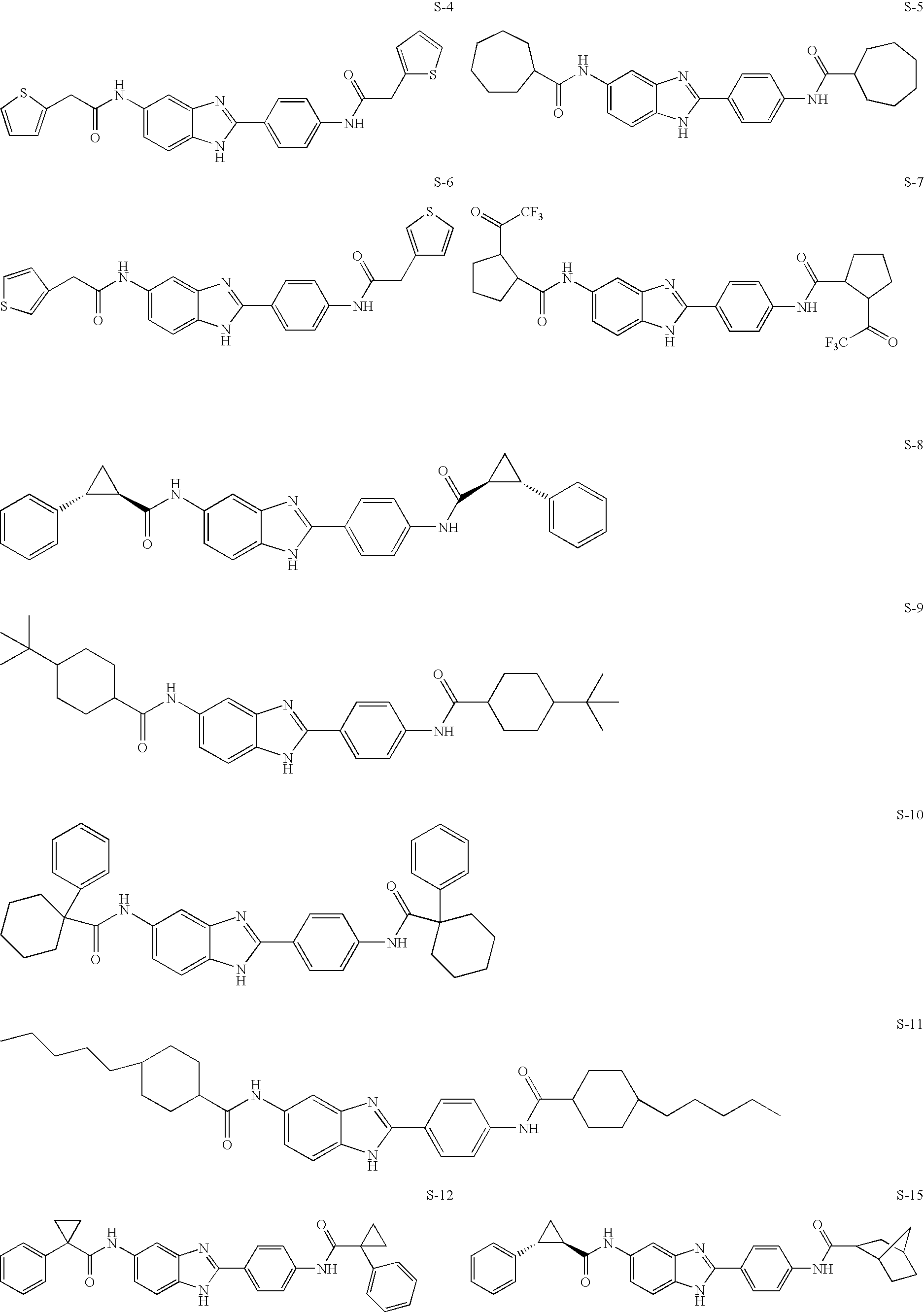

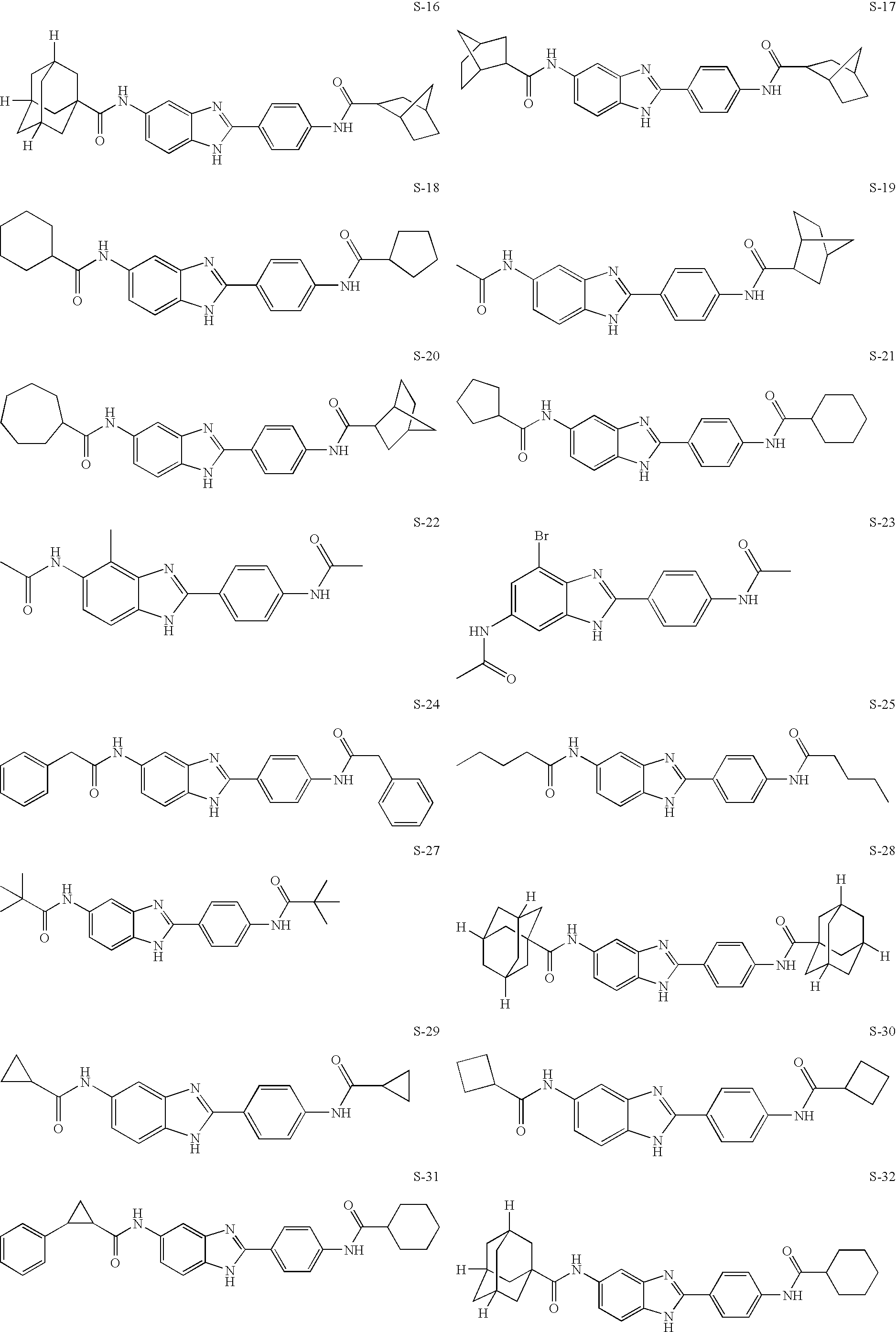

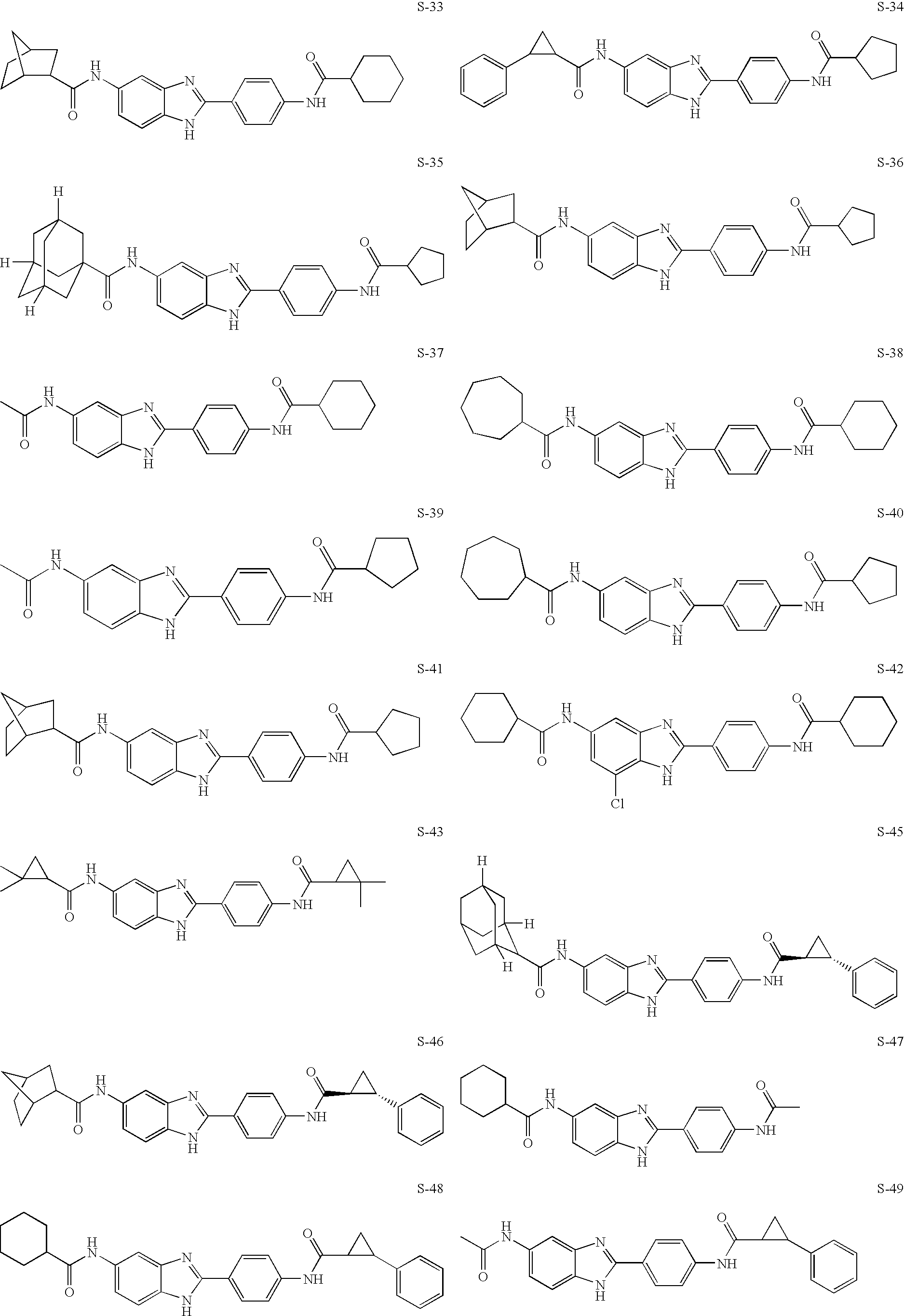

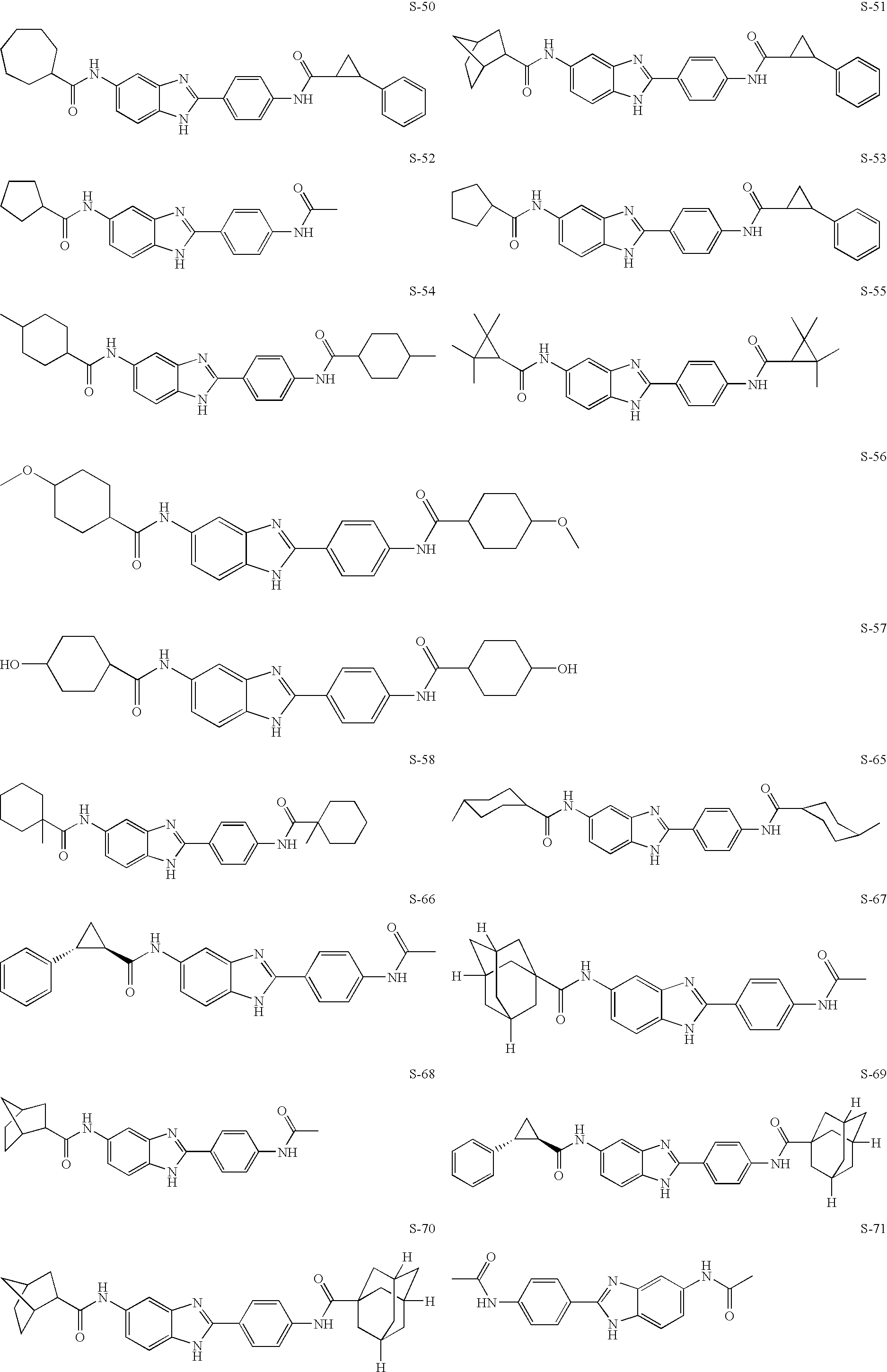

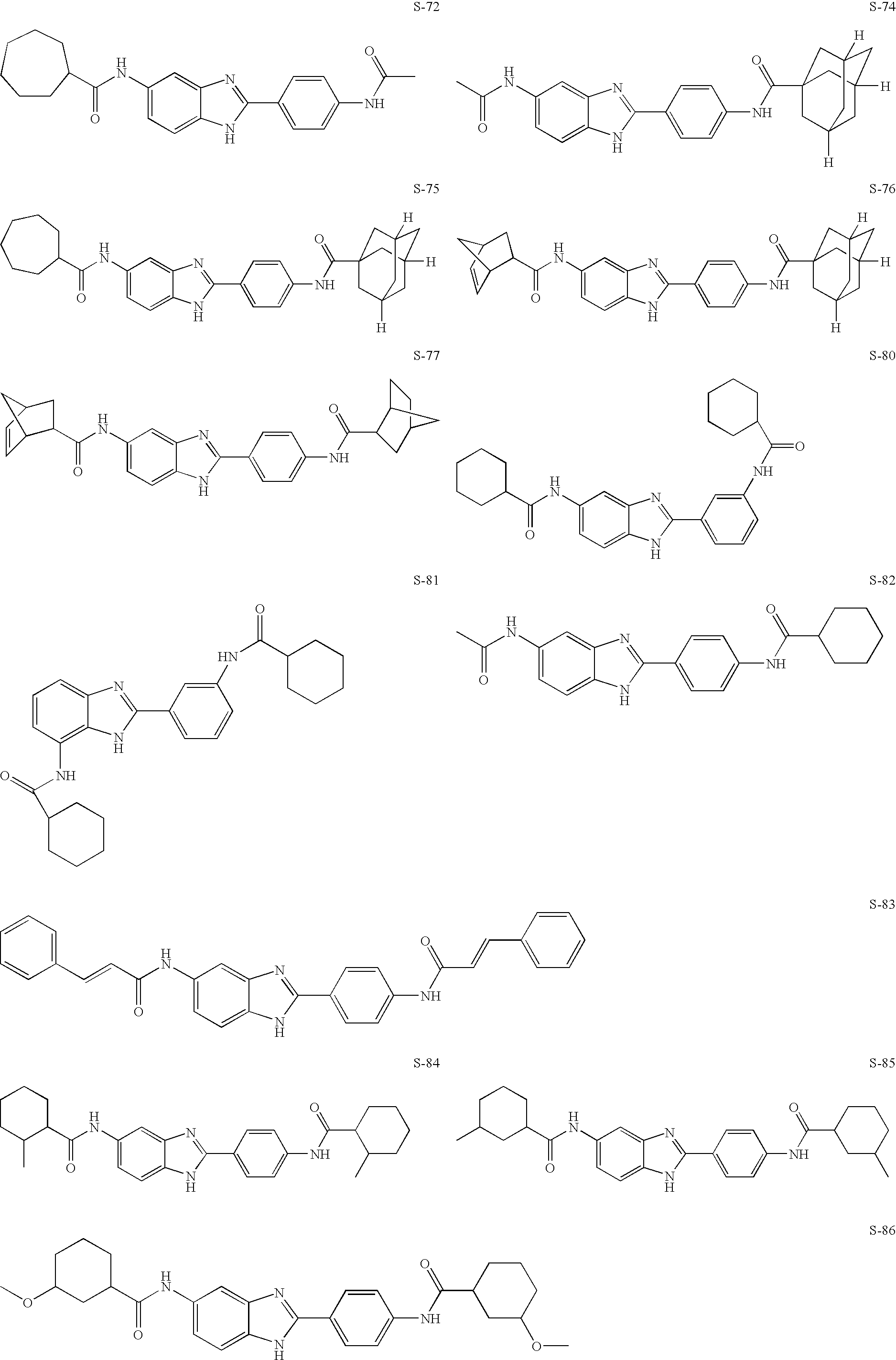

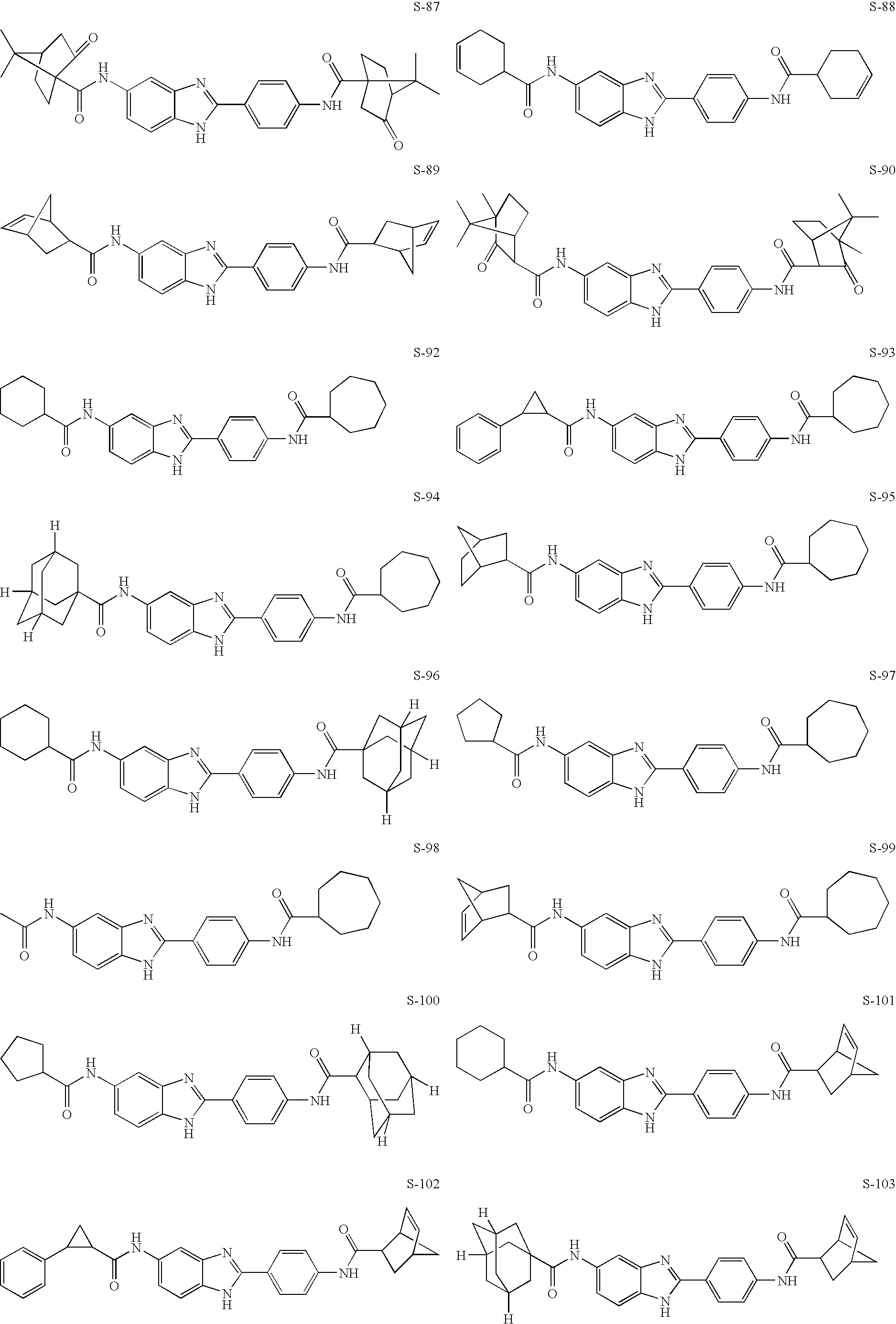

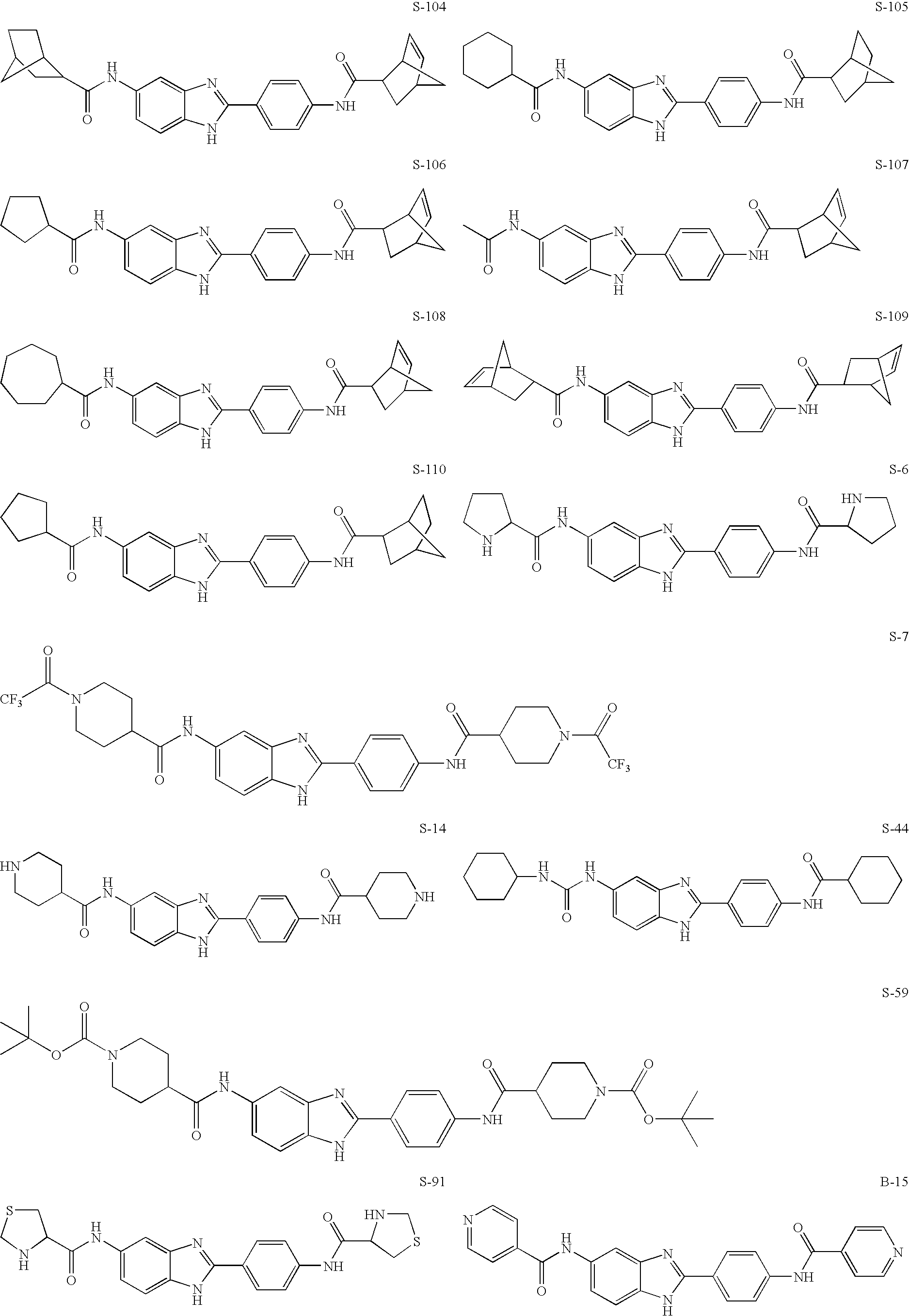

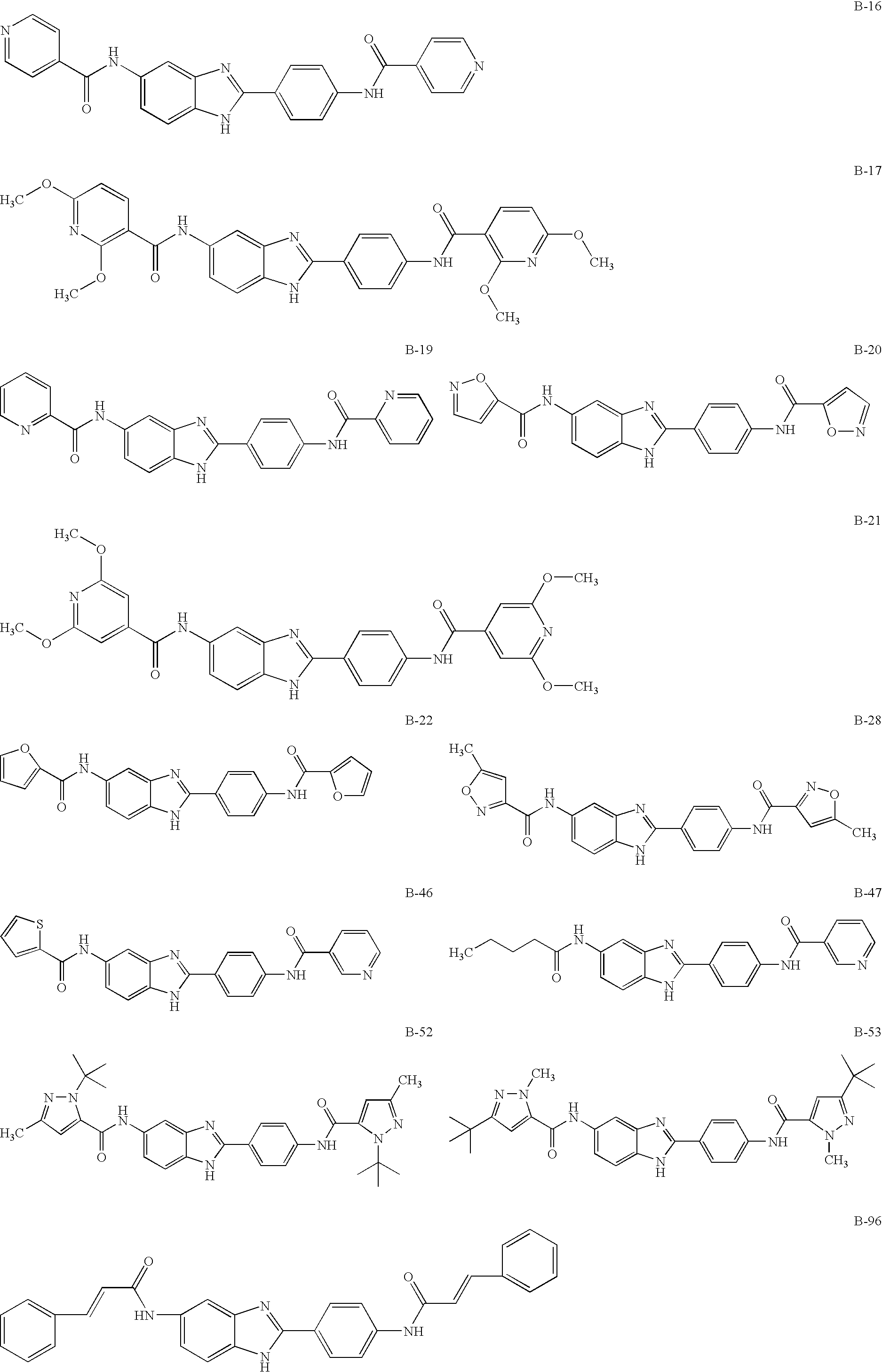

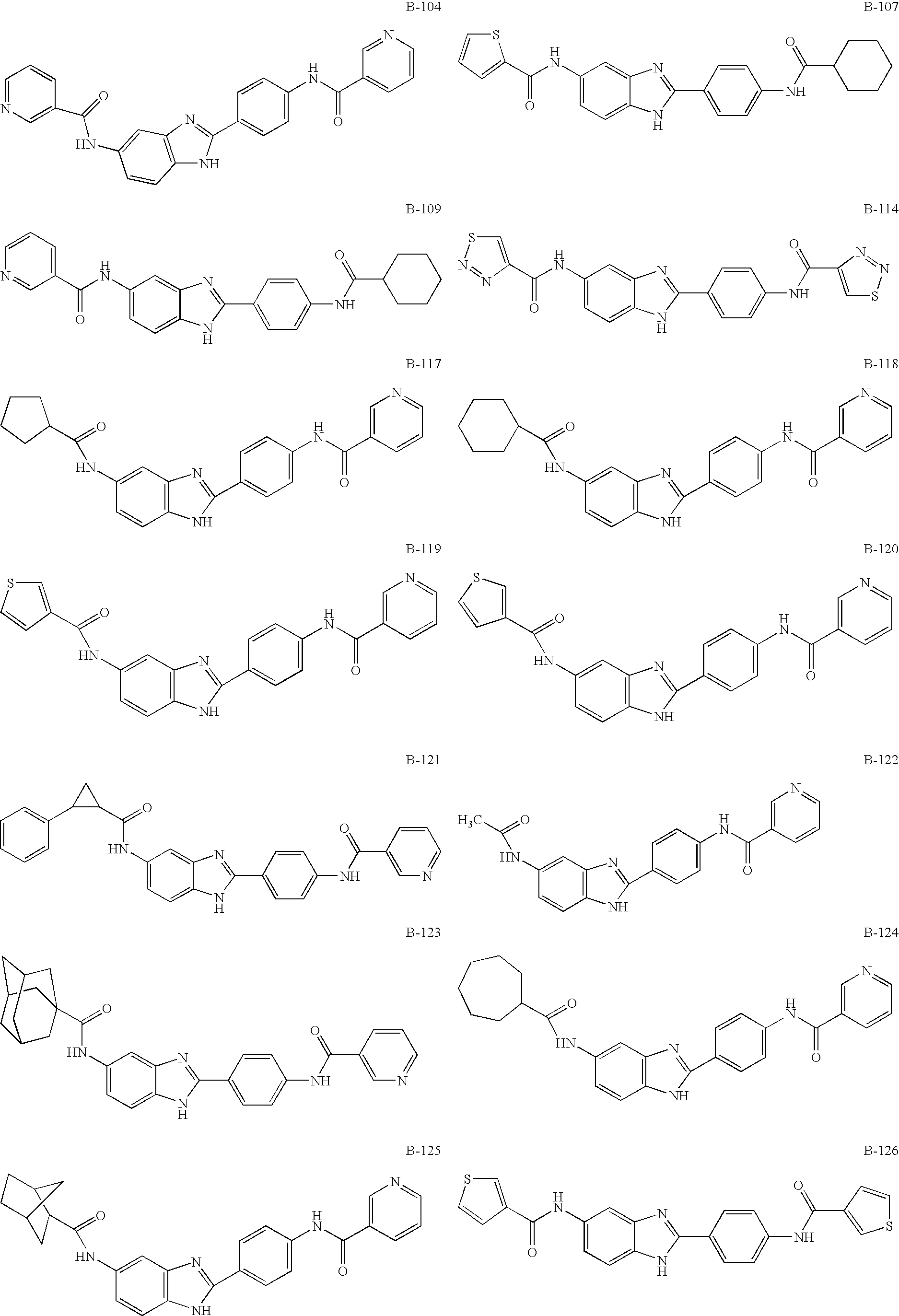

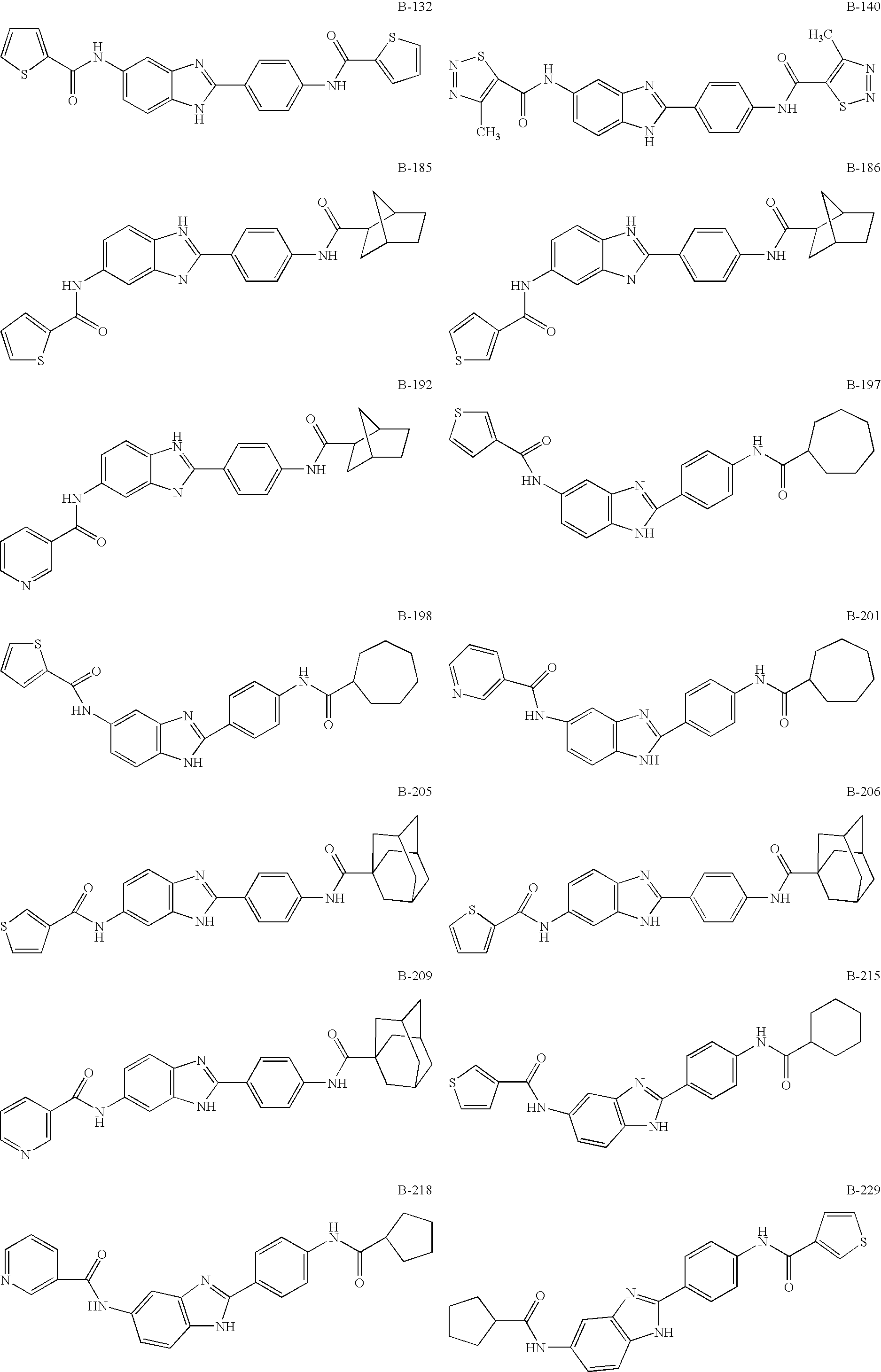

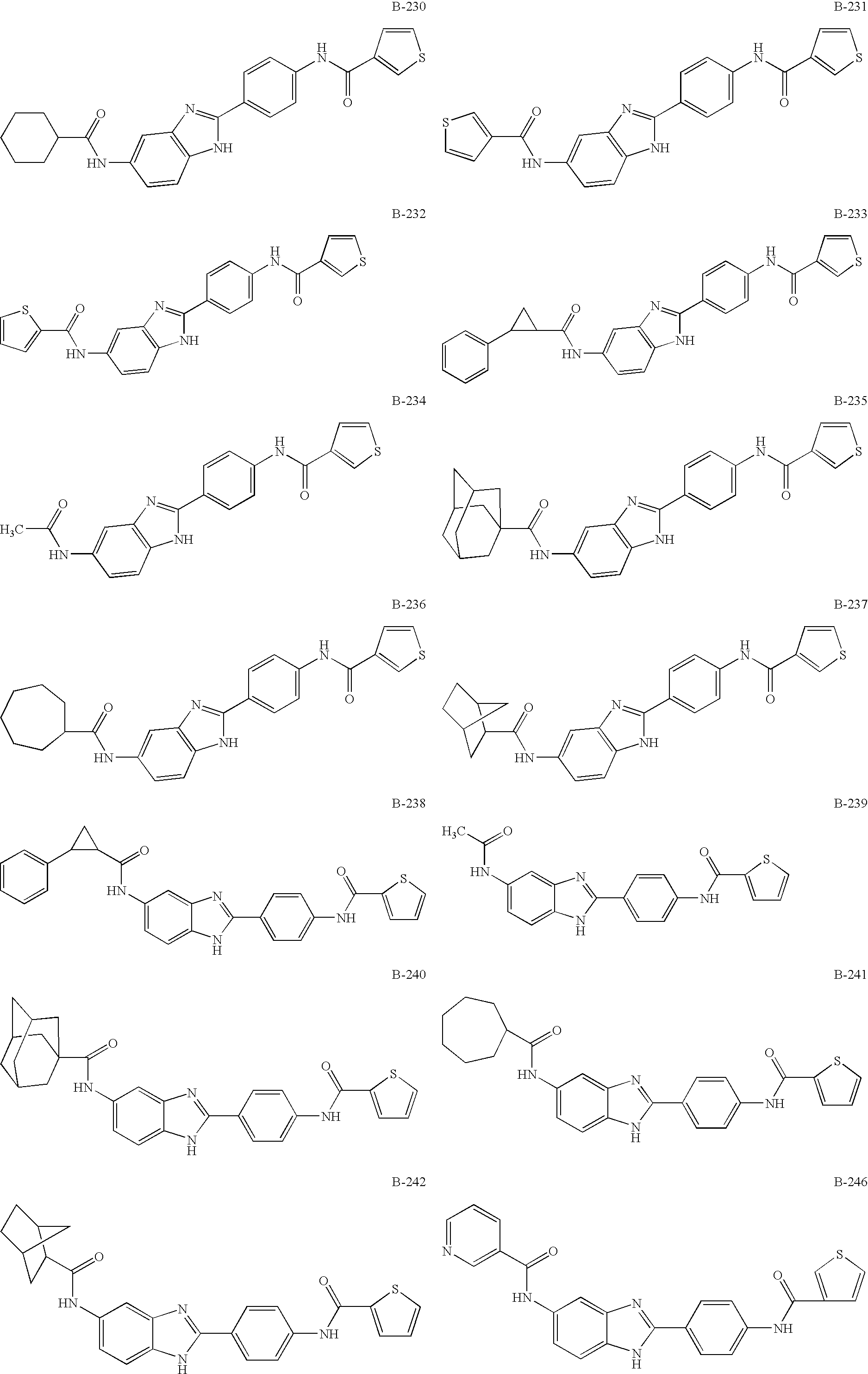

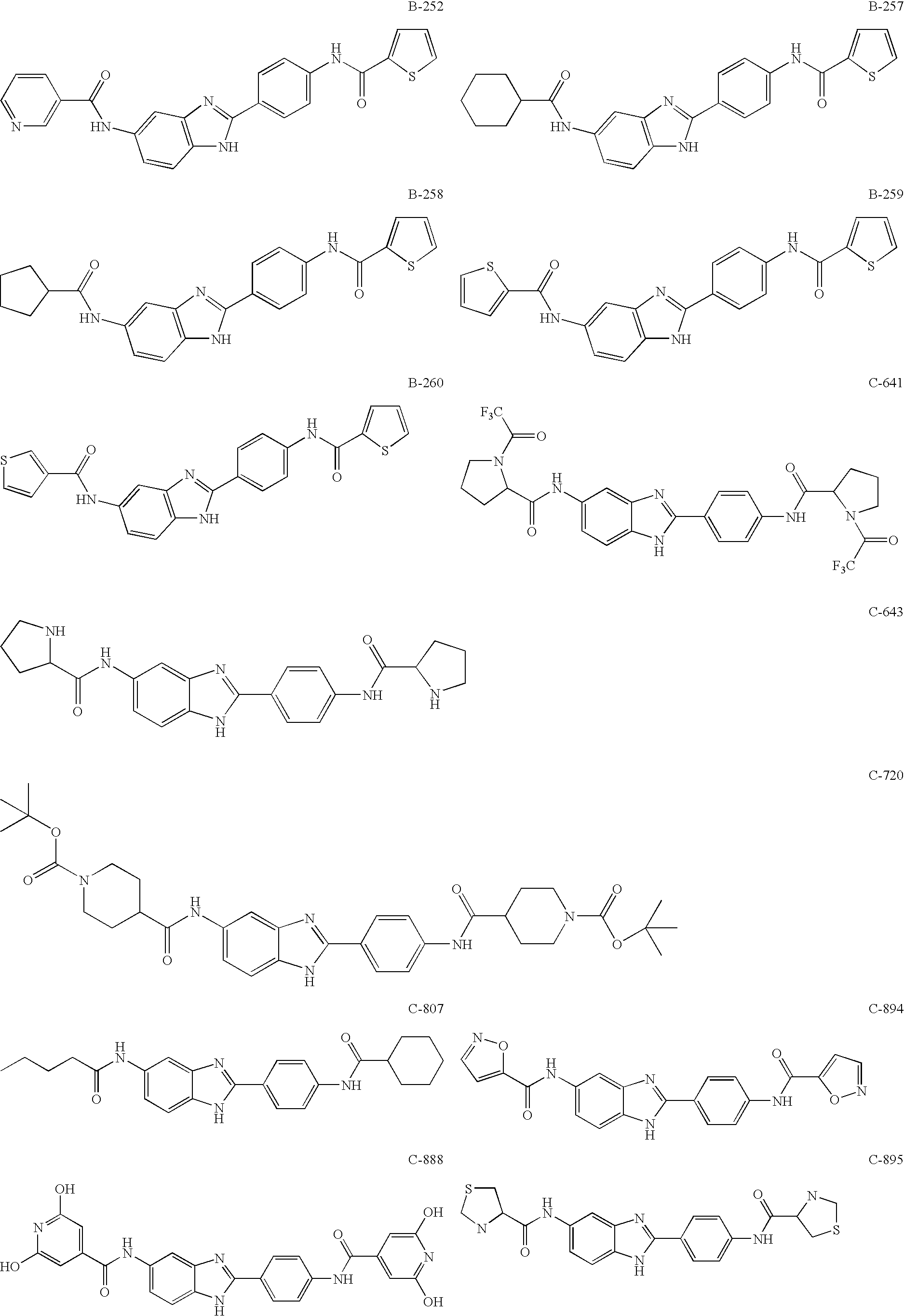

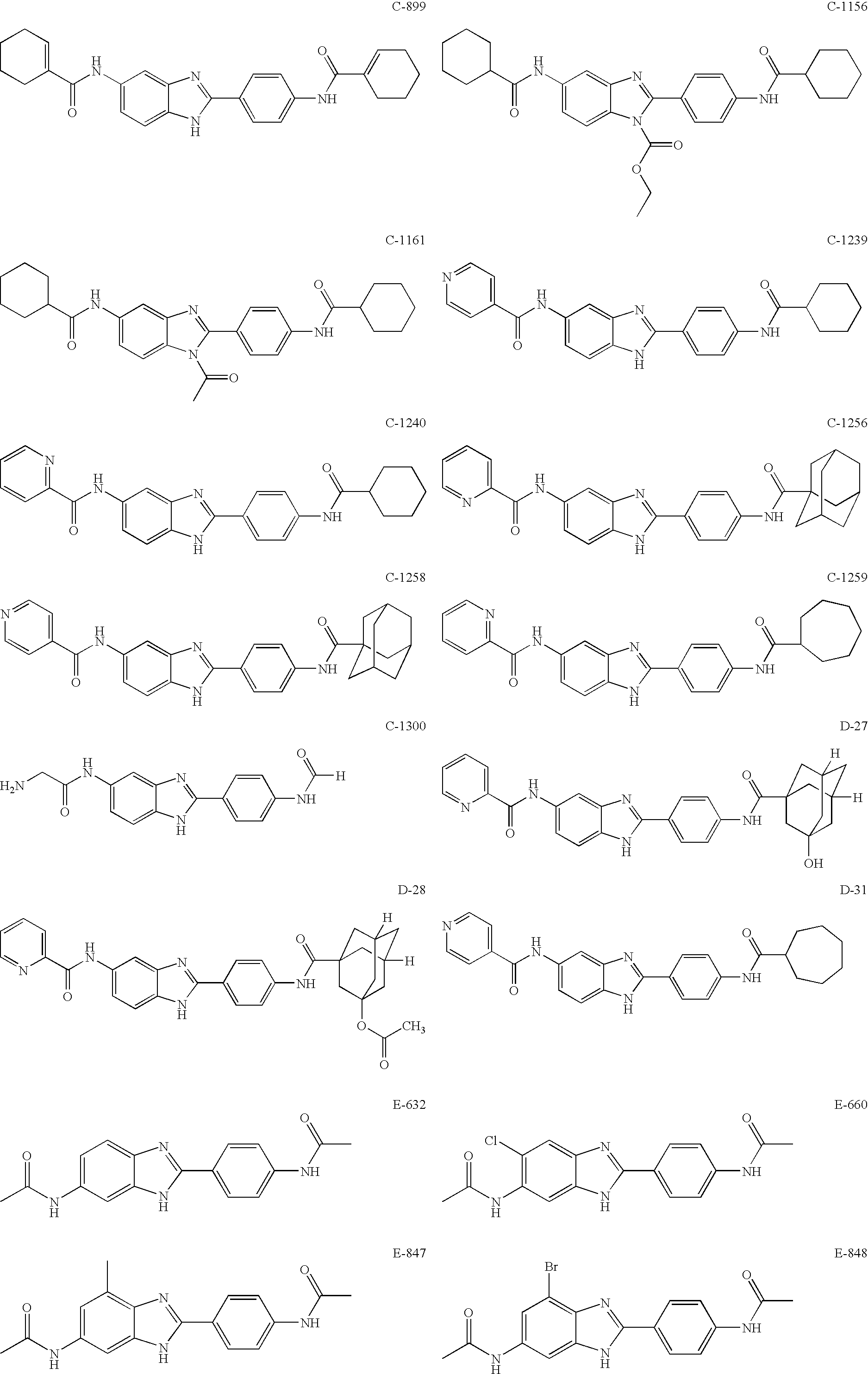

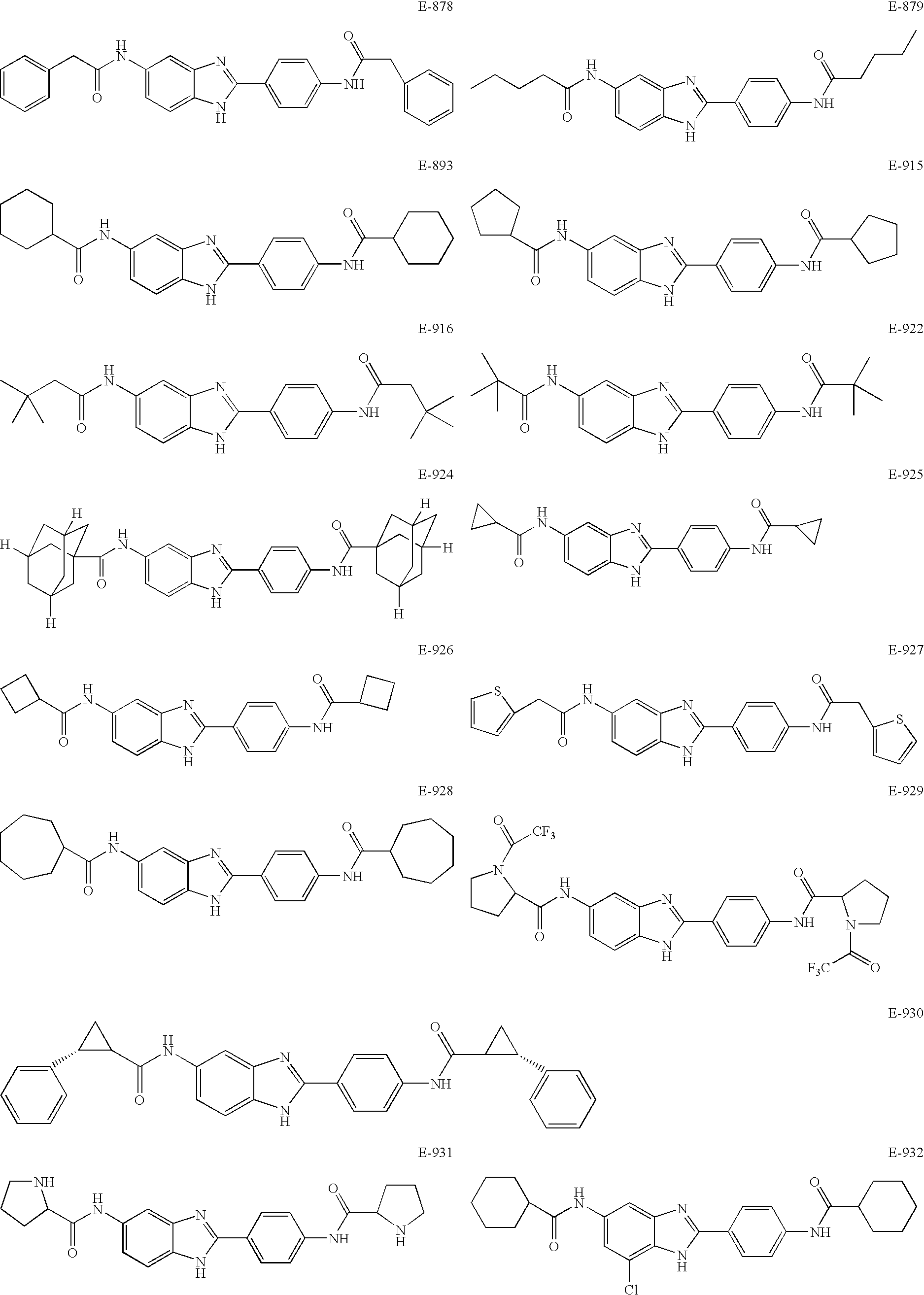

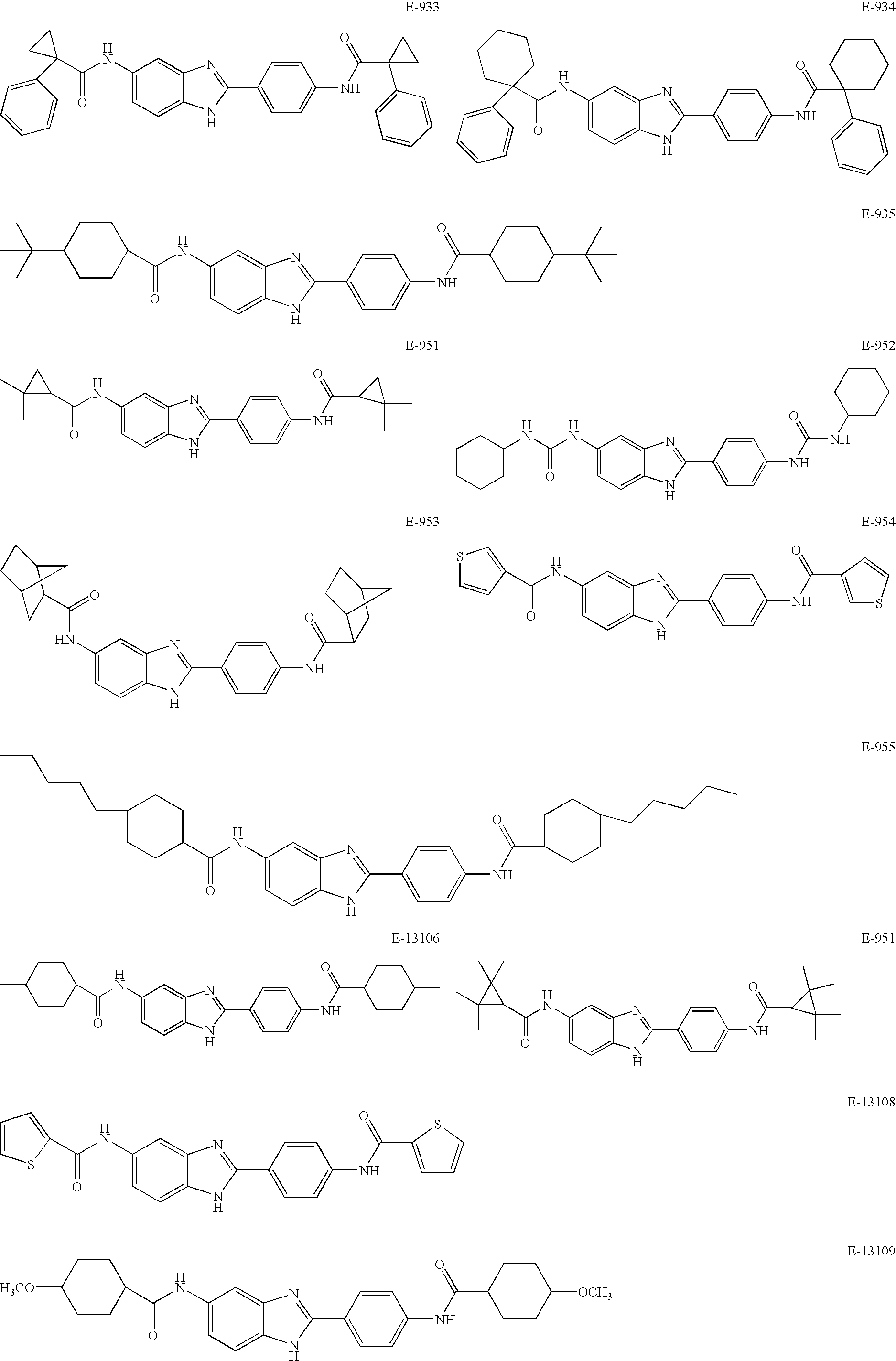

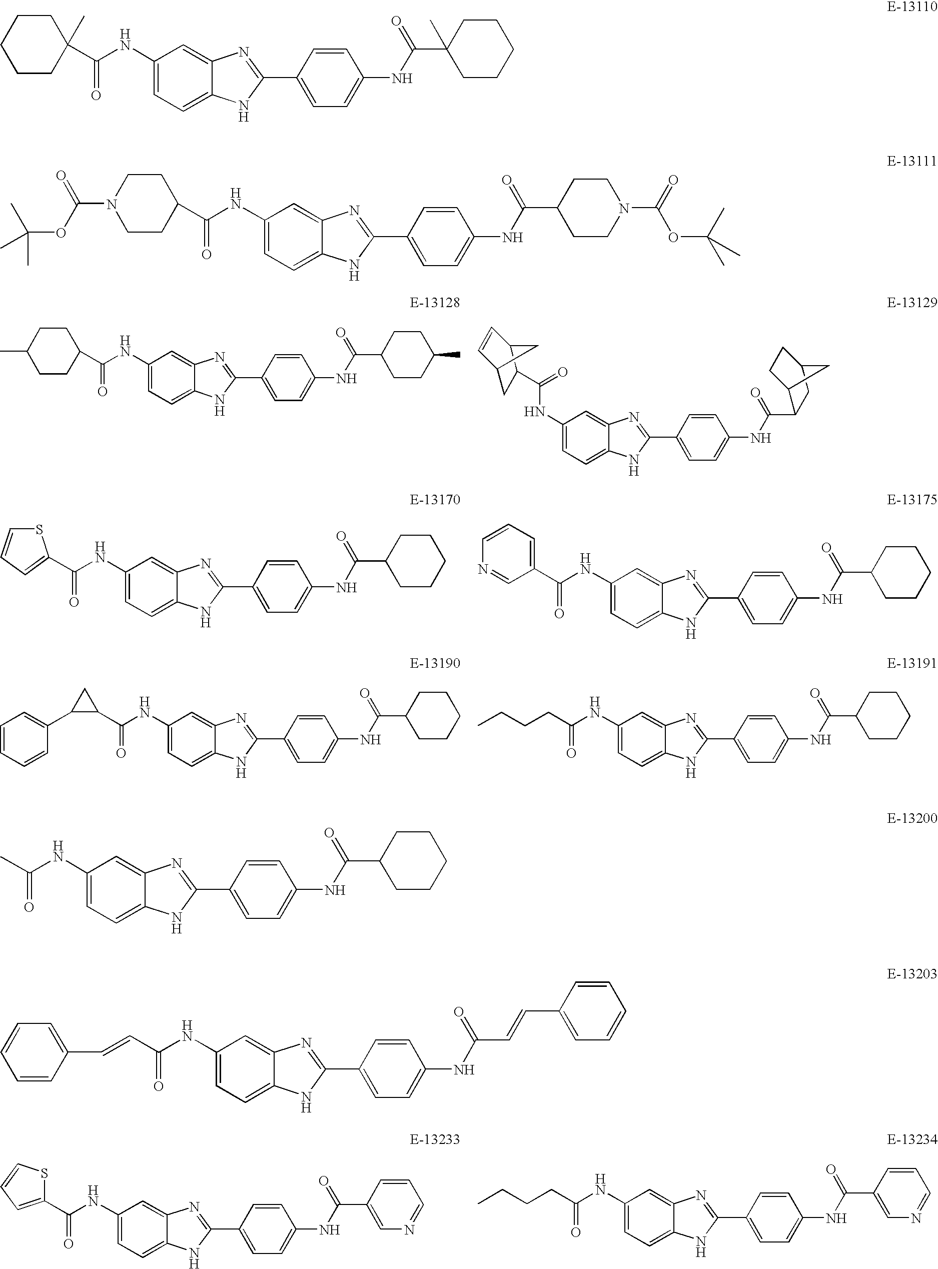

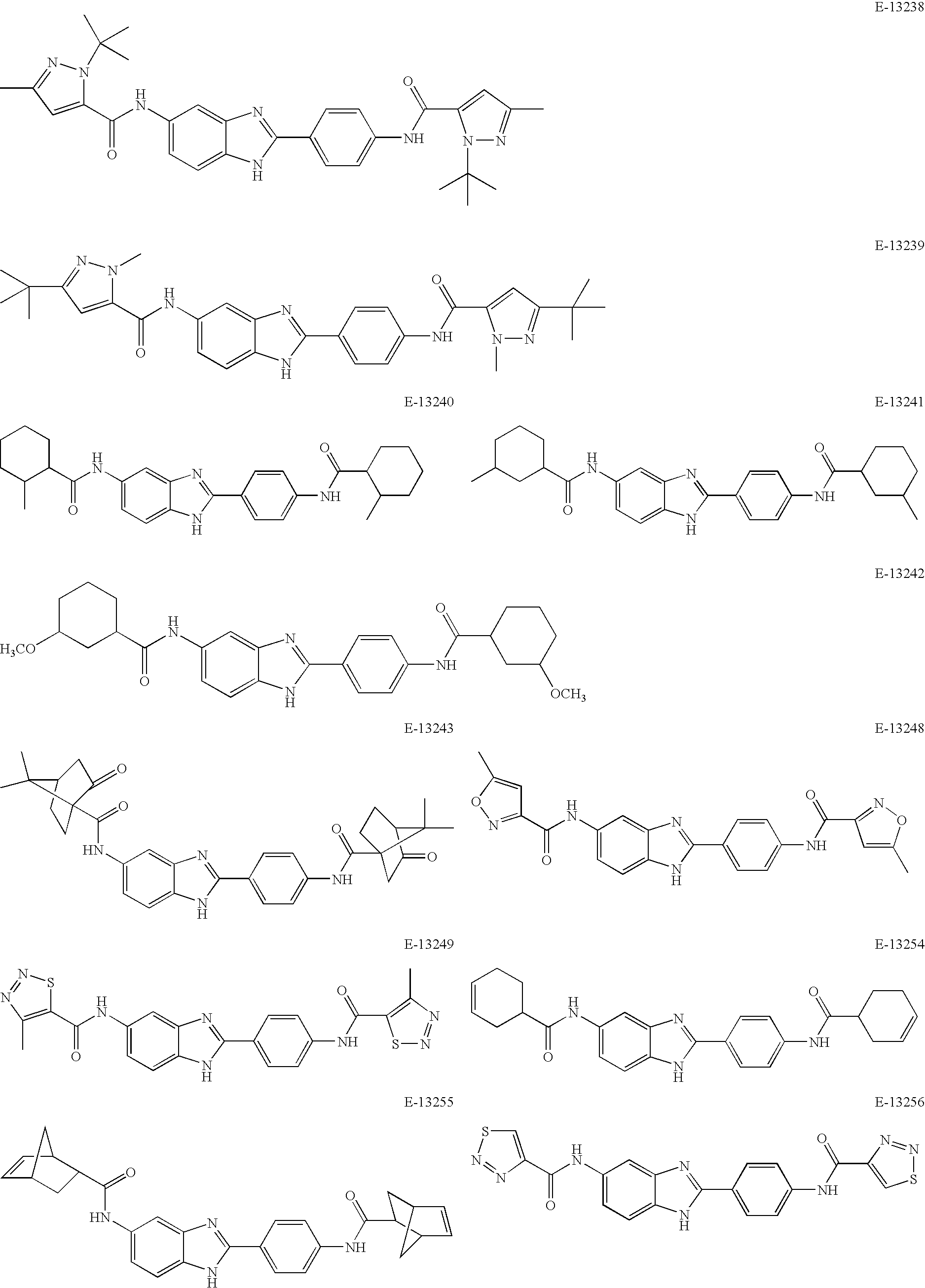

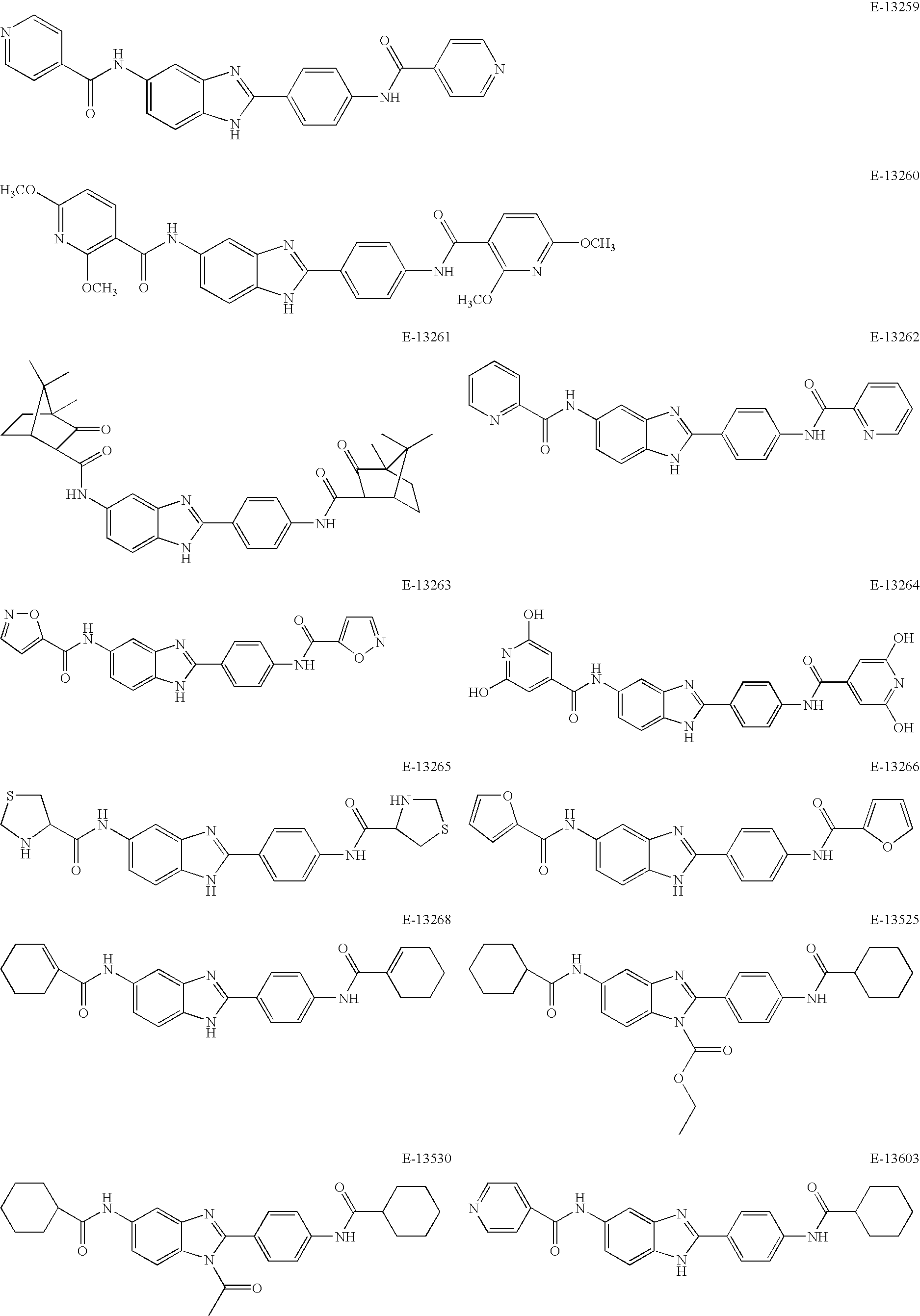

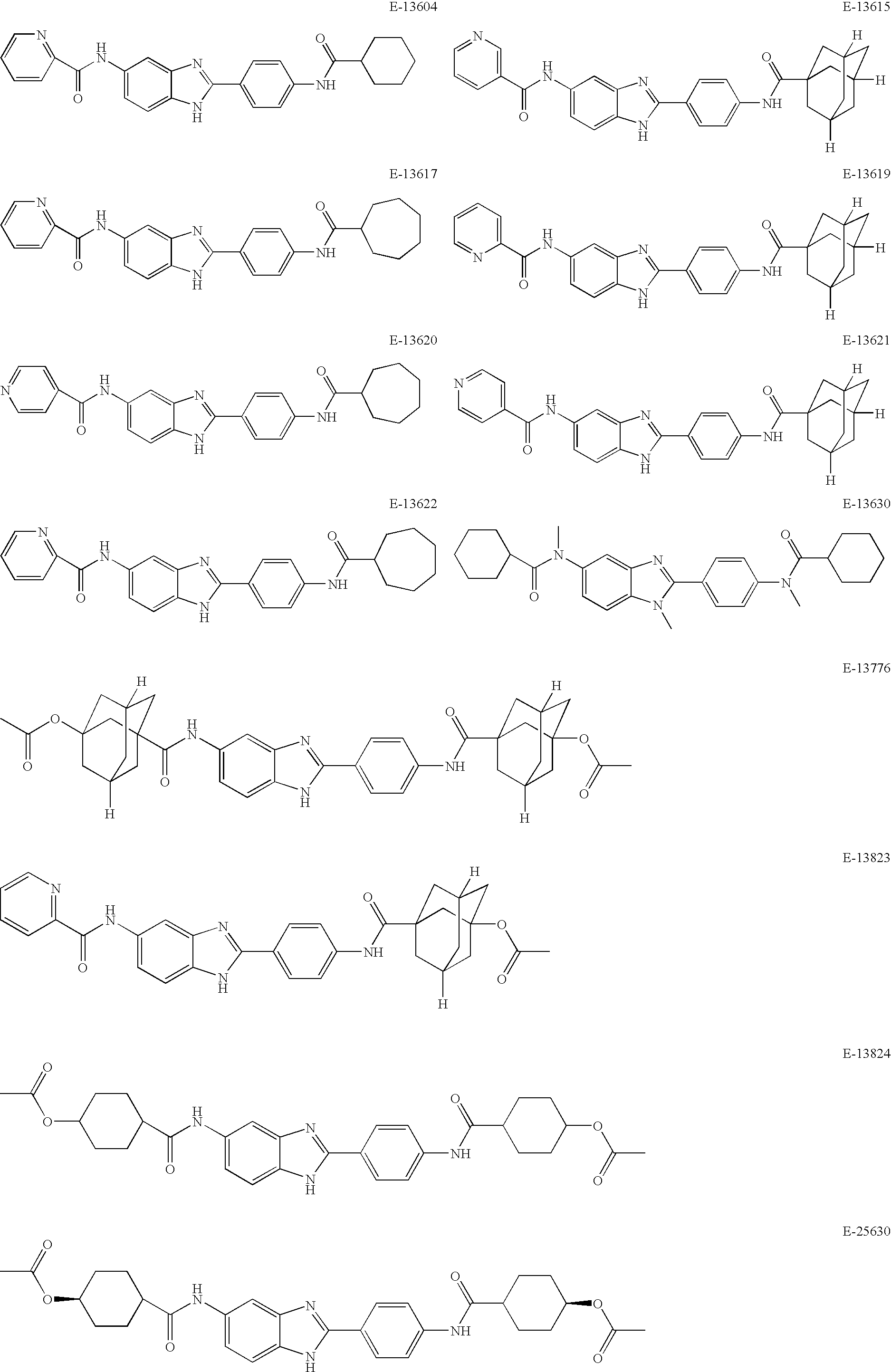

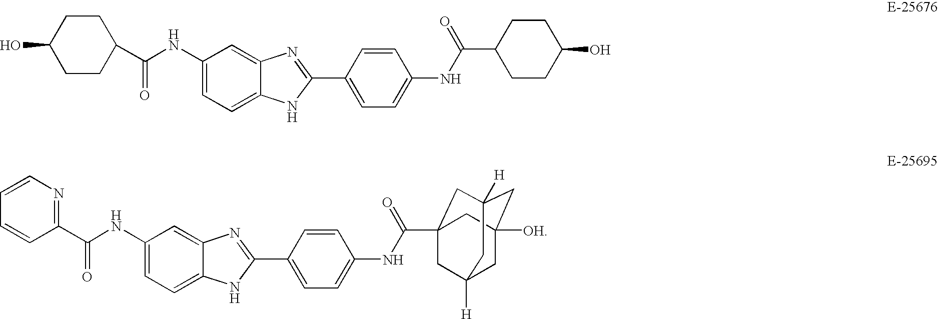

3. A method for the treatment of cancer comprising administration of a formulation comprising at least one compound of a pharmaceutical composition of the formulae: 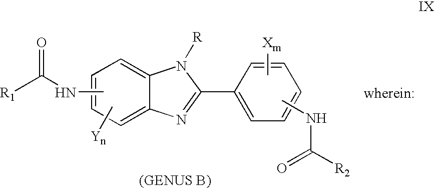
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;
n is an integer from one to three;
m is an integer from one to four;
R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and
R₁ and R₂ are independently selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, and substituted heteroarylcycloalkyl, heterocyclic ring, substituted heterocyclic ring, heteroatom, substituted heteroatom, aryl, and substituted aryl, wherein at least one of R₁ and R₂ is selected from aryl or substituted aryl.

4. The method of claim 3, wherein method for the treatment of cancer comprises administering a formulation comprising at least one compound selected from the group consisting of

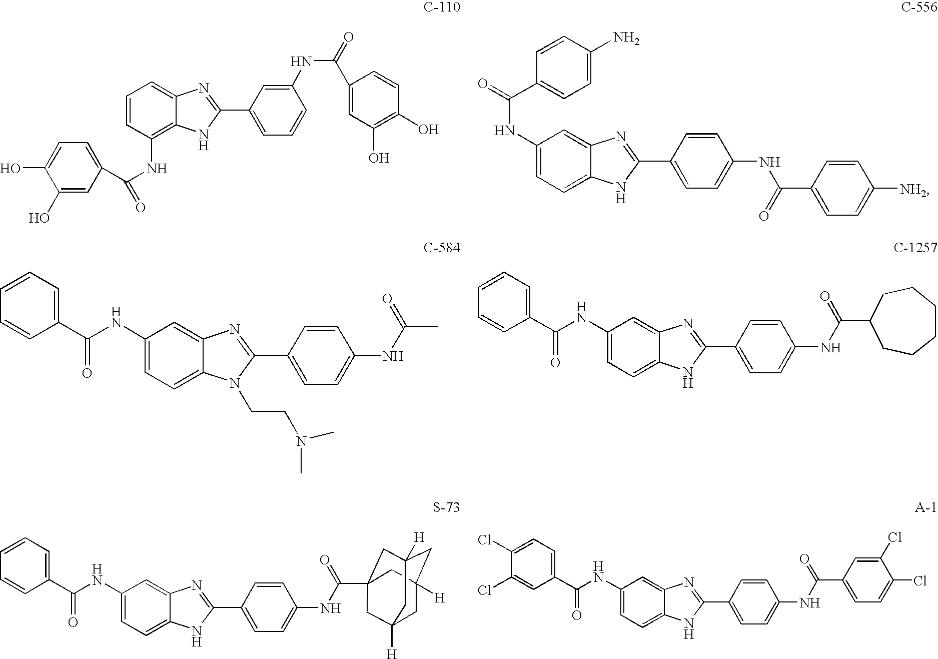

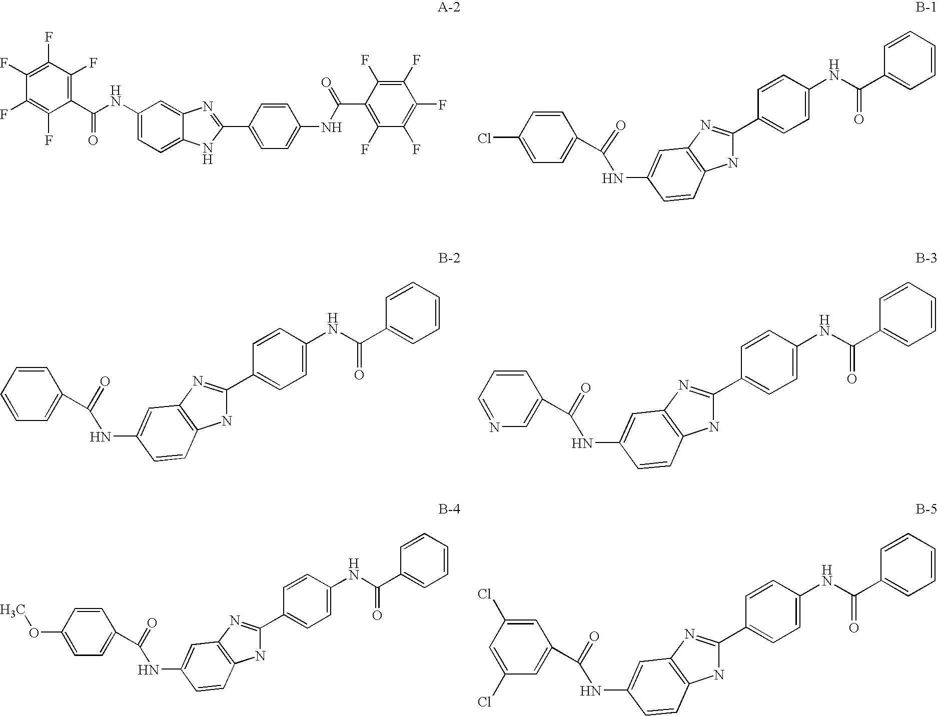

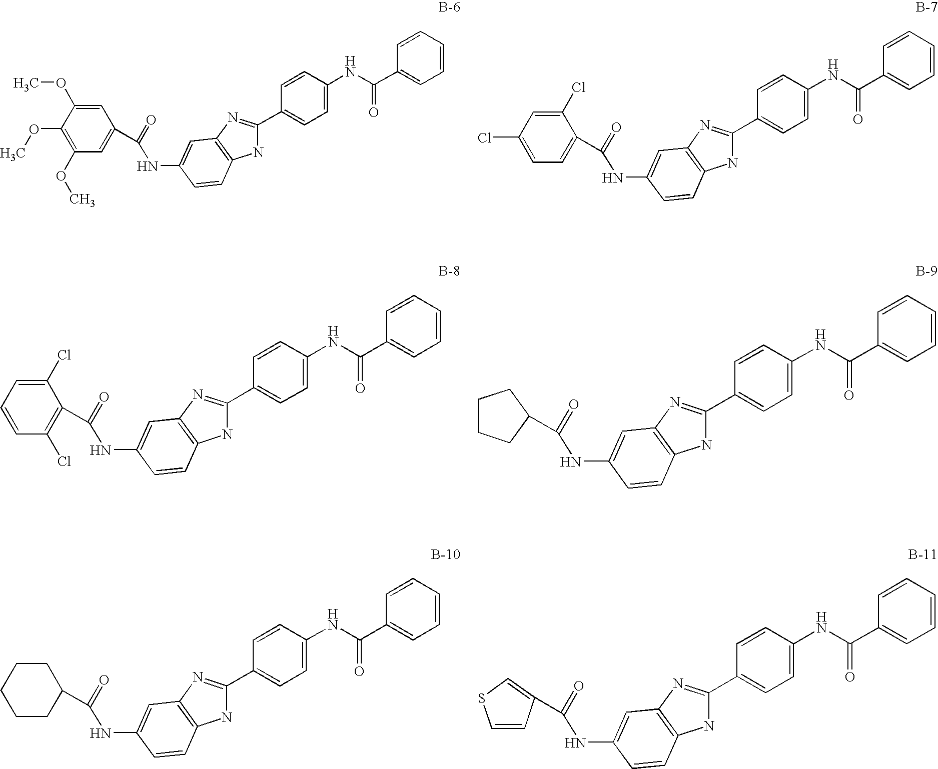

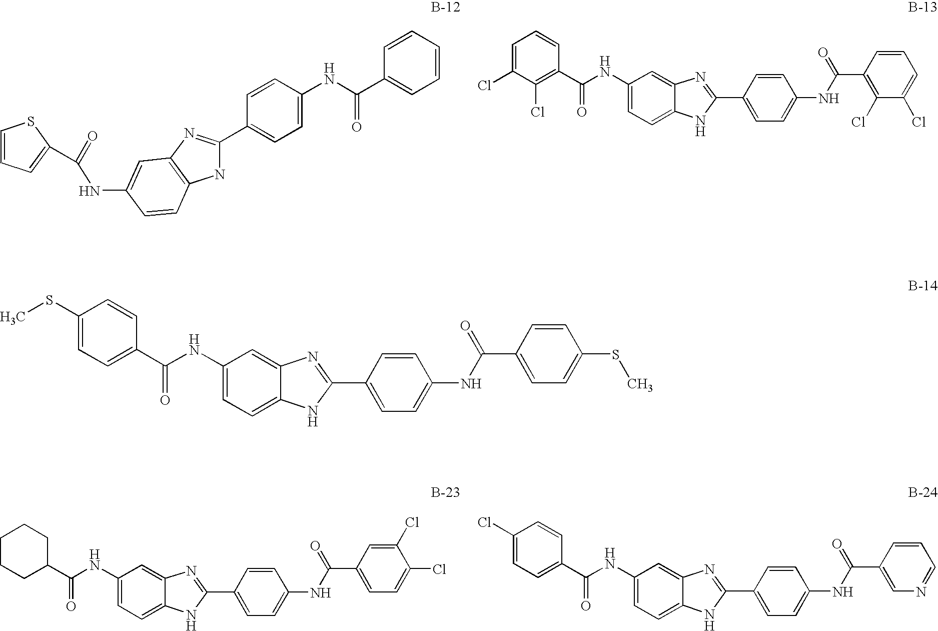

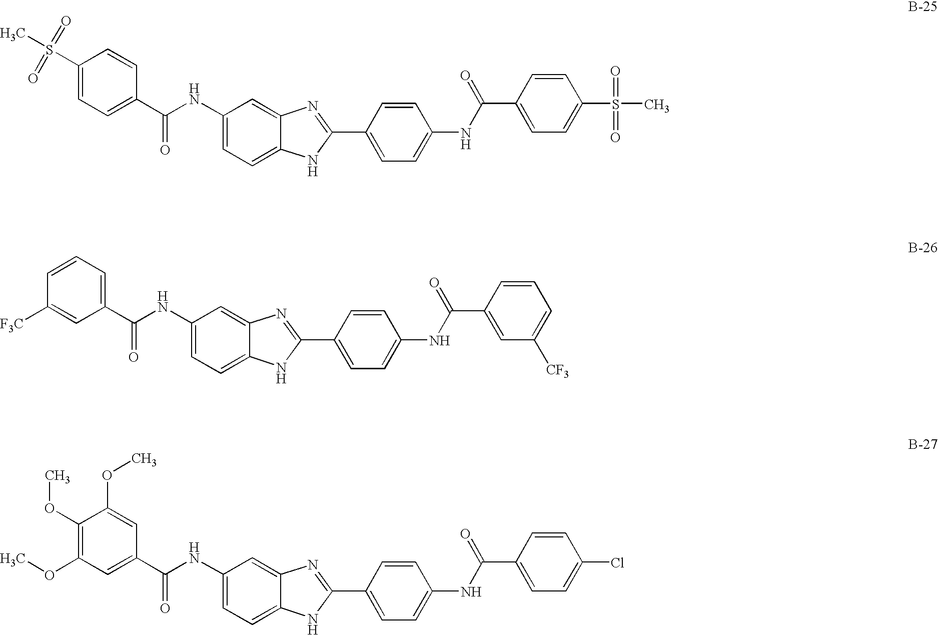

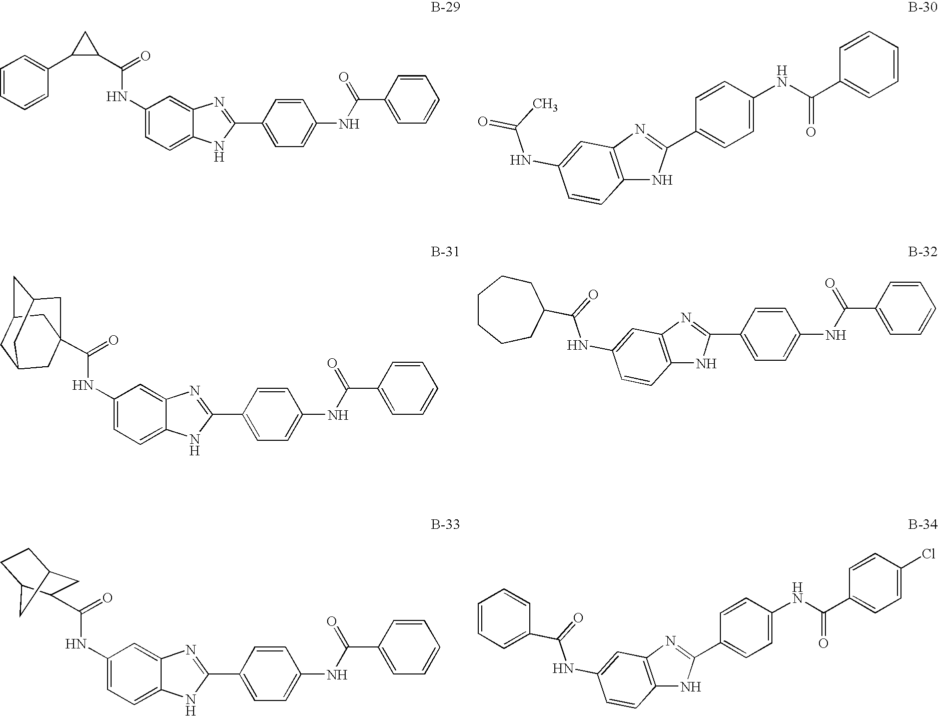

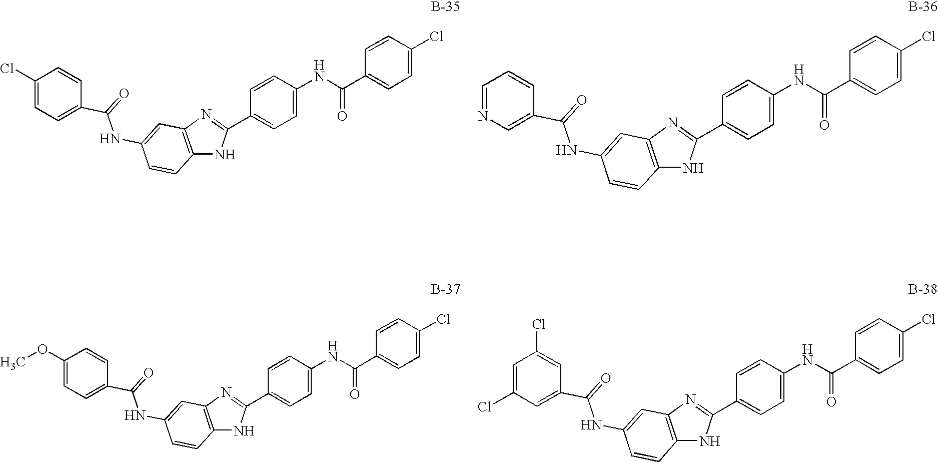

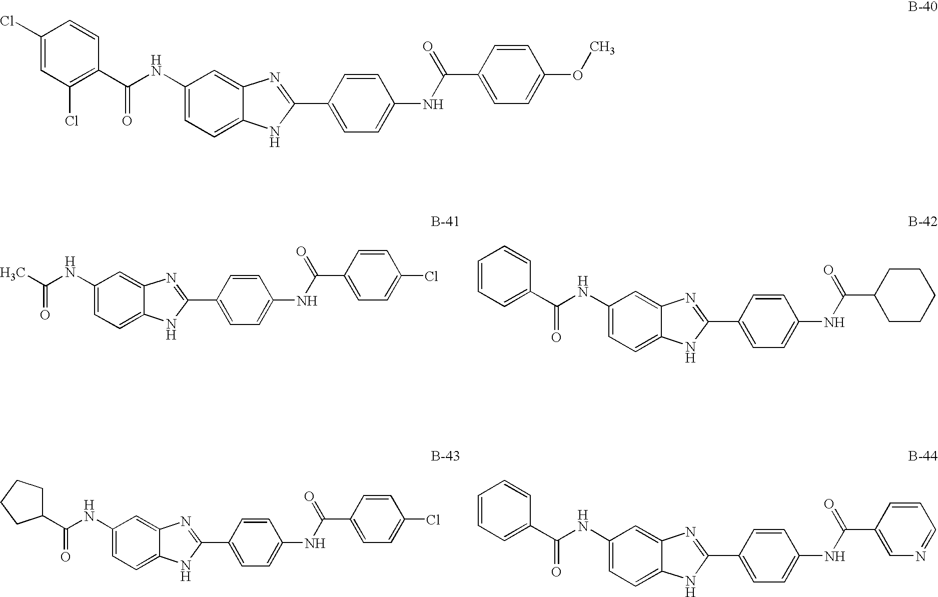

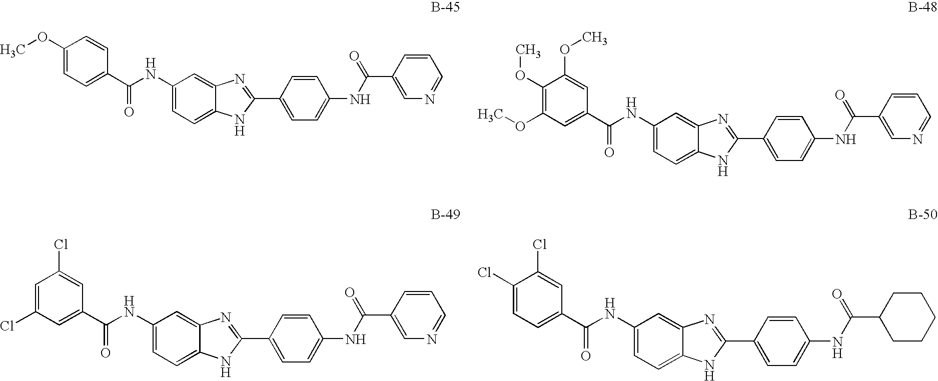

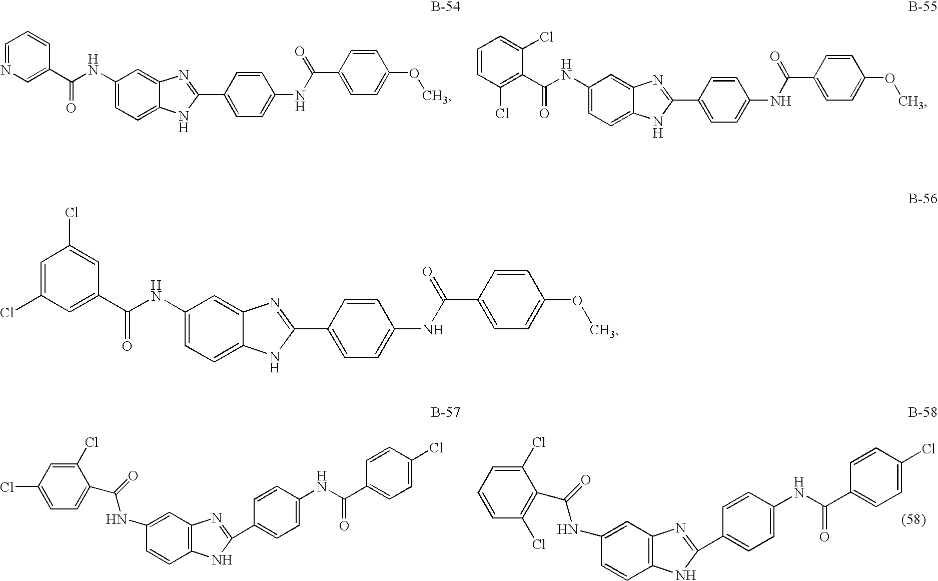

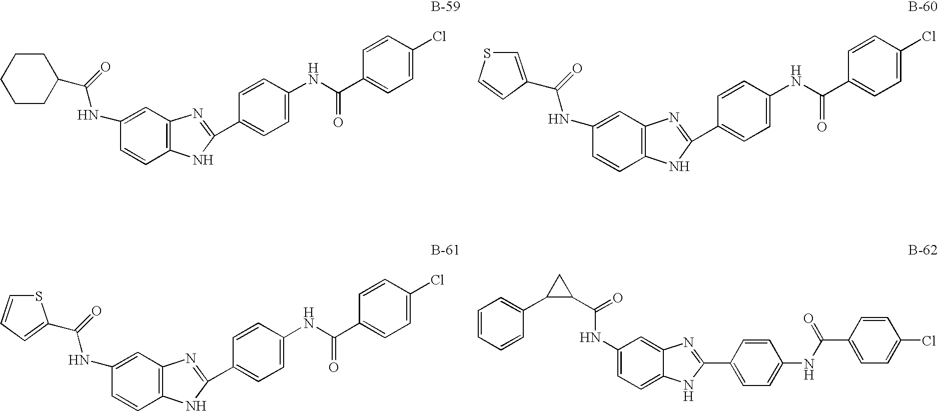

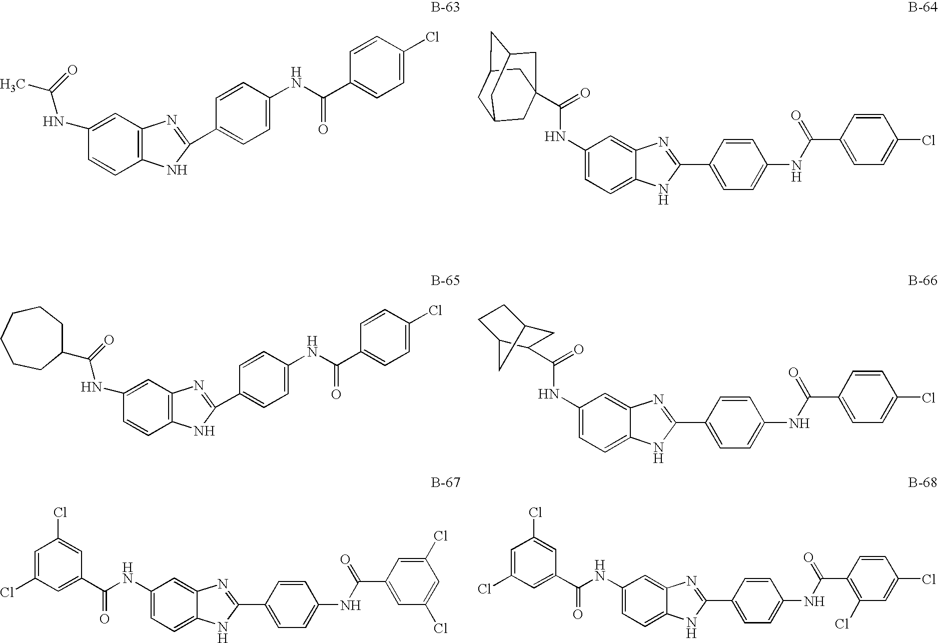

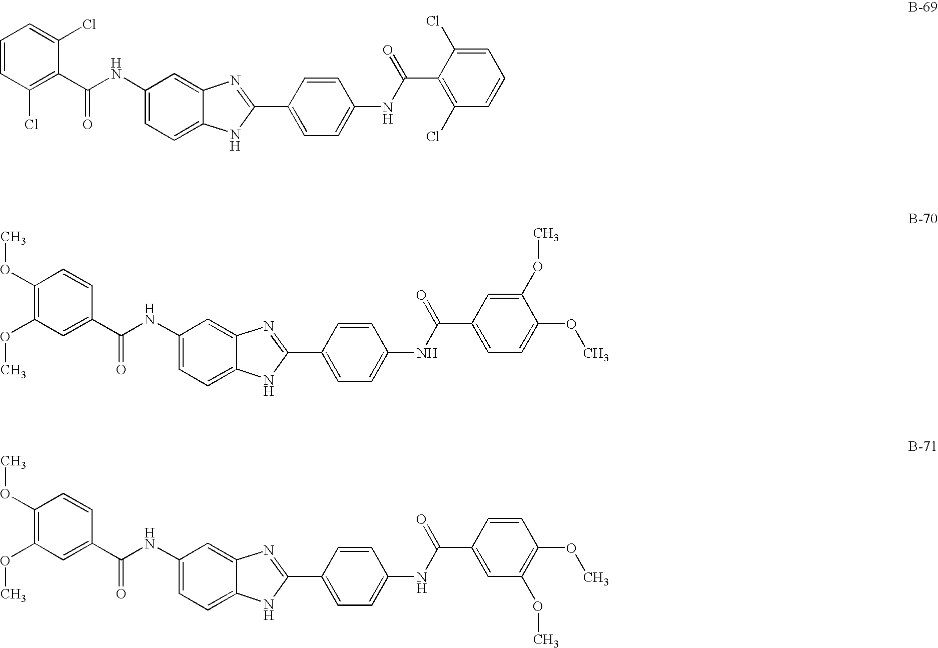

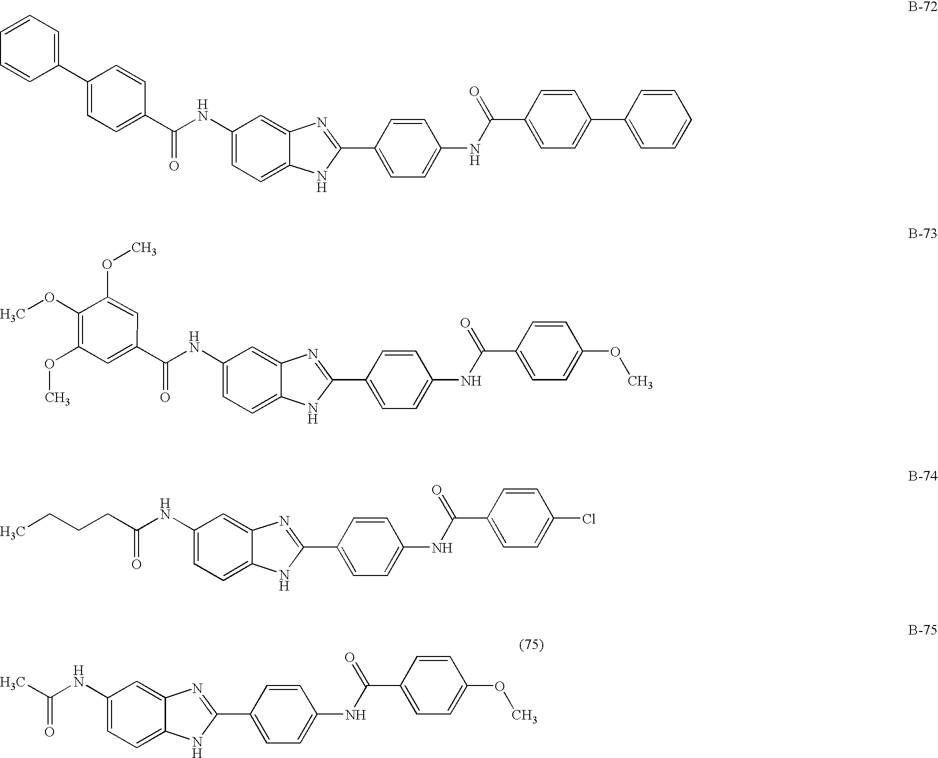

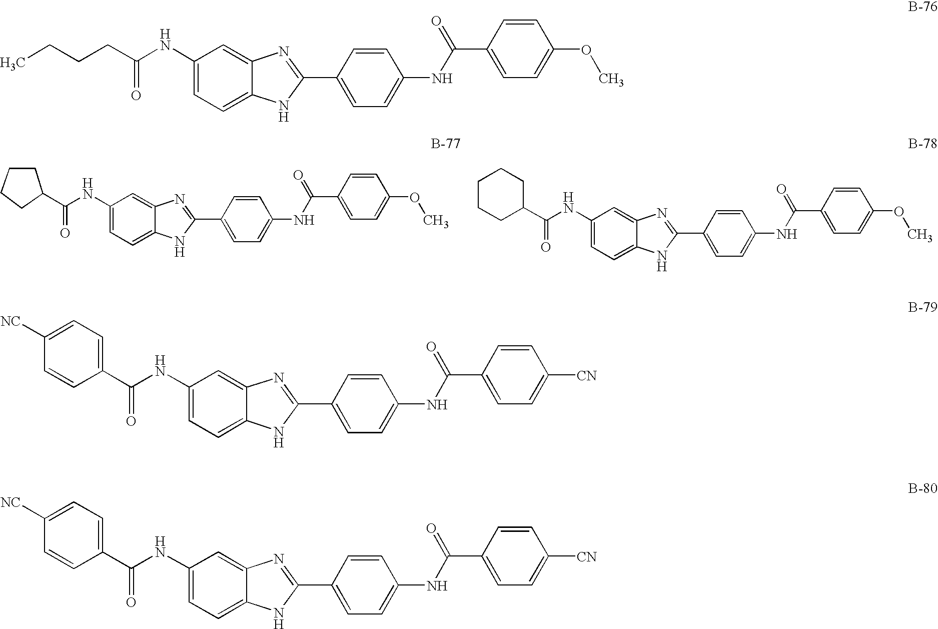

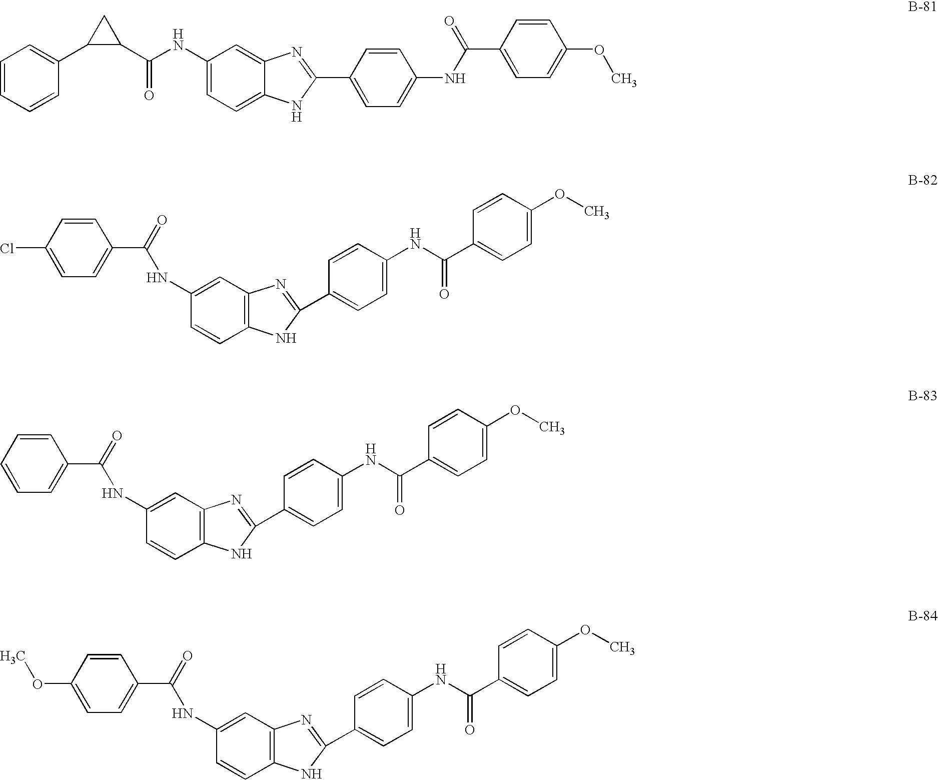

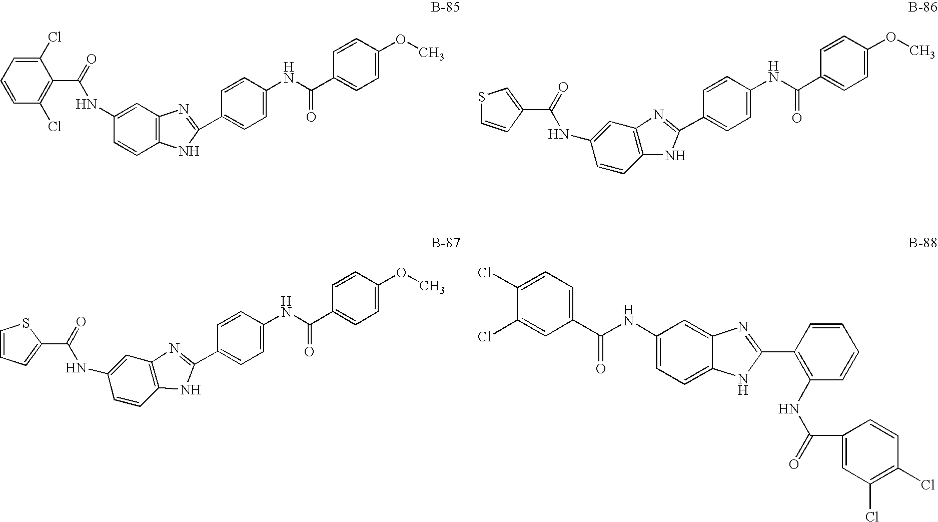

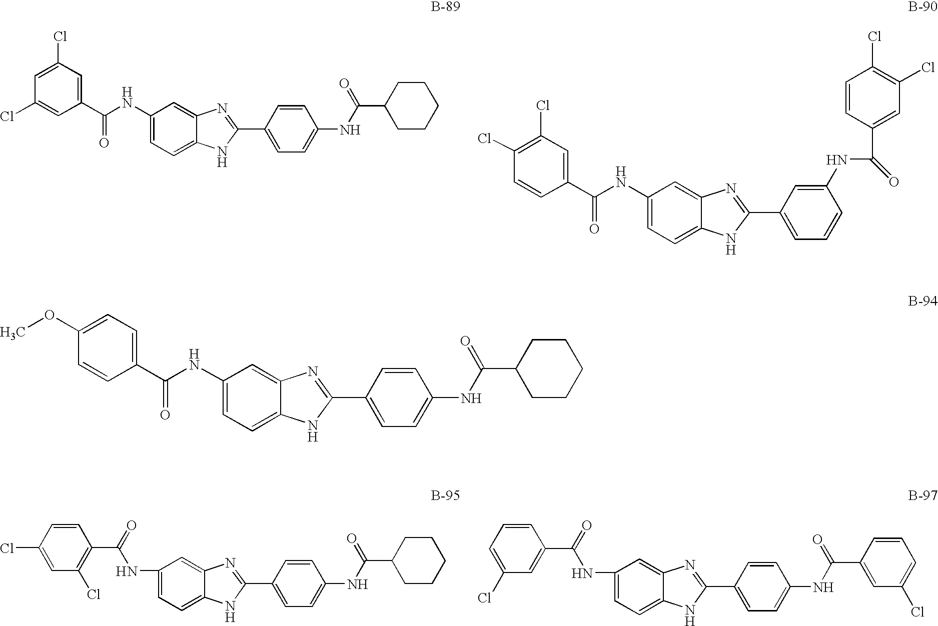

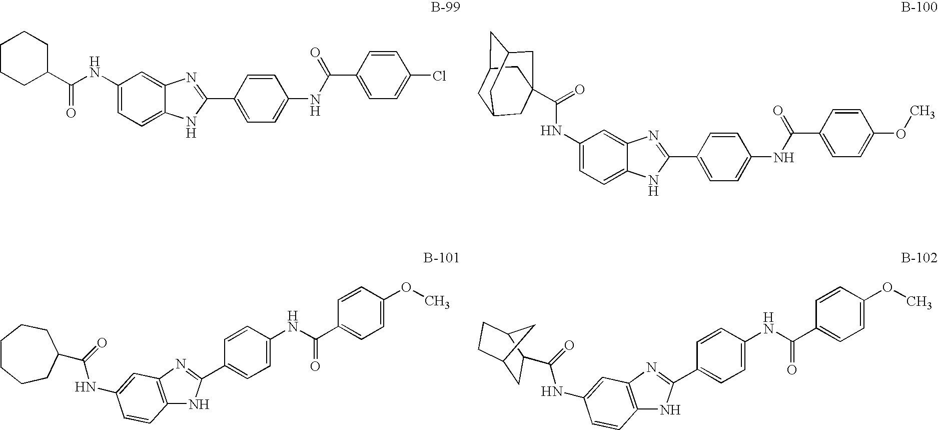

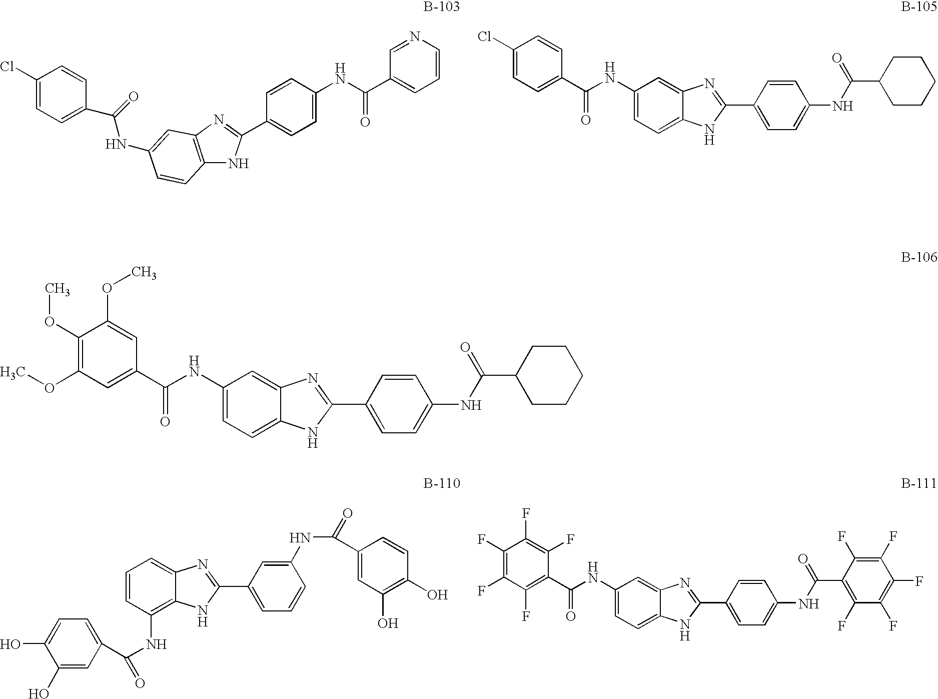

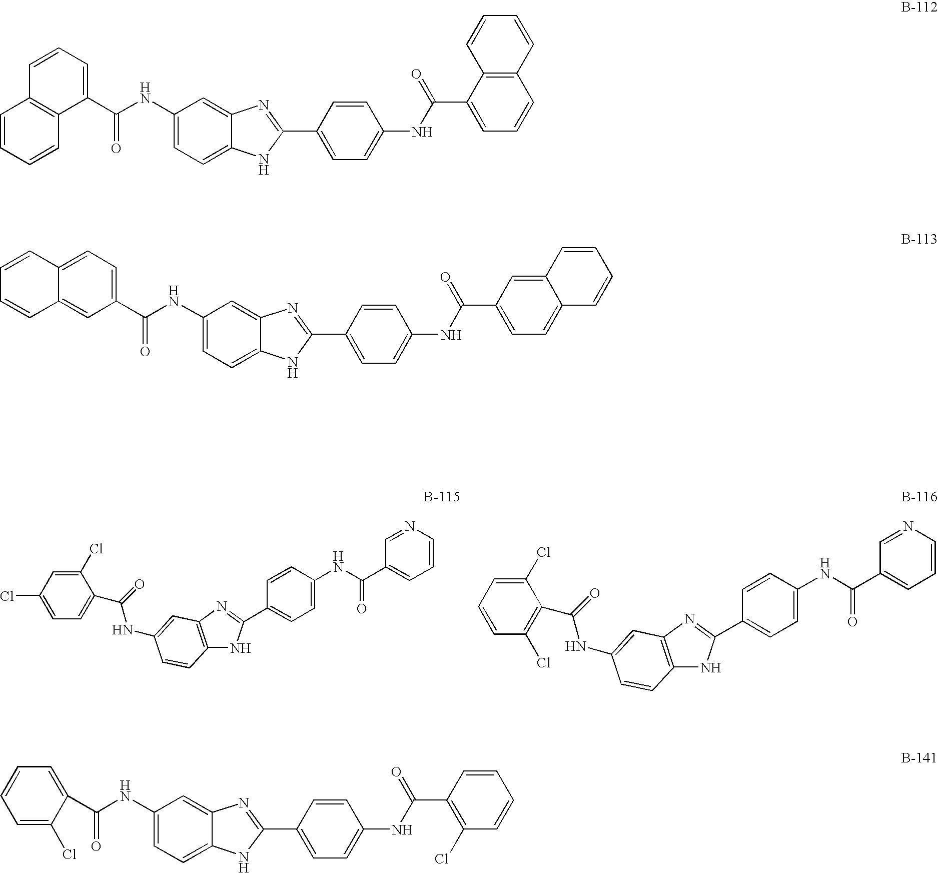

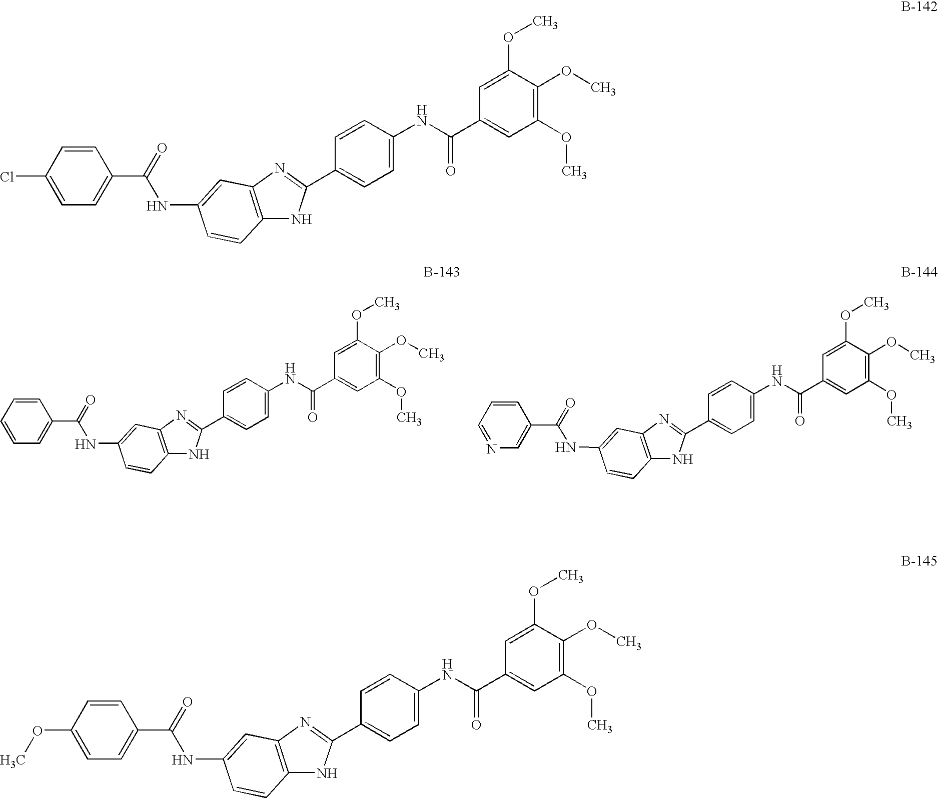

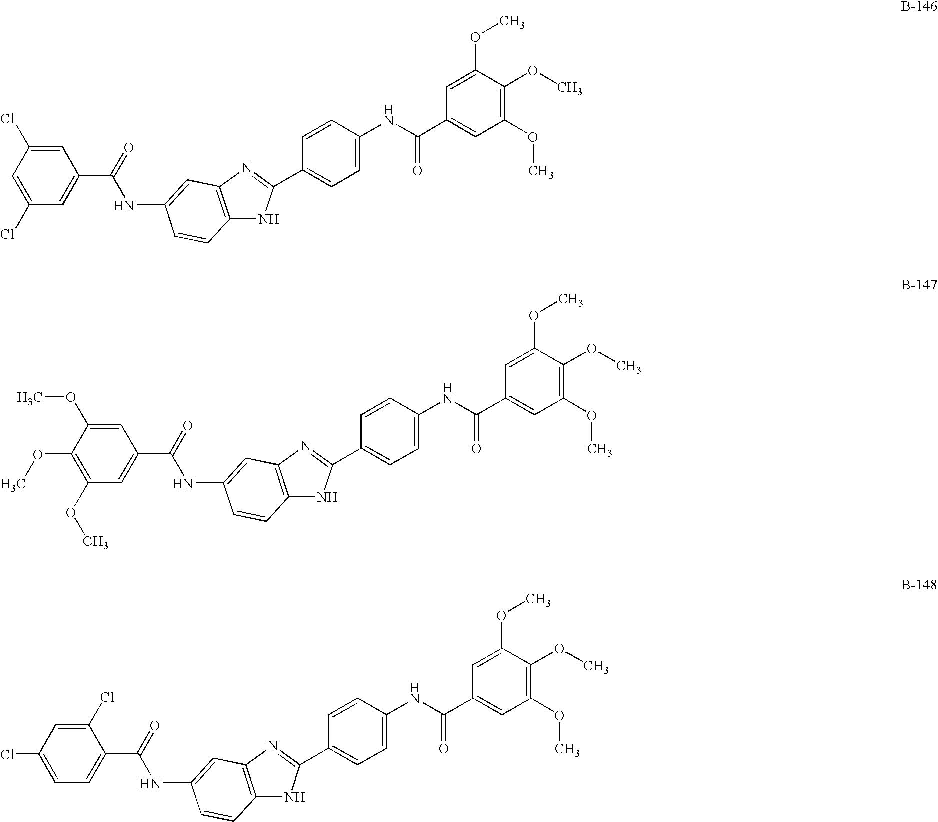

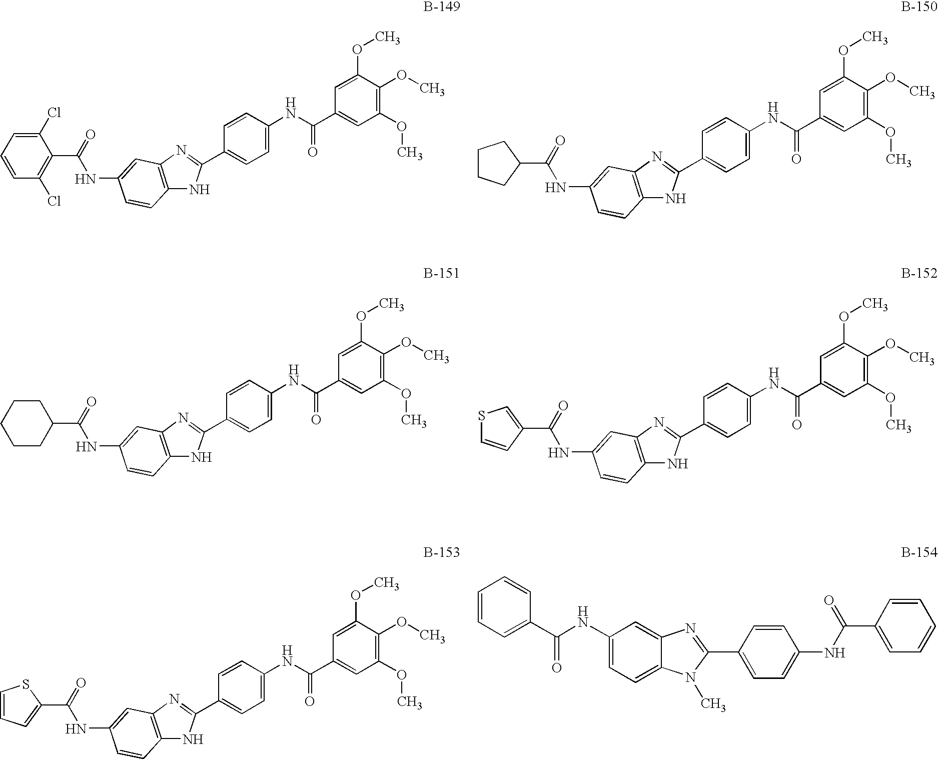

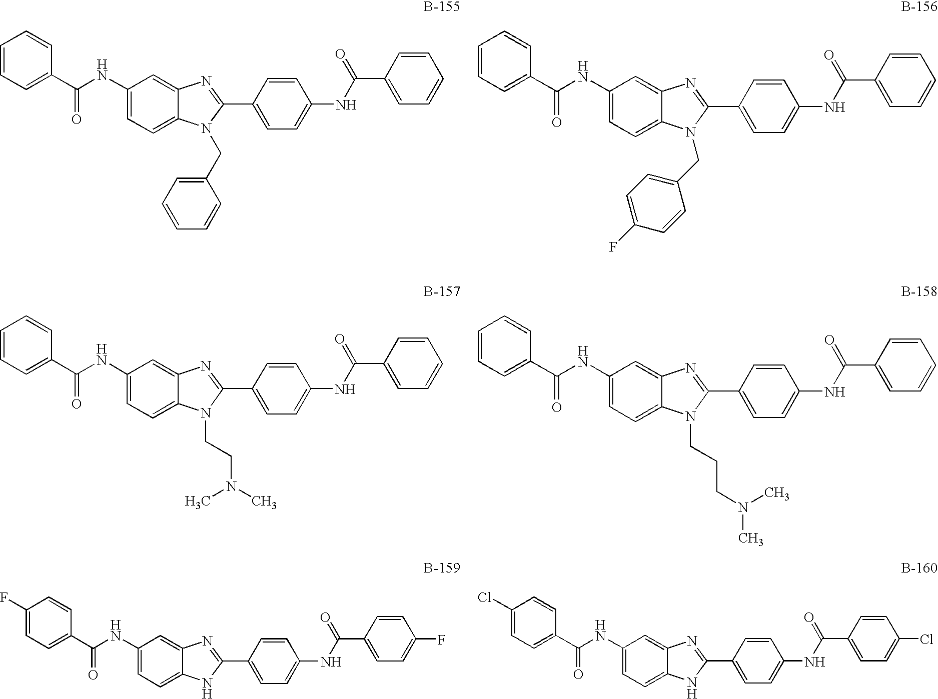

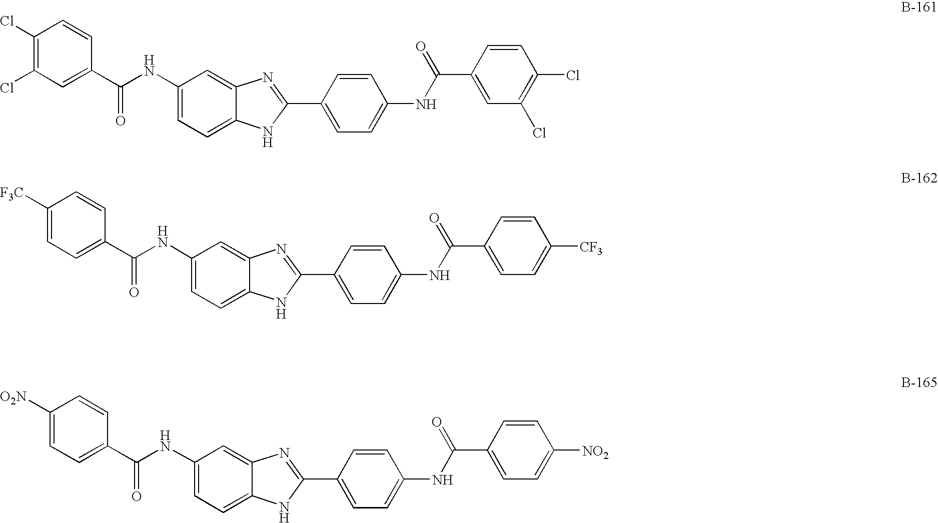

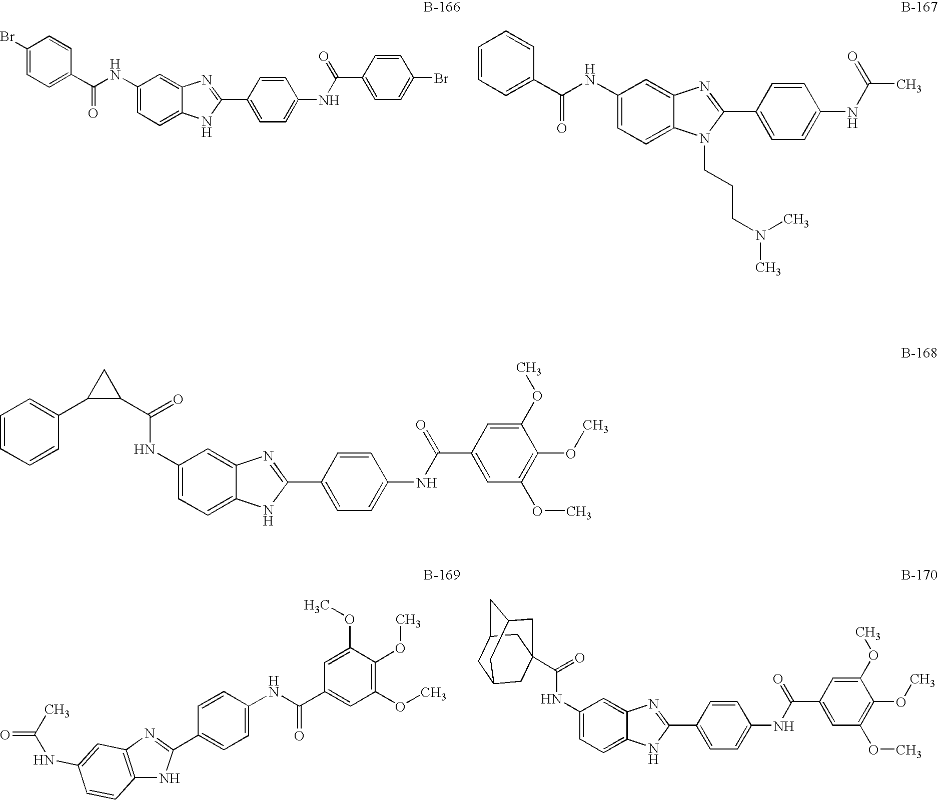

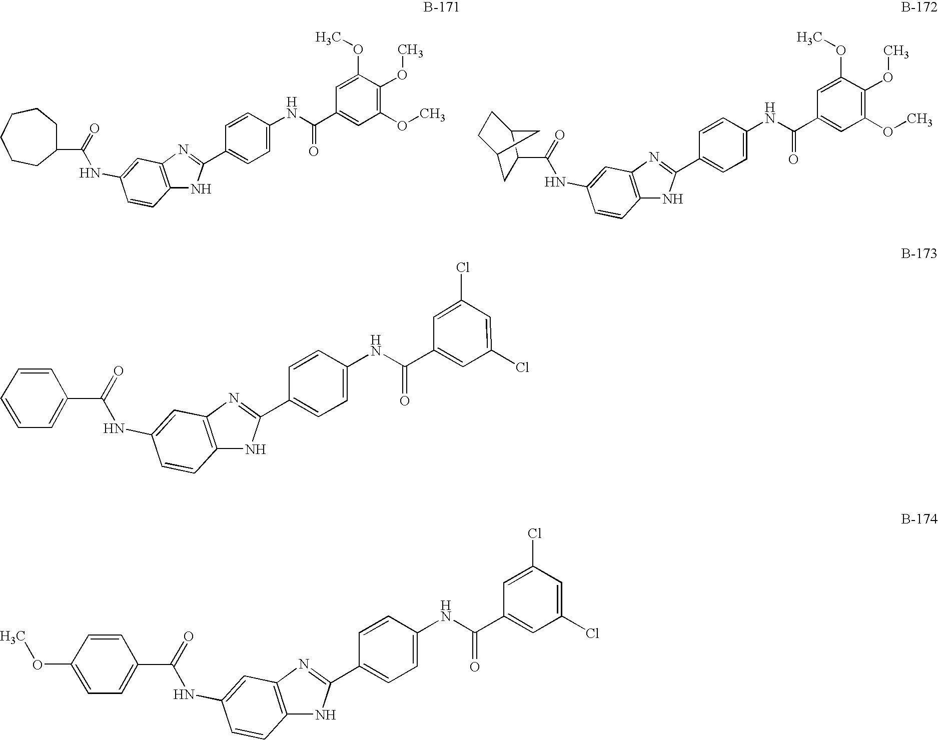

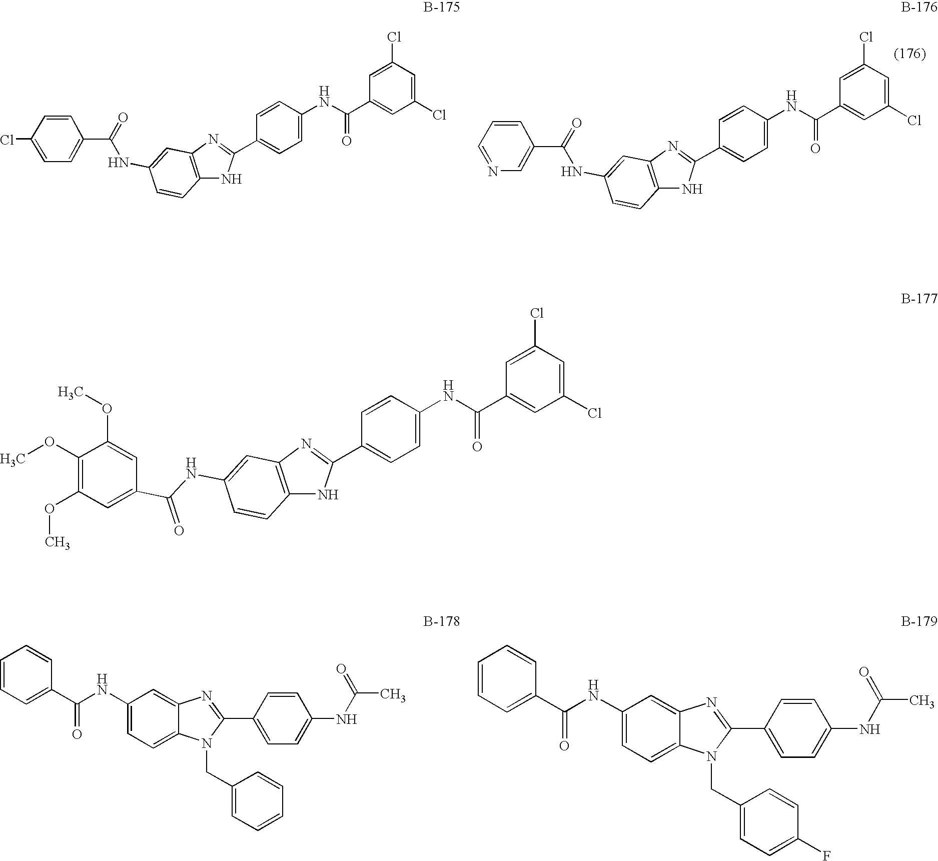

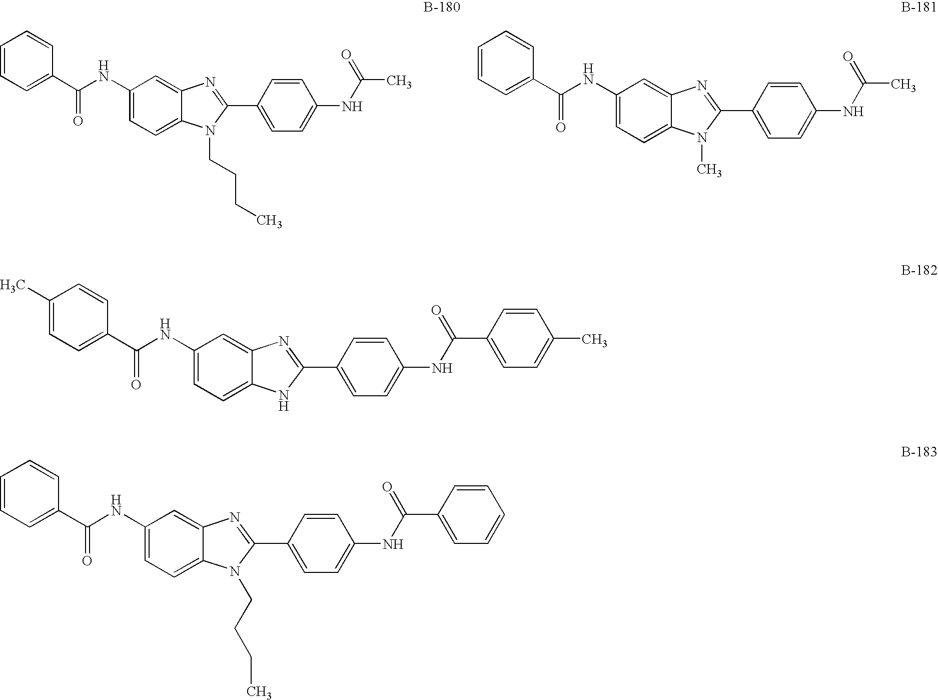

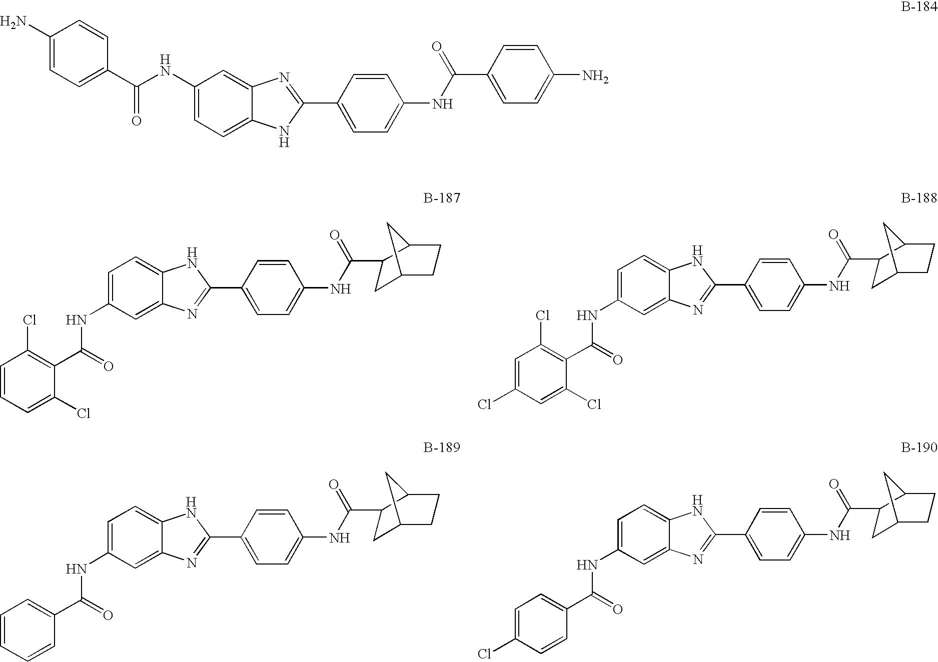

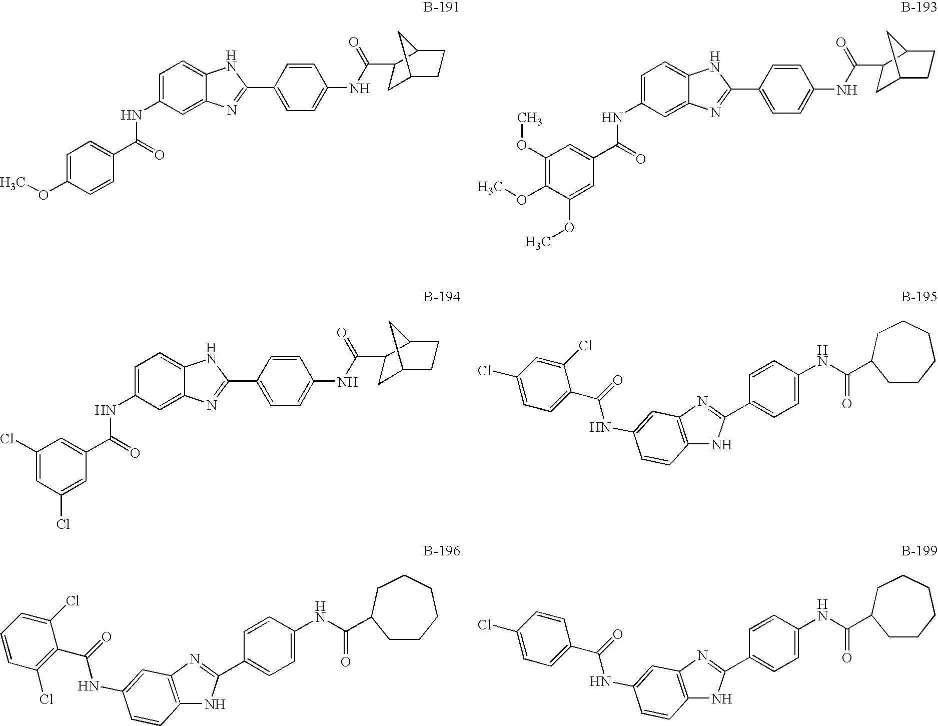

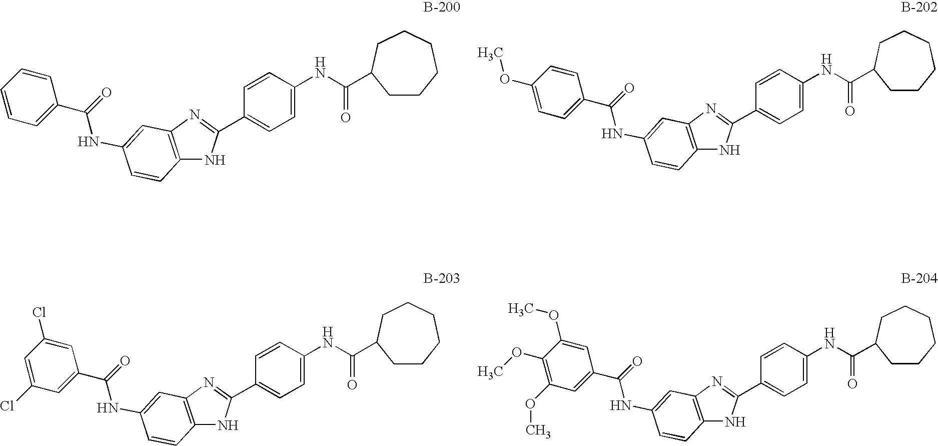

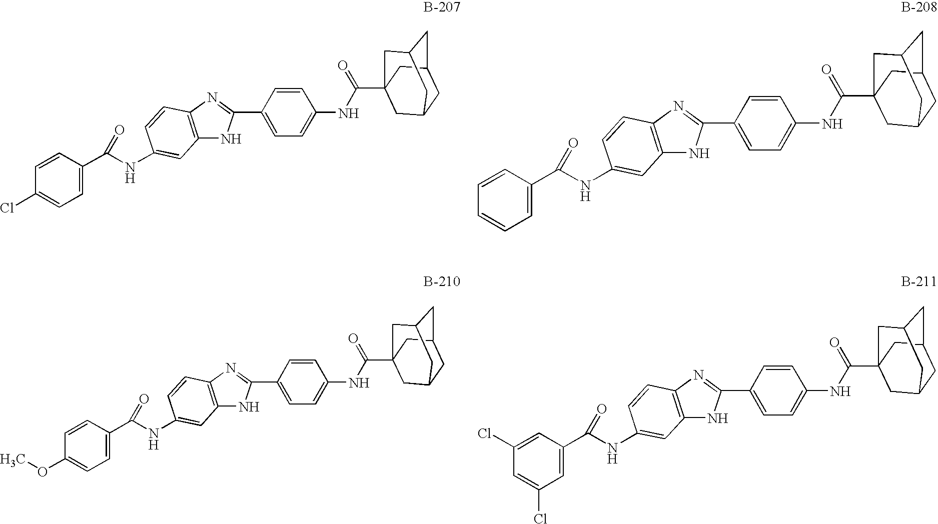

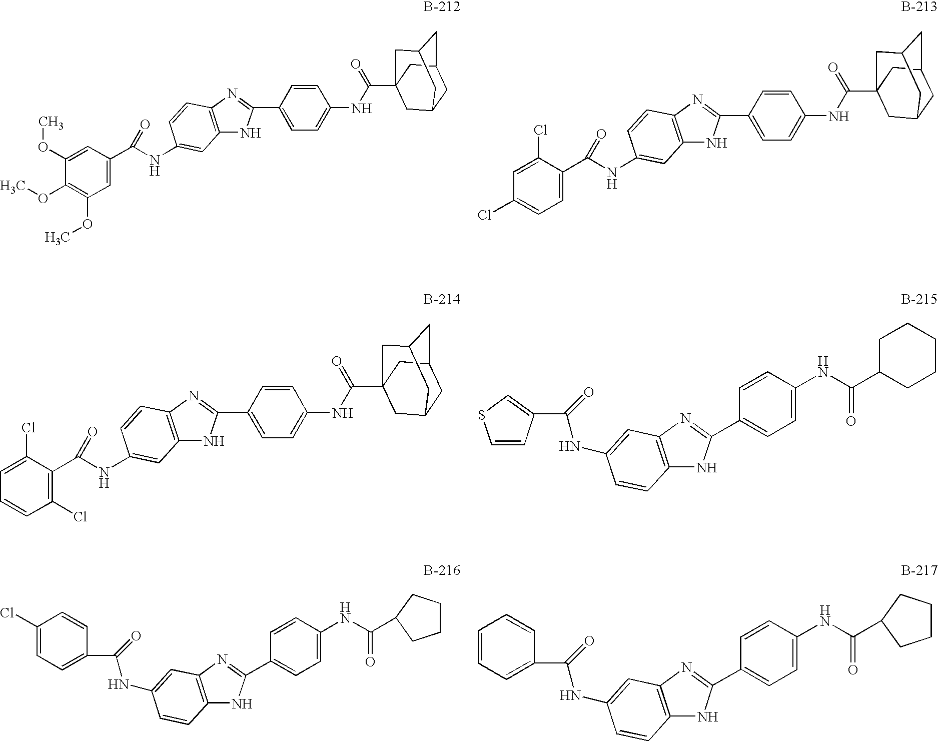

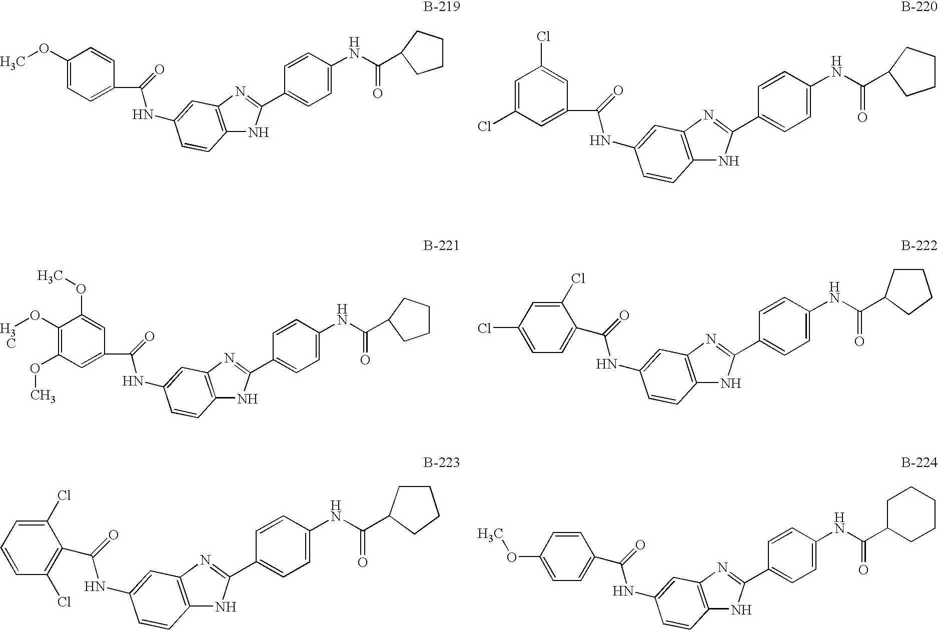

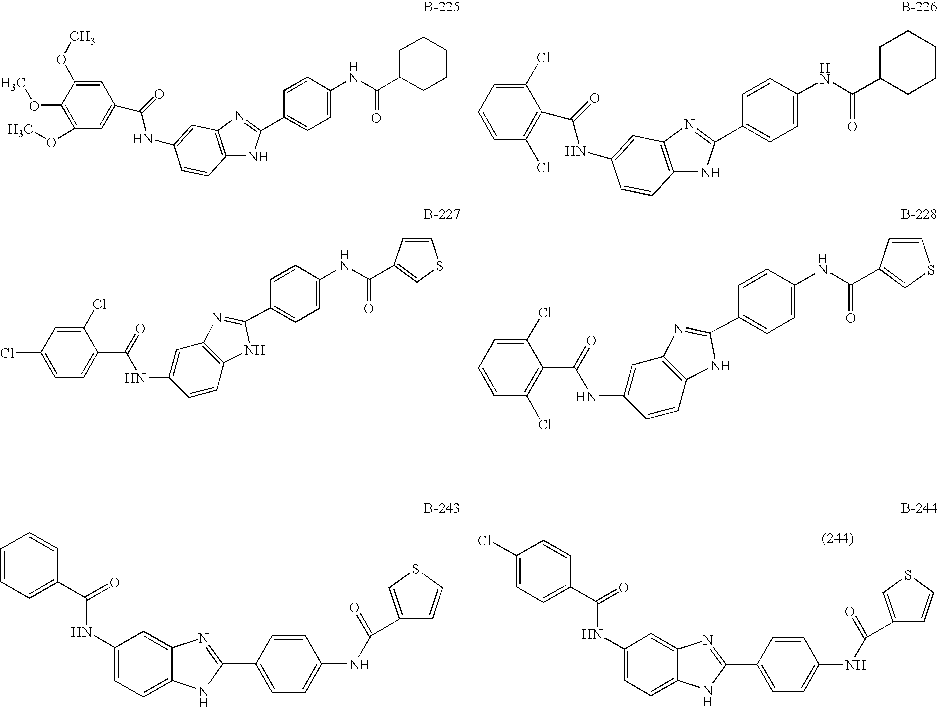

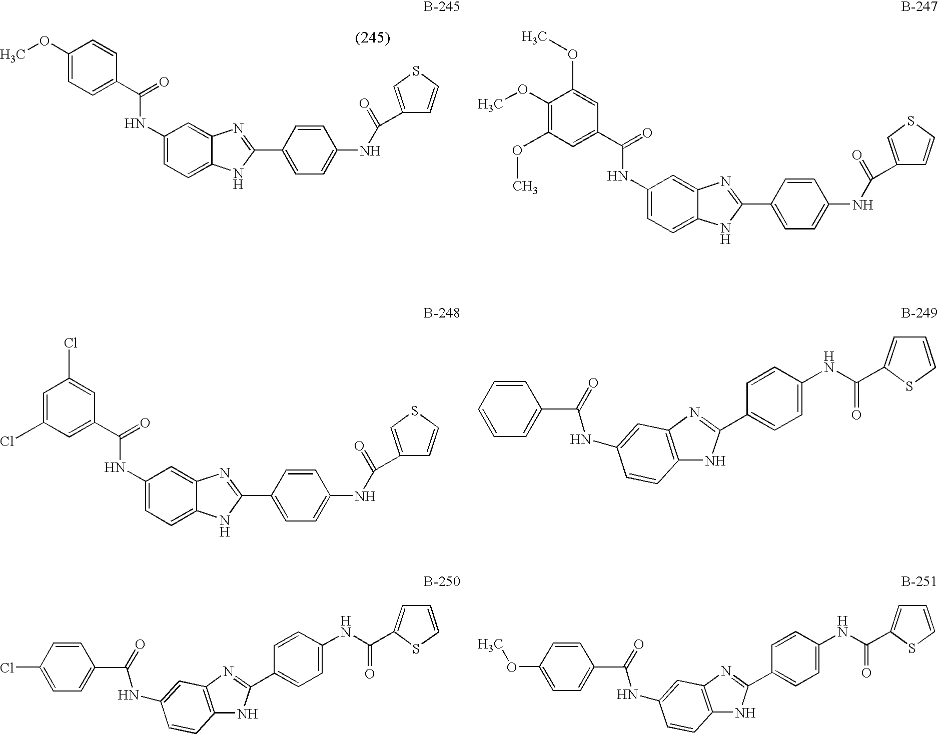

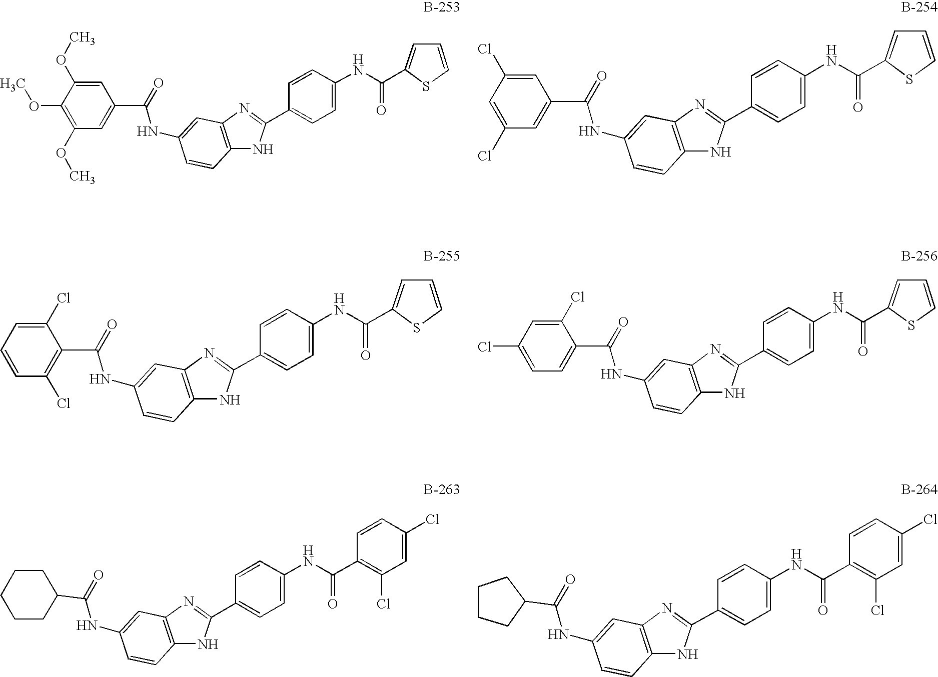

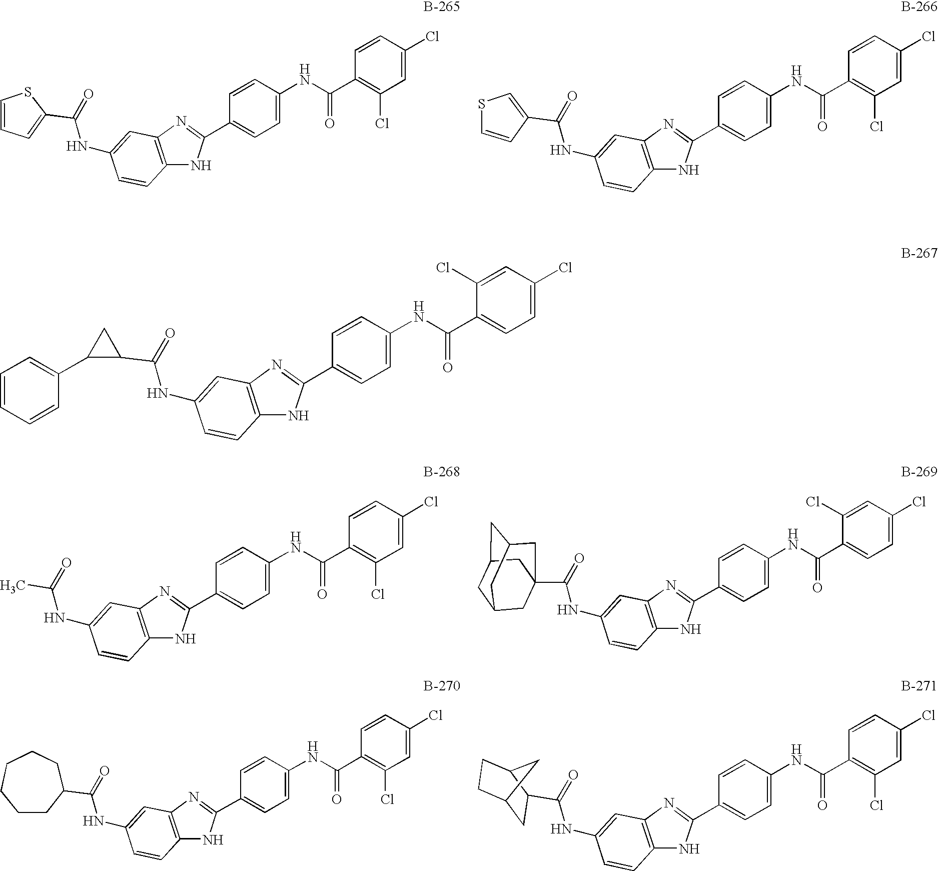

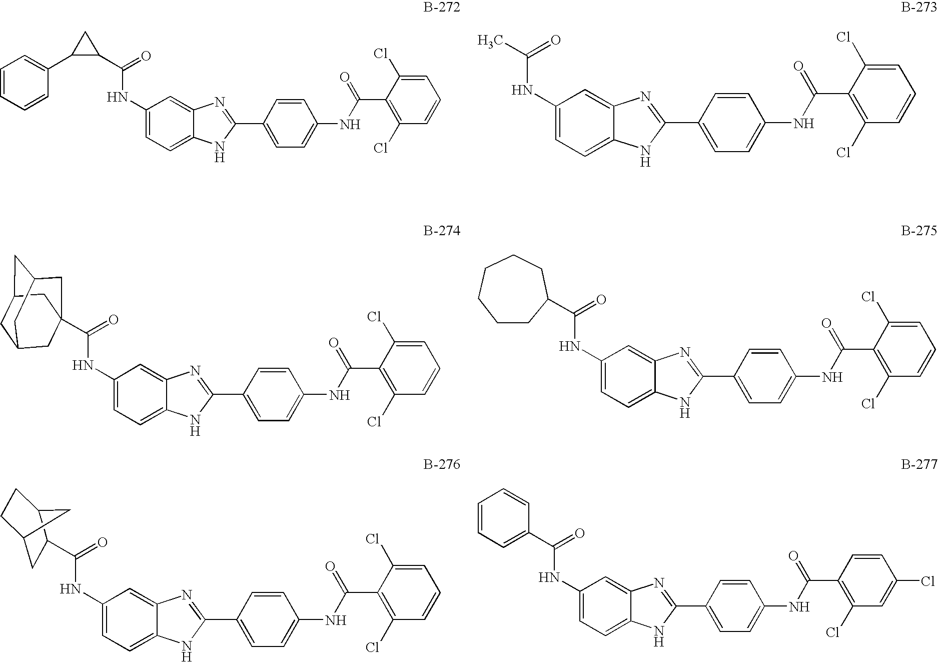

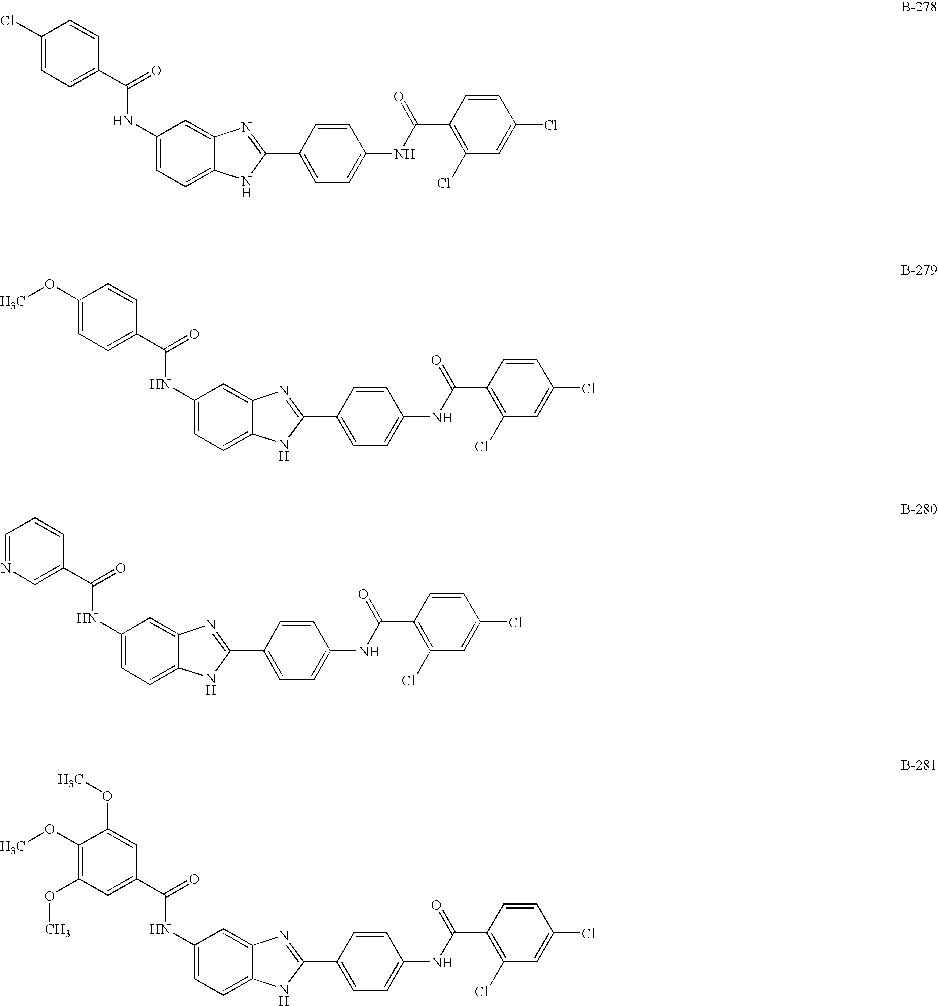

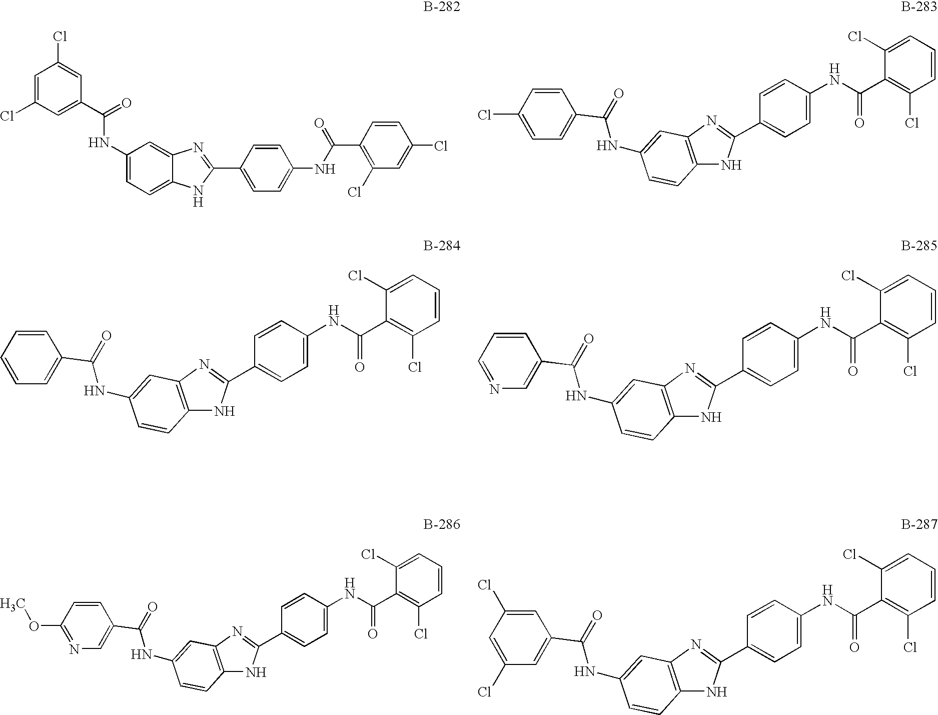

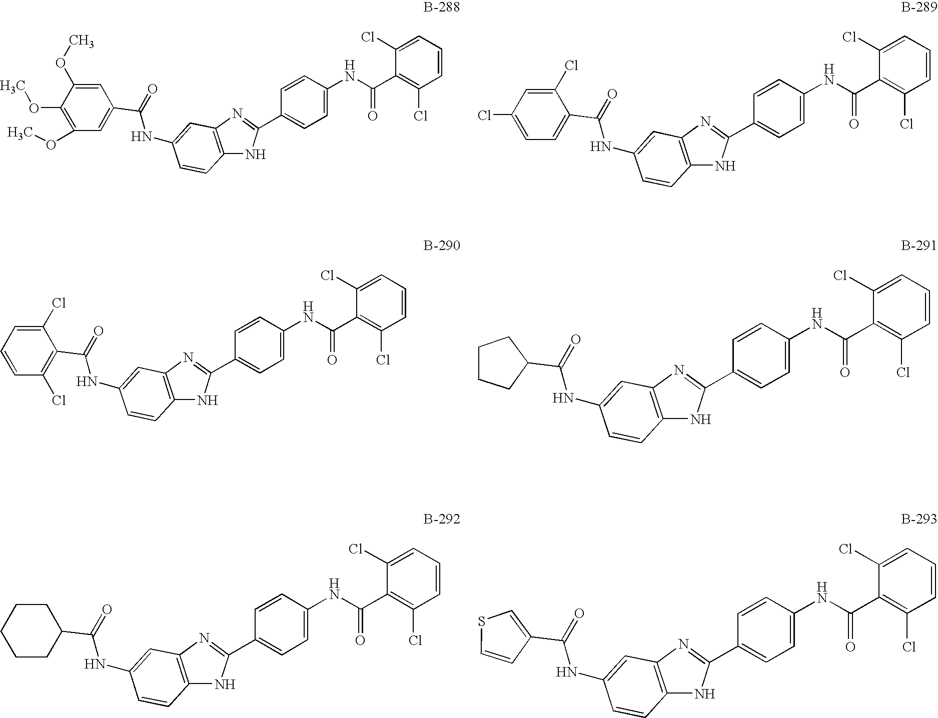

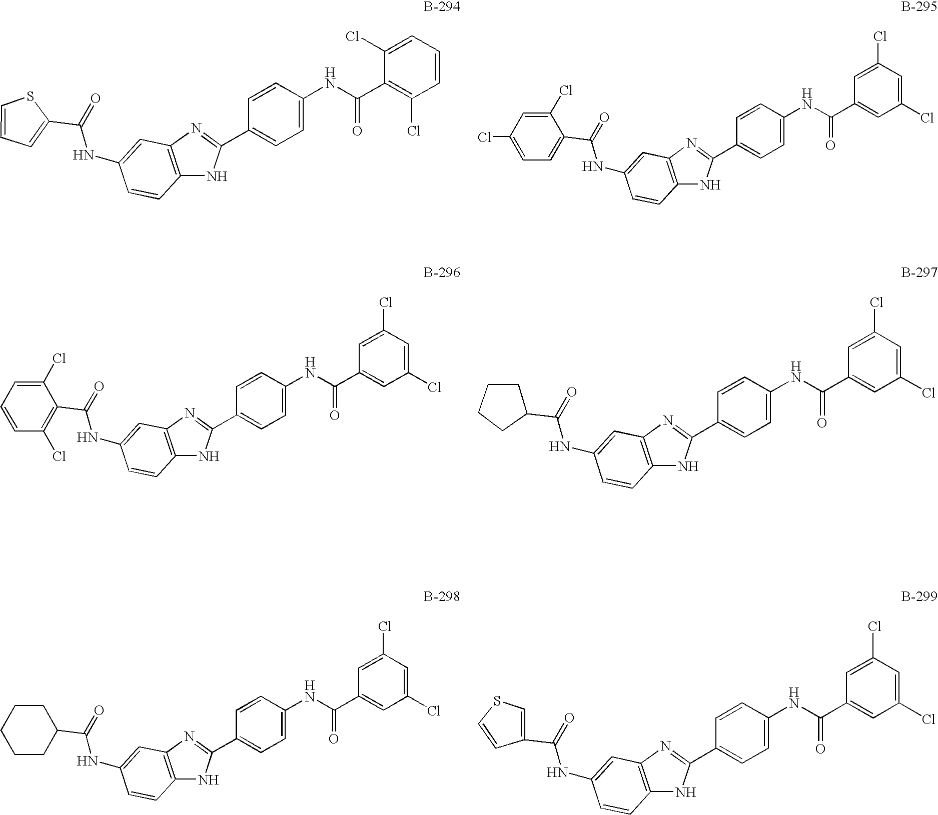

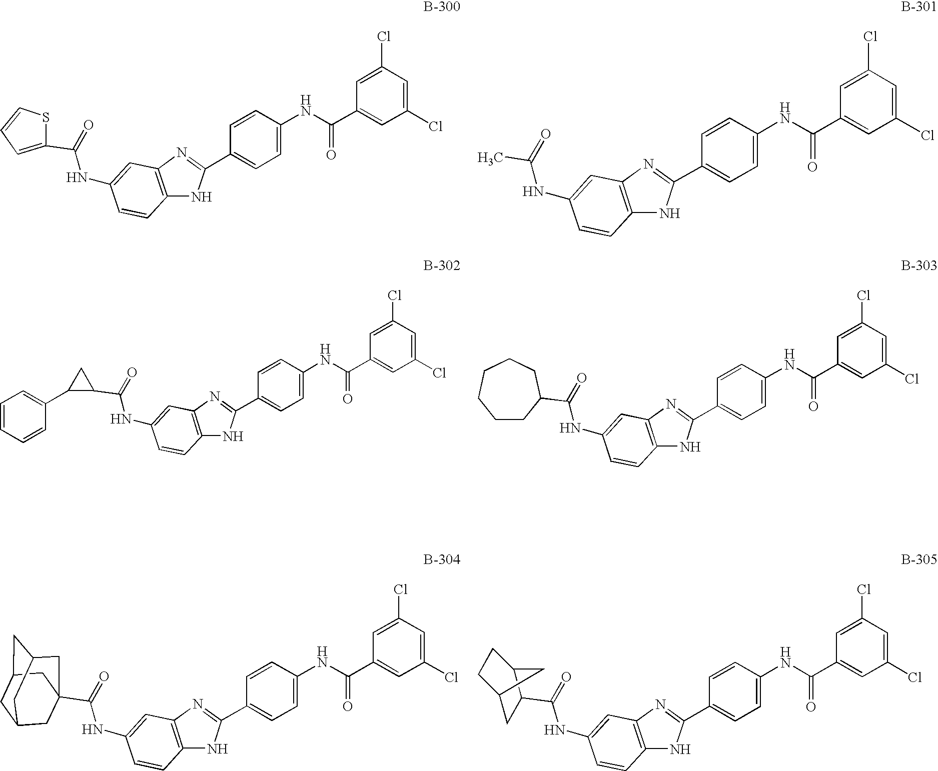

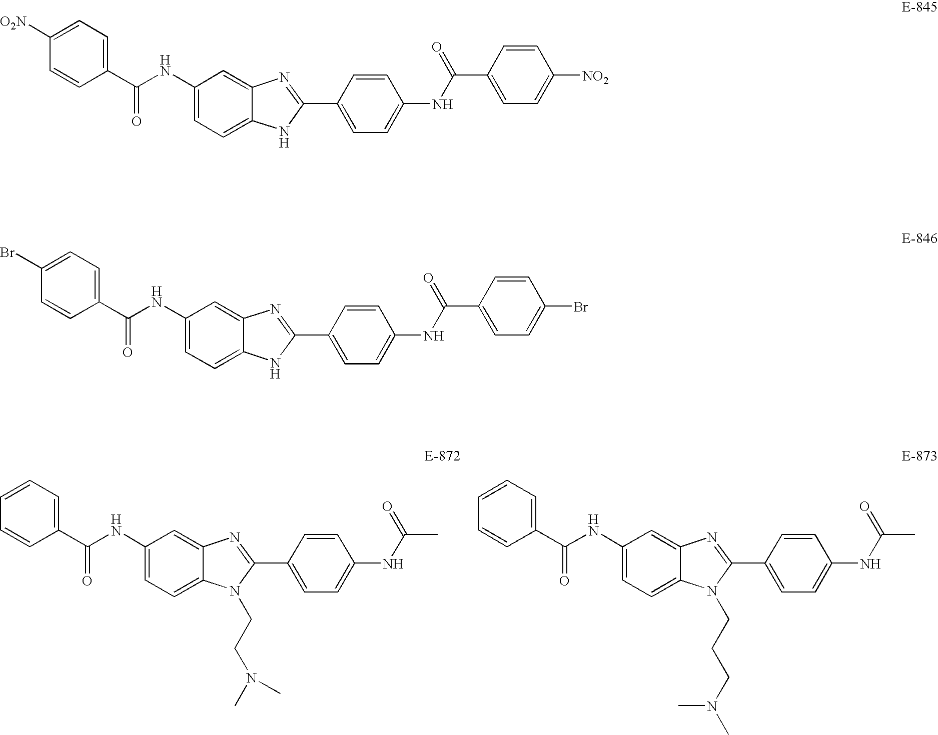

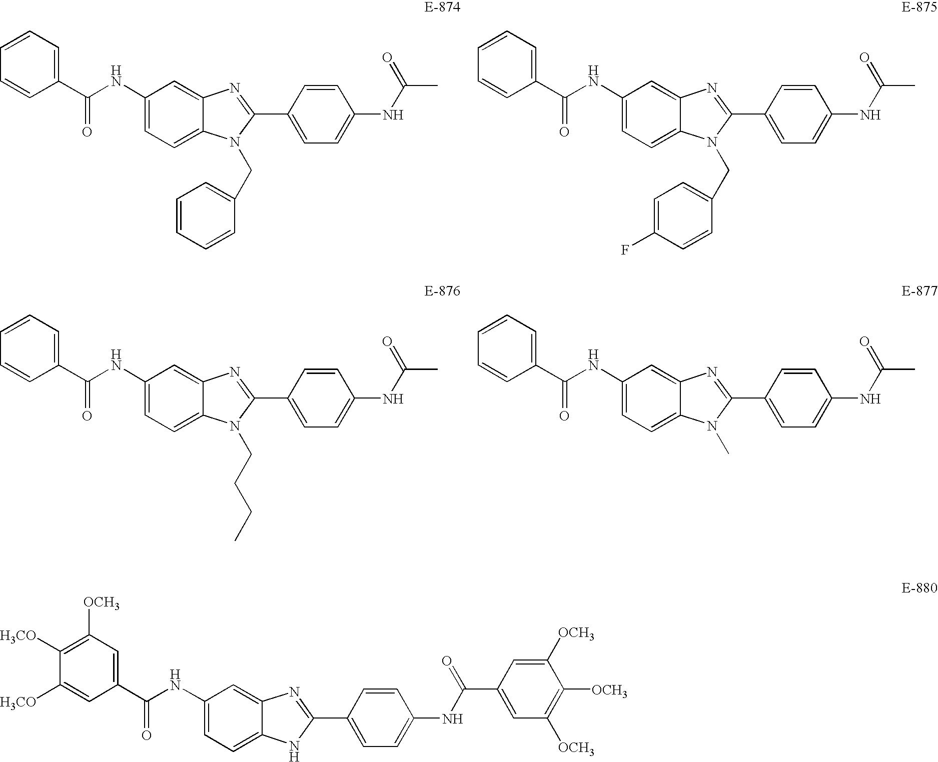

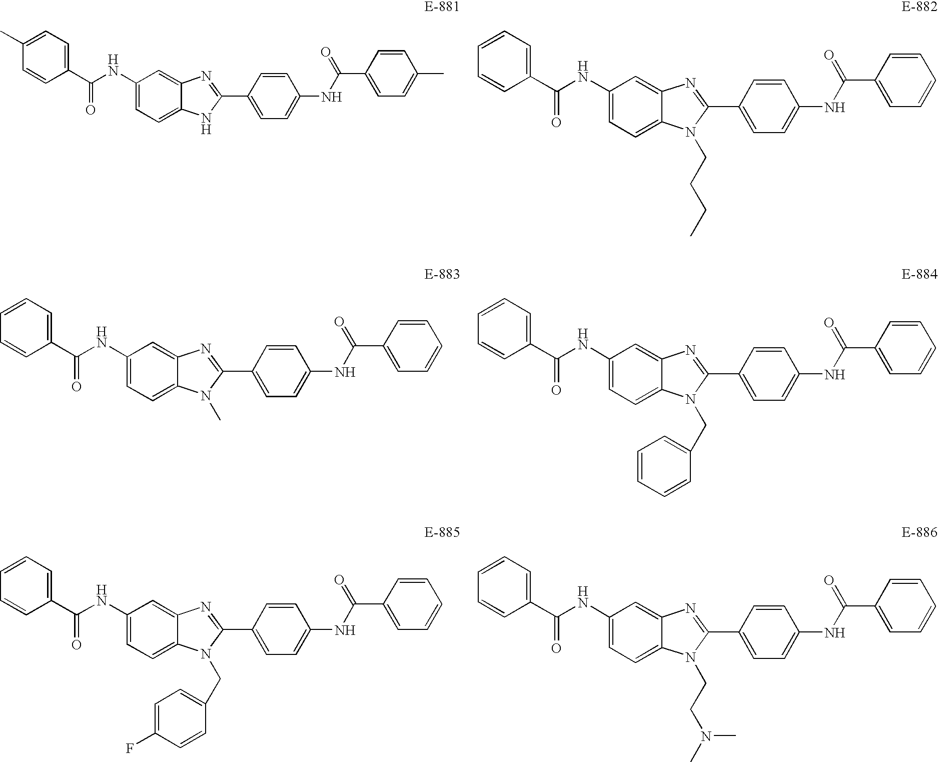

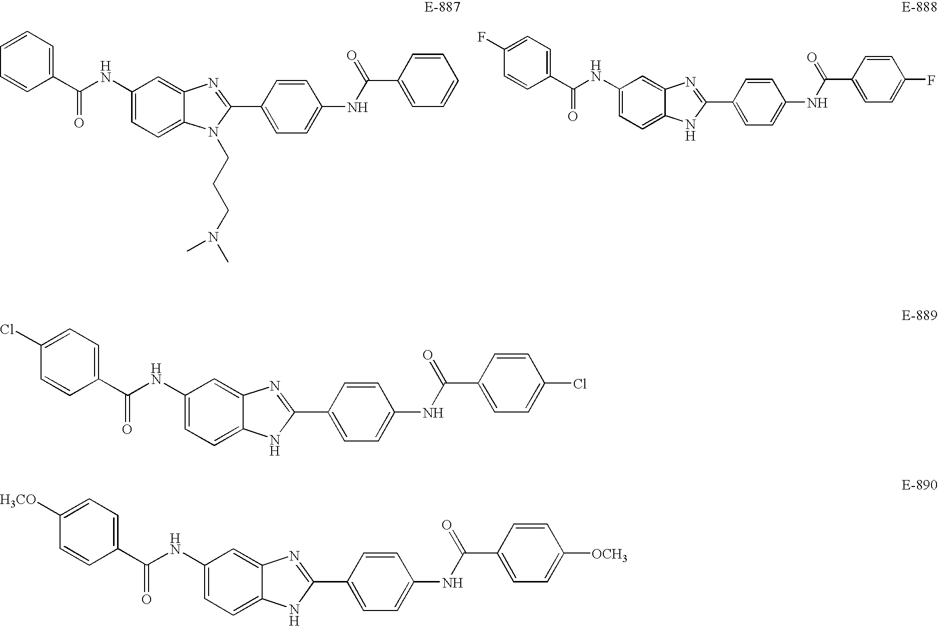

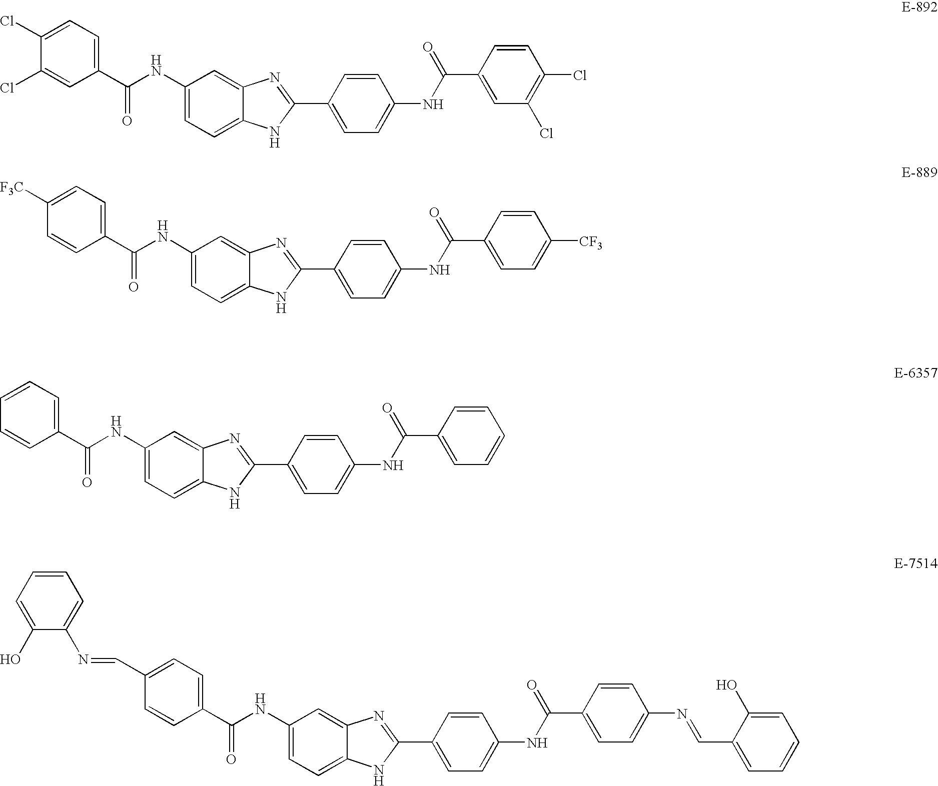

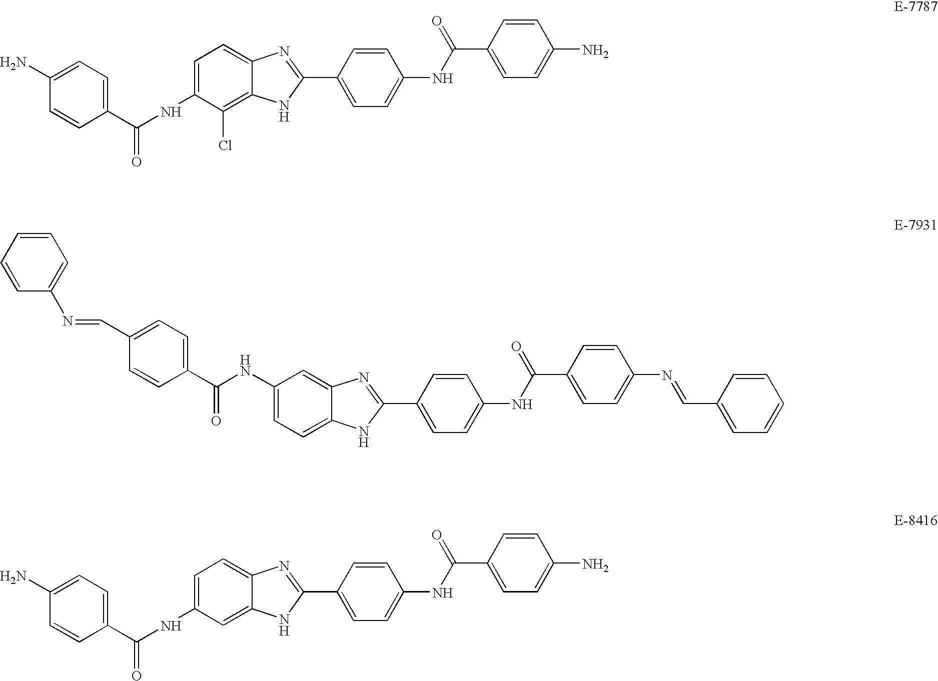

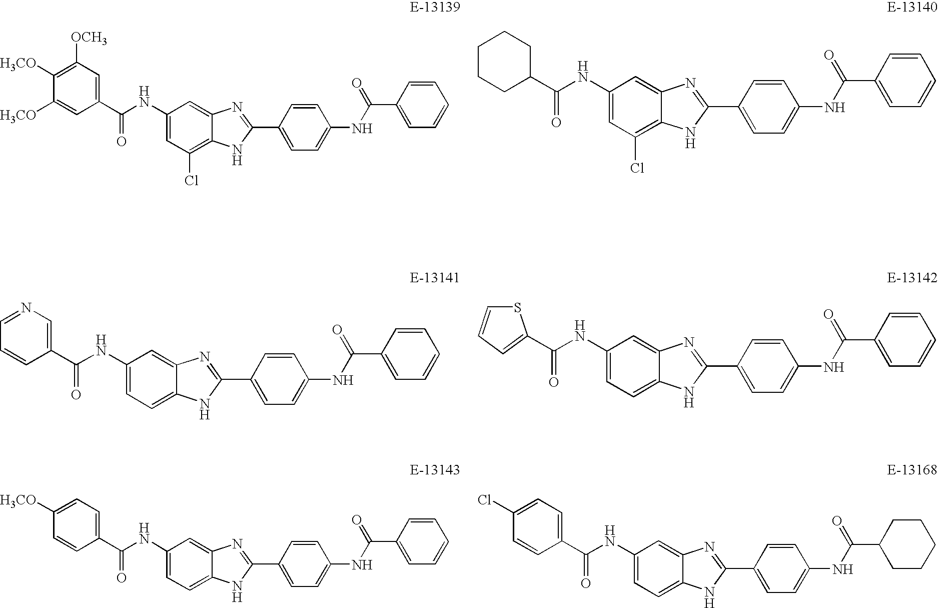

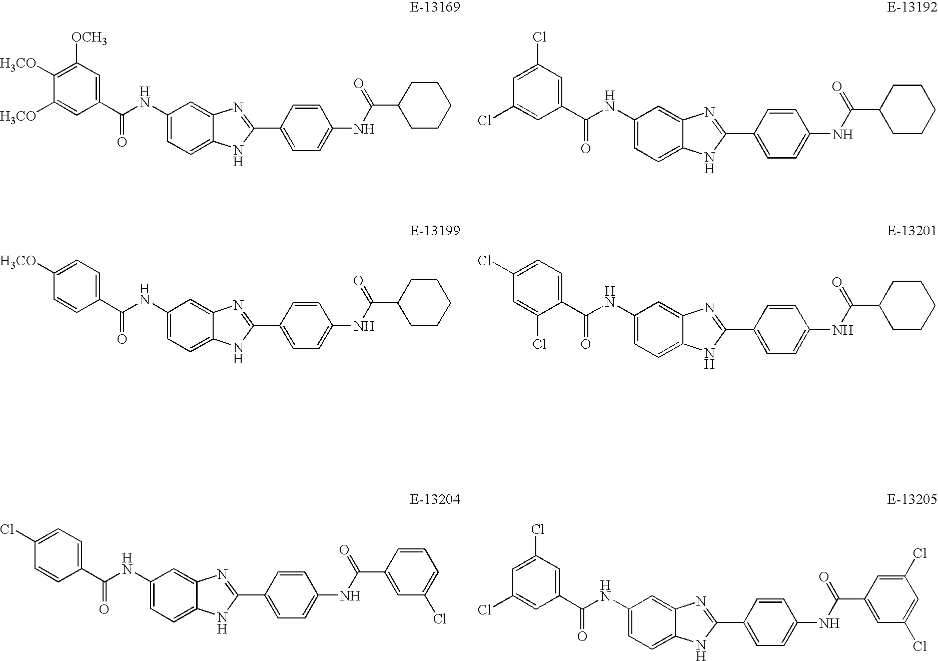

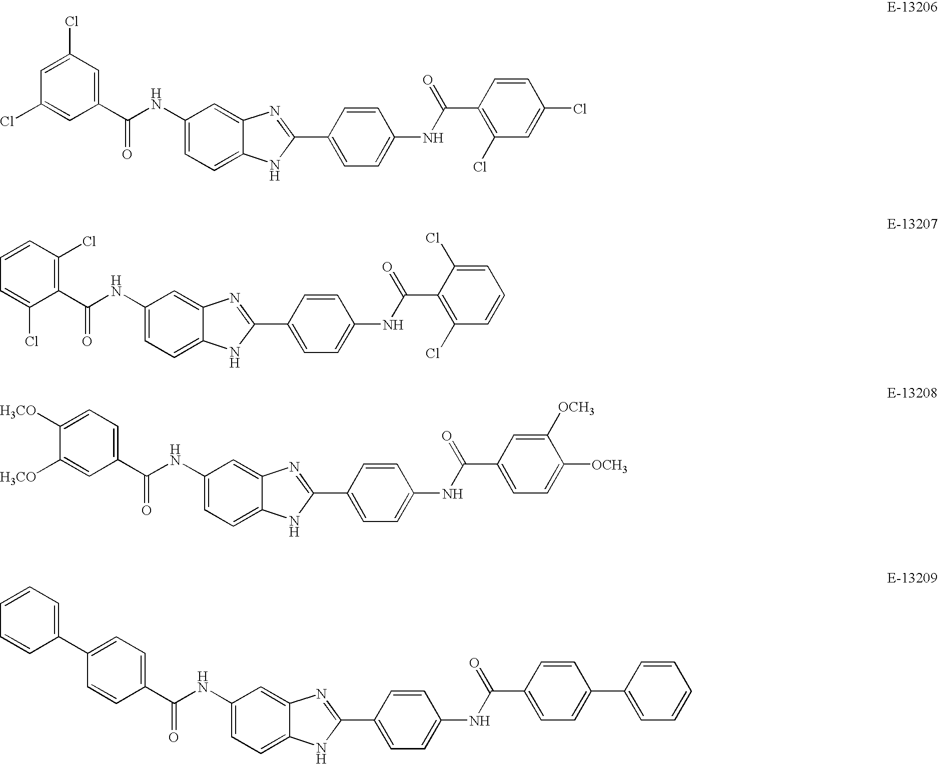

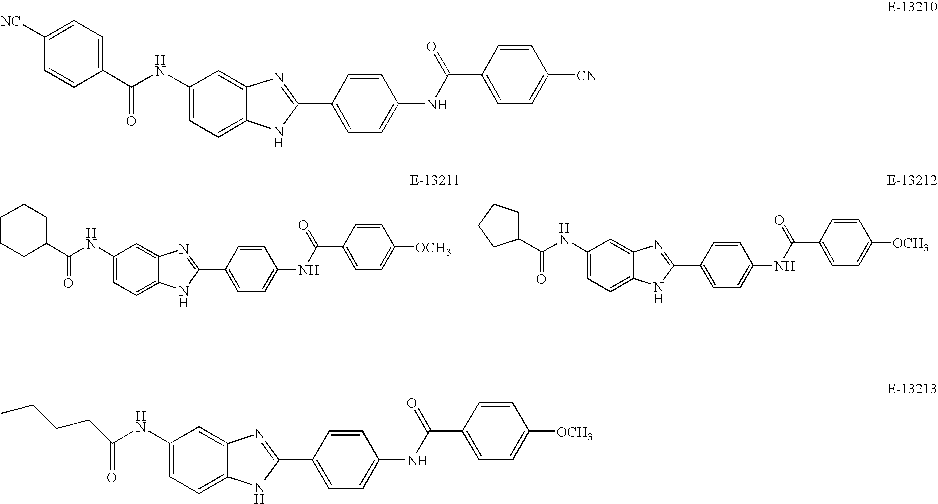

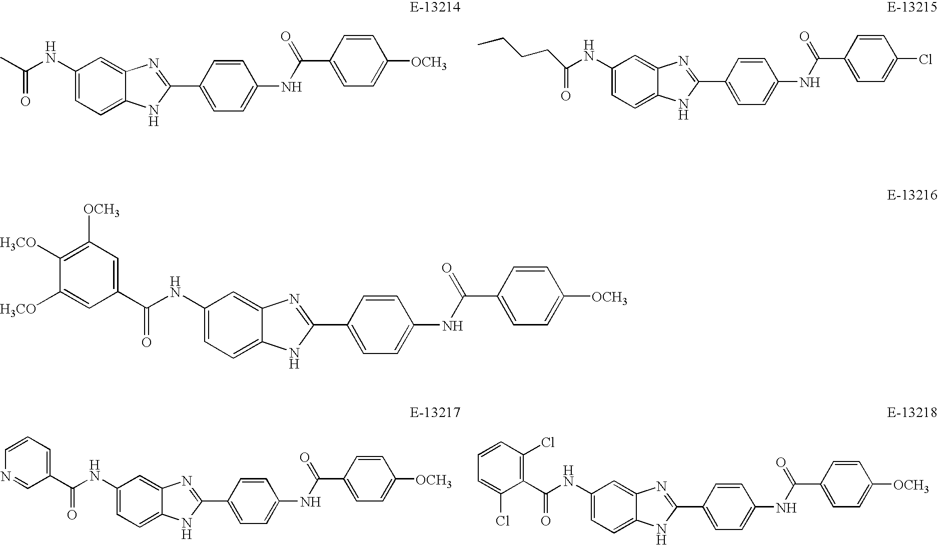

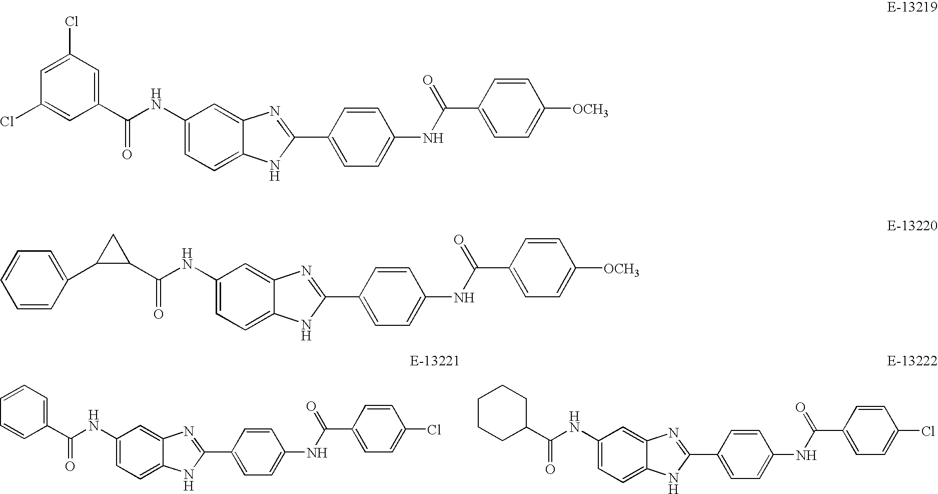

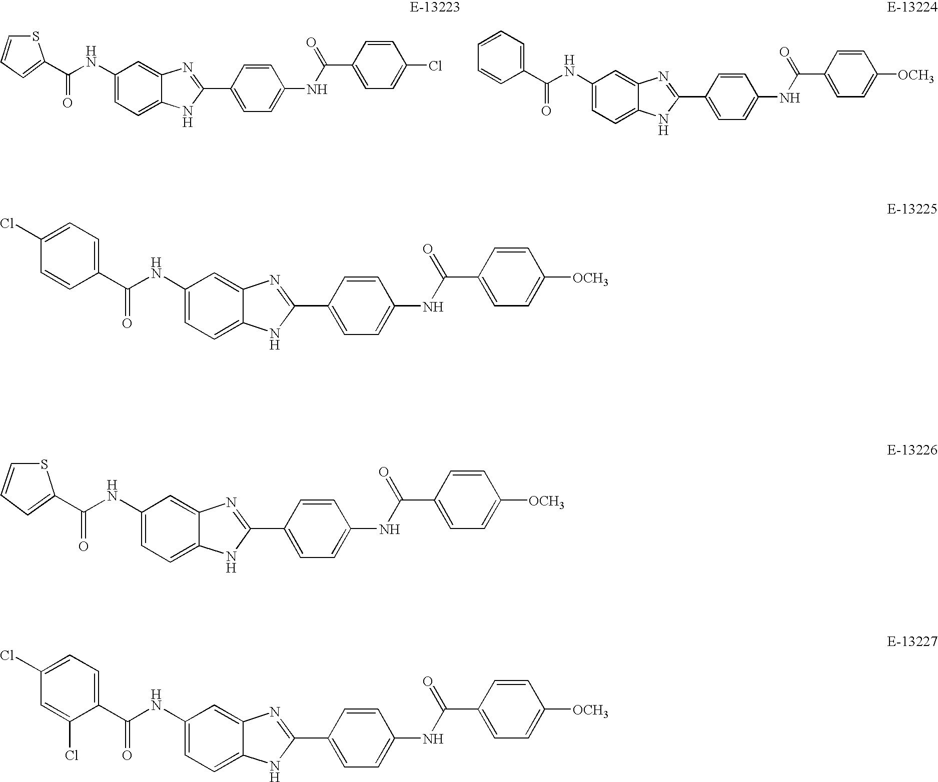

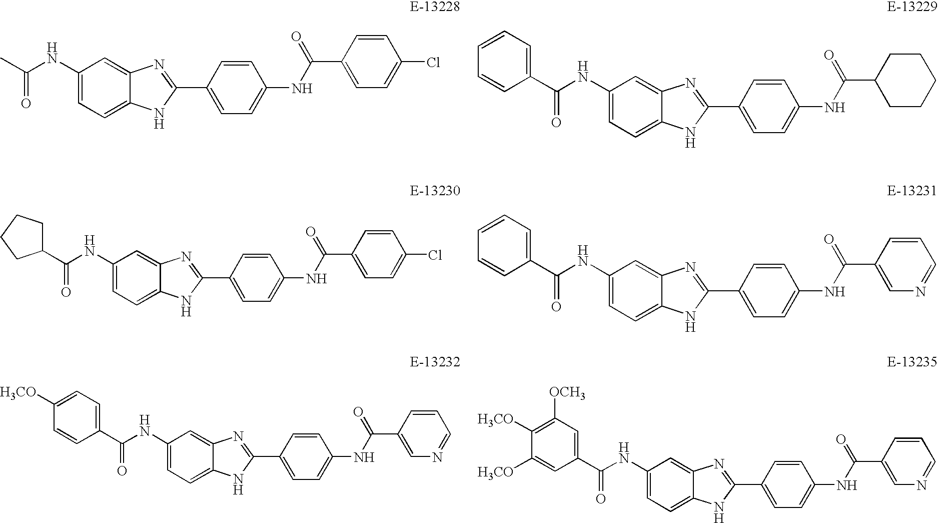

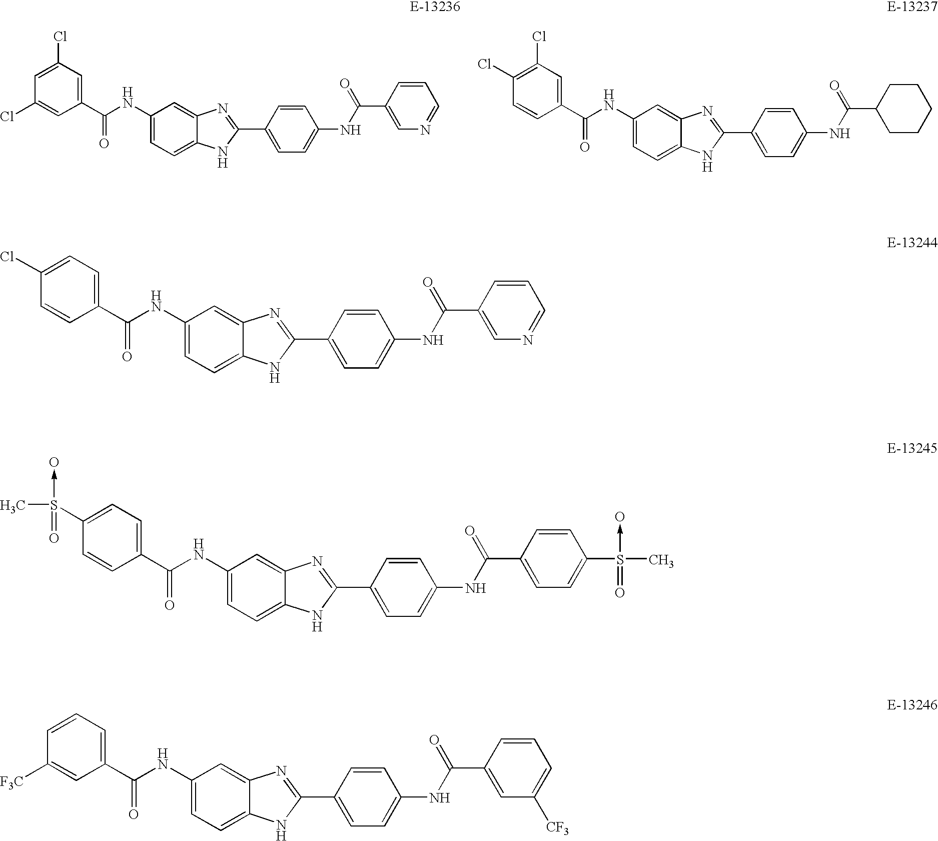

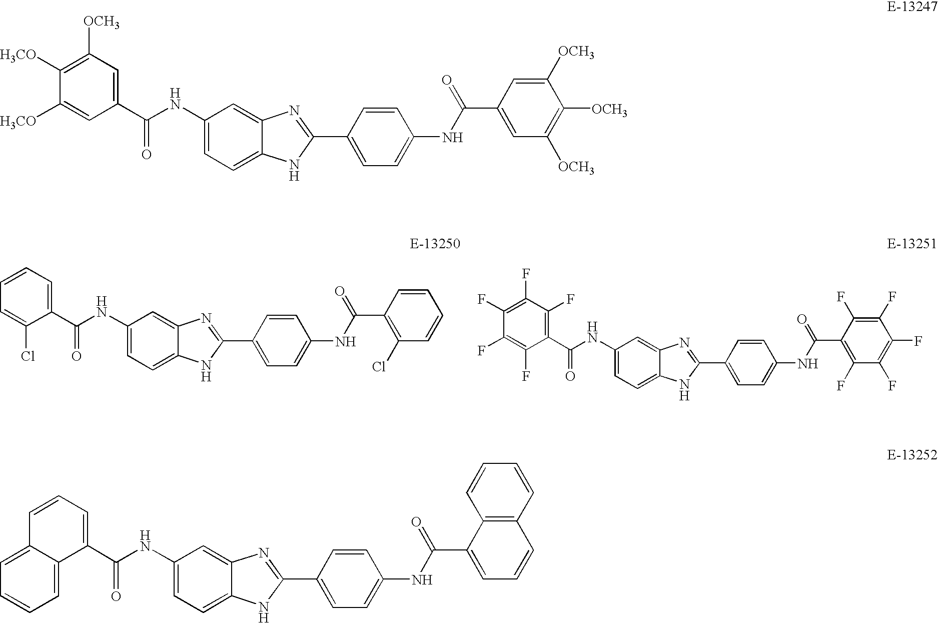

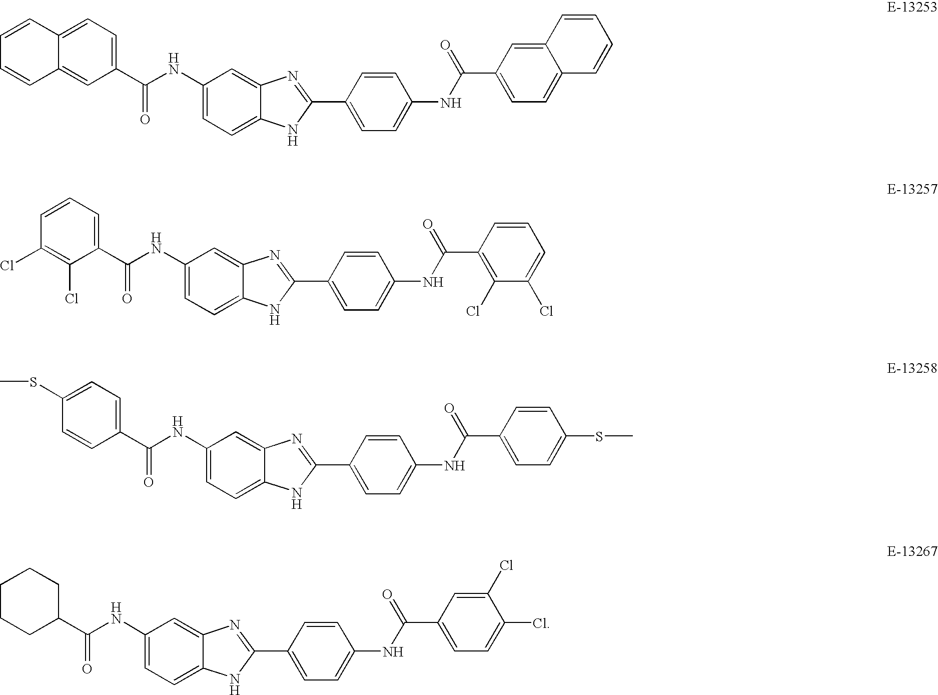

5. A method for the treatment of cancer comprising administration of a formulation comprising at least one compound of a pharmaceutical composition of the formulae: 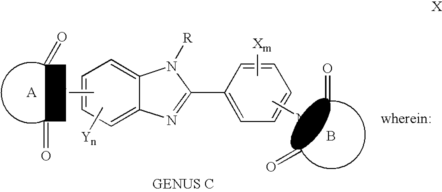
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;
n is an integer from one to four;
m is an integer from one to four;
R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and
A and B rings independently comprise unsubstituted or substituted carbon atoms ranging from four carbon atoms to ten carbon atoms.

6. The method of claim 5, wherein method for the treatment of cancer comprises administering a formulation comprising at least one compound selected from the group consisting of

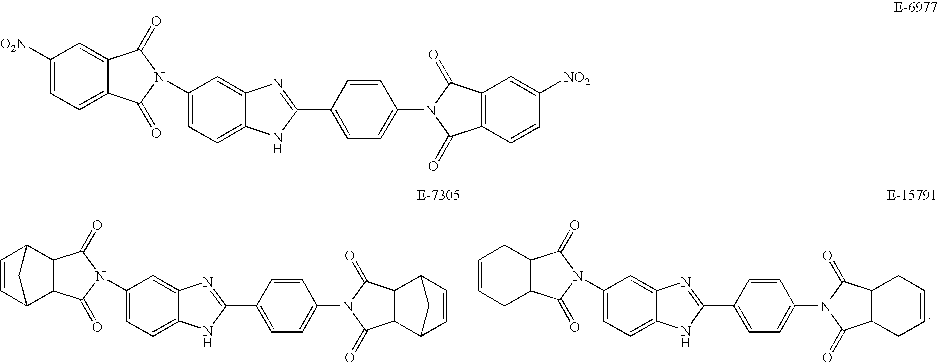

7. A method for the treatment of cancer comprising administration of a formulation comprising at least one compound of a pharmaceutical composition of the formulae: 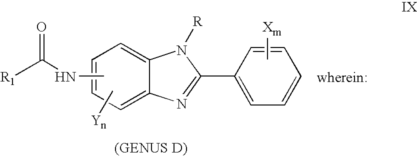
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;
n is an integer from one to three;
m is an integer from one to four;
R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and
R₁ is selected from the group consisting of H, alkyl, substituted alkyl, alkenyl, substituted alkenyl, alkynyl, substituted alkynyl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom.

8. The method of claim 7, wherein method for the treatment of cancer comprises administering a formulation comprising at least one compound selected from the group consisting of

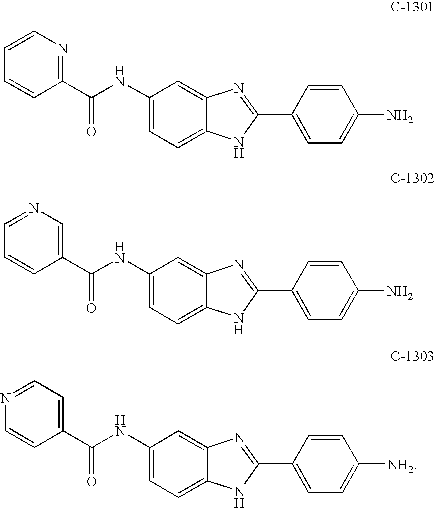

9. A method for the treatment of cancer comprising administration of a formulation comprising at least one compound of a pharmaceutical composition of the formulae: 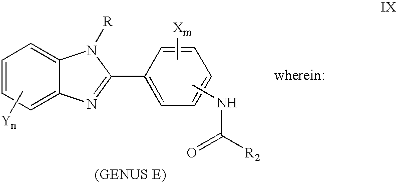
X and Y may be different or the same and are independently selected from the group consisting of H, halogen, alkyl, alkoxy, aryl, substituted aryl, fused aryl, hydroxy, amino, alkylamino, cycloalkyl, morpholine, thiomorpholine, nitro, cyano, CF₃, OCF₃, COR₂, COOR₂, CONH₂, CONHR₂, and NHCOR₂;
n is an integer from one to three;
m is an integer from one to four;
R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and
R₂ is selected from the group consisting of H, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, polycycloalkyl, substituted polycycloalkyl, polycycloalkenyl, substituted polycycloalkenyl, arylalkyl, substituted arylalkyl, heteroarylalkyl, substituted heteroarylalkyl, arylcycloalkyl, substituted arylcycloalkyl, heteroarylcycloalkyl, substituted heteroarylcycloalkyl, heterocyclic ring, substituted heterocyclic ring, heteroatom, and substituted heteroatom.

10. The method of claim 9, wherein method for the treatment of cancer comprises administering a formulation comprising at least one compound selected from the group consisting of

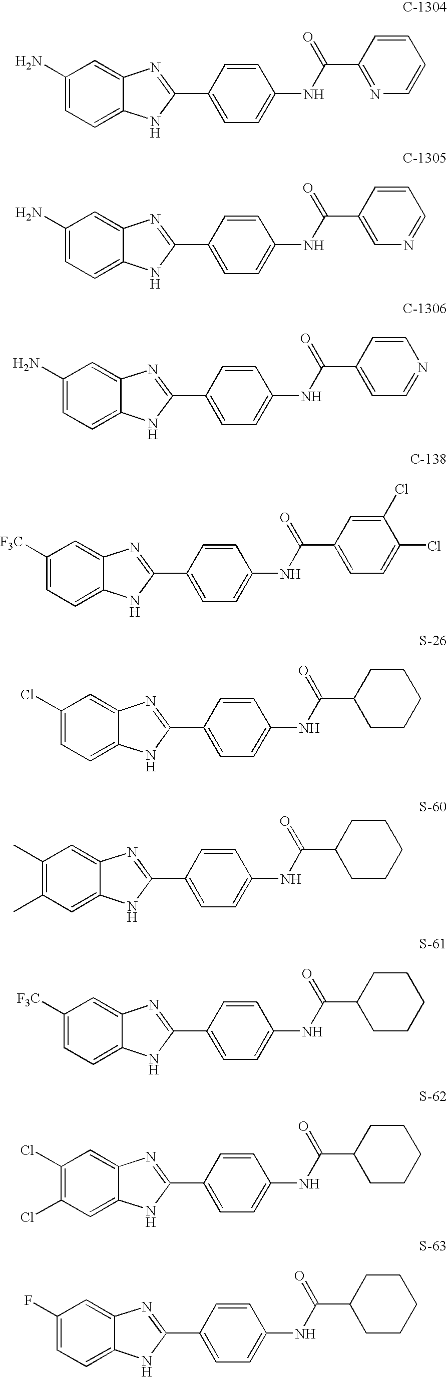

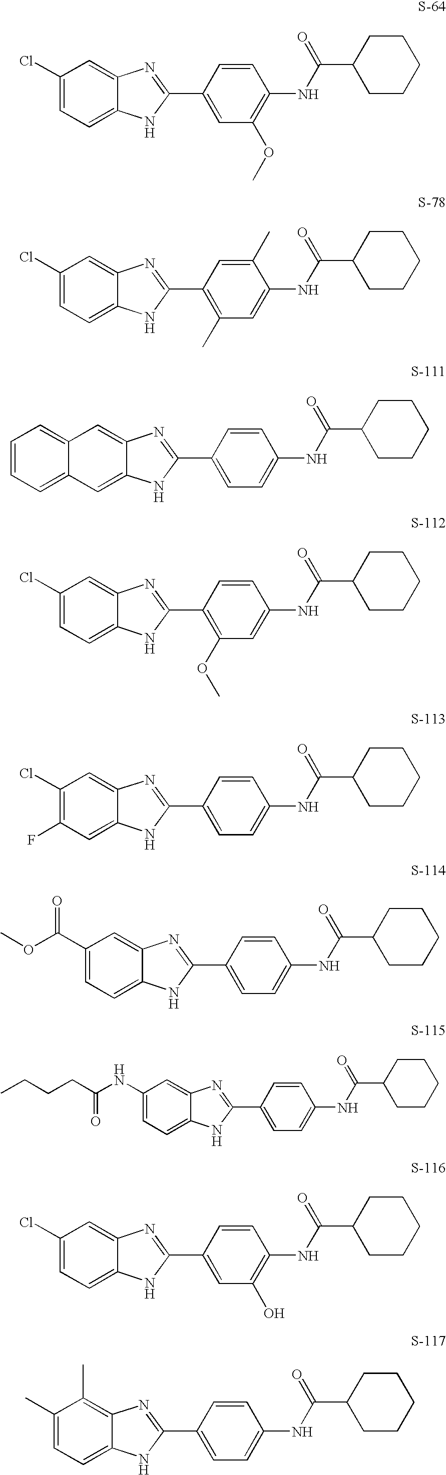

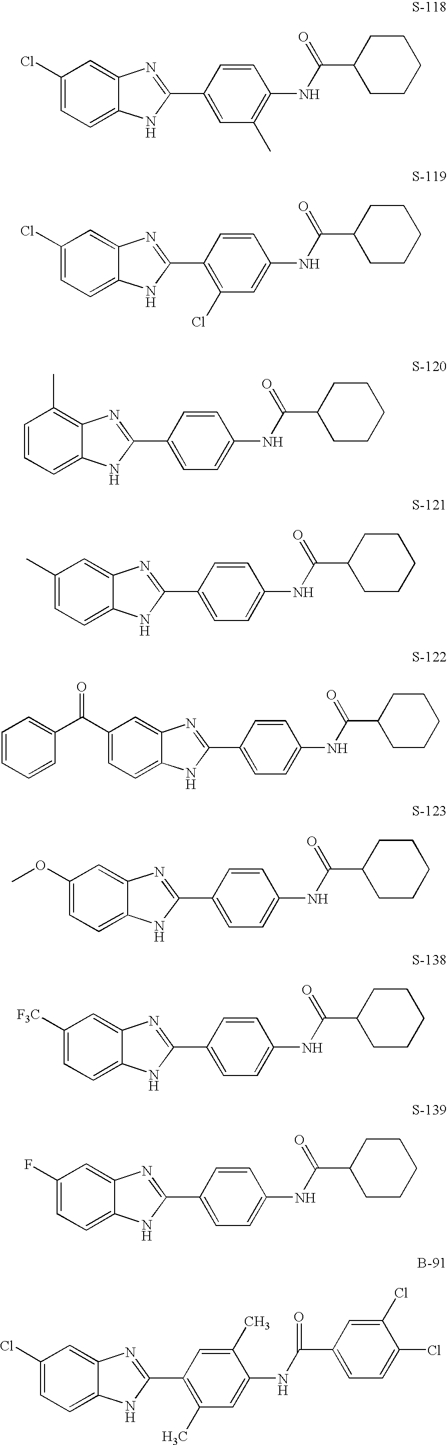

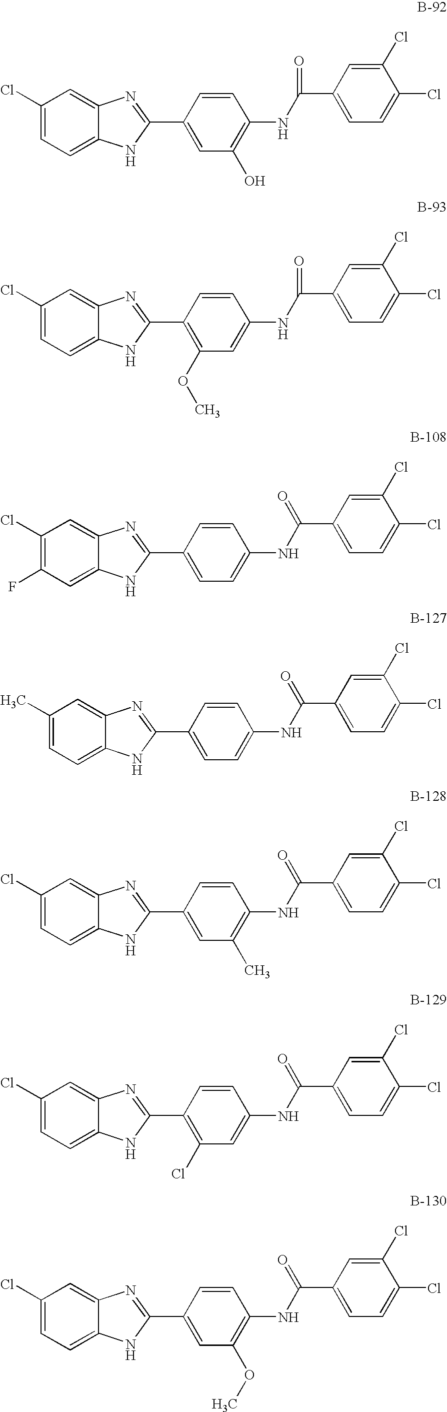

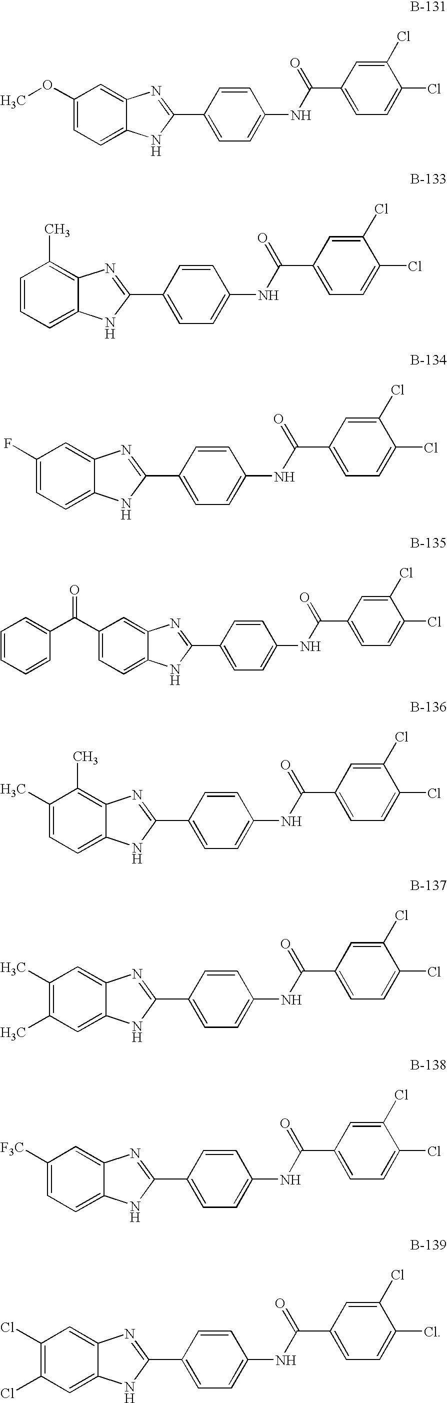

11. The method according to claims 1, 3, 5, 7, or 9, in which the method further comprises administering at least one additional ingredient which is active in reducing at least one symptom associated with said cellular proliferation.

12. The method according to claim 11, wherein said at least one additional ingredient is selected from the group consisting of antifungals, antivirals, antibiotics, anti-inflammatories, and anticancer agents.

13. The method according to claim 11, wherein said at least one additional ingredient is selected from the group consisting of alkylating agent, antimetabolite, DNA cutter, topoisomerase I poison, topoisomerase II poison, DNA binder, and spindle poison.

14. The method according to claims 1, 3, 5, 7, or 9, wherein said administering a formulation comprises providing to said mammal a dose of about 0.01 mg to about 100 mg per kg body weight per day.

15. The method according to claim 14, wherein said dose is administered in divided doses at regular periodic intervals.

16. The method according to claim 15, wherein said regular periodic intervals occur daily.

17-30. (canceled)

31. The method of claim 1, wherein the compound is

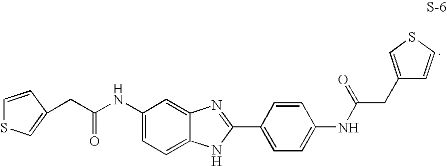

32. The method of claim 1, wherein the compound is

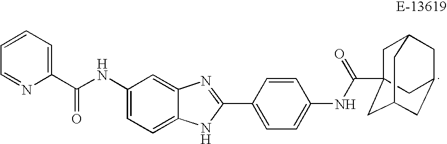

33. The method of claim 1, wherein the compound is selected from the group consisting of

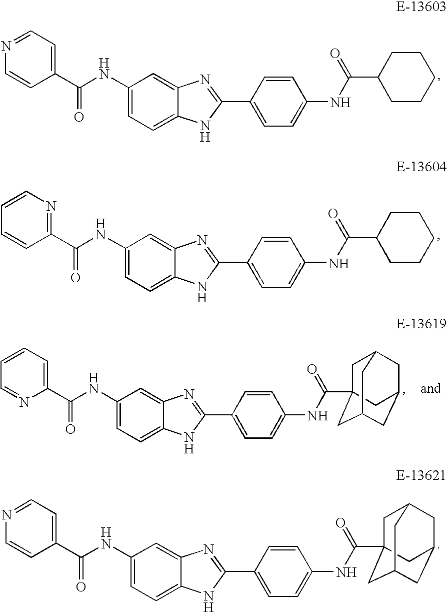

34. A composition comprising a compound selected from the group consisting

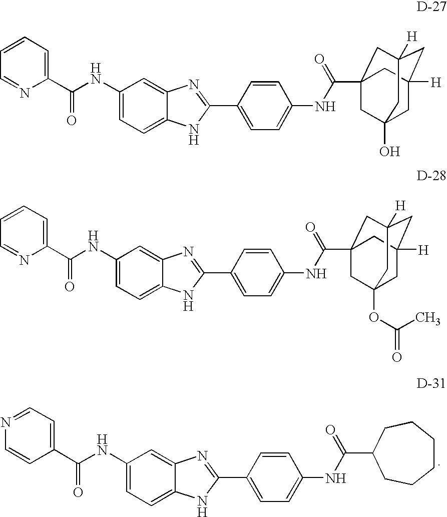

35. A composition comprising a compound selected from the group consisting

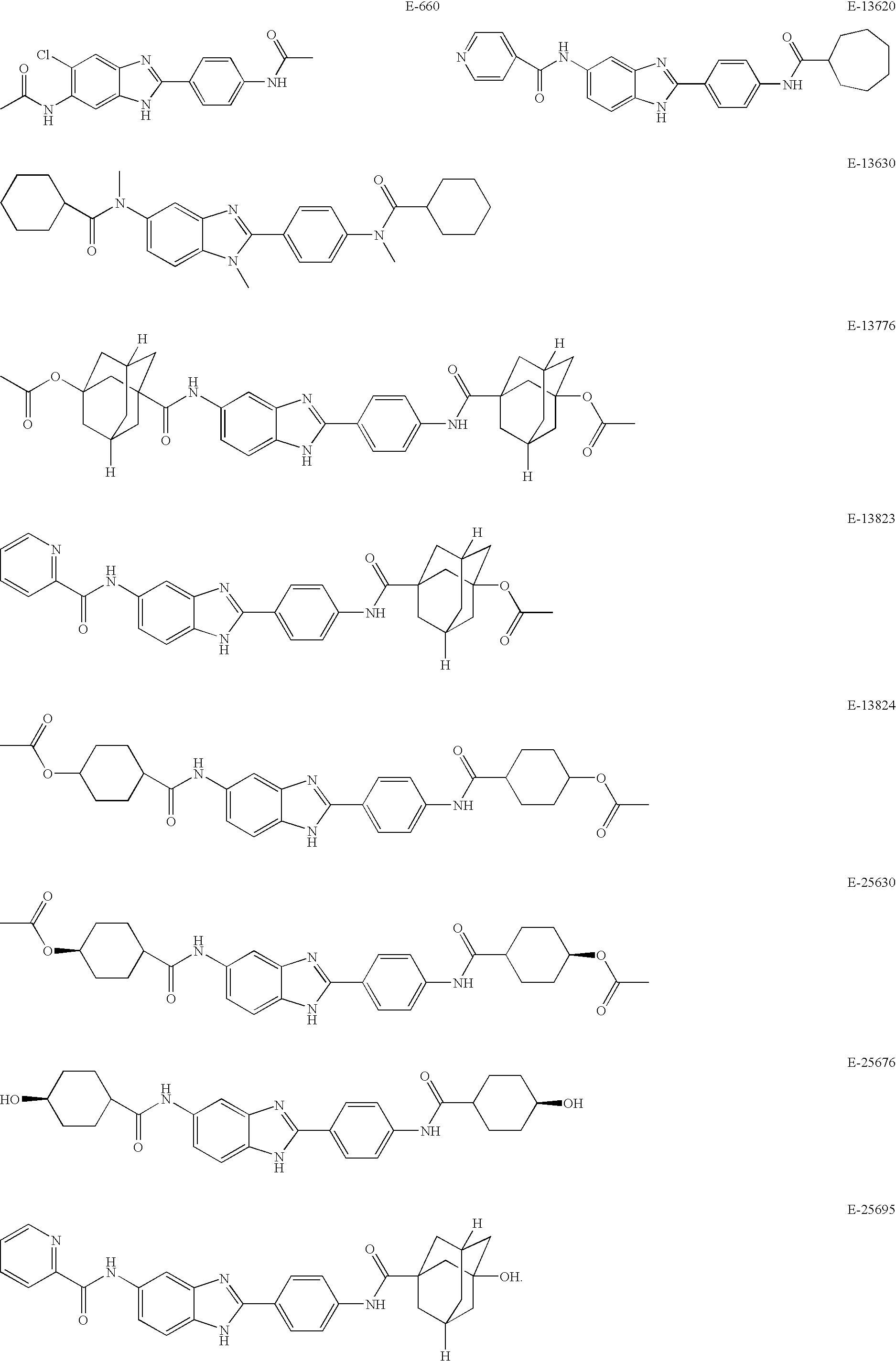

36. A composition comprising a compound selected from the group consisting of

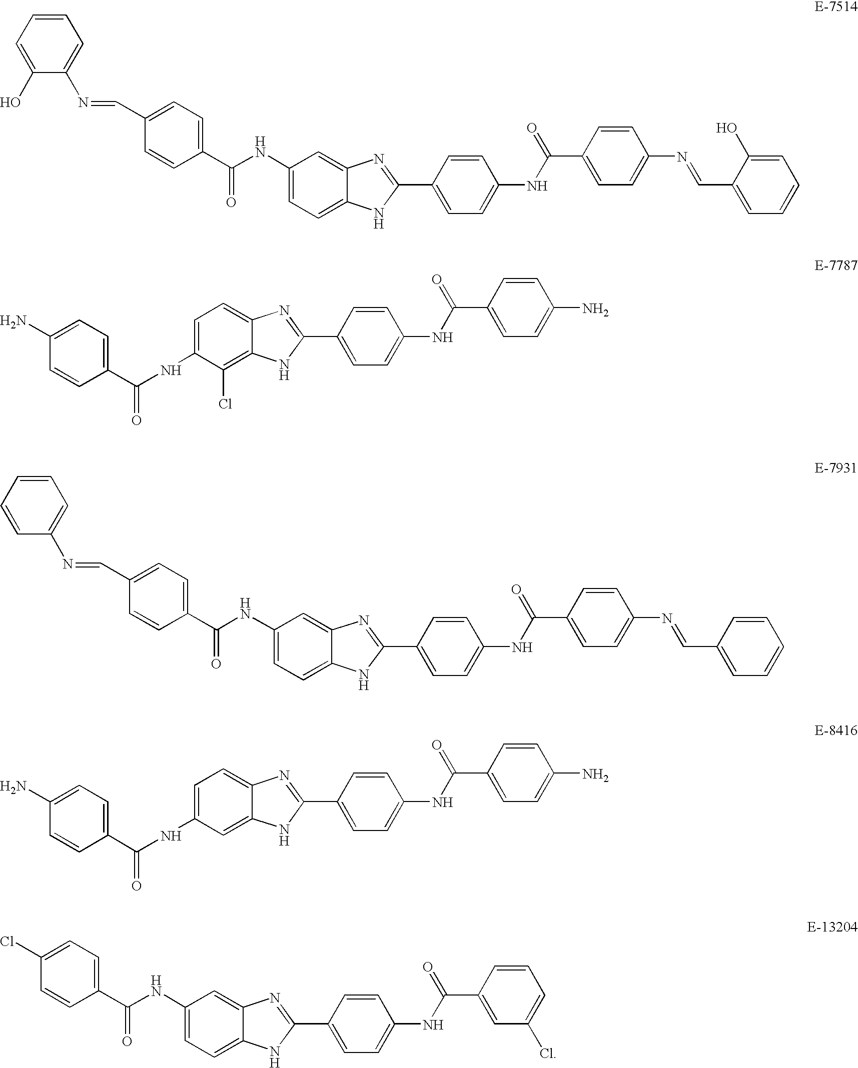

37. A composition comprising at least one compound of a pharmaceutical composition of the formulae: 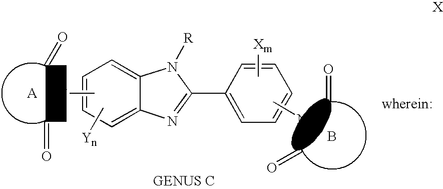
X and Y may be different or the same and are independently selected from the group consisting of H, halogen, alkyl, alkoxy, aryl, substituted aryl, hydroxy, amino, alkylamino, cycloalkyl, morpholine, thiomorpholine, nitro, cyano, CF₃, OCF₃, COR₁, COOR₁, CONH₂, CONHR₁, and NHCOR₁;
n is an integer from one to four;
m is an integer from one to four;
R is selected from the group consisting of H, CH₃, C₂H₅, C₃H₇, C₄H₉, CH₂Ph, CH₂C₆H₄—F(p-), COCH₃, COCH₂CH₃, CH₂CH₂N(CH₃)₂, and CH₂CH₂CH₂N(CH₃)₂; and
A and B rings independently comprise unsubstituted or substituted carbon atoms ranging from four carbon atoms to ten carbon atoms.

38. The composition of claim 37, wherein the composition comprises a compound selected from the group consisting of [chemical structure]
